(19) United States
(12) Patent Application Publication
(10) Pub. No.: US 2021/0187018 A1
(43) Pub. Date: Jun. 24, 2021

(54) CYTOBIOLOGICS AND THERAPEUTIC USES THEREOF

(71) Applicant: FLAGSHIP PIONEERING INNOVATIONS V, INC., Cambridge, MA (US)

(72) Inventors: Geoffrey A. von Maltzahn, Somerville, MA (US); John Miles Milwid, Denver, CO (US); Jacob Rosenblum Rubens, Cambridge, MA (US); Michael Travis Mee, Montreal (CA); Nathan Wilson Stebbins, Cambridge, MA (US); Molly Krisann Gibson, Medford, MA (US); Neal Francis Gordon, Brookline, MA (US); Bo Zhang, Lynnfield, MA (US); Kyle Marvin Trudeau, Boston, MA (US); Brigham Jay Hartley, Long Island City, NY (US); Tamar Rose Putiri, Milton, MA (US); Kiana Mahdaviani, Chestnut Hill, MA (US); Jagesh Vijaykumar Shah, Lexington, MA (US); Michael Connor, Cambridge, MA (US); Peter Anthony Jones, Medford, MA (US)

(73) Assignee: FLAGSHIP PIONEERING INNOVATIONS V, INC., Cambridge, MA (US)

(21) Appl. No.: 16/769,930

(22) PCT Filed: Dec. 7, 2018
(86) PCT No.: PCT/US2018/064571

§ 371 Date: Jun. 4, 2020

Related U.S. Application Data
(60) Provisional application No. 62/595,841, filed on Dec. 7, 2017.

(51) Int. Cl. A61K 35/12; A61K 38/45; A61K 9/127; A61K 39/385; C12N 15/113; A61K 39/00

(52) U.S. Cl. CPC A61K 35/12; A61K 38/45; A61K 9/127; A61K 39/385; C12N 2310/14; A61K 39/0011; A61K 2039/5156; C12N 2310/141; C12N 15/113

(57) ABSTRACT

This disclosure provides, e.g., cytobiologic compositions and methods of use thereof. The cytobiologics can be used, e.g., to deliver a protein or nucleic acid to a target cell.

### RELATED APPLICATIONS

This application claims priority to U.S. Ser. No. 62/595,841 filed Dec. 7, 2017, which is incorporated herein by reference in its entirety.

### BACKGROUND

An enucleated cell retains numerous biological properties but loses its ability to divide.

### SUMMARY OF THE INVENTION

The present disclosure provides, in some aspects, cytobiologics, e.g., enucleated cells or cells having an inactivated nucleus. The cytobiologic can be used, e.g., for delivery of a cargo in the lumen or lipid bilayer of the cytobiologic to a target cell. Cargo includes, e.g., therapeutic proteins, nucleic acids, and small molecules.

The present disclosure provides, in some aspects, a purified cytobiologic composition comprising a cytobiologic from a source cell, e.g., a mammalian source cell, e.g., a human source cell, wherein the cytobiologic has partial or complete nuclear inactivation (e.g., nuclear removal), and wherein one or more of:
i) the cytobiologic comprises an exogenous agent, e.g., a therapeutic agent, e.g., at a copy number of at least 1,000 copies, e.g., as measured by an assay of Example 31;
ii) the cytobiologic comprises a lipid wherein one or more of CL, Cer, DAG, HexCer, LPA, LPC, LPE, LPG, LPI, LPS, PA, PC, PE, PG, PI, PS, CE, SM and TAG is within 75% of the corresponding lipid level in the source cell;
iii) the cytobiologic comprises a proteomic composition similar to that of the source cell, e.g., using an assay of Example 30;
iv) the cytobiologic is capable of signal transduction, e.g., transmitting an extracellular signal, e.g., AKT phosphorylation in response to insulin, or glucose (e.g., labeled glucose, e.g., 2-NBDG) uptake in response to insulin, e.g., by at least 10% more than a negative control, e.g., an otherwise similar cytobiologic in the absence of insulin, e.g., using an assay of Example 48;
v) the cytobiologic targets a tissue, e.g., liver, lungs, heart, spleen, pancreas, gastrointestinal tract, kidney, testes, ovaries, brain, reproductive organs, central nervous system, peripheral nervous system, skeletal muscle, endothelium, inner ear, or eye, when administered to a subject, e.g., a mouse, e.g., wherein at least 0.1%, or 10%, of the cytobiologics in a population of administered cytobiologics are present in the target tissue after 24 hours, e.g., by an assay of Example 71; or
vi) the source cell is selected from a neutrophil, a granulocyte, a mesenchymal stem cell, a bone marrow stem cell, an induced pluripotent stem cell, an embryonic stem cell, a myeloblast, a myoblast, a hepatocyte, or a neuron e.g., retinal neuronal cell.

The present disclosure also provides, in some aspects, a purified cytobiologic composition comprising a cytobiologic, wherein:
i) the cytobiologic is from a source cell, e.g., a mammalian source cell, and
ii) the cytobiologic is an enucleated cell or a cell having partial or complete nuclear inactivation (e.g., nuclear removal).

The present disclosure also provides, in some aspects, a purified cytobiologic composition comprising a cytobiologic and an exogenous agent, e.g., a therapeutic agent, wherein:
iii) the cytobiologic is from a source cell, e.g., a mammalian source cell, and
iv) the cytobiologic is an enucleated cell or a cell having partial or complete nuclear inactivation (e.g., nuclear removal).

The present disclosure also provides, in some aspects, a purified cytobiologic composition, e.g., a frozen cytobiologic composition, comprising a cytobiologic, wherein:
i) the cytobiologic is from a source cell, e.g., a mammalian source cell, and
ii) the cytobiologic is an enucleated cell or a cell having partial or complete nuclear inactivation (e.g., nuclear removal), which is at a temperature of less than 4, 0, −4, −10, −12, −16, −20, −80, or −160 C.

In some embodiments, the cytobiologic is not from an erythroid cell or a platelet.

In some embodiments, one or more of the following is present:
i) the cytobiologic is not from an erythroid cell or a platelet;
ii) the cytobiologic comprises an enucleated cell;
iii) the cytobiologic comprises an inactivated nucleus;
iv) the cytobiologic comprises an exogenous agent or a therapeutic agent (e.g., an exogenous therapeutic agent) at a copy number of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies, e.g., as measured by an assay of Example 31;
v) the cytobiologic comprises a lipid composition substantially similar to that of the source cell or wherein one or more of CL, Cer, DAG, HexCer, LPA, LPC, LPE, LPG, LPI, LPS, PA, PC, PE, PG, PI, PS, CE, SM and TAG is within 10%, 15%, 20%, 25%, 30%, 35%, 40%, 45%, 50%, 55%, 60%, 65%, 70%, or 75% of the corresponding lipid level in the source cell;
vi) the cytobiologic comprises a proteomic composition similar to that of the source cell, e.g., using an assay of Example 30;
vii) the cytobiologic comprises a ratio of lipids to proteins that is within 10%, 20%, 30%, 40%, or 50% of the corresponding ratio in the source cell, e.g., as measured using an assay of Example 37;
viii) the cytobiologic comprises a ratio of proteins to nucleic acids (e.g., DNA) that is within 10%, 20%, 30%, 40%, or 50% of the corresponding ratio in the source cell, e.g., as measured using an assay of Example 38;
ix) the cytobiologic comprises a ratio of lipids to nucleic acids (e.g., DNA) that is within 10%, 20%, 30%, 40%, or 50% of the corresponding ratio in the source cell, e.g., as measured using an assay of Example 39;
x) the cytobiologic has a half-life in a subject, e.g., in a mouse, that is within 1%, 2%, 3%, 4%, 5%, 10%, 20%, 30%, 40%, 50%, 60%, 70%, 80%, 90%, 100% of the half life of a reference cell, e.g., the source cell, e.g., by an assay of Example 60;
xi) the cytobiologic transports glucose (e.g., labeled glucose, e.g., 2-NBDG) across a membrane, e.g., by at least 1%, 2%, 3%, 4%, 5%, 10%, 20%, 30%, 40%, 50%, 60%, 70%, 80%, 90%, 100% more than a negative control, e.g., an otherwise similar cytobiologic in the absence of glucose, e.g., as measured using an assay of Example 49;
xii) the cytobiologic comprises esterase activity in the lumen that is within 1%, 2%, 3%, 4%, 5%, 10%, 20%, 30%, 40%, 50%, 60%, 70%, 80%, 90%, or 100% of that of the esterase activity in a reference cell, e.g., the source cell or a mouse embryonic fibroblast, e.g., using an assay of Example 51;
xiii) the cytobiologic comprises a metabolic activity level that is within 1%, 2%, 3%, 4%, 5%, 10%, 20%, 30%, 40%, 50%, 60%, 70%, 80%, 90%, or 100% of the citrate synthase activity in a reference cell, e.g., the source cell, e.g., as described in Example 53;
xiv) the cytobiologic comprises a respiration level (e.g., oxygen consumption rate) that is within 1%, 2%, 3%, 4%, 5%, 10%, 20%, 30%, 40%, 50%, 60%, 70%, 80%, 90%, or 100% of the respiration level in a reference cell, e.g., the source cell, e.g., as described in Example 54;
xv) the cytobiologic comprises an Annexin-V staining level of at most 18,000, 17,000, 16,000, 15,000, 14,000, 13,000, 12,000, 11,000, or 10,000 MFI, e.g., using an assay of Example 55, or wherein the cytobiologic comprises an Annexin-V staining level at least 5%, 10%, 20%, 30%, 40%, or 50% lower than the Annexin-V staining level of an otherwise similar cytobiologic treated with menadione in the assay of Example 55, or wherein the cytobiologic comprises an Annexin-V staining level at least 5%, 10%, 20%, 30%, 40%, or 50% lower than the Annexin-V staining level of a macrophage treated with menadione in the assay of Example 55,
xvi) the cytobiologic has a miRNA content level of at least at least 1%, 2%, 3%, 4%, 5%, 10%, 20%, 30%, 40%, 50%, 60%, 70%, 80%, 90%, or greater than that of the source cell, e.g., by an assay of Example 27;
xvii) the cytobiologic has a soluble:non-soluble protein ratio is within 1%, 2%, 3%, 4%, 5%, 10%, 20%, 30%, 40%, 50%, 60%, 70%, 80%, 90%, or greater than that of the source cell, e.g., within 1%-2%, 2%-3%, 3%-4%, 4%-5%, 5%-10%, 10%-20%, 20%-30%, 30%-40%, 40%-50%, 50%-60%, 60%-70%, 70%-80%, or 80%-90% of that of the source cell, e.g., by an assay of Example 35;
xviii) the cytobiologic has an LPS level less than 5%, 1%, 0.5%, 0.01%, 0.005%, 0.0001%, 0.00001% or less of the lipid content of cytobiologic, e.g., as measured by an assay of Example 36; xix) the cytobiologic has an LPS level less than 5%, 1%, 0.5%, 0.01%, 0.005%, 0.0001%, 0.00001% or less of the LPS content of the source cell, e.g., as measured by mass spectrometry, e.g., in an assay of Example 36;
xx) the cytobiologic is capable of signal transduction, e.g., transmitting an extracellular signal, e.g., AKT phosphorylation in response to insulin, or glucose (e.g., labeled glucose, e.g., 2-NBDG) uptake in response to insulin, e.g., by at least 1%, 2%, 3%, 4%, 5%, 10%, 20%, 30%, 40%, 50%, 60%, 70%, 80%, 90%, 100% more than a negative control, e.g., an otherwise similar cytobiologic in the absence of insulin, e.g., using an assay of Example 48;
xxi) the cytobiologic targets a tissue, e.g., liver, lungs, heart, spleen, pancreas, gastrointestinal tract, kidney, testes, ovaries, brain, reproductive organs, central nervous system, peripheral nervous system, skeletal muscle, endothelium, inner ear, or eye, when administered to a subject, e.g., a mouse, e.g., wherein at least 0.1%, 0.5%, 1%, 1.5%, 2%, 2.5%, 3%, 4%, 5%, 10%, 20%, 30%, 40%, 50%, 60%, 70%, 80%, or 90% of the cytobiologics in a population of administered cytobiologics are present in the target tissue after 24, 48, or 72 hours, e.g., by an assay of Example 71;
xxii) the cytobiologic has juxtacrine-signaling level of at least 1%, 2%, 3%, 4%, 5%, 10%, 20%, 30%, 40%, 50%, 60%, 70%, 80%, 90%, or 100% greater than the level of juxtacrine signaling induced by a reference cell, e.g., the source cell or a bone marrow stromal cell (BMSC), e.g., by an assay of Example 56;
xxiii) the cytobiologic has paracrine-signaling level of at least 1%, 2%, 3%, 4%, 5%, 10%, 20%, 30%, 40%, 50%, 60%, 70%, 80%, 90%, 100% greater than the level of paracrine signaling induced by a reference cell, e.g., the source cell or a macrophage, e.g., by an assay of Example 57; xxiv) the cytobiologic polymerizes actin at a level within 1%, 2%, 3%, 4%, 5%, 10%, 20%, 30%, 40%, 50%, 60%, 70%, 80%, 90%, or 100% compared to the level of polymerized actin in a reference cell, e.g., the source cell or a C2C12 cell, e.g., by the assay of Example 58;
xxv) the cytobiologic has a membrane potential within about 1%, 2%, 3%, 4%, 5%, 10%, 20%, 30%, 40%, 50%, 60%, 70%, 80%, 90%, 100% of the membrane potential of a reference cell, e.g., the source cell or a C2C12 cell, e.g., by an assay of Example 59, or wherein the cytobiologic has a membrane potential of about −20 to −150 mV, −20 to −50 mV, −50 to −100 mV, or −100 to −150 mV;
xxvi) the cytobiologic is capable of extravasation from blood vessels, e.g., at a rate at least 1%, 2%, 5%, 10%, 20%, 30%, 40%, 50%, 60%, 70%, 80%, or 90% the rate of extravasation of the source cell, e.g., using an assay of Example 42, e.g., wherein the source cell is a neutrophil, lymphocyte, B cell, macrophage, or NK cell;
xxvii) the cytobiologic is capable of crossing a cell membrane, e.g., an endothelial cell membrane or the blood brain barrier;
xxviii) the cytobiologic is capable of secreting a protein, e.g., at a rate at least 1%, 2%, 3%, 4%, 5%, 10%, 20%, 30%, 40%, 50%, 60%, 70%, 80%, 90%, or 100% greater than a reference cell, e.g., a mouse embryonic fibroblast, e.g., using an assay of Example 47;
xxix) the cytobiologic meets a pharmaceutical or good manufacturing practices (GMP) standard; xxx) the cytobiologic was made according to good manufacturing practices (GMP);
xxxi) the cytobiologic has a pathogen level below a predetermined reference value, e.g., is substantially free of pathogens;
xxxii) the cytobiologic has a contaminant level below a predetermined reference value, e.g., is substantially free of contaminants;
xxxiii) the cytobiologic has low immunogenicity, e.g., as described herein;
xxxiv) the source cell is selected from a neutrophil, a granulocyte, a mesenchymal stem cell, a bone marrow stem cell, an induced pluripotent stem cell, an embryonic stem cell, a myeloblast, a myoblast, a hepatocyte, or a neuron e.g., retinal neuronal cell; or
xxxv) the source cell is other than a 293 cell, HEK cell, human endothelial cell, or a human epithelial cell, monocyte, macrophage, dendritic cell, or stem cell.

In some embodiments, one or more of the following is present:
i) the source cell is selected from an endothelial cell, a macrophage, a neutrophil, a granulocyte, a leukocyte, a stem cell (e.g., a mesenchymal stem cell, a bone marrow stem cell, an induced pluripotent stem cell, an embryonic stem cell), a myeloblast, a myoblast, a hepatocyte, or a neuron e.g., retinal neuronal cell;
ii) the organelle is selected from a mitochondrion, a Golgi apparatus, lysosome, endoplasmic reticulum, vacuole, endosome, acrosome, autophagosome, centriole, glycosome, glyoxysome, hydrogenosome, melanosome, mitosome, cnidocyst, peroxisome, proteasome, vesicle, and stress granule;
iii) the cytobiologic has a size of greater than 5 um, 10 um, 20 um, 50 um, or 100 um; i) the cytobiologic, composition, or preparation has a density of other than between 1.08 g/ml and 1.12 g/ml, e.g., the cytobiologic, composition, or preparation has a density of 1.25 g/ml+/−0.05, e.g., as measured by an assay of Example 21;
iv) the cytobiologic is not captured by the scavenger system in circulation or by Kupffer cells in the sinus of the liver;
v) the source cell is other than a 293 cell;
vi) the source cell is not transformed or immortalized;
vii) the source cell is transformed, or immortalized using a method other than adenovirus-mediated immortalization, e.g., immortalized by spontaneous mutation, or telomerase expression;
viii) the cytobiologic does not comprise Cre or GFP, e.g., EGFP;
ix) the cytobiologic further comprises an exogenous protein other than Cre or GFP, e.g., EGFP
x) the cytobiologic further comprises an exogenous nucleic acid (e.g., RNA, e.g., mRNA, miRNA, or siRNA) or an exogenous protein (e.g., an antibody, e.g., an antibody), e.g., in the lumen; or
xi) the cytobiologic does not comprise mitochondria.

The present disclosure also provides, in some aspects, a cytobiologic composition, comprising a plurality of cytobiologics described herein.

The present disclosure also provides, in some aspects, a pharmaceutical composition comprising the cytobiologic composition described herein and a pharmaceutically acceptable carrier.

The present disclosure also provides, in some aspects, a pharmaceutical composition suitable for administration to a human subject, comprising a cytobiologic and a pharmaceutically acceptable carrier, wherein:
i) the cytobiologic is from a source cell, e.g., a mammalian source cell, and
ii) the cytobiologic is an enucleated cell or a cell having partial or complete nuclear inactivation (e.g., nuclear removal).

This disclosure also provides, in certain aspects, a method of administering a cytobiologic composition to a human subject, a target tissue, or a cell, comprising administering to the human subject, or contacting the target tissue or the cell with, a cytobiologic composition comprising a plurality of cytobiologics described herein, a cytobiologic composition described herein, or a pharmaceutical composition described herein, thereby administering the cytobiologic composition to the subject. The disclosure also provides, in certain aspects, a method of administering a cytobiologic composition to a subject, e.g., a human subject, comprising administering to the subject a cytobiologic composition wherein: (i) the cytobiologic is from a source cell, e.g., a mammalian source cell, (ii) the cytobiologic is an enucleated cell or a cell having partial or complete nuclear inactivation (e.g., nuclear removal), and (iii) the cytobiologic is not from an erythroid cell or a platelet, thereby administering the cytobiologic composition to the subject.

This disclosure also provides, in certain aspects, a method of delivering a therapeutic agent (e.g., a polypeptide, a nucleic acid, a metabolite, an organelle, or a subcellular structure) to a subject, a target tissue, or a cell, comprising administering to the subject, or contacting the target tissue or the cell with, a cytobiologic composition comprising a plurality of cytobiologics described herein, a cytobiologic composition described herein, or a pharmaceutical composition described herein, wherein the cytobiologic composition is administered in an amount and/or time such that the therapeutic agent is delivered. The disclosure also provides, in certain aspects, a method of delivering a therapeutic agent to a subject, comprising administering to the subject a cytobiologic composition wherein: (i) the cytobiologic is from a source cell, e.g., a mammalian source cell, (ii) the cytobiologic is an enucleated cell or a cell having partial or complete nuclear inactivation (e.g., nuclear removal), (iii) the cytobiologic is not from an erythroid cell or a platelet, and (iv) the cytobiologic comprises the therapeutic agent, thereby delivering the therapeutic agent to the subject.

This disclosure also provides, in certain aspects, a method of modulating, e.g., enhancing, a biological function in a subject, a target tissue, or a cell, comprising administering to the subject, or contacting the target tissue or the cell with, a cytobiologic composition comprising a plurality of cytobiologics described herein, a cytobiologic composition described herein, or a pharmaceutical composition described herein, thereby modulating the biological function in the subject. The disclosure also provides, in certain aspects, a method of modulating, e.g., enhancing, a biological function in a subject, comprising administering to the subject a cytobiologic composition wherein: (i) the cytobiologic is from a source cell, e.g., a mammalian source cell, (ii) the cytobiologic is an enucleated cell or a cell having partial or complete nuclear inactivation (e.g., nuclear removal), and (iii) the cytobiologic is not from an erythroid cell or a platelet, thereby modulating the biological function in the subject.

This disclosure also provides, in certain aspects, a method of delivering or targeting a function to a subject, comprising administering to the subject a cytobiologic composition comprising a plurality of cytobiologics described herein which comprise the function, a cytobiologic composition described herein, or a pharmaceutical composition described herein, wherein the cytobiologic composition is administered in an amount and/or time such that the function in the subject is delivered or targeted. In embodiments, the subject has a cancer, an inflammatory disorder, autoimmune disease, a chronic disease, inflammation, damaged organ function, an infectious disease, a degenerative disorder, a genetic disease, or an injury.

The disclosure also provides, in certain aspects, a method of delivering or targeting a function to a subject, comprising administering to the subject a cytobiologic composition wherein: (i) the cytobiologic is from a source cell, e.g., a mammalian source cell, (ii) the cytobiologic is an enucleated cell or a cell having partial or complete nuclear inactivation (e.g., nuclear removal), and (iii) the cytobiologic is not from an erythroid cell or a platelet, thereby delivering or targeting the function to the subject.

The disclosure also provides, in some aspects, a method of manufacturing a cytobiologic composition, comprising:

a) providing a source cell, e.g., mammalian source cell;

b) producing a cytobiologic from the source cell; and

c) formulating the cytobiologic, e.g., as a pharmaceutical composition suitable for administration to a subject.

In some aspects, the present disclosure provides a method of manufacturing a cytobiologic composition, comprising:

a) providing a plurality of source cells, e.g., mammalian source cells;

b) producing at least 10⁵, 10⁶, 10⁷, 10⁸, 10⁹, 10¹⁰, 10¹¹, 10¹², 10¹³, 10¹⁴, or 10¹⁵ cytobiologics from the plurality of source cells, e.g., by enucleation.

In some aspects, the present disclosure provides a method of manufacturing a pharmaceutical cytobiologic composition, comprising:

a) providing a cytobiologic composition according to any of claims 1-82 or a pharmaceutical composition of claim 83 or 84; and

b) formulating the cytobiologic composition, e.g., as a pharmaceutical composition suitable for administration to a subject.

In some aspects, the present disclosure provides a method of manufacturing a cytobiologic composition, comprising:

a) providing, e.g., producing, a plurality of cytobiologics described herein or a cytobiologic composition described herein; and

b) assaying one or more cytobiologics from the cytobiologic composition or plurality to determine whether one or more (e.g., 2, 3, or more) standards are met. In embodiments, the standard(s) are chosen from:
i) the cytobiologic comprises a therapeutic agent at a copy number of at least, or no more than, 10, 50, 100, 500, 1,000, 2,000, 5,000, 10,000, 20,000, 50,000, 100,000, 200,000, 500,000 1,000,000, 5,000,000, 10,000,000, 50,000,000, 100,000,000, 500,000,000, or 1,000,000,000 copies, e.g., as measured by an assay of Example 31;
ii) the cytobiologic comprises a lipid composition substantially similar to that of the source cell or wherein one or more of CL, Cer, DAG, HexCer, LPA, LPC, LPE, LPG, LPI, LPS, PA, PC, PE, PG, PI, PS, CE, SM and TAG is within 10%, 15%, 20%, 25%, 30%, 35%, 40%, 45%, 50%, or 75% of the corresponding lipid level in the source cell;
iii) the cytobiologic comprises a proteomic composition similar to that of the source cell, e.g., using an assay of Example 30;
iv) the cytobiologic comprises a ratio of lipids to proteins that is within 10%, 20%, 30%, 40%, or 50% of the corresponding ratio in the source cell, e.g., as measured using an assay of Example 37;
v) the cytobiologic comprises a ratio of proteins to nucleic acids (e.g., DNA) that is within 10%, 20%, 30%, 40%, or 50% of the corresponding ratio in the source cell, e.g., as measured using an assay of Example 38;
vi) the cytobiologic comprises a ratio of lipids to nucleic acids (e.g., DNA) that is within 10%, 20%, 30%, 40%, or 50% of the corresponding ratio in the source cell, e.g., as measured using an assay of Example 39;
vii) the cytobiologic has a half-life in a subject, e.g., in a mouse, that is within 1%, 2%, 3%, 4%, 5%, 10%, 20%, 30%, 40%, 50%, 60%, 70%, 80%, 90%, 100% of the half life of a reference cell, e.g., the source cell, e.g., by an assay of Example 60;
viii) the cytobiologic transports glucose (e.g., labeled glucose, e.g., 2-NBDG) across a membrane, e.g., by at least 1%, 2%, 3%, 4%, 5%, 10%, 20%, 30%, 40%, 50%, 60%, 70%, 80%, 90%, 100% more than a negative control, e.g., an otherwise similar cytobiologic in the absence of glucose, e.g., as measured using an assay of Example 49;
ix) the cytobiologic comprises esterase activity in the lumen that is within 1%, 2%, 3%, 4%, 5%, 10%, 20%, 30%, 40%, 50%, 60%, 70%, 80%, 90%, or 100% of that of the esterase activity in a reference cell, e.g., the source cell or a mouse embryonic fibroblast, e.g., using an assay of Example 51;
x) the cytobiologic comprises a metabolic activity level that is within 1%, 2%, 3%, 4%, 5%, 10%, 20%, 30%, 40%, 50%, 60%, 70%, 80%, 90%, or 100% of the citrate synthase activity in a reference cell, e.g., the source cell, e.g., as described in Example 53; xi) the cytobiologic comprises a respiration level (e.g., oxygen consumption rate) that is within 1%, 2%, 3%, 4%, 5%, 10%, 20%, 30%, 40%, 50%, 60%, 70%, 80%, 90%, or 100% of the respiration level in a reference cell, e.g., the source cell, e.g., as described in Example 54;
xii) the cytobiologic comprises an Annexin-V staining level of at most 18,000, 17,000, 16,000, 15,000, 14,000, 13,000, 12,000, 11,000, or 10,000 MFI, e.g., using an assay of Example 55, or wherein the cytobiologic comprises an Annexin-V staining level at least 5%, 10%, 20%, 30%, 40%, or 50% lower than the Annexin-V staining level of an otherwise similar cytobiologic treated with menadione in the assay of Example 55, or wherein the cytobiologic comprises an Annexin-V staining level at least 5%, 10%, 20%, 30%, 40%, or 50% lower than the Annexin-V staining level of a macrophage treated with menadione in the assay of Example 55,
xiii) the cytobiologic has a miRNA content level of at least at least 1%, 2%, 3%, 4%, 5%, 10%, 20%, 30%, 40%, 50%, 60%, 70%, 80%, 90%, or greater than that of the source cell, e.g., by an assay of Example 27;
xiv) the cytobiologic has a soluble:non-soluble protein ratio is within 1%, 2%, 3%, 4%, 5%, 10%, 20%, 30%, 40%, 50%, 60%, 70%, 80%, 90%, or greater than that of the source cell, e.g., within 1%-2%, 2%-3%, 3%-4%, 4%-5%, 5%-10%, 10%-20%, 20%-30%, 30%-40%, 40%-50%, 50%-60%, 60%-70%, 70%-80%, or 80%-90% of that of the source cell, e.g., by an assay of Example 35;
xv) the cytobiologic has an LPS level less than 5%, 1%, 0.5%, 0.01%, 0.005%, 0.0001%, 0.00001% or less of the lipid content of cytobiologics, e.g., as measured by an assay of Example 36;
xvi) the cytobiologic has an LPS level less than 5%, 1%, 0.5%, 0.01%, 0.005%, 0.0001%, 0.00001% or less of the LPS content of the source cell, e.g., as measured by mass spectrometry, e.g., in an assay of Example 36;
xvii) the cytobiologic is capable of signal transduction, e.g., transmitting an extracellular signal, e.g., AKT phosphorylation in response to insulin, or glucose (e.g., labeled glucose, e.g., 2-NBDG) uptake in response to insulin, e.g., by at least 1%, 2%, 3%, 4%, 5%, 10%, 20%, 30%, 40%, 50%, 60%, 70%, 80%, 90%, 100% more than a negative control, e.g., an otherwise similar cytobiologic in the absence of insulin, e.g., using an assay of Example 48;
xviii) the cytobiologic has juxtacrine-signaling level of at least 1%, 2%, 3%, 4%, 5%, 10%, 20%, 30%, 40%, 50%, 60%, 70%, 80%, 90%, or 100% greater than the level of juxtacrine signaling induced by a reference cell, e.g., the source cell or a bone marrow stromal cell (BMSC), e.g., by an assay of Example 56;
xix) the cytobiologic has paracrine-signaling level of at least 1%, 2%, 3%, 4%, 5%, 10%, 20%, 30%, 40%, 50%, 60%, 70%, 80%, 90%, 100% greater than the level of paracrine signaling induced by a reference cell, e.g., the source cell or a macrophage, e.g., by an assay of Example 57;
xx) the cytobiologic polymerizes actin at a level within 1%, 2%, 3%, 4%, 5%, 10%, 20%, 30%, 40%, 50%, 60%, 70%, 80%, 90%, or 100% compared to the level of polymerized actin in a reference cell, e.g., the source cell or a C2C12 cell, e.g., by the assay of Example 58;
xxi) the cytobiologic has a membrane potential within about 1%, 2%, 3%, 4%, 5%, 10%, 20%, 30%, 40%, 50%, 60%, 70%, 80%, 90%, 100% of the membrane potential of a reference cell, e.g., the source cell or a C2C12 cell, e.g., by an assay of Example 59, or wherein the cytobiologic has a membrane potential of about −20 to −150 mV, −20 to −50 mV, −50 to −100 mV, or −100 to −150 mV;
xxii) the cytobiologic is capable of secreting a protein, e.g., at a rate at least 1%, 2%, 3%, 4%, 5%, 10%, 20%, 30%, 40%, 50%, 60%, 70%, 80%, 90%, or 100% greater than a reference cell, e.g., a mouse embryonic fibroblast, e.g., using an assay of Example 47; or
xxiii) the cytobiologic has low immunogenicity, e.g., as described herein; and

c) (optionally) approving the plurality of cytobiologic or cytobiologic composition for release if one or more of the standards is met.

The present disclosure also provides, in some aspects, a method of manufacturing a cytobiologic composition, comprising:

a) providing, e.g., producing, a plurality of cytobiologics described herein or a cytobiologic composition described herein; and

b) assaying one or more cytobiologic from the plurality or the cytobiologic composition to determine the presence or level of one or more of the following factors:
i) an immunogenic molecule, e.g., an immunogenic protein, e.g., as described herein;
ii) a pathogen, e.g., a bacterium or virus; or
iii) a contaminant; and

c) (optionally) approving the plurality of cytobiologics or cytobiologic composition for release if one or more of the factors is below a reference value.

Any of the aspects herein, e.g., the cytobiologics, cytobiologic compositions, and methods above, can be combined with one or more of the embodiments herein, e.g., an embodiment below.

In some embodiments, the cytobiologic is capable of delivering (e.g., delivers) a secreted agent, e.g., a secreted protein to a target site (e.g., an extracellular region). Similarly, in some embodiments, a method herein comprises delivering a secreted agent as described herein. In embodiments, the secreted protein is endogenous or exogenous. In embodiments, the secreted protein comprises a protein therapeutic, e.g., an antibody molecule, a cytokine, or an enzyme. In embodiments, the secreted protein comprises an autocrine signalling molecule or a paracrine signalling molecule. In embodiments, the secreted agent comprises a secretory granule.

In some embodiments, a cytobiologic is capable of modifying, e.g., modifies, a target tumor cell. Similarly, in some embodiments, a method herein comprises modifying a target tumor cell. In embodiments, the cytobiologic comprises an immunostimulatory ligand, an antigen presenting protein, or a pro-apoptotic protein.

In some embodiments, a cytobiologic comprises an agent that is immunomodulatory, e.g., immunostimulatory.

In some embodiments, the cytobiologic is capable of secreting (e.g., secretes) an agent, e.g., a protein. In some embodiments, the agent, e.g., secreted agent, is delivered to a target site in a subject. In some embodiments, the agent is a protein that can not be made recombinantly or is difficult to make recombinantly. In some embodiments, the cytobiologic that secretes a protein is from a source cell selected from an MSC or a chondrocyte.

In some embodiments, the cytobiologic comprises on its membrane one or more cell surface ligands (e.g., 1, 2, 3, 4, 5, 10, 20, 50, or more cell surface ligands). Similarly, in some embodiments, a method herein comprises presenting one or more cell surface ligands to a target cell. In some embodiments, the cytobiologic having a cell surface ligand is from a source cell chosen from a neutrophil (e.g., and the target cell is a tumor-infiltrating lymphocyte), dendritic cell (e.g., and the target cell is a naïve T cell), or neutrophil (e.g., and the target is a tumor cell or virus-infected cell). In some embodiments the cytobiologic comprises a membrane complex, e.g., a complex comprising at least 2, 3, 4, or 5 proteins, e.g., a homodimer, heterodimer, homotrimer, heterotrimer, homotetramer, or heterotetramer. In some embodiments, the cytobiologic comprises an antibody, e.g., a toxic antibody, e.g., the cytobiologic is capable of delivering the antibody to the target site, e.g., by homing to a target site. In some embodiments, the source cell is an NK cell or neutrophil.

In some embodiments, the cytobiologic is capable of causing cell death of the target cell. In some embodiments, the cytobiologic is from a NK source cell.

In some embodiments, a cytobiologic or target cell is capable of phagocytosis (e.g., of a pathogen). Similarly, in some embodiments, a method herein comprises causing phagocytosis.

In some embodiments, a cytobiologic senses and responds to its local environment. In some embodiments, the cytobiologic is capable of sensing level of a metabolite, interleukin, or antigen.

In embodiments, a cytobiologic is capable of chemotaxis, extravasation, or one or more metabolic activities. In embodiments, the metabolic activity is selected from kyneurinine, gluconeogenesis, prostaglandin fatty acid oxidation, adenosine metabolism, urea cycle, and thermogenic respiration. In some embodiments, the source cell is a neutrophil and the cytobiologic is capable of homing to a site of injury. In some embodiments, the source cell is a macrophage and the cytobiologic is capable of phagocytosis. In some embodiments, the source cell is a brown adipose tissue cell and the cytobiologic is capable of lipolysis.

In some embodiments, the cytobiologic comprises a plurality of agents (e.g., at least 2, 3, 4, 5, 10, 20, or 50 agents). In some embodiments, the first agent and the second agent form a complex, wherein optionally the complex further comprises one or more additional cell surface receptors. In some embodiments, the agent comprises or encodes an antigen or an antigen presenting protein. In an embodiment, the agent comprises a protein, nucleic acid, organelle, or metabolite.

In some embodiments, the cytobiologic comprises a membrane protein or a nucleic acid encoding the membrane protein.

In some embodiments, the subject is in need of regeneration. In some embodiments, the subject suffers from cancer, an autoimmune disease, an infectious disease, a metabolic disease, a neurodegenerative disease, or a genetic disease (e.g., enzyme deficiency).

In some embodiments:
ii) the source cell is other than a 293 cell, HEK cell, human endothelial cell, or a human epithelial cell;
iii) the cytobiologic, composition, or preparation has a density of other than between 1.08 g/ml and 1.12 g/ml, e.g.,
iv) the cytobiologic, composition, or preparation has a density of 1.25 g/ml+/−0.05, e.g., as measured by an assay of Example 21;
v) the cytobiologic is not captured by the scavenger system in circulation or by Kupffer cells in the sinus of the liver;
vi) the cytobiologic has a diameter of greater than 5 um, 6 um, 7 um, 8 um, 10 um, 20 um, 50 um, 100 um, 150 um, or 200 um.

In some embodiments, the cytobiologic comprises an enucleated cell. In some embodiments, the cytobiologic comprises an inactivated nucleus. In some embodiments, the cytobiologic does not comprise a functional nucleus.

In some embodiments, the cytobiologic comprises a therapeutic agent at a copy number of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies, e.g., as measured by an assay of Example 31. In some embodiments, the cytobiologic comprises a protein therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies, e.g., as measured by an assay of Example 31. In some embodiments, the cytobiologic comprises a nucleic acid therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cytobiologic comprises a DNA therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cytobiologic comprises an RNA therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cytobiologic comprises an exogenous therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cytobiologic comprises an exogenous protein therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, the cytobiologic comprises an exogenous nucleic acid (e.g., DNA or RNA) therapeutic agent at a copy number of at least 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies.

In some embodiments, the cytobiologic comprises a lipid composition substantially similar to that of the source cell or wherein one or more of CL, Cer, DAG, HexCer, LPA, LPC, LPE, LPG, LPI, LPS, PA, PC, PE, PG, PI, PS, CE, SM and TAG is within 10%, 15%, 20%, 25%, 30%, 35%, 40%, 45%, or 50% of the corresponding lipid level in the source cell.

In some embodiments, the cytobiologic has a ratio of cardiolipin:ceramide that is within 10%, 20%, 30%, 40%, or 50% of the ratio of cardiolipin:ceramide in the source cell; or has a ratio of cardiolipin:diacylglycerol that is within 10%, 20%, 30%, 40%, or 50% of the ratio of cardiolipin:diacylglycerol in the source cell; or has a ratio of cardiolipin:hexosylceramide that is within 10%, 20%, 30%, 40%, or 50% of the ratio of cardiolipin:hexosylceramide in the source cell; or has a ratio of cardiolipin:lysophosphatidate that is within 10%, 20%, 30%, 40%, or 50% of the ratio of cardiolipin:lysophosphatidate in the source cell; or has a ratio of cardiolipin:lyso-phosphatidylcholine that is within 10%, 20%, 30%, 40%, or 50% of the ratio of cardiolipin:lyso-phosphatidylcholine in the source cell; or has a ratio of cardiolipin:lyso-phosphatidylethanolamine that is within 10%, 20%, 30%, 40%, or 50% of the ratio of cardiolipin:lyso-phosphatidylethanolamine in the source cell; or has a ratio of cardiolipin:lyso-phosphatidylglycerol that is within 10%, 20%, 30%, 40%, or 50% of the ratio of cardiolipin:lyso-phosphatidylglycerol in the source cell; or has a ratio of cardiolipin:lyso-phosphatidylinositol that is within 10%, 20%, 30%, 40%, or 50% of the ratio of cardiolipin:lyso-phosphatidylinositol in the source cell; or has a ratio of cardiolipin:lyso-phosphatidylserine that is within 10%, 20%, 30%, 40%, or 50% of the ratio of cardiolipin:lyso-phosphatidylserine in the source cell; or has a ratio of cardiolipin:phosphatidate that is within 10%, 20%, 30%, 40%, or 50% of the ratio of cardiolipin:phosphatidate in the source cell; or has a ratio of cardiolipin:phosphatidylcholine that is within 10%, 20%, 30%, 40%, or 50% of the ratio of cardiolipin:phosphatidylcholine in the source cell; or has a ratio of cardiolipin:phosphatidylethanolamine that is within 10%, 20%, 30%, 40%, or 50% of the ratio of cardiolipin:phosphatidylethanolamine in the source cell; or has a ratio of cardiolipin:phosphatidylglycerol that is within 10%, 20%, 30%, 40%, or 50% of the ratio of cardiolipin:phosphatidylglycerol in the source cell; or has a ratio of cardiolipin:phosphatidylinositol that is within 10%, 20%, 30%, 40%, or 50% of the ratio of cardiolipin:phosphatidylinositol in the source cell; or has a ratio of cardiolipin:phosphatidylserine that is within 10%, 20%, 30%, 40%, or 50% of the ratio of cardiolipin:phosphatidylserine in the source cell; or has a ratio of cardiolipin:cholesterol ester that is within 10%, 20%, 30%, 40%, or 50% of the ratio of cardiolipin:cholesterol ester in the source cell; or has a ratio of cardiolipin:sphingomyelin that is within 10%, 20%, 30%, 40%, or 50% of the ratio of cardiolipin:sphingomyelin in the source cell; or has a ratio of cardiolipin:triacylglycerol that is within 10%, 20%, 30%, 40%, or 50% of the ratio of cardiolipin:triacylglycerol in the source cell; or has a ratio of phosphatidylcholine:ceramide that is within 10%, 20%, 30%, 40%, or 50% of the ratio of phosphatidylcholine:ceramide in the source cell; or has a ratio of phosphatidylcholine:diacylglycerol that is within 10%, 20%, 30%, 40%, or 50% of the ratio of phosphatidylcholine:diacylglycerol in the source cell; or has a ratio of phosphatidylcholine:hexosylceramide that is within 10%, 20%, 30%, 40%, or 50% of the ratio of phosphatidylcholine:hexosylceramide in the source cell; or has a ratio of phosphatidylcholine:lysophosphatidate that is within 10%, 20%, 30%, 40%, or 50% of the ratio of phosphatidylcholine:lysophosphatidate in the source cell; or has a ratio of phosphatidylcholine:lyso-phosphatidylcholine that is within 10%, 20%, 30%, 40%, or 50% of the ratio of phosphatidylcholine:lyso-phosphatidylcholine in the source cell; or has a ratio of phosphatidylcholine:lyso-phosphatidylethanolamine that is within 10%, 20%, 30%, 40%, or 50% of the ratio of phosphatidylcholine:lyso-phosphatidylethanolamine in the source cell; or has a ratio of phosphatidylcholine:lyso-phosphatidylglycerol that is within 10%, 20%, 30%, 40%, or 50% of the ratio of phosphatidylcholine:lyso-phosphatidylglycerol in the source cell; or has a ratio of phosphatidylcholine:lyso-phosphatidylinositol that is within 10%, 20%, 30%, 40%, or 50% of the ratio of phosphatidylcholine:lyso-phosphatidylinositol in the source cell; or has a ratio of phosphatidylcholine:lyso-phosphatidylserine that is within 10%, 20%, 30%, 40%, or 50% of the ratio of phosphatidylcholine:lyso-phosphatidylserine in the source cell; or has a ratio of phosphatidylcholine:phosphatidate that is within 10%, 20%, 30%, 40%, or 50% of the ratio of cardiolipin:phosphatidate in the source cell; or has a ratio of phosphatidylcholine:phosphatidylethanolamine that is within 10%, 20%, 30%, 40%, or 50% of the ratio of phosphatidylcholine:phosphatidylethanolamine in the source cell; or has a ratio of cardiolipin:phosphatidylglycerol that is within 10%, 20%, 30%, 40%, or 50% of the ratio of phosphatidylcholine:phosphatidylglycerol in the source cell; or has a ratio of phosphatidylcholine:phosphatidylinositol that is within 10%, 20%, 30%, 40%, or 50% of the ratio of phosphatidylcholine:phosphatidylinositol in the source cell; or has a ratio of phosphatidylcholine:phosphatidylserine that is within 10%, 20%, 30%, 40%, or 50% of the ratio of phosphatidylcholine:phosphatidylserine in the source cell; or has a ratio of phosphatidylcholine:cholesterol ester that is within 10%, 20%, 30%, 40%, or 50% of the ratio of phosphatidylcholine:cholesterol ester in the source cell; or has a ratio of phosphatidylcholine:sphingomyelin that is within 10%, 20%, 30%, 40%, or 50% of the ratio of phosphatidylcholine:sphingomyelin in the source cell; or has a ratio of phosphatidylcholine:triacylglycerol that is within 10%, 20%, 30%, 40%, or 50% of the ratio of phosphatidylcholine:triacylglycerol in the source cell; or has a ratio of phosphatidylethanolamine:ceramide that is within 10%, 20%, 30%, 40%, or 50% of the ratio of phosphatidylethanolamine:ceramide in the source cell; or has a ratio of phosphatidylethanolamine:diacylglycerol that is within 10%, 20%, 30%, 40%, or 50% of the ratio of phosphatidylethanolamine:diacylglycerol in the source cell; or has a ratio of phosphatidylethanolamine:hexosylceramide that is within 10%, 20%, 30%, 40%, or 50% of the ratio of phosphatidylethanolamine:hexosylceramide in the source cell; or has a ratio of phosphatidylethanolamine:lysophosphatidate that is within 10%, 20%, 30%, 40%, or 50% of the ratio of phosphatidylethanolamine:lysophosphatidate in the source cell; or has a ratio of phosphatidylethanolamine:lyso-phosphatidylcholine that is within 10%, 20%, 30%, 40%, or 50% of the ratio of phosphatidylethanolamine:lyso-phosphatidylcholine in the source cell; or has a ratio of phosphatidylethanolamine:lyso-phosphatidylethanolamine that is within 10%, 20%, 30%, 40%, or 50% of the ratio of phosphatidylethanolamine:lyso-phosphatidylethanolamine in the source cell; or has a ratio of phosphatidylethanolamine:lyso-phosphatidylglycerol that is within 10%, 20%, 30%, 40%, or 50% of the ratio of phosphatidylethanolamine:lyso-phosphatidylglycerol in the source cell; or has a ratio of phosphatidylethanolamine:lyso-phosphatidylinositol that is within 10%, 20%, 30%, 40%, or 50% of the ratio of phosphatidylethanolamine:lyso-phosphatidylinositol in the source cell; or has a ratio of phosphatidylethanolamine:lyso-phosphatidylserine that is within 10%, 20%, 30%, 40%, or 50% of the ratio of phosphatidylethanolamine:lyso-phosphatidylserine in the source cell; or has a ratio of phosphatidylethanolamine:phosphatidate that is within 10%, 20%, 30%, 40%, or 50% of the ratio of phosphatidylethanolamine:phosphatidate in the source cell; or has a ratio of phosphatidylethanolamine:phosphatidylglycerol that is within 10%, 20%, 30%, 40%, or 50% of the ratio of phosphatidylethanolamine:phosphatidylglycerol in the source cell; or has a ratio of phosphatidylethanolamine:phosphatidylinositol that is within 10%, 20%, 30%, 40%, or 50% of the ratio of phosphatidylethanolamine:phosphatidylinositol in the source cell; or has a ratio of phosphatidylethanolamine:phosphatidylserine that is within 10%, 20%, 30%, 40%, or 50% of the ratio of phosphatidylethanolamine:phosphatidylserine in the source cell; or has a ratio of phosphatidylethanolamine:cholesterol ester that is within 10%, 20%, 30%, 40%, or 50% of the ratio of phosphatidylethanolamine:cholesterol ester in the source cell; or has a ratio of phosphatidylethanolamine:sphingomyelin that is within 10%, 20%, 30%, 40%, or 50% of the ratio of phosphatidylethanolamine:sphingomyelin in the source cell; or has a ratio of phosphatidylethanolamine:triacylglycerol that is within 10%, 20%, 30%, 40%, or 50% of the ratio of phosphatidylethanolamine:triacylglycerol in the source cell; or has a ratio of phosphatidylserine:ceramide that is within 10%, 20%, 30%, 40%, or 50% of the ratio of phosphatidylserine:ceramide in the source cell; or has a ratio of phosphatidylserine:diacylglycerol that is within 10%, 20%, 30%, 40%, or 50% of the ratio of phosphatidylserine:diacylglycerol in the source cell; or has a ratio of phosphatidylserine:hexosylceramide that is within 10%, 20%, 30%, 40%, or 50% of the ratio of phosphatidylserine:hexosylceramide in the source cell; or has a ratio of phosphatidylserine:lysophosphatidate that is within 10%, 20%, 30%, 40%, or 50% of the ratio of phosphatidylserine:lysophosphatidate in the source cell; or has a ratio of phosphatidylserine:lyso-phosphatidylcholine that is within 10%, 20%, 30%, 40%, or 50% of the ratio of phosphatidylserine:lyso-phosphatidylcholine in the source cell; or has a ratio of phosphatidylserine:lyso-phosphatidylethanolamine that is within 10%, 20%, 30%, 40%, or 50% of the ratio of phosphatidylserine:lyso-phosphatidylethanolamine in the source cell; or has a ratio of phosphatidylserine:lyso-phosphatidylglycerol that is within 10%, 20%, 30%, 40%, or 50% of the ratio of phosphatidylserine:lyso-phosphatidylglycerol in the source cell; or has a ratio of phosphatidylserine:lyso-phosphatidylinositol that is within 10%, 20%, 30%, 40%, or 50% of the ratio of phosphatidylserine:lyso-phosphatidylinositol in the source cell; or has a ratio of phosphatidylserine:lyso-phosphatidylserine that is within 10%, 20%, 30%, 40%, or 50% of the ratio of phosphatidylserine:lyso-phosphatidylserine in the source cell; or has a ratio of phosphatidylserine:phosphatidate that is within 10%, 20%, 30%, 40%, or 50% of the ratio of phosphatidylserine:phosphatidate in the source cell; or has a ratio of phosphatidylserine:phosphatidylglycerol that is within 10%, 20%, 30%, 40%, or 50% of the ratio of phosphatidylserine:phosphatidylglycerol in the source cell; or has a ratio of phosphatidylserine:phosphatidylinositol that is within 10%, 20%, 30%, 40%, or 50% of the ratio of phosphatidylserine:phosphatidylinositol in the source cell; or has a ratio of phosphatidylserine:cholesterol ester that is within 10%, 20%, 30%, 40%, or 50% of the ratio of phosphatidylserine:cholesterol ester in the source cell; or has a ratio of phosphatidylserine:sphingomyelin that is within 10%, 20%, 30%, 40%, or 50% of the ratio of phosphatidylserine:sphingomyelin in the source cell; or has a ratio of phosphatidylserine:triacylglycerol that is within 10%, 20%, 30%, 40%, or 50% of the ratio of phosphatidylserine:triacylglycerol in the source cell; or has a ratio of sphingomyelin:ceramide that is within 10%, 20%, 30%, 40%, or 50% of the ratio of sphingomyelin:ceramide in the source cell; or has a ratio of sphingomyelin:diacylglycerol that is within 10%, 20%, 30%, 40%, or 50% of the ratio of sphingomyelin:diacylglycerol in the source cell; or has a ratio of sphingomyelin:hexosylceramide that is within 10%, 20%, 30%, 40%, or 50% of the ratio of sphingomyelin:hexosylceramide in the source cell; or has a ratio of sphingomyelin:lysophosphatidate that is within 10%, 20%, 30%, 40%, or 50% of the ratio of sphingomyelin:lysophosphatidate in the source cell; or has a ratio of sphingomyelin:lyso-phosphatidylcholine that is within 10%, 20%, 30%, 40%, or 50% of the ratio of sphingomyelin:lyso-phosphatidylcholine in the source cell; or has a ratio of sphingomyelin:lyso-phosphatidylethanolamine that is within 10%, 20%, 30%, 40%, or 50% of the ratio of sphingomyelin:lyso-phosphatidylethanolamine in the source cell; or has a ratio of sphingomyelin:lyso-phosphatidylglycerol that is within 10%, 20%, 30%, 40%, or 50% of the ratio of sphingomyelin:lyso-phosphatidylglycerol in the source cell; or has a ratio of sphingomyelin:lyso-phosphatidylinositol that is within 10%, 20%, 30%, 40%, or 50% of the ratio of sphingomyelin:lyso-phosphatidylinositol in the source cell; or has a ratio of sphingomyelin:lyso-phosphatidylserine that is within 10%, 20%, 30%, 40%, or 50% of the ratio of sphingomyelin:lyso-phosphatidylserine in the source cell; or has a ratio of sphingomyelin:phosphatidate that is within 10%, 20%, 30%, 40%, or 50% of the ratio of sphingomyelin:phosphatidate in the source cell; or has a ratio of sphingomyelin:phosphatidylglycerol that is within 10%, 20%, 30%, 40%, or 50% of the ratio of sphingomyelin:phosphatidylglycerol in the source cell; or has a ratio of sphingomyelin:phosphatidylinositol that is within 10%, 20%, 30%, 40%, or 50% of the ratio of sphingomyelin:phosphatidylinositol in the source cell; or has a ratio of sphingomyelin:cholesterol ester that is within 10%, 20%, 30%, 40%, or 50% of the ratio of sphingomyelin:cholesterol ester in the source cell; or has a ratio of sphingomyelin:triacylglycerol that is within 10%, 20%, 30%, 40%, or 50% of the ratio of sphingomyelin:triacylglycerol in the source cell; or has a ratio of cholesterol ester:ceramide that is within 10%, 20%, 30%, 40%, or 50% of the ratio of cholesterol ester:ceramide in the source cell; or has a ratio of cholesterol ester:diacylglycerol that is within 10%, 20%, 30%, 40%, or 50% of the ratio of cholesterol ester:diacylglycerol in the source cell; or has a ratio of cholesterol ester:hexosylceramide that is within 10%, 20%, 30%, 40%, or 50% of the ratio of cholesterol ester:hexosylceramide in the source cell; or has a ratio of cholesterol ester:lysophosphatidate that is within 10%, 20%, 30%, 40%, or 50% of the ratio of cholesterol ester:lysophosphatidate in the source cell; or has a ratio of cholesterol ester:lyso-phosphatidylcholine that is within 10%, 20%, 30%, 40%, or 50% of the ratio of cholesterol ester:lyso-phosphatidylcholine in the source cell; or has a ratio of cholesterol ester:lyso-phosphatidylethanolamine that is within 10%, 20%, 30%, 40%, or 50% of the ratio of cholesterol ester:lyso-phosphatidylethanolamine in the source cell; or has a ratio of cholesterol ester:lyso-phosphatidylglycerol that is within 10%, 20%, 30%, 40%, or 50% of the ratio of cholesterol ester:lyso-phosphatidylglycerol in the source cell; or has a ratio of cholesterol ester:lyso-phosphatidylinositol that is within 10%, 20%, 30%, 40%, or 50% of the ratio of cholesterol ester:lyso-phosphatidylinositol in the source cell; or has a ratio of cholesterol ester:lyso-phosphatidylserine that is within 10%, 20%, 30%, 40%, or 50% of the ratio of cholesterol ester:lyso-phosphatidylserine in the source cell; or has a ratio of cholesterol ester:phosphatidate that is within 10%, 20%, 30%, 40%, or 50% of the ratio of cholesterol ester:phosphatidate in the source cell; or has a ratio of cholesterol ester:phosphatidylglycerol that is within 10%, 20%, 30%, 40%, or 50% of the ratio of cholesterol ester:phosphatidylglycerol in the source cell; or has a ratio of cholesterol ester:phosphatidylinositol that is within 10%, 20%, 30%, 40%, or 50% of the ratio of cholesterol ester:phosphatidylinositol in the source cell; or has a ratio of cholesterol ester:triacylglycerol that is within 10%, 20%, 30%, 40%, or 50% of the ratio of cholesterol ester:triacylglycerol in the source cell.

In some embodiments, the cytobiologic comprises a proteomic composition similar to that of the source cell, e.g., using an assay of Example 30. In some embodiments, the cytobiologic comprises a ratio of lipids to proteins that is within 10%, 20%, 30%, 40%, or 50% of the corresponding ratio in the source cell, e.g., as measured using an assay of Example 37. In some embodiments, the cytobiologic comprises a ratio of proteins to nucleic acids (e.g., DNA or RNA) that is within 10%, 20%, 30%, 40%, or 50% of the corresponding ratio in the source cell, e.g., as measured using an assay of Example 38. In some embodiments, the cytobiologic comprises a ratio of proteins to DNA that is greater than the corresponding ratio in the source cell, e.g., at least 10%, 20%, 30%, 40%, 50%, 60%, 70%, 80%, or 90% greater, e.g., as measured using an assay of Example 38. In some embodiments, the cytobiologic comprises a ratio of lipids to nucleic acids (e.g., DNA) that is within 10%, 20%, 30%, 40%, or 50% of the corresponding ratio in the source cell, e.g., as measured using an assay of Example 39. In some embodiments, the cytobiologic comprises a ratio of lipids to nucleic acids (e.g., DNA) that is greater than the corresponding ratio in the source cell, e.g., at least 10%, 20%, 30%, 40%, 50%, 60%, 70%, 80%, or 90% greater, e.g., as measured using an assay of Example 39.

In some embodiments, the cytobiologic has a half-life in a subject, e.g., in a mouse, that is within 1%, 2%, 3%, 4%, 5%, 10%, 20%, 30%, 40%, 50%, 60%, 70%, 80%, 90%, 100% of the half life of a reference cell, e.g., the source cell, e.g., by an assay of Example 60. In some embodiments, the cytobiologic has a half-life in a subject, e.g., in a mouse, that is at least 1 hour, 2 hours, 3 hours, 4 hours, 5 hours, 6 hours, 12 hours, or 24 hours, e.g., in a human subject or in a mouse, e.g., by an assay of Example 60. In some embodiments, the cytobiologic has a half-life in a subject, e.g., in a mouse, that is less than 24 hours, 48 hours, or 72 hours, e.g., by an assay of Example 60. In some embodiments, the therapeutic agent has a half-life in a subject that is longer than the half-life of the cytobiologic, e.g., by at least 10%, 20%, 50%, 2-fold, 5-fold, or 10-fold. For instance, the cytobiologic may deliver the therapeutic agent to the target cell, and the therapeutic agent may be present after the cytobiologic is no longer present or detectable.

In some embodiments, the cytobiologic transports glucose (e.g., labeled glucose, e.g., 2-NBDG) across a membrane, e.g., by at least 1%, 2%, 3%, 4%, 5%, 10%, 20%, 30%, 40%, 50%, 60%, 70%, 80%, 90%, 100% more than a negative control, e.g., an otherwise similar cytobiologic in the absence of glucose, e.g., as measured using an assay of Example 49. In some embodiments, the cytobiologic comprises esterase activity in the lumen that is within 1%, 2%, 3%, 4%, 5%, 10%, 20%, 30%, 40%, 50%, 60%, 70%, 80%, 90%, or 100% of that of the esterase activity in a reference cell, e.g., the source cell or a mouse embryonic fibroblast, e.g., using an assay of Example 51. In some embodiments, the cytobiologic comprises a metabolic activity level that is within 1%, 2%, 3%, 4%, 5%, 10%, 20%, 30%, 40%, 50%, 60%, 70%, 80%, 90%, or 100% of the citrate synthase activity in a reference cell, e.g., the source cell, e.g., as described in Example 53. In some embodiments, the cytobiologic comprises a respiration level (e.g., oxygen consumption rate) that is within 1%, 2%, 3%, 4%, 5%, 10%, 20%, 30%, 40%, 50%, 60%, 70%, 80%, 90%, or 100% of the respiration level in a reference cell, e.g., the source cell, e.g., as described in Example 54. In some embodiments, the cytobiologic comprises an Annexin-V staining level of at most 18,000, 17,000, 16,000, 15,000, 14,000, 13,000, 12,000, 11,000, or 10,000 MFI, e.g., using an assay of Example 55, or wherein the cytobiologic comprises an Annexin-V staining level at least 5%, 10%, 20%, 30%, 40%, 50%, 60%, 70%, 80%, or 90% lower than the Annexin-V staining level of an otherwise similar cytobiologic treated with menadione in the assay of Example 55, or wherein the cytobiologic comprises an Annexin-V staining level at least 5%, 10%, 20%, 30%, 40%, 50%, 60%, 70%, 80%, or 90% lower than the Annexin-V staining level of a macrophage treated with menadione in the assay of Example 55.

In some embodiments, the cytobiologic has a miRNA content level of at least 1%, 2%, 3%, 4%, 5%, 10%, 20%, 30%, 40%, 50%, 60%, 70%, 80%, 90%, or greater than that of the source cell, e.g., by an assay of Example 27. In some embodiments, the cytobiologic has a miRNA content level of at least 1%, 2%, 3%, 4%, 5%, 10%, 20%, 30%, 40%, 50%, 60%, 70%, 80%, 90%, or greater of the miRNA content level of the source cell (e.g., up to 100% of the miRNA content level of the source cell), e.g., by an assay of Example 27. In some embodiments, the cytobiologic has a total RNA content level of at least 1%, 2%, 3%, 4%, 5%, 10%, 20%, 30%, 40%, 50%, 60%, 70%, 80%, 90%, or greater of the total RNA content level of the source cell (e.g., up to 100% of the total RNA content level of the source cell), e.g., as measured by an assay of Example 80. In some embodiments, the cytobiologic has a soluble:non-soluble protein ratio is within 1%, 2%, 3%, 4%, 5%, 10%, 20%, 30%, 40%, 50%, 60%, 70%, 80%, 90%, or greater than that of the source cell, e.g., within 1%-2%, 2%-3%, 3%-4%, 4%-5%, 5%-10%, 10%-20%, 20%-30%, 30%-40%, 40%-50%, 50%-60%, 60%-70%, 70%-80%, or 80%-90% of that of the source cell, e.g., by an assay of Example 35. In some embodiments, the cytobiologic has an LPS level less than 5%, 1%, 0.5%, 0.01%, 0.005%, 0.0001%, 0.00001% or less of the lipid content of cytobiologic, e.g., as measured by an assay of Example 36. In some embodiments, the cytobiologic has an LPS level less than 5%, 1%, 0.5%, 0.01%, 0.005%, 0.0001%, 0.00001% or less of the LPS content of the source cell, e.g., as measured by mass spectrometry, e.g., in an assay of Example 36. In some embodiments, the cytobiologic is capable of signal transduction, e.g., transmitting an extracellular signal, e.g., AKT phosphorylation in response to insulin, or glucose (e.g., labeled glucose, e.g., 2-NBDG) uptake in response to insulin, e.g., by at least 1%, 2%, 3%, 4%, 5%, 10%, 20%, 30%, 40%, 50%, 60%, 70%, 80%, 90%, 100% more than a negative control, e.g., an otherwise similar cytobiologic in the absence of insulin, e.g., using an assay of Example 48. In some embodiments, the cytobiologic targets a tissue, e.g., liver, lungs, heart, spleen, pancreas, gastrointestinal tract, kidney, testes, ovaries, brain, reproductive organs, central nervous system, peripheral nervous system, skeletal muscle, endothelium, inner ear, or eye, when administered to a subject, e.g., a mouse, e.g., wherein at least 0.1%, 0.5%, 1%, 1.5%, 2%, 2.5%, 3%, 4%, 5%, 10%, 15%, 20%, 25%, 30%, 35%, 40%, 50%, 60%, 70%, 80%, or 90% of the cytobiologics in a population of administered cytobiologics are present in the target tissue after 24, 48, or 72 hours, e.g., by an assay of Example 71. In some embodiments, the cytobiologic has a juxtacrine-signaling level of at least 1%, 2%, 3%, 4%, 5%, 10%, 20%, 30%, 40%, 50%, 60%, 70%, 80%, 90%, or 100% greater than the level of juxtacrine signaling induced by a reference cell, e.g., the source cell or a bone marrow stromal cell (BMSC), e.g., by an assay of Example 56. In some embodiments, the cytobiologic has a juxtacrine-signaling level of at least 1%, 2%, 3%, 4%, 5%, 10%, 20%, 30%, 40%, 50%, 60%, 70%, 80%, or 90% (e.g., up to 100%) greater than the level of juxtacrine signaling in a reference cell, e.g., the source cell or a bone marrow stromal cell (BMSC), e.g., by an assay of Example 56. In some embodiments, the cytobiologic has paracrine-signaling level of at least 1%, 2%, 3%, 4%, 5%, 10%, 20%, 30%, 40%, 50%, 60%, 70%, 80%, 90%, 100% greater than the level of paracrine signaling induced by a reference cell, e.g., the source cell or a macrophage, e.g., by an assay of Example 57. In some embodiments, the cytobiologic has paracrine-signaling level of at least 1%, 2%, 3%, 4%, 5%, 10%, 20%, 30%, 40%, 50%, 60%, 70%, 80%, or 90% (e.g., up to 100%) or the level of paracrine signaling induced by a reference cell, e.g., the source cell or a macrophage, e.g., by an assay of Example 57. In some embodiments, the cytobiologic polymerizes actin at a level within 1%, 2%, 3%, 4%, 5%, 10%, 20%, 30%, 40%, 50%, 60%, 70%, 80%, 90%, or 100% compared to the level of polymerized actin in a reference cell, e.g., the source cell or a C2C12 cell, e.g., by the assay of Example 58. In some embodiments, the cytobiologic has a membrane potential within about 1%, 2%, 3%, 4%, 5%, 10%, 20%, 30%, 40%, 50%, 60%, 70%, 80%, 90%, 100% of the membrane potential of a reference cell, e.g., the source cell or a C2C12 cell, e.g., by an assay of Example 59, or wherein the cytobiologic has a membrane potential of about −20 to −150 mV, −20 to −50 mV, −50 to −100 mV, or −100 to −150 mV, or wherein the cytobiologic has a membrane potential of less than −1 mv, −5 mv, −10 mv, −20 mv, −30 mv, −40 mv, −50 mv, −60 mv, −70 mv, −80 mv, −90 mv, −100 mv. In some embodiments, the cytobiologic is capable of extravasation from blood vessels, e.g., at a rate at least 1%, 2%, 5%, 10%, 20%, 30%, 40%, 50%, 60%, 70%, 80%, or 90% the rate of extravasation of the source cell, e.g., using an assay of Example 42, e.g., wherein the source cell is a neutrophil, lymphocyte, B cell, macrophage, or NK cell. In some embodiments, the cytobiologic is capable of chemotaxis, e.g., of at least 1%, 2%, 3%, 4%, 5%, 10%, 20%, 30%, 40%, 50%, 60%, 70%, 80%, or 90% (e.g., up to 100%) compared to a reference cell, e.g., a macrophage, e.g., using an assay of Example 43. In some embodiments, the cytobiologic is capable of phagocytosis, e.g., at least 1%, 2%, 3%, 4%, 5%, 10%, 20%, 30%, 40%, 50%, 60%, 70%, 80%, or 90% (e.g., up to 100%) compared to a reference cell, e.g., a macrophage, e.g., using an assay of Example 45. In some embodiments, the cytobiologic is capable of crossing a cell membrane, e.g., an endothelial cell membrane or the blood brain barrier. In some embodiments, the cytobiologic is capable of secreting a protein, e.g., at a rate at least 1%, 2%, 3%, 4%, 5%, 10%, 20%, 30%, 40%, 50%, 60%, 70%, 80%, 90%, or 100% greater than a reference cell, e.g., a mouse embryonic fibroblast, e.g., using an assay of Example 47. In some embodiments, the cytobiologic is capable of secreting a protein, e.g., at a rate at least 1%, 2%, 3%, 4%, 5%, 10%, 20%, 30%, 40%, 50%, 60%, 70%, 80%, or 90% (e.g., up to 100%) compared to a reference cell, e.g., a mouse embryonic fibroblast, e.g., using an assay of Example 47.

In some embodiments, the cytobiologic is not capable of transcription or has transcriptional activity of less than 1%, 2.5% 5%, 10%, 20%, 30%, 40%, 50%, 60%, 70%, 80%, or 90% of that of the transcriptional activity of a reference cell, e.g., the source cell, e.g., using an assay of Example 9. In some embodiments, the cytobiologic is not capable of nuclear DNA replication or has nuclear DNA replication of less than 1%, 2.5% 5%, 10%, 20%, 30%, 40%, 50%, 60%, 70%, 80%, or 90% of the nuclear DNA replication of a reference cell, e.g., the source cell, e.g., using an assay of Example 10. In some embodiments, the cytobiologic lacks chromatin or has a chromatin content of less than 1%, 2.5% 5%, 10%, 20%, 30%, 40%, 50%, 60%, 70%, 80%, or 90% of the of the chromatin content of a reference cell, e.g., the source cell, e.g., using an assay of Example 25.

In some embodiments, a characteristic of a cytobiologic is described by comparison to a reference cell. In embodiments, the reference cell is the source cell. In embodiments, the reference cell is a HeLa, HEK293, HFF-1, MRC-5, WI-38, IMR 90, IMR 91, PER.C6, HT-1080, or BJ cell. In some embodiments, a characteristic of a population of cytobiologics is described by comparison to a population of reference cells, e.g., a population of source cells, or a population of HeLa, HEK293, HFF-1, MRC-5, WI-38, IMR 90, IMR 91, PER.C6, HT-1080, or BJ cells.

In some embodiments, the cytobiologic meets a pharmaceutical or good manufacturing practices (GMP) standard. In some embodiments, the cytobiologic was made according to good manufacturing practices (GMP). In some embodiments, the cytobiologic has a pathogen level below a predetermined reference value, e.g., is substantially free of pathogens. In some embodiments, the cytobiologic has a contaminant level below a predetermined reference value, e.g., is substantially free of contaminants. In some embodiments, the cytobiologic has low immunogenicity, e.g., as described herein;

In some embodiments, the source cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), or an immortalized cell (e.g., HeLa, HEK293, HFF-1, MRC-5, WI-38, IMR 90, IMR 91, PER.C6, HT-1080, or BJ cell). In some embodiments, the source cell is other than a 293 cell, HEK cell, human endothelial cell, or a human epithelial cell, monocyte, macrophage, dendritic cell, or stem cell.

In some embodiments, the cytobiologic comprises a cargo, e.g., a therapeutic agent, e.g., an endogenous therapeutic agent or an exogenous therapeutic agent. In some embodiments, the therapeutic agent is chosen from one or more of a protein, e.g., an enzyme, a transmembrane protein, a receptor, an antibody; a nucleic acid, e.g., DNA, a chromosome (e.g. a human artificial chromosome), RNA, mRNA, siRNA, miRNA, or a small molecule. In some embodiments, the therapeutic agent is an organelle other than a mitochondrion, e.g., an organelle selected from: nucleus, Golgi apparatus, lysosome, endoplasmic reticulum, vacuole, endosome, acrosome, autophagosome, centriole, glycosome, glyoxysome, hydrogenosome, melanosome, mitosome, cnidocyst, peroxisome, proteasome, vesicle, and stress granule. In some embodiments, the organelle is a mitochondrion.

In some embodiments, the cytobiologic, composition, or preparation has a density of <1, 1-1.1, 1.05-1.15, 1.1-1.2, 1.15-1.25, 1.2-1.3, 1.25-1.35, or >1.35 g/ml, e.g., by an assay of Example 21.

In some embodiments, the cytobiologic composition comprises less than 0.01%, 0.05%, 0.1%, 0.5%, 1%, 1.5%, 2%, 2.5%, 3%, 4%, 5%, or 10% source cells by protein mass or less than 0.01%, 0.05%, 0.1%, 0.5%, 1%, 1.5%, 2%, 2.5%, 3%, 4%, 5%, or 10% of cells have a functional nucleus. In some embodiments, at least 10%, 20%, 30%, 40%, 50%, 60%, 70%, 80%, 90%, 95%, or 99% of cytobiologics in the cytobiologic composition comprise an organelle, e.g., a mitochondrion.

In some embodiments, the cytobiologic further comprises an exogenous therapeutic agent. In some embodiments, the exogenous therapeutic agent is chosen from one or more of a protein, e.g., an enzyme, a transmembrane protein, a receptor, an antibody; a nucleic acid, e.g., DNA, a chromosome (e.g. a human artificial chromosome), RNA, mRNA, siRNA, miRNA, or a small molecule.

In some embodiments, the cytobiologic or cytobiologic composition is refrigerated or frozen. In embodiments, the cytobiologic does not comprise a functional nucleus, or the cytobiologic composition comprises a cytobiologic without a functional nucleus. In embodiments, the cytobiologic composition comprises less than 0.01%, 0.05%, 0.1%, 0.5%, 1%, 1.5%, 2%, 2.5%, 3%, 4%, 5%, or 10% source cells by protein mass or less than 0.01%, 0.05%, 0.1%, 0.5%, 1%, 1.5%, 2%, 2.5%, 3%, 4%, 5%, or 10% of cells have a functional nucleus. In embodiments, the cytobiologic composition has been maintained at said temperature for at least 1, 2, 3, 6, or 12 hours; 1, 2, 3, 4, 5, or 6 days; 1, 2, 3, or 4 weeks; 1, 2, 3, or 6 months; or 1, 2, 3, 4, or 5 years. In embodiments, the cytobiologic composition has an activity of at least 50%, 60%, 70%, 80%, 90%, 95%, or 99% of the activity of the population before maintenance at said temperature, e.g., using an assay described herein.

In embodiments, the cytobiologic composition is stable at a temperature of less than 4 C for at least 1, 2, 3, 6, or 12 hours; 1, 2, 3, 4, 5, or 6 days; 1, 2, 3, or 4 weeks; 1, 2, 3, or 6 months; or 1, 2, 3, 4, or 5 years. In embodiments, the cytobiologic composition is stable at a temperature of less than −20 C for at least 1, 2, 3, 6, or 12 hours; 1, 2, 3, 4, 5, or 6 days; 1, 2, 3, or 4 weeks; 1, 2, 3, or 6 months; or 1, 2, 3, 4, or 5 years. In embodiments, the cytobiologic composition is stable at a temperature of less than −80 C for at least 1, 2, 3, 6, or 12 hours; 1, 2, 3, 4, 5, or 6 days; 1, 2, 3, or 4 weeks; 1, 2, 3, or 6 months; or 1, 2, 3, 4, or 5 years.

In embodiments, one or more of:
i) the source cell is other than a 293 cell;
ii) the source cell is not transformed or immortalized;
iii) the source cell is transformed or immortalized using a method other than adenovirus-mediated immortalization, e.g., immortalized by spontaneous mutation or telomerase expression;
iv) the therapeutic agent is other than Cre or EGFP;
v) the therapeutic agent is a nucleic acid (e.g., RNA, e.g., mRNA, miRNA, or siRNA) or an exogenous protein (e.g., an antibody, e.g., an antibody), e.g., in the lumen; or
vi) the cytobiologic does not comprise mitochondria.

In embodiments, one or more of:
i) the source cell is other than a 293 or HEK cell;
ii) the source cell is not transformed or immortalized;
iii) the source cell is transformed or immortalized using a method other than adenovirus-mediated immortalization, e.g., immortalized by spontaneous mutation or telomerase expression;
iv) the cytobiologic has a size of other than between 40 and 150 nm, e.g., greater than 150 nm, 200 nm, 300 n, 400 nm, or 500 nm.

In embodiments, one or more of:
i) the therapeutic agent is a soluble protein expressed by the source cell;
ii) the cytobiologic comprises in its lumen a polypeptide selected from an enzyme, antibody, or anti-viral polypeptide;
iii) the cytobiologic does not comprise an exogenous therapeutic transmembrane protein; or iv) the cytobiologic does not comprise CD63 or GLUT4.

In embodiments, the cytobiologic:
i) does not comprise a virus, is not infectious, or does not propagate in a host cell;
ii) is not a VLP (virus like particle);
iii) does not comprise a viral structural protein, e.g., a viral capsid protein, e.g., a viral nucleocapsid protein, or wherein the amount of viral capsid protein is less than 10%, 5%, 4%, 3%, 2%, 1%, 0.5%, 0.2%, or 0.1% of total protein, e.g., by an assay of Example 41;
iv) does not comprise a viral matrix protein;
v) does not comprise a viral non-structural protein;
vi) comprises less than 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, 1,000,000,000 copies per vesicle of a viral structural protein; or
vii) the cytobiologic is not a virosome.

In embodiments, the ratio of the copy number of the therapeutic agent or exogenous agent to the copy number of viral structural protein on the cytobiologic is at least 1000000:1, 100000:1, 10000:1, 1000:1, 100:1 and 50:1, 50:1 and 20:1, 20:1 and 10:1, 10:1 and 5:1, or 1:1. In embodiments, the ratio of the copy number of the therapeutic agent or exogenous agent to the copy number of viral structural protein on the cytobiologic is at least 1,000,000:1, 100,000:1, 10,000:1, 1,000:1, 100:1, 50:1, 20:1, 10:1, 5:1, or 1:1. In embodiments, the ratio of the copy number of the therapeutic agent or exogenous agent to the copy number of viral matrix protein on the cytobiologic is at least 1000000:1, 100000:1, 10000:1, 1000:1, 100:1 and 50:1, 50:1 and 20:1, 20:1 and 10:1, 10:1 and 5:1, or 1:1. In embodiments, the ratio of the copy number of the therapeutic agent or exogenous agent to the copy number of viral matrix protein on the cytobiologic is at least 1,000,000:1, 100,000:1, 10,000:1, 1,000:1, 100:1, 50:1, 20:1, 10:1, 5:1, or 1:1.

In embodiments, one or more of:
i) the cytobiologic does not comprise a water-immiscible droplet;
ii) the cytobiologic comprises an aqueous lumen and a hydrophilic exterior;
iii) the organelle is selected from a mitochondrion, Golgi apparatus, lysosome, endoplasmic reticulum, vacuole, endosome, acrosome, autophagosome, centriole, glycosome, glyoxysome, hydrogenosome, melanosome, mitosome, cnidocyst, peroxisome, proteasome, vesicle, and stress granule.

In embodiments, one or more of:
i) the cytobiologic was not made by loading the cytobiologic with a therapeutic or diagnostic substance;
ii) the source cell was not loaded with a therapeutic or diagnostic substance;
iii) the cytobiologic does not comprise doxorubicin, dexamethasone, cyclodextrin; polyethylene glycol, a micro RNA e.g., miR125, VEGF receptor, ICAM-1, E-selectin, iron oxide, a fluorescent protein e.g., GFP or RFP, a nanoparticle, or an RNase, or does not comprise an exogenous form of any of the foregoing; or
iv) the cytobiologic further comprises an exogenous therapeutic agent having one or more post-translational modifications, e.g., glycosylation.

In embodiments, the cytobiologic is unilamellar or multilamellar.

In embodiments, the cytobiologic has a size within about 0.01%, 0.05%, 0.1%, 0.5%, 1%, 2%, 3%, 4%, 5%, 10%, 20%, 30%, 40%, 50%, 60%, 70%, 80%, 90%, of that of the source cell, e.g., as measured by an assay of Example 18. In embodiments, the cytobiologic has a size that is less than about 0.01%, 0.05%, 0.1%, 0.5%, 1%, 2%, 3%, 4%, 5%, 10%, 20%, 30%, 40%, 50%, 60%, 70%, 80%, 90%, of that of the source cell, e.g., as measured by an assay of Example 18. In embodiments, the cytobiologic has a size within about 0.01%-0.05%, 0.05%-0.1%, 0.1%-0.5%, 0.5%-1%, 1%-2%, 2%-3%, 3%-4%, 4%-5%, 5%-10%, 10%-20%, 20%-30%, 30%-40%, 40%-50%, 50%-60%, 60%-70%, 70%-80%, or 80%-90% the size of the source cell, e.g., as measured by an assay of Example 18. In embodiments, the cytobiologic has a size that is less than about 0.01%-0.05%, 0.05%-0.1%, 0.1%-0.5%, 0.5%-1%, 1%-2%, 2%-3%, 3%-4%, 4%-5%, 5%-10%, 10%-20%, 20%-30%, 30%-40%, 40%-50%, 50%-60%, 60%-70%, 70%-80%, or 80%-90% of the size of the source cell, e.g., as measured by an assay of Example 18. In embodiments, the cytobiologic has a size that is about 0.01%-0.05%, 0.05%-0.1%, 0.1%-0.5%, 0.5%-1%, 1%-2%, 2%-3%, 3%-4%, 4%-5%, 5%-10%, 10%-20%, 20%-30%, 30%-40%, 40%-50%, 50%-60%, 60%-70%, 70%-80%, or 80%-90% of the size of the source cell, e.g., as measured by an assay of Example 18. In embodiments, the cytobiologic has a diameter of at least about 10 nm, 20 nm, 30 nm, 40 nm, 50 nm, 60 nm, 70 nm, 80 nm, 90 nm, 100 nm, 150 nm, 200 nm, or 250 nm, e.g., as measured by an assay of Example 20. In embodiments, the cytobiologic has a diameter of about 10 nm, 20 nm, 30 nm, 40 nm, 50 nm, 60 nm, 70 nm, 80 nm, 90 nm, 100 nm, 150 nm, 200 nm, or 250 nm (e.g., ±20%) e.g., as measured by an assay of Example 20. In embodiments, the cytobiologic has a diameter of at least about 500 nm, 750 nm, 1,000 nm, 1,500 nm, 2,000 nm, 2,500 nm, 3,000 nm, 5,000 nm, 10,000 nm, or 20,000 nm, e.g., as measured by an assay of Example 20. In embodiments, the cytobiologic has a diameter of about 500 nm, 750 nm, 1,000 nm, 1,500 nm, 2,000 nm, 2,500 nm, 3,000 nm, 5,000 nm, 10,000 nm, or 20,000 nm (e.g., ±20%), e.g., as measured by an assay of Example 20. In some embodiments, a population of cytobiologics has an average size of less than 80 nm, 100 nm, 200 nm, 500 nm, 1000 nm, 1200 nm, 1400 nm, or 1500 nm.

In embodiments, one or more of:
i) the cytobiologic is not an exosome;
ii) the cytobiologic is a microvesicle;
iii) the cytobiologic has a size of at least 80 nm, 100 nm, 200 nm, 500 nm, 1000 nm, 1200 nm, 1400 nm, or 1500 nm, or a population of cytobiologics has an average size of at least 80 nm, 100 nm, 200 nm, 500 nm, 1000 nm, 1200 nm, 1400 nm, or 1500 nm;
iv) the cytobiologic comprises one or more organelles, e.g., a mitochondrion, Golgi apparatus, lysosome, endoplasmic reticulum, vacuole, endosome, acrosome, autophagosome, centriole, glycosome, glyoxysome, hydrogenosome, melanosome, mitosome, cnidocyst, peroxisome, proteasome, vesicle, and stress granule;
v) the cytobiologic comprises a cytoskeleton or a component thereof, e.g., actin, Arp2/3, formin, coronin, dystrophin, keratin, myosin, or tubulin;
vi) the cytobiologic, composition, or preparation does not have a flotation density of 1.08-1.22 g/ml, or has a density of at least 1.18-1.25 g/ml, or 1.05-1.12 g/ml, e.g., in a sucrose gradient centrifugation assay, e.g., as described in Théry et al., “Isolation and characterization of exosomes from cell culture supernatants and biological fluids.” Curr Protoc Cell Biol. 2006 April; Chapter 3:Unit 3.22;
vii) the cytobiologic comprises a lipid bilayer that is enriched for ceramides or sphingomyelins or a combination thereof compared to the source cell, or the lipid bilayer is not enriched (e.g., is depleted) for glycolipids, free fatty acids, or phosphatidylserine, or a combination thereof, compared to the source cell;
viii) the cytobiologic comprises Phosphatidyl serine (PS) or CD40 ligand or both of PS and CD40 ligand, e.g., when measured in an assay of Example 40;
ix) the cytobiologic is enriched for PS compared to the source cell, e.g., in a population of cytobiologics at least 20%, 30%, 40%, 50%, 60%, 70%, 80%, or 90% are positive for PS by an assay of Kanada M, et al. (2015) Differential fates of biomolecules delivered to target cells via extracellular vesicles. Proc Natl Acad Sci USA 112:E1433-E1442;
x) the cytobiologic is substantially free of acetylcholinesterase (AChE), or contains less than 0.001, 0.002, 0.005, 0.01, 0.02, 0.05, 0.1, 0.2, 0.5, 1, 2, 5, 10, 20, 50, 100, 200, 500, or 1000 AChE activity units/ug of protein, e.g., by an assay of Example 52;
xi) the cytobiologic is substantially free of a Tetraspanin family protein (e.g., CD63, CD9, or CD81), an ESCRT-related protein (e.g., TSG101, CHMP4A-B, or VPS4B), Alix, TSG101, MHCI, MHCII, GP96, actinin-4, mitofilin, syntenin-1, TSG101, ADAM10, EHD4, syntenin-1, TSG101, EHD1, flotillin-1, heat-shock 70-kDa proteins (HSC70/HSP73, HSP70/HSP72), or any combination thereof, or contains less than 0.05%, 0.1%, 0.5%, 1%, 2%, 3%, 4%, 5%, 5%, or 10% of any individual exosomal marker protein and/or less than 0.05%, 0.1%, 0.5%, 1%, 2%, 3%, 4%, 5%, 10%, 15%, 20%, or 25% of total exosomal marker proteins of any of said proteins, or is de-enriched for any one or more of these proteins compared to the source cell, or is not enriched for any one or more of these proteins, e.g., by an assay of Example 32;
xii) the cytobiologic comprises a level of Glyceraldehyde 3-phosphate dehydrogenase (GAPDH) that is below 500, 250, 100, 50, 20, 10, 5, or 1 ng GAPDH/ug total protein or below the level of GAPDH in the source cell, e.g., less than 1%, 2.5%, 5%, 10%, 15%, 20%, 30%, 40%, 50%, 60%, 70%, 80%, or 90%, less than the level of GAPDH per total protein in ng/ug in the source cell, e.g., using an assay of Example 33;
xiii) the cytobiologic is enriched for one or more endoplasmic reticulum proteins (e.g., calnexin), one or more proteasome proteins, or one or more mitochondrial proteins, or any combination thereof, e.g., wherein the amount of calnexin is less than 500, 250, 100, 50, 20, 10, 5, or 1 ng Calnexin/ug total protein, or wherein the cytobiologic comprises less Calnexin per total protein in ng/ug compared to the source cell by 1%, 2.5%, 5%, 10%, 15%, 20%, 30%, 40%, 50%, 60%, 70%, 80%, or 90%, e.g., using an assay of Example 34;
xiv) the cytobiologic comprises an exogenous agent (e.g., an exogenous protein, mRNA, or siRNA) e.g., as measured using an assay of Example 27 or 28; or
xv) the cytobiologic can be immobilized on a mica surface by atomic force microscopy for at least 30 min, e.g., by an assay of Kanada M, et al. (2015) Differential fates of biomolecules delivered to target cells via extracellular vesicles. Proc Natl Acad Sci USA 112:E1433-E1442.

In embodiments, one or more of:
i) the cytobiologic is an exosome;
ii) the cytobiologic is not a microvesicle;
iii) the cytobiologic has a size of less than 80 nm, 100 nm, 200 nm, 500 nm, 1000 nm, 1200 nm, 1400 nm, or 1500 nm, or a population of cytobiologics has an average size of at least 80 nm, 100 nm, 200 nm, 500 nm, 1000 nm, 1200 nm, 1400 nm, or 1500 nm;
iv) the cytobiologic does not comprise an organelle;
v) the cytobiologic does not comprise a cytoskeleton or a component thereof, e.g., actin, Arp2/3, formin, coronin, dystrophin, keratin, myosin, or tubulin;
vi) the cytobiologic, composition, or preparation has flotation density of 1.08-1.22 g/ml, e.g., in a sucrose gradient centrifugation assay, e.g., as described in Théry et al., “Isolation and characterization of exosomes from cell culture supernatants and biological fluids.” Curr Protoc Cell Biol. 2006 April; Chapter 3:Unit 3.22;
vii) the lipid bilayer is not enriched (e.g., is depleted) for ceramides or sphingomyelins or a combination thereof compared to the source cell, or the lipid bilayer is enriched for glycolipids, free fatty acids, or phosphatidylserine, or a combination thereof, compared to the source cell; viii) the cytobiologic does not comprise, or is depleted for relative to the source cell, Phosphatidyl serine (PS) or CD40 ligand or both of PS and CD40 ligand, e.g., when measured in an assay of Example 40;
ix) the cytobiologic is not enriched (e.g., is depleted) for PS compared to the source cell, e.g., in a population of cytobiologics less than 20%, 30%, 40%, 50%, 60%, 70%, 80%, or 90% are positive for PS by an assay of Kanada M, et al. (2015) Differential fates of biomolecules delivered to target cells via extracellular vesicles. Proc Natl Acad Sci USA 112:E1433-E1442;
x) the cytobiologic comprises acetylcholinesterase (AChE), e.g. at least 0.001, 0.002, 0.005, 0.01, 0.02, 0.05, 0.1, 0.2, 0.5, 1, 2, 5, 10, 20, 50, 100, 200, 500, or 1000 AChE activity units/ug of protein, e.g., by an assay of Example 52;
xi) the cytobiologic comprises a Tetraspanin family protein (e.g., CD63, CD9, or CD81), an ESCRT-related protein (e.g., TSG101, CHMP4A-B, or VPS4B), Alix, TSG101, MHCI, MHCII, GP96, actinin-4, mitofilin, syntenin-1, TSG101, ADAM10, EHD4, syntenin-1, TSG101, EHD1, flotillin-1, heat-shock 70-kDa proteins (HSC70/HSP73, HSP70/HSP72), or any combination thereof, e.g., contains more than 0.05%, 0.1%, 0.5%, 1%, 2%, 3%, 4%, 5%, 5%, or 10% of any individual exosomal marker protein and/or less than 0.05%, 0.1%, 0.5%, 1%, 2%, 3%, 4%, 5%, 10%, 15%, 20%, or 25% of total exosomal marker proteins of any of said proteins, or is enriched for any one or more of these proteins compared to the source cell, e.g., by an assay of Example 32;
xii) the cytobiologic comprises a level of Glyceraldehyde 3-phosphate dehydrogenase (GAPDH) that is above 500, 250, 100, 50, 20, 10, 5, or 1 ng GAPDH/ug total protein or below the level of GAPDH in the source cell, e.g., at least 1%, 2.5%, 5%, 10%, 15%, 20%, 30%, 40%, 50%, 60%, 70%, 80%, or 90%, greater than the level of GAPDH per total protein in ng/ug in the source cell, e.g., using an assay of Example 33;
xiii) the cytobiologic is not enriched for (e.g., is depleted for) one or more endoplasmic reticulum proteins (e.g., calnexin), one or more proteasome proteins, or one or more mitochondrial proteins, or any combination thereof, e.g., wherein the amount of calnexin is less than 500, 250, 100, 50, 20, 10, 5, or 1 ng Calnexin/ug total protein, or wherein the cytobiologic comprises less Calnexin per total protein in ng/ug compared to the source cell by 1%, 2.5%, 5%, 10%, 15%, 20%, 30%, 40%, 50%, 60%, 70%, 80%, or 90%, e.g., using an assay of Example 34; or
xiv) the cytobiologic can not be immobilized on a mica surface by atomic force microscopy for at least 30 min, e.g., by an assay of Kanada M, et al. (2015) Differential fates of biomolecules delivered to target cells via extracellular vesicles. Proc Natl Acad Sci USA 112:E1433-E1442.

In embodiments, one or more of:
i) the cytobiologic does not comprise a VLP;
ii) the cytobiologic does not comprise a virus;
iii) the cytobiologic does not comprise a replication-competent virus;
iv) the cytobiologic does not comprise a viral protein, e.g., a viral structural protein, e.g., a capsid protein or a viral matrix protein;
v) the cytobiologic does not comprise a capsid protein from an enveloped virus;
vi) the cytobiologic does not comprise a nucleocapsid protein; or
vii) the cytobiologic does not comprise a viral fusogen.

In embodiments, the cytobiologic comprises cytosol.

In embodiments, one or more of:
i) the cytobiologic or the source cell does not form a teratoma when implanted into subject, e.g., by an assay of Example 74;

ii) the cytobiologic is capable of chemotaxis, e.g., of within 1%, 2%, 3%, 4%, 5%, 10%, 20%, 30%, 40%, 50%, 60%, 70%, 80%, 90%, 100% or greater than a reference cell, e.g., a macrophage, e.g., using an assay of Example 43;
iii) the cytobiologic is capable of homing, e.g., at the site of an injury, wherein the cytobiologic is from a human cell, e.g., using an assay of Example 44, e.g., wherein the source cell is a neutrophil; or
iv) the cytobiologic is capable of phagocytosis, e.g., wherein phagocytosis by the cytobiologic is detectable within 0.5, 1, 2, 3, 4, 5, or 6 hours in using an assay of Example 45, e.g., wherein the source cell is a macrophage.

In embodiments, the cytobiologic or cytobiologic composition retains one, two, three, four, five six or more of any of the characteristics for 5 days or less, e.g., 4 days or less, 3 days or less, 2 days or less, 1 day or less, e.g., about 12-72 hours, after administration into a subject, e.g., a human subject.

In embodiments, the cytobiologic has one or more of the following characteristics:
a) comprises one or more endogenous proteins from a source cell, e.g., membrane proteins or cytosolic proteins;
b) comprises at least 10, 20, 50, 100, 200, 500, 1000, 2000, or 5000 different proteins; c) comprises at least 1, 2, 5, 10, 20, 50, or 100 different glycoproteins;
d) at least 10%, 20%, 30%, 40%, 50%, 60%, 70%, 80%, or 90% by mass of the proteins in the cytobiologic are naturally-occurring proteins;
e) comprises at least 10, 20, 50, 100, 200, 500, 1000, 2000, or 5000 different RNAs; or
f) comprises at least 2, 3, 4, 5, 10, or 20 different lipids, e.g., selected from CL, Cer, DAG, HexCer, LPA, LPC, LPE, LPG, LPI, LPS, PA, PC, PE, PG, PI, PS, CE, SM and TAG.

In embodiments, the cytobiologic has been manipulated to have, or the cytobiologic is not a naturally occurring cell and has, or wherein the nucleus does not naturally have one, two, three, four, five or more of the following properties:
a) the partial nuclear inactivation results in a reduction of at least 50%, 60%, 70%, 80%, 90% or more in nuclear function, e.g., a reduction in transcription or DNA replication, or both, e.g., wherein transcription is measured by an assay of Example 9 and DNA replication is measured by an assay of Example 10;
b) the cytobiologic is not capable of transcription or has transcriptional activity of less than 1%, 2.5% 5%, 10%, 20%, 30%, 40%, 50%, 60%, 70%, 80%, or 90% of that of the transcriptional activity of a reference cell, e.g., the source cell, e.g., using an assay of Example 9;
c) the cytobiologic is not capable of nuclear DNA replication or has nuclear DNA replication of less than 1%, 2.5% 5%, 10%, 20%, 30%, 40%, 50%, 60%, 70%, 80%, or 90% of the nuclear DNA replication of a reference cell, e.g., the source cell, e.g., using an assay of Example 10;
d) the cytobiologic lacks chromatin or has a chromatin content of less than 1%, 2.5% 5%, 10%, 20%, 30%, 40%, 50%, 60%, 70%, 80%, or 90% of the of the chromatin content of a reference cell, e.g., the source cell, e.g., using an assay of Example 25;
e) the cytobiologic lacks a nuclear membrane or has less than 50%, 40%, 30%, 20%, 10%, 5%, 4%, 3%, 2%, or 1% the amount of nuclear membrane of a reference cell, e.g., the source cell or a Jurkat cell, e.g., by an assay of Example 24;
f) the cytobiologic lacks functional nuclear pore complexes or has reduced nuclear import or export activity, e.g., by at least 50%, 40%, 30%, 20%, 10%, 5%, 4%, 3%, 2%, or 1% by an assay of Example 24, or the cytobiologic lacks on or more of a nuclear pore protein, e.g., NUP98 or Importin 7.
g) the cytobiologic does not comprise histones or has histone levels less than 1%, 2%, 3%, 4%, 5%, 10%, 20%, 30%, 40%, 50%, 60%, 70%, 80%, or 90% of the histone level of the source cell (e.g., of H1, H2a, H2b, H3, or H4), e.g., by an assay of Example 25;
h) the cytobiologic comprises less than 20, 10, 5, 4, 3, 2, or 1 chromosome;
i) nuclear function is eliminated;
j) the cytobiologic is an enucleated mammalian cell;
k) the nucleus is removed or inactivated, e.g., extruded by mechanical force, by radiation or by chemical ablation; or
l) the cytobiologic is from a mammalian cell having DNA that is completely or partially removed, e.g., during interphase or mitosis.

In embodiments, the cytobiologic comprises mtDNA or vector DNA. In embodiments, the cytobiologic does not comprise DNA.

In embodiments, the source cell is a primary cell, immortalized cell or a cell line (e.g., myelobast cell line, e.g., C2C12). In embodiments, the cytobiologic is from a source cell having a modified genome, e.g., having reduced immunogenicity (e.g., by genome editing, e.g., to remove an MHC protein). In embodiments, the source cell is from a cell culture treated with an immunosuppressive agent. In embodiments, the source cell is substantially non-immunogenic, e.g., using an assay described herein. In embodiments, the source cell comprises an exogenous agent, e.g., a therapeutic agent. In embodiments, the source cell is a recombinant cell.

In embodiments, the cytobiologic further comprises an exogenous agent, e.g., a therapeutic agent, e.g., a protein or a nucleic acid (e.g., a DNA, a chromosome (e.g. a human artificial chromosome), an RNA, e.g., an mRNA or miRNA). In embodiments, the exogenous agent is present at at least, or no more than, 10, 20, 50, 100, 200, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies comprised by the cytobiologic. In embodiments, the exogenous agent is present at at an average level of at least, or no more than, 10, 20, 50, 100, 200, 500, 1,000, 2,000, 5,000, 10,000, 20,000, 50,000, 100,000, 200,000, 500,000 or 1,000,000 copies per cytobiologic. In embodiments, the cytobiologic has an altered, e.g., increased or decreased level of one or more endogenous molecule, e.g., protein or nucleic acid, e.g., due to treatment of the source cell, e.g., mammalian source cell with a siRNA or gene editing enzyme. In embodiments, the endogenous molecule is present at at least, or no more than, 10, 20, 50, 100, 200, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies comprised by the cytobiologic. In embodiments, the endogenous molecule is present at an average level of at least, or no more than, 10, 20, 50, 100, 200, 500, 1,000, 2,000, 5,000, 10,000, 20,000, 50,000, 100,000, 200,000, 500,000 or 1,000,000 copies per cytobiologic. In embodiments, the endogenous molecule (e.g., an RNA or protein) is present at a concentration of at least 1, 2, 3, 4, 5, 10, 20, 50, 100, 500, 10³, 5.0×10³, 10⁴, 5.0×10⁴, 10⁵, 5.0×10⁵, 10⁶, 5.0×10⁶, 1.0×10⁷, 5.0×10⁷, or 1.0×10⁸, greater than its concentration in the source cell.

In embodiments, the agent, e.g., therapeutic agent, is selected from a protein, protein complex (e.g., comprising at least 2, 3, 4, 5, 10, 20, or 50 proteins, e.g., at least at least 2, 3, 4, 5, 10, 20, or 50 different proteins) polypeptide, nucleic acid (e.g., DNA, chromosome, or RNA, e.g., mRNA, siRNA, or miRNA) or small molecule. In embodiments, the exogenous agent comprises a site-specific nuclease, e.g., Cas9 molecule, TALEN, or ZFN.

In embodiments, the cytobiologic comprises a fusogen. In embodiments, the fusogen is a viral fusogen or a mammalian fusogen. In embodiments, the fusogen is a protein fusogen, lipid fusogen, chemical fusogen, or small molecule fusogen.

In embodiments, the cytobiologic binds to or acts on a target cell. In embodiments, the target cell is other than a HeLa cell, or the target cell is not transformed or immortalized.

In some embodiments involving cytobiologic compositions, the plurality of cytobiologics are the same. In some embodiments, the plurality of cytobiologics are different. In some embodiments the plurality of cytobiologics are from one or more source cells. In some embodiments at least 50%, 60%, 70%, 80%, 90%, 95%, or 99% of cytobiologics in the plurality have a diameter within 10%, 20%, 30%, 40%, or 50% of the mean diameter of the cytobiologics in the cytobiologic composition. In some embodiments at least 50%, 60%, 70%, 80%, 90%, 95%, or 99% of cytobiologics in the plurality have a volume within 10%, 20%, 30%, 40%, or 50% of the mean volume of the cytobiologics in the cytobiologics composition. In some embodiments, the cytobiologic composition has less than about 90%, 80%, 70%, 60%, 50%, 40%, 30%, 20%, 10%, 5%, variability in size distribution within 10%, 50%, or 90% of the source cell population variability in size distribution, e.g., based on Example 19. In some embodiments, at least 50%, 60%, 70%, 80%, 90%, 95%, or 99% of cytobiologics in the plurality have a copy number of the therapeutic agent within 10%, 20%, 30%, 40%, 50%, 60%, 70%, 80%, or 90% of the mean therapeutic agent copy number in the cytobiologics in the cytobiologic composition. In some embodiments, the cytobiologic composition comprises at least 10⁵, 10⁶, 10⁷, 10⁸, 10⁹, or 10¹⁰ cytobiologics. In some embodiments, the cytobiologic composition is in a volume of at least 1 ul, 2 ul, 5 ul, 10 ul, 20 ul, 50 ul, 100 ul, 200 ul, 500 ul, 1 ml, 2 ml, 5 ml, or 10 ml.

In embodiments, a pharmaceutical composition described herein has one or more of the following characteristics:
a) the pharmaceutical composition meets a pharmaceutical or good manufacturing practices (GMP) standard;
b) the pharmaceutical composition was made according to good manufacturing practices (GMP);
c) the pharmaceutical composition has a pathogen level below a predetermined reference value, e.g., is substantially free of pathogens;
d) the pharmaceutical composition has a contaminant level below a predetermined reference value, e.g., is substantially free of contaminants; or
e) the pharmaceutical composition has low immunogenicity, e.g., as described herein.

In embodiments, the biological function is selected from:
a) modulating, e.g., inhibiting or stimulating, an enzyme;
b) modulating, e.g., increasing or decreasing levels of, a molecule (e.g., a protein, nucleic acid, or metabolite, drug, or toxin) in the subject, e.g., by inhibiting or stimulating synthesis or by inhibiting or stimulating degradation of the factor;
c) modulating, e.g., increasing or decreasing, viability of a target cell or tissue; or d) modulating a protein state, e.g., increasing or decreasing phosphorylation of the protein, or modulating the protein conformation;
e) promoting healing of an injury;
f) modulating, e.g., increasing or decreasing, an interaction between two cells;
g) modulating, e.g., promoting or inhibiting, cell differentiation;
h) altering distribution of a factor (e.g., a protein, nucleic acid, metabolite, drug, or toxin) in the subject;
i) modulating, e.g. increasing or decreasing, an immune response; or
j) modulating, e.g. increasing or decreasing, recruitment of cells to a target tissue.

In some embodiments of the therapeutic methods herein, the plurality of cytobiologics has a local effect. In some embodiments, the plurality of cytobiologics has a distal effect.

In some embodiments, the subject has a cancer, an inflammatory disorder, autoimmune disease, a chronic disease, inflammation, damaged organ function, an infectious disease, metabolic disease, degenerative disorder, genetic disease (e.g., a genetic deficiency or a dominant genetic disorder), or an injury. In some embodiments, the subject has an infectious disease and the cytobiologic comprises an antigen for the infectious disease. In some embodiments, the subject has a genetic deficiency and the cytobiologic comprises a protein for which the subject is deficient, or a nucleic acid (e.g., mRNA) encoding the protein, or a DNA encoding the protein, or a chromosome encoding the protein, or a nucleus comprising a nucleic acid encoding the protein. In some embodiments, the subject has a dominant genetic disorder, and the cytobiologic comprises a nucleic acid inhibitor (e.g., siRNA or miRNA) of the dominant mutant allele. In some embodiments, the subject has a dominant genetic disorder, and the cytobiologic comprises a nucleic acid inhibitor (e.g., siRNA or miRNA) of the dominant mutant allele, and the cytobiologic also comprises an mRNA encoding a non-mutated allele of the mutated gene that is not targeted by the nucleic acid inhibitor. In some embodiments, the subject is in need of vaccination. In some embodiments, the subject is in need of regeneration, e.g., of an injured site.

In some embodiments, the cytobiologic composition is administered to the subject at least 1, 2, 3, 4, or 5 times.

In some embodiments, the cytobiologic composition is administered to the subject systemically (e.g., orally, parenterally, subcutaneously, intravenously, intramuscularly, intraperitoneally) or locally. In some embodiments, the cytobiologic composition is administered to the subject such that the cytobiologic composition reaches a target tissue selected from liver, lungs, heart, spleen, pancreas, gastrointestinal tract, kidney, testes, ovaries, brain, reproductive organs, central nervous system, peripheral nervous system, skeletal muscle, endothelium, inner ear, or eye. In some embodiments (e.g., wherein the subject has an autoimmune disease), the cytobiologic composition is co-administered with an immunosuppressive agent, e.g., a glucocorticoid, cytostatic, antibody, or immunophilin modulator. In some embodiments (e.g., wherein the subject has a cancer or an infectious disease), the cytobiologic composition is co-administered with an immunostimulatory agent, e.g., an adjuvant, interleukin, cytokine, or chemokine. In some embodiments, administration of the cytobiologic composition results in upregulation or downregulation of a gene in a target cell in the subject, e.g., wherein the cytobiologic comprises a transcriptional activator or repressor, a translational activator or repressor, or an epigenetic activator or repressor.

In some embodiments of the methods of making herein, the method comprises inactivating the nucleus of the source cell.

In embodiments, the cytobiologic composition comprises at least 10⁵, 10⁶, 10⁷, 10⁸, 10⁹, 10¹⁰, 10¹¹, 10¹², 10¹³, 10¹⁴, or 10¹⁵ cytobiologics. In embodiments, the cytobiologic composition comprises at least 10 ml, 20 ml, 50 ml, 100 ml, 200 ml, 500 ml, 1 L, 2 L, 5 L, 10 L, 20 L, or 50 L. In embodiments, the method comprises enucleating the mammalian cell, e.g., by chemical enucleation, use of mechanical force e.g. use of a filter or centrifuge, at least partial disruption of the cytoskeleton, or a combination thereof. In embodiments, the method comprises expressing a fusogen or other membrane protein in the source cell. In embodiments, the method comprises one or more of: vesiculation, hypotonic treatment, extrusion, or centrifugation. In embodiments, the method comprises genetically expressing an exogenous agent in the source cell or loading the exogenous agent into the source cell or cytobiologic. In embodiments, the method comprises contacting the source cell with DNA encoding a polypeptide agent, e.g., before inactivating the nucleus, e.g., enucleating the source cell. In embodiments, the method comprises contacting the source cell with RNA encoding a polypeptide agent, e.g., before or after inactivating the nucleus, e.g., enucleating the source cell. In embodiments, the method comprises introducing a therapeutic agent (e.g., a nucleic acid or protein) into a cytobiologic, e.g., by electroporation.

In embodiments, the cytobiologic is from a mammalian cell having a modified genome, e.g., to reduce immunogenicity (e.g., by genome editing, e.g., to remove an MHC protein). In embodiments, the method further comprises contacting the source cell of step a) with an immunosuppressive agent, e.g., before or after inactivating the nucleus, e.g., enucleating the cell.

In some embodiments, if a detectable level, e.g., a value above a reference value, is determined, a sample containing the plurality of cytobiologics or cytobiologic composition is discarded.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of May 8, 2017. When one gene or protein references a plurality of sequence accession numbers, all of the sequence variants are encompassed. In addition, the materials, methods, and examples are illustrative only and not intended to be limiting.

### BRIEF DESCRIPTION OF THE DRAWINGS

The following detailed description of the invention will be better understood when read in conjunction with the appended drawings. For the purpose of illustrating the invention, there are shown in the drawings described herein certain embodiments, which are presently exemplified. It should be understood, however, that the invention is not limited to the precise arrangement and instrumentalities of the embodiments shown in the drawings.

FIG. 1 quantifies staining of cytobiologics with a dye for endoplasmic reticulum.

FIG. 2 quantifies staining of cytobiologics with a dye for mitochondria.

FIG. 3 quantifies staining of cytobiologics with a dye for lysosomes.

FIG. 4 quantifies staining of cytobiologics with a dye for F-actin.

FIG. 5 shows microscopy images of the indicated tissues from mice injected with cytobiologics. White indicates represent RFP-fluorescent cells, indicating delivery of a protein cargo to the cells in vivo.

FIG. 6 shows microscopy images of tdTomato fluorescence in murine muscle tissue, indicating delivery of a protein cargo to muscle cells by cytobiologics.

FIG. 7 is a series of images showing successful delivery of fusogenic cytobiologics to murine tissues in vivo by the indicated routes of administration, resulting in expression of luciferase by targeted cells.

### DETAILED DESCRIPTION

The invention describes cytobiologics, e.g., enucleated cells or cells having an inactivated nucleus. The cytobiologic can be used, e.g., for delivery of a cargo in the lumen or lipid bilayer of the cytobiologic to a target cell. Cargo includes, e.g., therapeutic proteins, nucleic acids, and small molecules.

### Definitions

As used herein, a “cell membrane” refers to a membrane derived from a cell, e.g., a source cell or a target cell.

As used herein, a “chondrisome” is a subcellular apparatus derived and isolated or purified from the mitochondrial network of a natural cell or tissue source. A “chondrisome preparation” has bioactivity (can interact with, or have an effect on, a cell or tissue) and/or pharmaceutical activity.

As used herein, “cytobiologic” refers to a portion of a cell that comprises a lumen and a cell membrane, or a cell having partial or complete nuclear inactivation. In some embodiments, the cytobiologic comprises one or more of a cytoskeleton component, an organelle, and a ribosome. In embodiments, the cytobiologic is an enucleated cell, a microvesicle, or a cell ghost.

As used herein, “cytosol” refers to the aqueous component of the cytoplasm of a cell. The cytosol may comprise proteins, RNA, metabolites, and ions.

An “exogenous agent” as used herein, refers to an agent that: i) does not naturally exist, such as a protein that has a sequence that is altered (e.g., by insertion, deletion, or substitution) relative to an endogenous protein, or ii) does not naturally occur in the naturally occurring source cell of the cytobiologic in which the exogenous agent is disposed.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., the lumen of the cytobiologic and a cytoplasm of a target cell). In some embodiments, the fusogen comprises a complex of two or more proteins, e.g., wherein neither protein has fusogenic activity alone.

As used herein, “membrane enclosed preparation” refers to a bilayer of amphipathic lipids enclosing a cargo in a lumen or cavity. In some embodiments, the cargo is exogenous to the lumen or cavity. In other embodiments, the cargo is endogenous to the lumen or cavity, e.g., endogenous to a source cell.

As used herein, “mitochondrial biogenesis” denotes the process of increasing biomass of mitochondria. Mitochondrial biogenesis includes increasing the number and/or size of mitochondria in a cell.

As used herein, the term “purified” means altered or removed from the natural state. For example, a cell or cell fragment naturally present in a living animal is not “purified,” but the same cell or cell fragment partially or completely separated from the coexisting materials of its natural state is “purified.” A purified cytobiologic composition can exist in substantially pure form, or can exist in a non-native environment such as, for example, a culture medium such as a culture medium comprising cells.

As used herein, a “source cell” refers to a cell from which a cytobiologic is derived.

### Cytobiologics

In one embodiment, the cytobiologic is a vesicle from MSCs or astrocytes.

In one embodiment, the cytobiologic is an exosome.

Exemplary exosomes and other membrane-enclosed bodies are described, e.g., in US2016137716, which is herein incorporated by reference in its entirety. In some embodiments, the cytobiologic comprises a vesicle that is, for instance, obtainable from a cell, for instance a microvesicle, an exosome, an apoptotic body (from apoptotic cells), a microparticle (which may be derived from e.g. platelets), an ectosome (derivable from, e.g., neutrophiles and monocytes in serum), a prostatosome (obtainable from prostate cancer cells), a cardiosome (derivable from cardiac cells), and the like.

Exemplary exosomes and other membrane-enclosed bodies are also described in WO/2017/161010, WO/2016/077639, US20160168572, US20150290343, and US20070298118, each of which is incorporated by reference herein in its entirety. In some embodiments, the cytobiologic comprises an extracellular vesicle, nanovesicle, or exosome. In embodiment the cytobiologic comprises an extracellular vesicle, e.g., a cell-derived vesicle comprising a membrane that encloses an internal space and has a smaller diameter than the cell from which it is derived. In embodiments the extracellular vesicle has a diameter from 20 nm to 1000 nm. In embodiments the cytobiologic comprises an apoptotic body, a fragment of a cell, a vesicle derived from a cell by direct or indirect manipulation, a vesiculated organelle, and a vesicle produced by a living cell (e.g., by direct plasma membrane budding or fusion of the late endosome with the plasma membrane). In embodiments the extracellular vesicle is derived from a living or dead organism, explanted tissues or organs, or cultured cells. In embodiments, the cytobiologic comprises a nanovesicle, e.g., a cell-derived small (e.g., between 20-250 nm in diameter, or 30-150 nm in diameter) vesicle comprising a membrane that encloses an internal space, and which is generated from said cell by direct or indirect manipulation. The production of nanovesicles can, in some instances, result in the destruction of the source cell. The nanovesicle may comprise a lipid or fatty acid and polypeptide. In embodiments, the cytobiologic comprises an exosome. In embodiments, the exosome is a cell-derived small (e.g., between 20-300 nm in diameter, or 40-200 nm in diameter) vesicle comprising a membrane that encloses an internal space, and which is generated from said cell by direct plasma membrane budding or by fusion of the late endosome with the plasma membrane. In embodiments, production of exosomes does not result in the destruction of the source cell. In embodiments, the exosome comprises lipid or fatty acid and polypeptide.

Exemplary exosomes and other membrane-enclosed bodies are also described in US 20160354313, which is herein incorporated by reference in its entirety. In embodiments, the cytobiologic comprises a Biocompatible Delivery Module, an exosome (e.g., about 30 nm to about 200 nm in diameter), a microvesicle (e.g., about 100 nm to about 2000 nm in diameter) an apoptotic body (e.g., about 300 nm to about 2000 nm in diameter), a membrane particle, a membrane vesicle, an exosome-like vesicle, an ectosome-like vesicle, an ectosome, or an exovesicle.

In one embodiment, the cytobiologic is microvesicle. In one embodiment, the cytobiologic is a cell ghost. In one embodiment, the vesicle is a plasma membrane vesicle, e.g. a giant plasma membrane vesicle.

Cytobiologics can be made from several different types of lipids, e.g., amphipathic lipids, such as phospholipids. The cytobiologic may comprise a lipid bilayer as the outermost surface. This bilayer may be comprised of one or more lipids of the same or different type. Examples include without limitation phospholipids such as phosphocholines and phosphoinositols. Specific examples include without limitation DMPC, DOPC, and DSPC.

A cytobiologic may be mainly comprised of natural phospholipids and lipids such as 1,2-distearoryl-sn-glycero-3-phosphatidyl choline (DSPC), sphingomyelin, egg phosphatidylcholines and monosialoganglioside. In embodiments, a cytobiologic comprises only phospholipids and is less stable in plasma. However, manipulation of the lipid membrane with cholesterol can, in embodiments, increase stability and reduce rapid release of the encapsulated bioactive compound into the plasma. In some embodiments, the cytobiologic comprises 1,2-dioleoyl-sn-glycero-3-phosphoethanolamine (DOPE), e.g., to increase stability (see, e.g., Spuch and Navarro, Journal of Drug Delivery, vol. 2011, Article ID 469679, 12 pages, 2011. doi:10.1155/2011/469679 for review).

In some embodiments, cytobiologics comprise or are enriched for lipids that affect membrane curvature (see, e.g., Thiam et al., Nature Reviews Molecular Cell Biology, 14(12): 775-785, 2013). Some lipids have a small hydrophilic head group and large hydrophobic tails, which facilitate the formation of a fusion pore by concentrating in a local region. In some embodiments, cytobiologics comprise or are enriched for negative-curvature lipids, such as cholesterol, phosphatidylethanolamine (PE), diglyceride (DAG), phosphatidic acid (PA), fatty acid (FA). In some embodiments, cytobiologics do not comprise, are depleted of, or have few positive-curvature lipids, such as lysophosphatidylcholine (LPC), phosphatidylinositol (PtdIns), lysophosphatidic acid (LPA), lysophosphatidylethanolamine (LPE), monoacylglycerol (MAG).

In some embodiments, the lipids are added to a cytobiologic. In some embodiments, the lipids are added to source cells in culture which incorporate the lipids into their membranes prior to or during the formation of a cytobiologic. In some embodiments, the lipids are added to the cells or cytobiologic in the form of a liposome. In some embodiments, methyl-betacyclodextrane (mβ-CD) is used to enrich or deplete lipids (see, e.g., Kainu et al, Journal of Lipid Research, 51(12): 3533-3541, 2010).

Cytobiologics may comprise without limitation DOPE (dioleoylphosphatidylethanolamine), DOTMA, DOTAP, DOTIM, DDAB, alone or together with cholesterol to yield DOPE and cholesterol, DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although formation of cytobiologics can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

In another embodiment, lipids may be used to form cytobiologics. Lipids including, but are not limited to, DLin-KC2-DMA4, C12-200 and colipids disteroylphosphatidyl choline, cholesterol, and PEG-DMG may be formulated (see, e.g., Novobrantseva, Molecular Therapy-Nucleic Acids (2012) 1, e4; doi:10.1038/mtna.2011.3) using a spontaneous vesicle formation procedure. Tekmira publications describe various aspects of lipid vesicles and lipid vesicle formulations (see, e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos. 1766035; 1519714; 1781593 and 1664316), all of which are herein incorporated by reference and may be used and/or adapted to the present invention.

In some embodiments, a cytobiologic described herein may include one or more polymers. The polymers may be biodegradable. Biodegradable polymer vesicles may be synthesized using methods known in the art. Exemplary methods for synthesizing polymer vesicles are described by Bershteyn et al., Soft Matter 4:1787-1787, 2008 and in US 2008/0014144 A1, the specific teachings of which relating to microparticle synthesis are incorporated herein by reference.

Exemplary synthetic polymers which can be used include without limitation aliphatic polyesters, polyethylene glycol (PEG), poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone), and natural polymers such as albumin, alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

### Fusogens

In some embodiments, the cytobiologic described herein (e.g., comprising a vesicle or a portion of a cell) includes one or more fusogens, e.g., to facilitate the fusion of the cytobiologic to a membrane, e.g., a cell membrane. Also these compositions may include surface modifications made during or after synthesis to include one or more fusogens, e.g., fusogens may be complementary to a target cell. The surface modification may comprise a modification to the membrane, e.g., insertion of a lipid or protein into the membrane. Fusogens include without limitation protein based, lipid based, and chemical based fusogens.

In some embodiments, the cytobiologic does not comprise a fusogen. In some embodiments, the cytobiologic does not comprise an exogenous fusogen.

### Methods of Generating Cytobiologics

Compositions of cytobiologics may be generated from cells in culture, for example cultured mammalian cells, e.g., cultured human cells. The cells may be progenitor cells or non-progenitor (e.g., differentiated) cells. The cells may be primary cells or cell lines (e.g., a mammalian, e.g., human, cell line described herein). In embodiments, the cultured cells are progenitor cells, e.g., bone marrow stromal cells, marrow derived adult progenitor cells (MAPCs), endothelial progenitor cells (EPC), blast cells, intermediate progenitor cells formed in the subventricular zone, neural stem cells, muscle stem cells, satellite cells, liver stem cells, hematopoietic stem cells, bone marrow stromal cells, epidermal stem cells, embryonic stem cells, mesenchymal stem cells, umbilical cord stem cells, precursor cells, muscle precursor cells, myoblast, cardiomyoblast, neural precursor cells, glial precursor cells, neuronal precursor cells, hepatoblasts.

In some embodiments, the source cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), or an immortalized cell (e.g., HeLa, HEK293, HFF-1, MRC-5, WI-38, IMR 90, IMR 91, PER.C6, HT-1080, or BJ cell).

The cultured cells may be from epithelial, connective, muscular, or nervous tissue or cells, and combinations thereof. Cytobiologics can be generated from cultured cells from any eukaryotic (e.g., mammalian) organ system, for example, from the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof. In embodiments, the cells are from a highly mitotic tissue (e.g., a highly mitotic healthy tissue, such as epithelium, embryonic tissue, bone marrow, intestinal crypts). In embodiments, the tissue sample is a highly metabolic tissue (e.g., skeletal tissue, neural tissue, cardiomyocytes).

In some embodiments, the cells are from a young donor, e.g., a donor 25 years, 20 years, 18 years, 16 years, 12 years, 10 years, 8 years of age, 5 years of age, 1 year of age, or less. In some embodiments, the cells are from fetal tissue.

In some embodiments, the cells are derived from a subject and administered to the same subject or a subject with a similar genetic signature (e.g., MHC-matched).

In certain embodiments, the cells have telomeres of average size greater than 3000, 4000, 5000, 6000, 7000, 8000, 9000, or 10000 nucleotides in length (e.g., between 4,000-10,000 nucleotides in length, between 6,000-10,000 nucleotides in length).

Cytobiologics may be generated from cells generally cultured according to methods known in the art. In some embodiments, the cells may be cultured in 2 or more “phases”, e.g., a growth phase, wherein the cells are cultured under conditions to multiply and increase biomass of the culture, and a “production” phase, wherein the cells are cultured under conditions to alter cell phenotype (e.g., to maximize mitochondrial phenotype, to increase number or size of mitochondria, to increase oxidative phosphorylation status). There may also be an “expression” phase, wherein the cells are cultured under conditions to maximize expression of an agent, e.g., an exogenous agent, and to restrict unwanted fusion in other phases.

In some embodiments, cytobiologics are generated from cells synchronized, e.g., during a growth phase or the production phase. For example, cells may be synchronized at G1 phase by elimination of serum from the culture medium (e.g., for about 12-24 hours) or by the use in the culture media of DNA synthesis inhibitors such as thymidine, aminopterin, hydroxyurea and cytosine arabinoside. Additional methods for mammalian cell cycle synchronization are known and disclosed, e.g., in Rosner et al. 2013. Nature Protocols 8:602-626 (specifically Table 1 in Rosner).

In some embodiments, the cells can be evaluated and optionally enriched for a desirable phenotype or genotype for use as a source for cytobiologic composition as described herein. For example, cells can be evaluated and optionally enriched, e.g., before culturing, during culturing (e.g., during a growth phase or a production phase) or after culturing but before cytobiologic production, for example, for one or more of: membrane potential (e.g., a membrane potential of −5 to −200 mV; cardiolipin content (e.g., between 1-20% of total lipid); cholesterol, phosphatidylethanolamine (PE), diglyceride (DAG), phosphatidic acid (PA), or fatty acid (FA) content; genetic quality >80%, >85%, >90%; or cargo expression or content.

In some embodiments, cytobiologics are generated from a cell clone identified, chosen, or selected based on a desirable phenotype or genotype for use as a source for cytobiologic composition described herein. For example, a cell clone is identified, chosen, or selected based on low mitochondrial mutation load, long telomere length, differentiation state, or a particular genetic signature (e.g., a genetic signature to match a recipient).

A cytobiologic composition described herein may be comprised of cytobiologics from one cellular or tissue source, or from a combination of sources. For example, a cytobiologic composition may comprise cytobiologics from xenogeneic sources (e.g., animals, tissue culture of the aforementioned species' cells), allogeneic, autologous, from specific tissues resulting in different protein concentrations and distributions (liver, skeletal, neural, adipose, etc.), from cells of different metabolic states (e.g., glycolytic, respiring). A composition may also comprise cytobiologics in different metabolic states, e.g. coupled or uncoupled, as described elsewhere herein.

In some embodiments, cytobiologics are generated by inducing budding of an exosome, microvesicle, membrane vesicle, extracellular membrane vesicle, plasma membrane vesicle, giant plasma membrane vesicle, apoptotic body, mitoparticle, pyrenocyte, lysosome, or other membrane enclosed vesicle.

In some embodiments, cytobiologicss are generated by inducing cell enucleation. Enucleation may be performed using assays such as genetic, chemical (e.g., using Actinomycin D, see Bayona-Bafaluyet al., “A chemical enucleation method for the transfer of mitochondrial DNA to ρ° cells” Nucleic Acids Res. 2003 Aug. 15; 31(16): e98), mechanical methods (e.g., squeezing or aspiration, see Lee et al., “A comparative study on the efficiency of two enucleation methods in pig somatic cell nuclear transfer: effects of the squeezing and the aspiration methods.” Anim Biotechnol. 2008; 19(2):71-9), or combinations thereof. Enucleation refers not only to a complete removal of the nucleus but also the displacement of the nucleus from its typical location such that the cell contains the nucleus but it is non-functional.

In embodiments, making a cytobiologic comprises producing cell ghosts, giant plasma membrane vesicle, or apoptotic bodies. In embodiments, a cytobiologic composition comprises one or more of cell ghosts, giant plasma membrane vesicle, and apoptotic bodies.

In some embodiments, cytobiologics are generated by inducing cell fragmentation. In some embodiments, cell fragmentation can be performed using the following methods, including, but not limited to: chemical methods, mechanical methods (e.g., centrifugation (e.g., ultracentrifugation, or density centrifugation), freeze-thaw, or sonication), or combinations thereof.

In an embodiment, a cytobiologic can be generated from a source cell, e.g., as described herein, by any one, all of, or a combination of the following methods:

i) inducing budding of a mitoparticle, exosome, or other membrane enclosed vesicle;
ii) inducing nuclear inactivation, e.g., enucleation, by any of the following methods or a combination thereof:

a) a genetic method;

b) a chemical method, e.g., using Actinomycin D; or

c) a mechanical method, e.g., squeezing or aspiration; or

iii) inducing cell fragmentation, e.g., by any of the following methods or a combination thereof:

a) a chemical method;

b) a mechanical method, e.g., centrifugation (e.g., ultracentrifugation or density centrifugation); freeze thaw; or sonication.

For avoidance of doubt, it is understood that in many cases the source cell actually used to make the cytobiologic will not be available for testing after the cytobiologic is made. Thus, a comparison between a source cell and a cytobiologic does not need to assay the source cell that was actually modified (e.g., enucleated) to make the cytobiologic. Rather, cells otherwise similar to the source cell, e.g., from the same culture, the same genotype same tissue type, or any combination thereof, can be assayed instead.

### Modifications to Cells Prior to Cytobiologic Generation

In one aspect, a modification is made to a cell, such as modification of a subject, tissue or cell, prior to cytobiologic generation. Such modifications can be effective to, e.g., alter structure or function of the cargo, or structure or function of the target cell.

### Physical Modifications

In some embodiments, a cell is physically modified prior to generating the cytobiologic.

In some embodiments, a cell is treated with a chemical agent prior to generating the cytobiologic.

In some embodiments, the cell is physically modified prior to generating the cytobiologic with one or more covalent or non-covalent attachment sites for synthetic or endogenous small molecules or lipids on the cell surface that enhance targeting of the cytobiologic to an organ, tissues, or cell-type.

In embodiments, a cytobiologic comprises increased or decreased levels of an endogenous molecule. For instance, the cytobiologic may comprise an endogenous molecule that also naturally occurs in the naturally occurring source cell but at a higher or lower level than in the cytobiologic. In some embodiments, the polypeptide is expressed from an exogenous nucleic acid in the source cell or cytobiologic. In some embodiments, the polypeptide is isolated from a source and loaded into or conjugated to a source cell or cytobiologic.

In some embodiments, a cell is treated with a chemical agent prior to generating the cytobiologic to increase the expression or activity of an endogenous agent in the cell. In one embodiment, the small molecule may increase expression or activity of a transcriptional activator of the endogenous agent. In another embodiment, the small molecule may decrease expression or activity of a transcriptional repressor of the endogenous agent. In yet another embodiment, the small molecule is an epigenetic modifier that increases expression of the endogenous agent.

In some embodiments, the cell is physically modified with, e.g., CRISPR activators, to prior to generating the cytobiologic to add or increase the concentration of an agent.

In some embodiments, the cell is physically modified to increase or decrease the quantity, or enhance the structure or function of organelles, e.g., mitochondria, Golgi apparatus, endoplasmic reticulum, intracellular vesicles (such as lysosomes, autophagosomes).

### Genetic Modifications

In some embodiments, a cell is genetically modified prior to generating the cytobiologic to increase the expression of an endogenous agent in the cell. In one embodiment, the genetic modification may increase expression or activity of a transcriptional activator of the endogenous agent. In another embodiment, the genetic modification may decrease expression or activity of a transcriptional repressor of the endogenous agent. In some embodiments the activator or repressor is a nuclease-inactive cas9 (dCas9) linked to a transcriptional activator or repressor that is targeted to the endogenous agent by a guide RNA. In yet another embodiment, the genetic modification epigenetically modifies an endogenous gene to increase its expression. In some embodiments the epigenetic activator a nuclease-inactive cas9 (dCas9) linked to an epigenetic modifier that is targeted to the endogenous agent by a guide RNA.

In some embodiments, a cell is genetically modified prior to generating the cytobiologic to increase the expression of an exogenous agent in the cell, e.g., delivery of a transgene. In some embodiments, a nucleic acid, e.g., DNA, mRNA or siRNA, is transferred to the cell prior to generating the cytobiologic, e.g., to increase or decrease the expression of a cell surface molecule (protein, glycan, lipid or low molecular weight molecule) used for organ, tissue, or cell targeting. In some embodiments, the nucleic acid targets a repressor of an agent, e.g., an shRNA, siRNA construct. In some embodiments, the nucleic acid encodes an inhibitor of a repressor or an agent.

In some embodiments, the method comprises introducing an exogenous nucleic acid encoding an agent into the source cell. The exogenous nucleic acid may be, e.g., DNA or RNA. In some embodiments, the exogenous DNA may be linear DNA, circular DNA, or an artificial chromosome. In some embodiments the DNA is maintained episomally. In some embodiments the DNA is integrated into the genome. The exogenous RNA may be chemically modified RNA, e.g., may comprise one or more backbone modification, sugar modifications, noncanonical bases, or caps. Backbone modifications include, e.g., phosphorothioate, N3′ phosphoramidite, boranophosphate, phosphonoacetate, thio-PACE, morpholino phosphoramidites, or PNA. Sugar modifications include, e.g., 2′-O-Me, 2′F, 2′F-ANA, LNA, UNA, and 2′-O-MOE. Noncanonical bases include, e.g., 5-bromo-U, and 5-iodo-U, 2,6-diaminopurine, C-5 propynyl pyrimidine, difluorotoluene, difluorobenzene, dichlorobenzene, 2-thiouridine, pseudouridine, and dihydrouridine. Caps include, e.g., ARCA. Additional modifications are discussed, e.g., in Deleavey et al., “Designing Chemically Modified Oligonucleotides for Targeted Gene Silencing” Chemistry & Biology Volume 19, Issue 8, 24 Aug. 2012, Pages 937-954, which is herein incorporated by reference in its entirety.

In some embodiments, a cell is genetically modified prior to generating the cytobiologic to alter (i.e., upregulate or downregulate) the expression of signaling pathways (e.g., the Wnt/Beta-catenin pathway). In some embodiments, a cell is genetically modified prior to generating the cytobiologic to alter (e.g., upregulate or downregulate) the expression of a gene or genes of interest. In some embodiments, a cell is genetically modified prior to generating the cytobiologic to alter (e.g., upregulate or downregulate) the expression of a nucleic acid (e.g. a miRNA or mRNA) or nucleic acids of interest. In some embodiments, nucleic acids, e.g., DNA, mRNA or siRNA, are transferred to the cell prior to generating the cytobiologic, e.g., to increase or decrease the expression of signaling pathways, genes, or nucleic acids. In some embodiments, the nucleic acid targets a repressor of a signaling pathway, gene, or nucleic acid, or represses a signaling pathway, gene, or nucleic acid. In some embodiments, the nucleic acid encodes a transcription factor that upregulates or downregulates a signaling pathway, gene, or nucleic acid. In some embodiments the activator or repressor is a nuclease-inactive cas9 (dCas9) linked to a transcriptional activator or repressor that is targeted to the signaling pathway, gene, or nucleic acid by a guide RNA. In yet another embodiment, the genetic modification epigenetically modifies an endogenous signaling pathway, gene, or nucleic acid to its expression. In some embodiments the epigenetic activator a nuclease-inactive cas9 (dCas9) linked to a epigenetic modifier that is targeted to the signaling pathway, gene, or nucleic acid by a guide RNA. In some embodiments, a cell's DNA is edited prior to generating the cytobiologic to alter (e.g., upregulate or downregulate) the expression of signaling pathways (e.g. the Wnt/Beta-catenin pathway), gene, or nucleic acid. In some embodiments, the DNA is edited using a guide RNA and CRISPR-Cas9/Cpf1 or other gene editing technology.

A cell may be genetically modified using recombinant methods. A nucleic acid sequence coding for a desired gene can be obtained using recombinant methods, such as, for example by screening libraries from cells expressing the gene, by deriving the gene from a vector known to include the same, or by isolating directly from cells and tissues containing the same, using standard techniques. Alternatively, a gene of interest can be produced synthetically, rather than cloned.

Expression of natural or synthetic nucleic acids is typically achieved by operably linking a nucleic acid encoding the gene of interest to a promoter, and incorporating the construct into an expression vector. The vectors can be suitable for replication and integration in eukaryotes. Typical cloning vectors contain transcription and translation terminators, initiation sequences, and promoters useful for expression of the desired nucleic acid sequence.

In some embodiments, a cell may be genetically modified with one or more expression regions, e.g., a gene. In some embodiments, the cell may be genetically modified with an exogenous gene (e.g., capable of expressing an exogenous gene product such as an RNA or a polypeptide product) and/or an exogenous regulatory nucleic acid. In some embodiments, the cell may be genetically modified with an exogenous sequence encoding a gene product that is endogenous to a target cell and/or an exogenous regulatory nucleic acid capable of modulating expression of an endogenous gene. In some embodiments, the cell may be genetically modified with an exogenous gene and/or a regulatory nucleic acid that modulates expression of an exogenous gene. In some embodiments, the cell may be genetically modified with an exogenous gene and/or a regulatory nucleic acid that modulates expression of an endogenous gene. It will be understood by one of skill in the art that the cell described herein may be genetically modified to express a variety of exogenous genes that encode proteins or regulatory molecules, which may, e.g., act on a gene product of the endogenous or exogenous genome of a target cell. In some embodiments, such genes confer characteristics to the cytobiologic, e.g., modulate its activity towards a target cell. In some embodiments, the cell may be genetically modified to express an endogenous gene and/or regulatory nucleic acid. In some embodiments, the endogenous gene or regulatory nucleic acid modulates the expression of other endogenous genes. In some embodiments, the cell may be genetically modified to express an endogenous gene and/or regulatory nucleic acid which is expressed differently (e.g., inducibly, tissue-specifically, constitutively, or at a higher or lower level) than a version of the endogenous gene and/or regulatory nucleic acid on other chromosomes.

The promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a tissue-specific promoter, metallothionine promoter, a glucocorticoid promoter, a progesterone promoter, and a tetracycline promoter. In some embodiments, expression of an agent is upregulated before cytobiologics are generated, e.g., 3, 6, 9, 12, 24, 26, 48, 60, or 72 hours before cytobiologics are generated.

The expression vector to be introduced into the source can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes may be used for identifying potentially transfected cells and for evaluating the functionality of regulatory sequences. In general, a reporter gene is a gene that is not present in or expressed by the recipient source and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, a cell may be genetically modified to alter expression of one or more proteins. Expression of the one or more proteins may be modified for a specific time, e.g., development or differentiation state of the source. In one embodiment, the invention includes cytobiologics generated from a source of cells genetically modified to alter expression of one or more proteins. Expression of the one or more proteins may be restricted to a specific location(s) or widespread throughout the source.

In some embodiments, cells may be engineered to express a cytosolic enzyme (e.g., proteases, phosphatases, kinases, demethylases, methyltransferases, acetylases) that targets a protein. In some embodiments, the cytosolic enzyme affects one or more proteins by altering post-translational modifications. Post-translational protein modifications of proteins may affect responsiveness to nutrient availability and redox conditions, and protein-protein interactions. In one embodiment, the invention includes a cytobiologic comprising one or more proteins with altered post-translational modifications, e.g., an increase or a decrease in post-translational modifications by at least 10%, 15%, 20%, 30%, 40%, 50%, 60%, 75%, 80%, 90% or more.

Methods of introducing a modification into a cell include physical, biological and chemical methods. See, for example, Geng. & Lu, Microfluidic electroporation for cellular analysis and delivery. Lab on a Chip. 13(19):3803-21. 2013; Sharei, A. et al. A vector-free microfluidic platform for intracellular delivery. PNAS vol. 110 no. 6. 2013; Yin, H. et al., Non-viral vectors for gene-based therapy. Nature Reviews Genetics. 15: 541-555. 2014. Suitable methods for modifying a cell for use in generating the cytobiologics described herein include, for example, diffusion, osmosis, osmotic pulsing, osmotic shock, hypotonic lysis, hypotonic dialysis, ionophoresis, electroporation, sonication, microinjection, calcium precipitation, membrane intercalation, lipid mediated transfection, detergent treatment, viral infection, receptor mediated endocytosis, use of protein transduction domains, particle firing, membrane fusion, freeze-thawing, mechanical disruption, and filtration.

Confirming the presence of a genetic modification includes a variety of assays. Such assays include, for example, molecular biological assays, such as Southern and Northern blotting, RT-PCR and PCR; biochemical assays, such as detecting the presence or absence of a particular peptide, e.g., by immunological means (ELISAs and Western blots) or by assays described herein.

### Modifications to Mitochondrial Biogenesis

In some embodiments, a method described herein comprises:

(a) providing a plurality of source cells that has been contacted with a modulator of mitochondrial biogenesis, e.g., contacting a plurality of source cells with a modulator of mitochondrial biogenesis (e.g., (i) an agent that modulates mtDNA amplification, (ii) an agent that modulates mitochondrial lipid synthesis, or (iii) an agent that modulates production of nuclear-encoded mitochondrial proteins or a combination thereof), and

(b) separating cytobiologics from the plurality of cells.

In embodiments, the modulator of mitochondrial biogenesis upregulates or stimulates mitochondrial biogenesis. In other embodiments, the modulator of mitochondrial biogenesis downregulates or inhibits mitochondrial biogenesis.

In embodiments, the agent that modulates mtDNA amplification is an agent that promotes or inhibits mtDNA amplification. In embodiments, the agent that modulates mitochondrial lipid synthesis is an agent that promotes or inhibits mitochondrial lipid synthesis. In embodiments, the agent that modulates production of nuclear-encoded mitochondrial proteins is an agent that promotes or inhibits production of nuclear-encoded mitochondrial proteins.

In embodiments, the agent that promotes mtDNA amplification comprises: a protein that participates in mtDNA amplification, a protein that upregulates a protein that participates in mtDNA replication, or a deoxyribonucleotide or precursor thereof. In embodiments, the agent that promotes mitochondrial lipid synthesis is a lipid synthesis gene. In embodiments, the agent that promotes production of nuclear-encoded mitochondrial proteins is a transcription factor.

In embodiments, the agent that inhibits mtDNA amplification comprises: an inhibitor of a protein that participates in mtDNA amplification (e.g., a topoisomerase inhibitor, an intercalating agent, a siRNA that downregulates a protein that participates in mtDNA amplification, a targeted nuclease that downregulates a protein that participates in mtDNA amplification, a CRISPR/Cas9 molecule that that interferes with a gene for protein that participates in mtDNA amplification), a protein that downregulates a protein that participates in mtDNA replication, or a deoxyribonucleotide analog or precursor thereof. In embodiments, the agent that inhibits mitochondrial lipid synthesis is an inhibitor of a lipid synthesis gene. In embodiments, the agent that inhibits production of nuclear-encoded mitochondrial proteins is a transcriptional repressor.

In embodiments, modulating mitochondrial biogenesis comprises modulating a protein of Table 4. In embodiments, modulating mitochondrial biogenesis comprises modulating upregulating, downregulating, stimulating, or inhibiting a direct control gene (e.g., a master regulator or DNA binding factor). In embodiments, modulating mitochondrial biogenesis comprises upregulating, downregulating, stimulating, or inhibiting a direct control gene of Table 4 (e.g., a master regulator of Table 4 or a DNA binding factor of Table 4). In embodiments, modulating mitochondrial biogenesis comprises upregulating, downregulating, stimulating, or inhibiting an indirect control gene (e.g., an activator or inhibitor). In embodiments, modulating mitochondrial biogenesis comprises upregulating, downregulating, stimulating, or inhibiting an indirect control gene of Table 4 (e.g., an activator of Table 4 or an inhibitor of Table 4). In embodiments, modulating mitochondrial biogenesis comprises upregulating or downregulating a metabolite, e.g., a metabolite of Table 4.

In embodiments, an agent that promotes or inhibits synthesis of a mitochondrial lipid is capable of causing, or results in, an altered proportion of lipids in the mitochondrial membrane. In embodiments, the agent that modulates synthesis of a mitochondrial lipid results in an increase or decrease in the proportion of one of the following mitochondrial lipids: cardiolipin, phosphatidylglycerol, phosphatidylethanolamine, phosphatidic acid, CDP-diacylglycerol, phosphatidylcholine, phosphatidylserine, phosphatidylinositol, cholesterol, or ceramide e.g., by at least 10%, 20%, 30%, 40%, 50%, 60%, 70%, 80%, or 90%.

In some embodiments, the method comprises providing one, two, or all three of (i), (ii), and (iii). In some embodiments, the method comprises providing two of (i), (ii), and (iii), e.g., (i) and (ii), (i) and (iii), or (ii) and (iii). In some embodiments, the method comprises providing one of one, two, or all three of (i), (ii), and (iii) at a level sufficient to stimulate mitochondrial biogenesis.

In embodiments, the method comprises modulating (e.g., stimulating) mtDNA amplification (e.g., by at least 10%, 20%, 30%, 40%, 50%, 60%, 70%, 80%, or 90%). In embodiments, modulating mtDNA amplification occurs without detectable modulation (e.g. stimulation) of one or both of lipid synthesis and production of nuclear encoded mitochondrial proteins. In embodiments, the method comprises modulating (e.g., stimulating) lipid synthesis (e.g., by at least 10%, 20%, 30%, 40%, 50%, 60%, 70%, 80%, or 90%). In embodiments, modulating occurs without detectable modulation (e.g. stimulation) of one or both of mtDNA amplification and production of nuclear encoded mitochondrial proteins. In embodiments, the method comprises modulating (e.g., stimulating) production of nuclear encoded mitochondrial proteins (e.g., by at least 10%, 20%, 30%, 40%, 50%, 60%, 70%, 80%, or 90%). In embodiments, modulating production of nuclear encoded mitochondrial proteins occurs without detectable modulation (e.g. stimulation) of one or both of lipid synthesis and mtDNA amplification.

In embodiments, the method comprises modulating (e.g., stimulating) mtDNA amplification and lipid synthesis (e.g., each independently by at least 10%, 20%, 30%, 40%, 50%, 60%, 70%, 80%, or 90%). In embodiments, modulating mtDNA amplification and lipid synthesis occurs without detectable modulation (e.g. stimulation) of production of nuclear encoded mitochondrial proteins. In embodiments, the method comprises modulating (e.g., stimulating) mtDNA amplification and production of nuclear encoded mitochondrial proteins (e.g., each independently by at least 10%, 20%, 30%, 40%, 50%, 60%, 70%, 80%, or 90%). In embodiments, modulating mtDNA amplification and production of nuclear encoded mitochondrial proteins occurs without detectable modulation (e.g. stimulation) of lipid synthesis. In embodiments, the method comprises modulating (e.g., stimulating) lipid synthesis and production of nuclear encoded mitochondrial proteins (e.g., each independently by at least 10%, 20%, 30%, 40%, 50%, 60%, 70%, 80%, or 90%). In embodiments, modulating lipid synthesis and production of nuclear encoded mitochondrial proteins occurs without detectable modulation (e.g. stimulation) of mtDNA amplification.

In embodiments, the method comprises modulating (e.g., stimulating) mtDNA amplification, lipid synthesis, and production of nuclear encoded mitochondrial proteins (e.g., each independently by at least 10%, 20%, 30%, 40%, 50%, 60%, 70%, 80%, or 90%).

In embodiments, the modulator of mitochondrial biogenesis is a stimulator of mitochondrial biogenesis. In embodiments, the modulator of mitochondrial biogenesis is a stimulator of browning. In embodiments, the stimulator of browning is PGC1a. In embodiments, the stimulator of browning is quinone, FGF21, irisin, apelin, or isoproterenol. In embodiments, the plurality of source cells or a cytobiologic composition derived from the plurality of source cells is assayed for browning, e.g., by ELISA for UCP1 expression, e.g., as described in Spaethling et al “Single-cell transcriptomics and functional target validation of brown adipocytes show their complex roles in metabolic homeostasis.” in: FASEB Journal, Vol. 30, Issue 1, pp. 81-92, 2016.

In embodiments, the plurality of source cells or a cytobiologic composition derived from the plurality is assayed for the presence or level of mtDNA amplification, mitochondrial lipid synthesis, or production of nuclear-encoded mitochondrial proteins, or any combination thereof.

The source cell may be contacted with a modulator of mitochondrial biogenesis in an amount and for a time sufficient to increase mitochondrial biogenesis in the source cell (e.g., by at least 10%, 15%, 20%, 30%, 40%, 50%, 60%, 75%, 80%, 90% or more). Such modulator of mitochondrial biogenesis are described, e.g., in Cameron et al. 2016. Development of Therapeutics That Induce Mitochondrial Biogenesis for the Treatment of Acute and Chronic Degenerative Diseases. DOI: 10. 1021/acs.jmedchem.6b00669. In embodiments, the modulator of mitochondrial biogenesis is added to the source cell culture during the growth phase and/or during the production phase. In embodiments, the modulator of mitochondrial biogenesis is added when the source cell culture has a predetermined target density.

In one embodiment, the modulator of mitochondrial biogenesis is an agent extracted from a natural product or its synthetic equivalent, sufficient to increase mitochondrial biogenesis in the source cell. Examples of such agents include resveratrol, epicatechin, curcumin, a phytoestrogen (e.g., genistein, daidzein, pyrroloquinoline, quinone, coumestrol and equol).

In another embodiment, the modulator of mitochondrial biogenesis is a metabolite sufficient to increase mitochondrial biogenesis in the source cell, mitochondria in the source cell, e.g., a primary or secondary metabolite. Such metabolites, e.g., primary metabolites include alcohols such as ethanol, lactic acid, and certain amino acids and secondary metabolites include organic compounds produced through the modification of a primary metabolite, are described in “Primary and Secondary Metabolites.” Boundless Microbiology. Boundless, 26 May 2016.

In one embodiment, the modulator of mitochondrial biogenesis is an energy source sufficient to increase mitochondrial biogenesis in the source cell, or mitochondria in the source cell, e.g., sugars, ATP, redox cofactors as NADH and FADH2. Such energy sources, e.g., pyruvate or palmitate, are described in Mehlman, M. Energy Metabolism and the Regulation of Metabolic Processes in Mitochondria; Academic Press, 1972.

In one embodiment, the modulator of mitochondrial biogenesis is a transcription factor modulator sufficient to increase mitochondrial biogenesis in the source cell. Examples of such transcription factor modulators include: thiazolidinediones (e.g., rosiglitazone, pioglitazone, troglitazone and ciglitazone), estrogens (e.g., 1713-Estradiol, progesterone) and estrogen receptor agonists; SIRT1 Activators (e.g., SRT1720, SRT1460, SRT2183, SRT2104).

In one embodiment, the modulator of mitochondrial biogenesis is a kinase modulator sufficient to increase mitochondrial biogenesis in the source cell. Examples include: AMPK and AMPK activators such as AICAR, metformin, phenformin, A769662; and ERK1/2 inhibitors, such as U0126, trametinib.

In one embodiment, the modulator of mitochondrial biogenesis is a cyclic nucleotide modulator sufficient to increase mitochondrial biogenesis in the source cell. Examples include modulators of the NO-cGMP-PKG pathway (for example nitric oxide (NO) donors, such as sodium nitroprusside, (±)S-nitroso-N-acetylpenicillamine (SNAP), diethylamine NONOate (DEA-NONOate), diethylenetriamine-NONOate (DETA-NONOate); sGC stimulators and activators, such as cinaciguat, riociguat, and BAY 41-2272; and phosphodiesterase (PDE) inhibitors, such as zaprinast, sildenafil, udenafil, tadalafil, and vardenafil) and modulators of the cAMP-PKA-CREB Axis, such as phosphodiesterase (PDE) inhibitors such as rolipram.

In one embodiment, the modulator of mitochondrial biogenesis is a modulator of a G protein coupled receptor (GPCR) such as a GPCR ligand sufficient to increase mitochondrial biogenesis in the source cell.

In one embodiment, the modulator of mitochondrial biogenesis is a modulator of a cannabinoid-1 receptor sufficient to increase mitochondrial biogenesis in the source cell. Examples include taranabant and rimonobant.

In one embodiment, the modulator of mitochondrial biogenesis is a modulator of a 5-Hydroxytryptamine receptor sufficient to increase mitochondrial biogenesis in the source cell. Examples include alpha-methyl-5-hydroxytryptamine, DOI, CP809101, SB242084, serotonin reuptake inhibitors such as fluoxetine, alpha-methyl 5HT, 1-(2,5-dimethoxy-4-iodophenyl)-2-aminopropane, LY334370, and LY344864.

In one embodiment, the modulator of mitochondrial biogenesis is a modulator of a beta adrenergic receptor sufficient to increase mitochondrial biogenesis in the source cell. Examples include epinephrine, norepinephrine, isoproterenol, metoprolol, formoterol, fenoterol and procaterol.

In one embodiment, the source cells are modified, e.g., genetically modified, to express a transcriptional activator of mitochondrial biogenesis, e.g., a transcription factor or transcriptional coactivator such as PGC1α. In some embodiments, the cells express PGC1α (e.g., over express an endogenous, or express an exogenous, PGC1α).

TABLE 4
Transcriptional Control of Mitochondrial Biogenesis. See, e.g., Scarpulla et al., “Transcriptional
integration of mitochondrial biogenesis” Trends in Endocrinology & Metabolism, Volume 23,
Issue 9, p459-466, September 2012; Hock et al. “Transcriptional control of mitochondrial biogenesis
and function.” Annu Rev Physiol. 2009; 71: 177-203. Santra et al., “Ketogenic Treatment
Reduces Deleted Mitochondrial DNAs in Cultured Human Cells” Ann Neurol. 2004 November; 56(5):
662-9. Kanabus et al., “The pleiotropic effects of decanoic acid treatment on mitochondrial
function in fibroblasts from patients with complex I deficient Leigh syndrome” J Inherit
Metab Dis. 2016 May; 39(3): 415-26, each of which is herein incorporated by reference in its entirety.

| | |
|---|---|
| Gene | Target or function controlled |

Direct control genes
Master regulators

| | |
|---|---|
| PGC-1a | Master regulator, co-activator for PPAR-delta, a, gamma; ERRa, b, gamma; |
| | GABP; NRF-1; YY1; CREB; c-MYC |
| PGC-1b | Master regulator, co-activator for PPAR-delta, a, gamma; ERRa, b, gamma; |
| | GABP; NRF-1; YY1; CREB; c-MYC |
| RIP140 | Co-repressor with PPAR-delta, a, gamma and ERRa, beta, gamma |
| PRC | Master regulator, co-activator for PPAR-delta, a, gamma; ERRa, b, gamma; |
| | GABP; NRF-1; YY1; CREB; c-MYC |

DNA binding factors

| | |
|---|---|
| RXR (Retinoid X receptor) | Fatty Acid Beta Oxidation & Uncoupling protein |
| PPARa | Fatty Acid Beta Oxidation & Uncoupling protein |
| PPAR-delta | Fatty Acid Beta Oxidation & Uncoupling protein |
| PPAR-gamma | Uncoupling protein |
| NRF-1 | Maintenance of mtDNA and expression of ETC; mtDNA transcription; |
| | mitochondrial import |
| NRF-2 | Maintenance of mtDNA and expression of multiple ETC components |
| ERR (a, B and gamma) | Through interactions with PGC1a, regulated expression of fatty acid B-ox, |
| | Mitochondrial dynamics (fission/fusion); ETC; mtDNA replication and |
| | transcription; mitochondrial import |
| GABP | Maintenance of mtDNA and expression of ETC; mtDNA transcription; |
| | mitochondrial import |
| YY1 | Maintenance of mtDNA and expression of ETC; mtDNA transcription; |
| | mitochondrial import |
| c-MYC | Maintenance of mtDNA and expression of ETC; mtDNA transcription; |
| | mitochondrial import |
| CREB | Maintenance of mtDNA and expression of ETC; mtDNA transcription; |
| | mitochondrial import |

Indirect control genes
Inhibitors

| | |
|---|---|
| SRC-3 | Acetylates and inhibits PGC-1a |
| GCN5 | Acetylates and inhibits PGC-1a |
| AKT | |
| SCF-cdc4 | |
| MYBBP1a | |

Activators

| | |
|---|---|
| SIRT1 | Deacetylates and activates PCG-1a |
| AMPK | Phosphorylates and activates PGC-1a |
| Cdk/cyclin H/MAT1 | |
| PRMT1 | |
| GSK-3B | |

Indirect control genes for other processes

| | |
|---|---|
| SIRT3 | Controls mtSOD2 and GSH/GPX to inhibit ROS levels |

Metabolites stimulating biogenesis

| Name | Class |
|---|---|
| β-hydroxybutyrate (BHB) | Ketone body |
| Acetoacetate (ACA) | Ketone Body |
| decanoic acid (C10) | Medium chain triglyceride |
| octanoic acid | Medium chain triglyceride |

### Cytobiologic Modifications

In one aspect, a modification is made to the cytobiologic. Such modifications can be effective to, e.g., improve targeting, function, or structure.

In some embodiments, a ligand is conjugated to the surface of the cytobiologic via a functional chemical group (carboxylic acids, aldehydes, amines, sulfhydryls and hydroxyls) that is present on the surface of the cytobiologic.

Such reactive groups include without limitation maleimide groups. As an example, cytobiologics may be synthesized to include maleimide conjugated phospholipids such as without limitation DSPE-MaL-PEG2000.

In some embodiments, a small molecule or lipid, synthetic or native, may be covalently or non-covalent linked to the surface of the cytobiologic.

In some embodiments, the cytobiologic is modified by loading with modified proteins (e.g., enable novel functionality, alter post-translational modifications, bind to the mitochondrial membrane and/or mitochondrial membrane proteins, form a cleavable protein with a heterologous function, form a protein destined for proteolytic degradation, assay the agent's location and levels, or deliver the agent as a carrier). In one embodiment, the invention includes a cytobiologic loaded with modified proteins.

In some embodiments, an exogenous protein is non-covalently bound to the cytobiologic. The protein may include a cleavable domain for release. In one embodiment, the invention includes a cytobiologic comprising an exogenous protein with a cleavable domain.

In some embodiments, the cytobiologic is modified with a protein destined for proteolytic degradation. A variety of proteases recognize specific protein amino acid sequences and target the proteins for degradation. These protein degrading enzymes can be used to specifically degrade proteins having a proteolytic degradation sequence. In one embodiment, the invention includes a cytobiologic comprising modulated levels of one or more protein degrading enzymes, e.g., an increase or a decrease in protein degrading enzymes by at least 10%, 15%, 20%, 30%, 40%, 50%, 60%, 75%, 80%, 90% or more.

As described herein, non-fusogen additives may be added to the cytobiologic to modify their structure and/or properties. For example, either cholesterol or sphingomyelin may be added to the membrane to help stabilize the structure and to prevent the leakage of the inner cargo. Further, membranes can be prepared from hydrogenated egg phosphatidylcholine or egg phosphatidylcholine, cholesterol, and dicetyl phosphate. (see, e.g., Spuch and Navarro, Journal of Drug Delivery, vol. 2011, Article ID 469679, 12 pages, 2011. doi:10.1155/2011/469679 for review).

In some embodiments, the cytobiologic comprises one or more targeting groups (e.g., a targeting protein) on the exterior surface to target a specific cell or tissue type (e.g., cardiomyocytes). These targeting groups include without limitation receptors, ligands, antibodies, and the like. These targeting groups bind their partner on the target cells' surface. In embodiments, the targeting protein is specific for a cell surface marker on a target cell described herein, e.g., a skin cell, cardiomyocyte, hepatocyte, intestinal cell (e.g., cell of the small intestine), pancreatic cell, brain cell, prostate cell, lung cell, colon cell, or bone marrow cell.

In some embodiments, the cytobiologic described herein is functionalized with a diagnostic agent. Examples of diagnostic agents include, but are not limited to, commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium.

Another example of introducing functional groups to the cytobiologic is during post-preparation, by direct crosslinking cytobiologic and ligands with homo- or heterobifunctional crosslinkers. This procedure may use a suitable chemistry and a class of crosslinkers (CDI, EDAC, glutaraldehydes, etc. as discussed herein) or any other crosslinker that couples a ligand to the cytobiologic surface via chemical modification of the cytobiologic surface after preparation. This also includes a process whereby amphiphilic molecules such as fatty acids, lipids or functional stabilizers may be passively adsorbed and adhered to the cytobiologic surface, thereby introducing functional end groups for tethering to ligands.

### Cargo

In some embodiments, a cytobiologic described herein includes a cargo, e.g., subcellular cargo.

In some embodiments, a cytobiologic described herein includes a cargo, e.g., a therapeutic agent, e.g., an endogenous therapeutic agent or an exogenous therapeutic agent.

In some embodiments, the cargo is not expressed naturally in the cell from which the cytobiologic is derived. In some embodiments, the cargo is expressed naturally in the cell from which the cytobiologic is derived. In some embodiments, the cargo is a mutant of a wild type nucleic acid or protein expressed naturally in the cell from which the cytobiologic is derived or is a wild type of a mutant nucleic acid or protein expressed naturally in the cell from which the cytobiologic is derived.

In some embodiments, the cargo is loaded into the cytobiologic via expression in the cell from which the cytobiologic is derived (e.g. expression from DNA introduced via transfection, transduction, or electroporation). In some embodiments, the cargo is expressed from DNA integrated into the genome or maintained episosomally. In some embodiments, expression of the cargo is constitutive. In some embodiments, expression of the cargo is induced. In some embodiments, expression of the cargo is induced immediately prior to generating the cytobiologic.

In some embodiments, the cargo is loaded into the cytobiologic via electroporation into the cytobiologic itself or into the cell from which the cytobiologic is derived. In some embodiments, the cargo is loaded into the cytobiologic via transfection into the cytobiologic itself or into the cell from which the cytobiologic is derived.

In some embodiments, a cytobiologic composition (e.g., a pharmaceutical composition) comprises one or more of a chondrisome (e.g., as described in international application, PCT/US16/64251), a mitochondrion, an organelle (e.g., Mitochondria, Lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome, proteasome, vesicle, stress granule, and networks of organelles), or an enucleated cell, e.g., an enucleated cell comprising any of the foregoing.

In embodiments, the chondrisome has one or more of the properties as described, for example, in international application, PCT/US16/64251, which is herein incorporated by reference in its entirety, including the Examples and the Summary of the Invention.

In some embodiments, the cargo may include one or more nucleic acid sequences, one or more polypeptides, a combination of nucleic acid sequences and/or polypeptides, one or more organelles, and any combination thereof. In some embodiments, the cargo may include one or more cellular components. In some embodiments, the cargo includes one or more cytosolic and/or nuclear components.

In some embodiments, the cargo includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprogramming RNAs, aptamers, and any combination thereof. In some embodiments, the nucleic acid is a wild-type nucleic acid. In some embodiments, the protein is a mutant nucleic acid. In some embodiments the nucleic acid is a fusion or chimera of multiple nucleic acid sequences.

In some embodiments, DNA in the cytobiologic or DNA in the cell that the cytobiologic is derived from is edited to correct a genetic mutation using a gene editing technology, e.g. a guide RNA and CRISPR-Cas9/Cpf1, or using a different targeted endonuclease (e.g., Zinc-finger nucleases, transcription-activator-like nucleases (TALENs)). In some embodiments, the genetic mutation is linked to a disease in a subject. Examples of edits to DNA include small insertions/deletions, large deletions, gene corrections with template DNA, or large insertions of DNA. In some embodiments, gene editing is accomplished with non-homologous end joining (NHEJ) or homology directed repair (HDR). In some embodiments, the edit is a knockout. In some embodiments, the edit is a knock-in. In some embodiments, both alleles of DNA are edited. In some embodiments, a single allele is edited. In some embodiments, multiple edits are made. In some embodiments, the cytobiologic or cell is derived from a subject, or is genetically matched to the subject, or is immunologically compatible with the subject (e.g. having similar MHC).

In some embodiments, the cargo may include a nucleic acid. For example, the cargo may comprise RNA to enhance expression of an endogenous protein, or a siRNA or miRNA that inhibits protein expression of an endogenous protein. For example, the endogenous protein may modulate structure or function in the target cells. In some embodiments, the cargo may include a nucleic acid encoding an engineered protein that modulates structure or function in the target cells. In some embodiments, the cargo is a nucleic acid that targets a transcriptional activator that modulate structure or function in the target cells.

In some embodiments, the cargo includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, and any combination thereof. In some embodiments the protein targets a protein in the cell for degredation. In some embodiments the protein targets a protein in the cell for degredation by localizing the protein to the proteasome. In some embodiments, the protein is a wild-type protein. In some embodiments, the protein is a mutant protein. In some embodiments the protein is a fusion or chimeric protein.

In some embodiments, the cargo includes a small molecule, e.g., ions (e.g. Ca²⁺, Cl⁻, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical that interacts with a target in the cell. In some embodiments the small molecule targets a protein in the cell for degredation. In some embodiments the small molecule targets a protein in the cell for degredation by localizing the protein to the proteasome. In some embodiments that small molecule is a proteolysis targeting chimera molecule (PROTAC).

In some embodiments, the cargo includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

In some embodiments, the cargo includes one or more organelles, e.g., chondrisomes, mitochondria, lysosomes, nucleus, cell membrane, cytoplasm, endoplasmic reticulum, ribosomes, vacuoles, endosomes, spliceosomes, polymerases, capsids, acrosome, autophagosome, centriole, glycosome, glyoxysome, hydrogenosome, melanosome, mitosome, myofibril, cnidocyst, peroxisome, proteasome, vesicle, stress granule, networks of organelles, and any combination thereof.

In some embodiments, the cargo is enriched at the cytobiologic or cell membrane. In some embodiments, the cargo is enriched by targeting to the membrane via a peptide signal sequence. In some embodiments, the cargo is enriched by binding with a membrane associated protein, lipid, or small molecule. In some embodiments, the cargo is enriched by dimerizing with a membrane associated protein, lipid, or small molecule. In some embodiments the cargo is chimeric (e.g. a chimeric protein, or nucleic acid) and comprises a domain that mediates binding or dimerization with a membrane associated protein, lipid, or small molecule. Membrane-associated proteins of interest include, but are not limited to, any protein having a domain that stably associates, e.g., binds to, integrates into, etc., a cell membrane (i.e., a membrane-association domain), where such domains may include myristoylated domains, farnesylated domains, transmembrane domains, and the like. Specific membrane-associated proteins of interest include, but are not limited to; myristoylated proteins, e.g., p 60 v-src and the like; farnesylated proteins, e.g., Ras, Rheh and CENP-E,F, proteins binding specific lipid bilayer components e.g. AnnexinV, by binding to phosphatidyl-serine, a lipid component of the cell membrane bilayer and the like; membrane anchor proteins; transmembrane proteins, e.g., transferrin receptors and portions thereof; and membrane fusion proteins. In some embodiment, the membrane associated protein contains a first dimerization domain. The first dimerization domain may be, e.g., a domain that directly binds to a second dimerization domain of a cargo or binds to a second dimerization domain via a dimerization mediator. In some embodiments the cargo contains a second dimerization domain. The second dimerization domain may be, e.g., a domain that dimerizes (e.g., stably associates with, such as by non-covalent bonding interaction, either directly or through a mediator) with the first dimerization domain of the membrane associated protein either directly or through a dimerization mediator. With respect to the dimerization domains, these domains are domains that participate in a binding event, either directly or via a dimerization mediator, where the binding event results in production of the desired multimeric, e.g., dimeric, complex of the membrane associated and target proteins. The first and second dimerization domains may be homodimeric, such that they are made up of the same sequence of amino acids, or heterodimeric, such that they are made up of differing sequences of amino acids. Dimerization domains may vary, where domains of interest include, but are not limited to: ligands of target biomolecules, such as ligands that specifically bind to particular proteins of interest (e.g., protein:protein interaction domains), such as SH2 domains, Paz domains, RING domains, transcriptional activator domains, DNA binding domains, enzyme catalytic domains, enzyme regulatory domains, enzyme subunits, domains for localization to a defined cellular location, recognition domains for the localization domain, the domains listed at URL: pawsonlab.mshrion.ca/index.php?option=com_content&task=view&id=30&Itemid=63/, etc. In some embodiments the first dimerization domain binds nucleic acid (e.g. mRNA, miRNA, siRNA, DNA) and the second dimerization domain is a nucleic acid sequence present on the cargo (e.g. the first dimerization domain is MS2 and the second dimerization domain is the high affinity binding loop of MS2 RNA). Any convenient compound that functions as a dimerization mediator may be employed. A wide variety of compounds, including both naturally occurring and synthetic substances, can be used as dimerization mediators. Applicable and readily observable or measurable criteria for selecting a dimerization mediator include: (A) the ligand is physiologically acceptable (i.e., lacks undue toxicity towards the cell or animal for which it is to be used); (B) it has a reasonable therapeutic dosage range; (C) it can cross the cellular and other membranes, as necessary (where in some instances it may be able to mediate dimerization from outside of the cell), and (D) binds to the target domains of the chimeric proteins for which it is designed with reasonable affinity for the desired application. A first desirable criterion is that the compound is relatively physiologically inert, but for its dimerization mediator activity. In some instances, the ligands will be non-peptide and non-nucleic acid, Additional dimerization domains are described, e.g., in US20170087087 and US20170130197, each of which is herein incorporated by reference in its entirety.

### Characteristics of Chondrisomes

In one aspect, the cytobiologic composition, e.g., a pharmaceutical, comprises isolated chondrisomes (e.g., a chondrisome preparation), derived from a cellular source of mitochondria.

In another aspect, the cytobiologic composition, e.g., a pharmaceutical composition, comprises isolated, modified chondrisomes (e.g., modified chondrisome preparation) derived from a cellular source of mitochondria.

In another aspect, the cytobiologic composition, e.g., a pharmaceutical composition, comprises chondrisomes (e.g., chondrisome preparation) expressing an exogenous protein.

Additional features and embodiments including chondrisomes (e.g., chondrisome preparations), methods, and uses disclosed herein include one or more of the following.

In some embodiments, the chondrisome (or the chondrisomes in the composition) has one or more (2, 3, 4, 5, 6, 7, 8, 9 or more, e.g., all) of the following characteristics:

outer chondrisome membrane integrity wherein the composition exhibits <20% (e.g., <15%, <10%, <5%, <4%, <3%, <2%, <1%) increase in oxygen consumption rate over state 4 rate following addition of reduced cytochrome c;

genetic quality >80%, e.g., >85%, >90%, >95%, >97%, >98%, >99%, wherein “genetic quality” of a chondrisome preparation means, for all the loci described in Table 5, the percent of sequencing reads mapping to the wild type allele;

glutamate/malate RCR 3/2 of 1-15, e.g., 2-15, 5-15, 2-10, 2-5, 10-15;

glutamate/malate RCR 3/4o of 1-30, 1-20, 2-20, 5-20, 3-15, 10-30;

succinate/rotenone RCR 3/2 of 1-15, 2-15, 5-15, 1-10, 10-15;

succinate/rotenone RCR 3/4o of 1-30, 1-20, 2-20, 5-20, 3-15, 10-30;

palmitoyl carnitine and malate RCR3/2 state 3/state 2 respiratory control ratio (RCR 3/2) of 1-10 (e.g., 1-5);

cardiolipin content 0.05-25 (0.1-20, 0.5-20, 1-20, 5-20, 5-25, 1-25, 10-25, 15-25) 100*pmol/pmol total lipid;

genomic concentration 0.001-2 (e.g., 0.001-1, 0.01-1, 0.01-0.1, 0.01-0.05, 0.1-0.2) mtDNA ug/mg protein; or

relative ratio of mtDNA/nuclear DNA of >1000 (e.g., >1,500, >2000, >2,500, >3,000, >4,000, >5000, >10,000, >25,000, >50,000, >100,000, >200,000, >500,000).

In some embodiments, the chondrisome (or the chondrisomes in the composition) has one or more (2, 3, 4, 5, 6 or more) of the following characteristics:

the chondrisomes in the composition have a mean average size between 150-1500 nm, e.g., between 200-1200 nm, e.g., between 500-1200 nm, e.g., 175-950 nm;

the chondrisomes in the composition have a polydispersity (D90/D10) between 1.1 to 6, e.g., between 1.5-5. In embodiments, chondrisomes in the composition from a cultured cell source (e.g., cultured fibroblasts) have a polydispersity (D90/D10) between 2-5, e.g., between 2.5-5; outer chondrisome membrane integrity wherein the composition exhibits <20% (e.g., <15%, <10%, <5%, <4,%, <3%, <2%, <1%) increase in oxygen consumption rate over state 4 rate following addition of reduced cytochrome c;

complex I level of 1-8 mOD/ug total protein, e.g., 3-7 mOD/ug total protein, 1-5 mOD/ug total protein. In embodiments, chondrisomes of a preparation from a cultured cell source (e.g., cultured fibroblasts) have a complex I level of 1-5 mOD/ug total protein;

complex II level of 0.05-5 mOD/ug total protein, e.g., 0.1-4 mOD/ug total protein, e.g., 0.5-3 mOD/ug total protein. In embodiments, chondrisomes of a preparation from a cultured cell source (e.g., cultured fibroblasts) have a complex II level of 0.05-1 mOD/ug total protein;

complex III level of 1-30 mOD/ug total protein, e.g., 2-30, 5-10, 10-30 mOD/ug total protein. In embodiments, chondrisomes from a cultured cell source (e.g., cultured fibroblasts) have a complex III level of 1-5 mOD/ug total protein;

complex IV level of 4-50 mOD/ug total protein, e.g., 5-50, e.g., 10-50, 20-50 mOD/ug total protein. In embodiments, chondrisomes from a cultured cell source (e.g., cultured fibroblasts) have a complex IV level of 3-10 mOD/ug total protein;

genomic concentration 0.001-2 (e.g., 0.001-1, 0.01-1, 0.01-0.1, 0.01-.05, 0.1-0.2) mtDNA ug/mg protein;

membrane potential of the preparation is between −5 to −200 mV, e.g., between −100 to −200 mV, −50 to −200 mV, −50 to −75 mV, −50 to −100 mV. In some embodiments, membrane potential of the preparation is less than −150 mV, less than −100 mV, less than −75 mV, less than −50 mV, e.g., −5 to −20 mV;

a protein carbonyl level of less than 100 nmol carbonyl/mg chondrisome protein (e.g., less than 90 nmol carbonyl/mg chondrisome protein, less than 80 nmol carbonyl/mg chondrisome protein, less than 70 nmol carbonyl/mg chondrisome protein, less than 60 nmol carbonyl/mg chondrisome protein, less than 50 nmol carbonyl/mg chondrisome protein, less than 40 nmol carbonyl/mg chondrisome protein, less than 30 nmol carbonyl/mg chondrisome protein, less than 25 nmol carbonyl/mg chondrisome protein, less than 20 nmol carbonyl/mg chondrisome protein, less than 15 nmol carbonyl/mg chondrisome protein, less than 10 nmol carbonyl/mg chondrisome protein, less than 5 nmol carbonyl/mg chondrisome protein, less than 4 nmol carbonyl/mg chondrisome protein, less than 3 nmol carbonyl/mg chondrisome protein;

<20% mol/mol ER proteins (e.g., >15%, >10%, >5%, >3%, >2%, >1%) mol/mol ER proteins;

>5% mol/mol mitochondrial proteins (proteins identified as mitochondrial in the MitoCarta database (Calvo et al., NAR 20151 doi:10.1093/nar/gkv1003)), e.g., >10%, >15%, >20%, >25%, >30%, >35%, >40%; >50%, >55%, >60%, >65%, >70%, >75%, >80%; >90% mol/mol mitochondrial proteins);

>0.05% mol/mol of MT-CO₂, MT-ATP6, MT-ND5 and MT-ND6 protein (combined) (e.g., >0.1%; >05%, >1%, >2%, >3%, >4%, >5%, >7, >8%, >9%, >10, >15% mol/mol of MT-CO₂, MT-ATP6, MT-ND5 and MT-ND6 protein);

genetic quality >80%, e.g., >85%, >90%, >95%, >97%, >98%, >99%;

relative ratio mtDNA/nuclear DNA is >1000 (e.g., >1,500, >2000, >2,500, >3,000, >4,000, >5000, >10,000, >25,000, >50,000, >100,000, >200,000, >500,000);

endotoxin level <0.2 EU/ug protein (e.g., <0.1, 0.05, 0.02, 0.01 EU/ug protein);

substantially absent exogenous non-human serum;

glutamate/malate RCR 3/2 of 1-15, e.g., 2-15, 5-15, 2-10, 2-5, 10-15;

glutamate/malate RCR 3/4o of 1-30, 1-20, 2-20, 5-20, 3-15, 10-30;

succinate/rotenone RCR 3/2 of 1-15, 2-15, 5-15, 1-10, 10-15;

succinate/rotenone RCR 3/4o of 1-30, 1-20, 2-20, 5-20, 3-15, 10-30;

complex I activity of 0.05-100 nmol/min/mg total protein (e.g., 0.05-50, 0.05-20, 0.5-10, 0.1-50, 1-50, 2-50, 5-100, 1-20 nmol/min/mg total protein);

complex II activity of 0.05-50 nmol/min/mg total protein (e.g., 0.05-50, 0.05-20, 0.5-10, 0.1-50, 1-50, 2-50, 5-50, 1-20 nmol/min/mg total protein);

complex III activity of 0.05-20 nmol/min/mg total protein (e.g., 0.05-50, 0.05-20, 0.5-10, 0.1-50, 1-50, 2-50, 5-100, 1-20 nmol/min/mg total protein);

complex IV activity of 0.1-50 nmol/min/mg total protein (e.g., 0.05-50, 0.05-20, 0.5-10, 0.1-50, 1-50, 2-50, 5-50, 1-20 nmol/min/mg total protein);

complex V activity of 1-500 nmol/min/mg total protein (e.g., 10-500, 10-250, 10-200, 100-500 nmol/min/mg total protein);

reactive oxygen species (ROS) production level of 0.01-50 pmol H₂O₂/ug protein/hr (e.g., 0.05-40, 0.05-25, 1-20, 2-20, 0.05-20, 1-20 pmol H₂O₂/ug protein/hr);

citrate synthase activity of 0.05-5 (e.g., 0.5-5, 0.5-2, 1-5, 1-4) mOD/min/ug total protein;

alpha ketoglutarate dehydrogenase activity of 0.05-10 (e.g., 0.1-10, 0.1-8, 0.5-8, 0.1-5, 0.5-5, 0.5-3, 1-3) mOD/min/ug total protein;

creatine kinase activity of 0.1-100 (e.g., 0.5-50, 1-100, 1-50, 1-25, 1-15, 5-15) mOD/min/ug total protein;

pyruvate dehydrogenase activity of 0.1-10 (e.g., 0.5-10, 0.5-8, 1-10, 1-8, 1-5, 2-3) mOD/min/ug total protein;

aconitase activity of 0.1-50 (e.g., 5-50, 0.1-2, 0.1-20, 0.5-30) mOD/min/ug total protein. In embodiments, aconitase activity in a chondrisome preparation from platelets is between 0.5-5 mOD/min/ug total protein. In embodiments, aconitase activity in a chondrisome preparation from cultured cells, e.g., fibroblasts, is between 5-50 mOD/min/ug total protein;

maximal fatty acid oxidation level of 0.05-50 (e.g., 0.05-40, 0.05-30, 0.05-10, 0.5-50, 0.5-25, 0.5-10, 1-5) pmol O₂/min/ug chondrisome protein;

palmitoyl carnitine & malate RCR3/2 state 3/state 2 respiratory control ratio (RCR 3/2) of 1-10 (e.g., 1-5);

electron transport chain efficiency of 1-1000 (e.g., 10-1000, 10-800, 10-700, 50-1000, 100-1000, 500-1000, 10-400, 100-800) nmol Om/min/mg protein/ΔGATP (in kcal/mol);

total lipid content of 50,000-2,000,000 pmol/mg (e.g., 50,000-1,000,000; 50,000-500,000 pmol/mg);

double bonds/total lipid ratio of 0.8-8 (e.g., 1-5, 2-5, 1-7, 1-6) pmol/pmol;

phospholipid/total lipid ratio of 50-100 (e.g., 60-80, 70-100, 50-80) 100*pmol/pmol;

phosphosphingolipid/total lipid ratio of 0.2-20 (e.g., 0.5-15, 0.5-10, 1-10, 0.5-10, 1-5, 5-20) 100*pmol/pmol;

ceramide content 0.05-5 (e.g., 0.1-5, 0.1-4, 1-5, 0.05-3) 100*pmol/pmol total lipid;

cardiolipin content 0.05-25 (0.1-20, 0.5-20, 1-20, 5-20, 5-25, 1-25, 10-25, 15-25) 100*pmol/pmol total lipid;

lyso-phosphatidylcholine (LPC) content of 0.05-5 (e.g., 0.1-5, 1-5, 0.1-3, 1-3, 0.05-2) 100*pmol/pmol total lipid;

lyso-phosphatidylethanolamine (LPE) content of 0.005-2 (e.g., 0.005-1, 0.05-2, 0.05-1) 100*pmol/pmol total lipid;

phosphatidylcholine (PC) content of 10-80 (e.g., 20-60, 30-70, 20-80, 10-60 m 30-50) 100*pmol/pmol total lipid;

phosphatidylcholine-ether (PC 0-) content 0.1-10 (e.g., 0.5-10, 1-10, 2-8, 1-8) 100*pmol/pmol total lipid;

phosphatidylethanolamine (PE) content 1-30 (e.g., 2-20, 1-20, 5-20) 100*pmol/pmol total lipid;

phosphatidylethanolamine-ether (PE 0-) content 0.05-30 (e.g., 0.1-30, 0.1-20, 1-20, 0.1-5, 1-10, 5-20) 100*pmol/pmol total lipid;

phosphatidylinositol (PI) content 0.05-15 (e.g., 0.1-15, 0.1-10, 1-10, 0.1-5, 1-10, 5-15) 100*pmol/pmol total lipid;

phosphatidylserine (PS) content 0.05-20 (e.g., 0.1-15, 0.1-20, 1-20, 1-10, 0.1-5, 1-10, 5-15) 100*pmol/pmol total lipid;

sphingomyelin (SM) content 0.01-20 (e.g., 0.01-15, 0.01-10, 0.5-20, 0.5-15, 1-20, 1-15, 5-20) 100*pmol/pmol total lipid;

triacylglycerol (TAG) content 0.005-50 (e.g., 0.01-50, 0.1-50, 1-50, 5-50, 10-50, 0.005-30, 0.01-25, 0.1-30) 100*pmol/pmol total lipid;

PE:LPE ratio 30-350 (e.g., 50-250, 100-200, 150-300);

PC:LPC ratio 30-700 (e.g., 50-300, 50-250, 100-300, 400-700, 300-500, 50-600, 50-500, 100-500, 100-400);

PE 18:n (n>0) content 0.5-20% (e.g., 1-20%, 1-10%, 5-20%, 5-10%, 3-9%) pmol AA/pmol lipid class;

PE 20:4 content 0.05-20% (e.g., 1-20%, 1-10%, 5-20%, 5-10%) pmol AA/pmol lipid class; PC 18:n (n>0) content 5-50% (e.g., 5-40%, 5-30%, 20-40%, 20-50%) pmol AA/pmol lipid class;

PC 20:4 content 1-20% (e.g., 2-20%, 2-15%, 5-20%, 5-15%) pmol AA/pmol lipid class.

In certain embodiments, the chondrisome (or the chondrisomes in the composition) has one or more of the following characteristics upon administration to a recipient cell, tissue or subject (a control may be a negative control (e.g., a control tissue or subject that has not been administered a composition), or a baseline prior to administration, e.g., a cell, tissue or subject prior to administration of the composition):

Increases basal respiration of recipient cells at least 10% (e.g., >15%, >20%, >30%, >40%, >50%, >60%, >70%, >80%, >90%) relative to a control;

chondrisomes in the composition are taken up by at least 1% (e.g., at least 2%, 5%, 10%, 20%, 30%, 40%, 50%, 60%, 70%) of recipient cells;

chondrisomes in the composition are taken up and maintain membrane potential in recipient cells;

chondrisomes in the composition persist in recipient cells at least 6 hours, e.g., at least 12 hours, 18 hours, 24 hours, 2 days, 3 days, 4 days, a week, 2 weeks, a month, 2 months, 3 months, 6 months;

increase ATP levels in a recipient cell, tissue or subject (e.g., by at least 5%, 10%, 15%, 20%, 25%, 30%, 35%, 40%, 45%, 50%, 60%, 70%, 80%, 90%, or more, e.g., compared to a reference value, e.g., a control value, e.g., an untreated control);

decrease apotosis in a recipient cell, tissue or subject (e.g., by at least 5%, 10%, 15%, 20%, 25%, 30%, 35%, 40%, 45%, 50%, 60%, 70%, 80%, 90%, or more, e.g., compared to a reference value, e.g., a control value, e.g., an untreated control);

decrease cellular lipid levels in a recipient cell, tissue or subject (e.g., by at least 5%, 10%, 15%, 20%, 25%, 30%, 35%, 40%, 45%, 50%, 60%, 70%, 80%, 90%, or more, e.g., compared to a reference value, e.g., a control value, e.g., an untreated control);

increase membrane potential in a recipient cell, tissue or subject (e.g., by at least 5%, 10%, 15%, 20%, 25%, 30%, 35%, 40%, 45%, 50%, 60%, 70%, 80%, 90%, or more, e.g., compared to a reference value, e.g., a control value, e.g., an untreated control);

increase uncoupled respiration in a recipient cell, tissue or subject (e.g., by at least 5%, 10%, 15%, 20%, 25%, 30%, 35%, 40%, 45%, 509%, 60%, 70%, 80%, 90%, or more, e.g., compared to a reference value, e.g., a control value, e.g., an untreated control);

increase PI3K activity in a recipient cell, tissue or subject (e.g., by at least 5%, 10%, 15%, 20%, 25%, 30%, 35%, 40%, 45%, 509%, 60%, 70%, 80%, 90%, or more, e.g., compared to a reference value, e.g., a control value, e.g., an untreated control);

reduce reductive stress in a recipient cell, tissue or subject (e.g., by at least 5%, 10%, 15%, 20%, 25%, 30%, 35%, 40%, 45%, 509%, 60%, 70%, 80%, 90%, or more, e.g., compared to a reference value, e.g., a control value, e.g., an untreated control);

decrease reactive oxygen species (e.g. H₂O₂) in the cell, tissue of subject (e.g., in serum of a target subject) (e.g., by at least 5%, 10%, 15%, 20%, 25%, 30%, 35%, 40%, 45%, 509%, 60%, 70%, 80%, 90%, or more, e.g., compared to a reference value, e.g., a control value, e.g., an untreated control);

decrease cellular lipid levels of recipient cells at least 5% (e.g., >10%, >15%, >20%, >30%, >40%, >50%, >60%, >70%, >80%, >90%) relative to a control;

increases uncoupled respiration of recipient cells at least 5% (e.g., >10%, >15%, >20%, >30%, >40%, >50%, >60%, >70%, >80%, >90%) relative to a control;

decrease mitochondrial permeability transition pore (MPTP) formation in recipient cells at least 5% and does not increase more than 10% relative to a control;

increase Akt levels in recipient cells at least 10% (e.g., >10%, >15%, >20%, >30%, >40%, >50%, >60%, >70%, >80%, >90%) relative to a control;

decrease total NAD/NADH ratio in recipient cells at least 5% (e.g., >10%, >15%, >20%, >30%, >40%, >50%, >60%, >70%, >80%, >90%) relative to a control;

reduce ROS levels in recipient cells at least 5% (e.g., >10%, >15%, >20%, >30%, >40%, >50%, >60%, >70%, >80%, >90%) relative to a control;

increase fractional shortening in subject with cardiac ischemia at least 5% (e.g., >10%, >15%, >20%, >30%, >40%, >50%, >60%, >70%, >80%, >90%) relative to a control;

increase end diastolic volume in subject with cardiac ischemia at least 5% (e.g., >10%, >15%, >20%, >30%, >40%, >50%, >60%, >70%, >80%, >90%) relative to a control;

decrease end systolic volume in subject with cardiac ischemia at least 5% (e.g., >10%, >15%, >20%, >30%, >40%, >50%, >60%, >70%, >80%, >90%) relative to a control;

decrease infarct area of ischemic heart at least 5% (e.g., >10%, >15%, >20%, >30%, >40%, >50%, >60%, >70%, >80%, >90%) relative to a control;

increase stroke volume in subject with cardiac ischemia at least 5% (e.g., >10%, >15%, >20%, >30%, >40%, >50%, >60%, >70%, >80%, >90%) relative to a control;

increase ejection fraction in subject with cardiac ischemia at least 5% (e.g., >10%, >15%, >20%, >30%, >40%, >50%, >60%, >70%, >80%, >90%) relative to a control;

increase cardia output in subject with cardiac ischemia at least 5% (e.g., >10%, >15%, >20%, >30%, >40%, >50%, >60%, >70%, >80%, >90%) relative to a control;

increase cardiac index in subject with cardiac ischemia at least 5% (e.g., >10%, >15%, >20%, >30%, >40%, >50%, >60%, >70%, >80%, >90%) relative to a control;

decrease serum CKNB levels in subject with cardiac ischemia at least 5% (e.g., >10%, >15%, >20%, >30%, >40%, >50%, >60%, >70%, >80%, >90%) relative to a control;

decrease serum cTnI levels in subject with cardiac ischemia at least 5% (e.g., >10%, >15%, >20%, >30%, >40%, >50%, >60%, >70%, >80%, >90%) relative to a control;

decrease serum hydrogen peroxide in subject with cardiac ischemia at least 5% (e.g., >10%, >15%, >20%, >30%, >40%, >50%, >60%, >70%, >80%, >90%) relative to a control;

decrease serum cholesterol levels and/or triglycerides in a subject at least 5% (e.g., >10%, >15%, >20%, >30%, >40%, >50%, >60%, >70%, >80%, >90%) relative to a control.

In some embodiments, the cytobiologic comprises a chondrisome, e.g., isolated chondrisomes from a mitochondrial source, having one or more of the following characteristics:

the chondrisomes in the composition have a mean average size between 150-1500 nm;

the chondrisomes in the composition have a polydispersity (D90/D10) between 1.1 to 6;

outer chondrisome membrane integrity of the chondrisomes in the composition exhibits <20% increase in oxygen consumption rate over state 4 rate following addition of reduced cytochrome c;

complex I level of 1-8 mOD/ug total protein;

complex II level of 0.05-5 mOD/ug total protein;

complex III level of 1-30 mOD/ug total protein;

complex IV level of 4-50 mOD/ug total protein;

genomic concentration 0.001-2 mtDNA ug/mg protein; and/or

membrane potential of the chondrisomes in the composition is between −5 to −200 mV.

In some embodiments, the cytobiologic comprises a chondrisome, e.g., isolated chondrisomes from a mitochondrial source, having one or more of the following characteristics:

a protein carbonyl level of less than 100 nmol carbonyl/mg chondrisome protein.

<20% mol/mol ER proteins

>5% mol/mol mitochondrial proteins (MitoCarta);

>0.05% mol/mol of MT-CO₂, MT-ATP6, MT-ND5 and MT-ND6 protein;

genetic quality >80%;

relative ratio mtDNA/nuclear DNA>1000;

endotoxin level <0.2 EU/ug protein; and/or

substantially absent exogenous non-human serum.

In some embodiments, the cytobiologic comprises a chondrisome, e.g., isolated chondrisomes from a mitochondrial source, having one or more of the following characteristics:

glutamate/malate RCR 3/2 of 1-15;

glutamate/malate RCR 3/4o of 1-30;

succinate/rotenone RCR 3/2 of 1-15;

succinate/rotenone RCR 3/4o of 1-30;

complex I activity of 0.05-100 nmol/min/mg total protein;

complex II activity of 0.05-50 nmol/min/mg total protein;

complex III activity of 0.05-20 nmol/min/mg total protein;

complex IV activity of 0.1-50 nmol/min/mg total protein;

complex V activity of 1-500 nmol/min/mg total protein;

reactive oxygen species (ROS) production level of 0.01-50 pmol H₂O₂/ug protein/hr;

citrate synthase activity of 0.05-5 mOD/min/ug total protein;

alpha ketoglutarate dehydrogenase activity of 0.05-10 mOD/min/ug total protein;

creatine kinase activity of 0.1-100 mOD/min/ug total protein;

pyruvate dehydrogenase activity of 0.1-10 mOD/min/ug total protein;

aconitase activity of 0.1-50 mOD/min/ug total protein;

maximal fatty acid oxidation level of 0.05-50 pmol O₂/min/ug chondrisome protein;

palmitoyl carnitine & malate RCR3/2 state 3/state 2 respiratory control ratio (RCR 3/2) of 1-10; and/or

electron transport chain efficiency of 1-1000 nmol O₂/min/mg protein/ΔGATP (in kcal/mol).

In some embodiments, the cytobiologic comprises chondrisomes, e.g., isolated chondrisomes from a mitochondrial source, having one or more of the following characteristics:

total lipid content of 50,000-2,000,000 pmol/mg;

double bonds/total lipid ratio of 0.8-8 pmol/pmol;

phospholipid/total lipid ratio of 50-100 100*pmol/pmol;

phosphosphingolipid/total lipid ratio of 0.2-20 100*pmol/pmol;

ceramide content 0.05-5 100*pmol/pmol total lipid;

cardiolipin content 0.05-25 100*pmol/pmol total lipid;

lyso-phosphatidylcholine (LPC) content of 0.05-5 100*pmol/pmol total lipid;

lyso-phosphatidylethanolamine (LPE) content of 0.005-2 100*pmol/pmol total lipid;

phosphatidylcholine (PC) content of 10-80 100*pmol/pmol total lipid;

phosphatidylcholine-ether (PC O−) content 0.1-10 100*pmol/pmol total lipid;

phosphatidylethanolamine (PE) content 1-30 100*pmol/pmol total lipid;

phosphatidylethanolamine-ether (PE O−) content 0.05-30 100*pmol/pmol total lipid;

phosphatidylinositol (PI) content 0.05-15 100*pmol/pmol total lipid;

phosphatidylserine (PS) content 0.05-20 100*pmol/pmol total lipid;

sphingomyelin (SM) content 0.01-20 100*pmol/pmol total lipid;

triacylglycerol (TAG) content 0.005-50 100*pmol/pmol total lipid;

PE:LPE ratio 30-350;

PC:LPC ratio 30-700;

PE 18:n (n>0) content 0.5-20% pmol AA/pmol lipid class;

PE 20:4 content 0.05-20% pmol AA/pmol lipid class;

PC 18:n (n>0) content 5-50% pmol AA/pmol lipid class; and/or

PC 20:4 content 1-20%.

In some embodiments, the cytobiologic comprises a chondrisome, e.g., isolated chondrisomes from a mitochondrial source, having one or more of the following characteristics:

increases basal respiration of recipient cells at least 10%;

chondrisomes in the composition are taken up by at least 1% of recipient cells;

chondrisomes in the composition are taken up and maintain membrane potential in recipient cells;

chondrisomes in the composition persist in recipient cells at least 6 hours;

decrease cellular lipid levels of recipient cells at least 5%;

increases uncoupled respiration of recipient cells at least 5%;

decreases mitochondrial permeability transition pore (MPTP) formation in recipient cells at least 5% and does not increase more than 10%;

increases Akt levels in recipient cells at least 10%;

decreases total NAD/NADH ratio in recipient cells at least 5%; and/or

reduces ROS levels in recipient cells at least 5%.

In some embodiments, a cytobiologic comprising a chondrisome further has one or more of the following characteristics:

increases fractional shortening in subject with cardiac ischemia at least 5%;

increases end diastolic volume in subject with cardiac ischemia at least 5%;

decreases end systolic volume in subject with cardiac ischemia at least 5%;

decreases infarct area of ischemic heart at least 5%;

increases stroke volume in subject with cardiac ischemia at least 5%;

increases ejection fraction in subject with cardiac ischemia at least 5%;

increases cardia output in subject with cardiac ischemia at least 5%;

increases cardiac index in subject with cardiac ischemia at least 5%;

decreases serum CKNB levels in subject with cardiac ischemia at least 5%;

decreases serum cTnI levels in subject with cardiac ischemia at least 5%; and/or decreases serum hydrogen peroxide in subject with cardiac ischemia at least 5%.

In embodiments, the cytobiologic comprising a chondrisome is stable for at least 6 hours, 12 hours, 24 hours, 48 hours, 72 hours, 96 hours, 5 days, 7 days, 10 days, 14 days, 21 days, 30 days, 45 days, 60 days, 90 days, 120 days, 180 days, or longer (for example, at 4° C., 0° C., −4° C., or −20° C., −80° C.).

In embodiments, the cytobiologic comprising an agent (e.g., a chondrisome) may comprise, e.g., a natural, synthetic or engineered encapsulation material such as a lipid based material, vesicle, exosome, lipid raft, clathrin coated vesicle, or platelet (mitoparticle), MSC or astrocyte microvesicle membrane.

In embodiments, the cytobiologic comprising a chondrisome is in a composition at between 150-20,000 ug protein/ml; between 150-15,000 ug/ml; 200-15,000 ug/ml; 300-15,000 ug/ml; 500-15,000 ug/ml; 200-10,000 ug/ml; 200-5,000 ug/ml; 300-10,000 ug/ml; >200 ug/ml; >250 ug/ml; >300 ug/ml; >350 ug/ml; >400 ug/ml; >450 ug/ml; >500 ug/ml; >600 ug/ml; >700 ug/ml; >800 ug/ml; >900 ug/ml; >1 mg/ml; >2 mg/ml; >3 mg/ml; >4 mg/ml; >5 mg/ml; >6 mg/ml; >7 mg/ml; >8 mg/ml; >9 mg/ml; >10 mg/ml; >11 mg/ml; >12 mg/ml; >14 mg/ml; >15 mg/ml (and, e.g., <20 mg/ml).

In embodiments, the cytobiologic comprising a chondrisome does not produce an undesirable immune response in a recipient animal, e.g., a recipient mammal such as a human (e.g., does not significantly increase levels of IL-1-beta, IL-6, GM-CSF, TNF-alpha, or lymph node size, in the recipient).

Modifications to the cargo include, for example, modifications to chondrisomes or the source of chondrisomes as described in international application, PCT/US16/64251. In some embodiments, the cytobiologic comprises a chondrisome made using a method of making a pharmaceutical composition described herein.

In some embodiments, a cytobiologic composition described herein, e.g., a cytobiologic composition comprising mitochondria or chondrisomes, is capable of one or more of (e.g., 2, 3, or 4 of):

a) increasing maximal respiration in a target cell, e.g., wherein the increase in maximal respiration is at least 10%, 20%, 30%, 40%, 50%, 60%, 70%, 75% 80%, 90%, 2-fold, 3-fold, 4-fold, or 5-fold, or from 10%-20%, 20%-30%, 30%-40%, 40%-50%, 50%-60%, 60%-70%, 70%-80%, 80%-90%, 90%-100%, 1-fold-2-fold, 2-fold-3-fold, 3-fold-4-fold, or 4-fold-5-fold;

b) increasing spare respiratory capacity in a target cell, e.g., wherein the increase in spare respiratory capacity is at least 10%, 20%, 30%, 40%, 50%, 60%, 70%, 80%, 90%, 2-fold, 3-fold, 4-fold, or 5-fold, or from 10%-20%, 20%-30%, 30%-40%, 40%-50%, 50%-60%, 60%-70%, 70%-80%, 80%-90%, 90%-100%, 1-fold-2-fold, 2-fold-3-fold, 3-fold-4-fold, or 4-fold-5-fold;

c) stimulating mitochondrial biogenesis in a target cell, e.g., wherein stimulating mitochondrial biogenesis comprises increasing mitochondrial biomass by at least 10%, 20%, 30%, 40%, 50%, 60%, 70%, 80%, 90%, 2-fold, 3-fold, 4-fold, or 5-fold, or from 10%-20%, 20%-30%, 30%-40%, 40%-50%, 50%-60%, 60%-70%, 70%-80%, 80%-90%, 90%-100%, 1-fold-2-fold, 2-fold-3-fold, 3-fold-4-fold, or 4-fold-5-fold; or

d) modulating (e.g., stimulating or inhibiting) transcription of a nuclear gene in a target cell, e.g., wherein the change in transcript levels of the nuclear gene is at least 10%, 20%, 30%, 40%, 50%, 60%, 70%, 80%, 90%, 2-fold, 3-fold, 4-fold, or 5-fold, or from 10%-20%, 20%-30%, 30%-40%, 40%-50%, 50%-60%, 60%-70%, 70%-80%, 80%-90%, 90%-100%, 1-fold-2-fold, 2-fold-3-fold, 3-fold-4-fold, or 4-fold-5-fold.

### Immunogenicity

In some embodiments of any of the aspects described herein, the cytobiologic composition is substantially non-immunogenic. Immunogenicity can be quantified, e.g., as described herein.

In some embodiments, the cytobiologic composition has membrane symmetry of a cell which is, or is known to be, substantially non-immunogenic, e.g., a stem cell, mesenchymal stem cell, induced pluripotent stem cell, embryonic stem cell, sertoli cell, or retinal pigment epithelial cell. In some embodiments, the cytobiologic has an immunogenicity no more than 5%, 10%, 20%, 30%, 40%, or 50% greater than the immunogenicity of a stem cell, mesenchymal stem cell, induced pluripotent stem cell, embryonic stem cell, sertoli cell, or retinal pigment epithelial cell as measured by an assay described herein.

In some embodiments, the cytobiologic composition comprises elevated levels of an immunosuppressive agent as compared to a reference cell, e.g., an unmodified cell otherwise similar to the source cell, or a Jurkat cell. In some embodiments, the elevated level is at least 5%, 10%, 20%, 30%, 40%, 50%, 60%, 70%, 80%, 90%, 2-fold, 3-fold, 5-fold, 10-fold, 20-fold, 50-fold, or 100-fold. In some embodiments, the cytobiologic composition comprises an immunosuppressive agent that is absent from the reference cell. In some embodiments, the cytobiologic composition comprises reduced levels of an immune activating agent as compared to a reference cell, e.g., an unmodified cell otherwise similar to the source cell, or a Jurkat cell. In some embodiments, the reduced level is at least 5%, 10%, 20%, 30%, 40%, 50%, 60%, 70%, 80%, 90%, 95%, 98%, or 99% compared to the reference cell. In some embodiments, the immune activating agent is substantially absent from the cytobiologic.

In some embodiments, the cytobiologic composition comprises a membrane with composition substantially similar, e.g., as measured by proteomics, to that of a source cell, e.g., a substantially non-immunogenic source cell. In some embodiments, the cytobiologic composition comprises a membrane comprising at least 1%, 2%, 3%, 4%, 5%, 10%, 20%, 30%, 40%, 50%, 60%, 70%, 80%, 90%, 95%, 99%, or 100% of the membrane proteins of the source cell. In some embodiments, the cytobiologic composition comprises a membrane comprising membrane proteins expressed at, at least 1%, 2%, 3%, 4%, 5%, 10%, 20%, 30%, 40%, 50%, 60%, 70%, 80%, 90%, 95%, 99%, or 100% of the level of expression of the membrane proteins on a membrane of the source cell.

In some embodiments, the cytobiologic composition, or the source cell from which the cytobiologic composition is derived from, has one, two, three, four, five, six, seven, eight, nine, ten, eleven, twelve, or more of the following characteristics:

a. less than 50%, 40%, 30%, 20%, 15%, 10%, or 5% or lesser expression of MHC class I or MHC class II, compared to a reference cell, e.g., an unmodified cell otherwise similar to the source cell, or a HeLa cell;

b. less than 50%, 40%, 30%, 20%, 15%, 10%, or 5% or lesser expression of one or more co-stimulatory proteins including but not limited to: LAG3, ICOS-L, ICOS, Ox40L, OX40, CD28, B7, CD30, CD30L 4-1BB, 4-1BBL, SLAM, CD27, CD70, HVEM, LIGHT, B7-H3, or B7-H4, compared to a reference cell, e.g., an unmodified cell otherwise similar to the source cell, or a reference cell described herein;

c. expression of surface proteins which suppress macrophage engulfment e.g., CD47, e.g., detectable expression by a method described herein, e.g., more than 1.5-fold, 2-fold, 3-fold, 4-fold, 5-fold, 10-fold, or more expression of the surface protein which suppresses macrophage engulfment, e.g., CD47, compared to a reference cell, e.g., an unmodified cell otherwise similar to the source cell, or a Jurkat cell;

d. expression of soluble immunosuppressive cytokines, e.g., IL-10, e.g., detectable expression by a method described herein, e.g., more than 1.5-fold, 2-fold, 3-fold, 4-fold, 5-fold, 10-fold, or more expression of soluble immunosuppressive cytokines, e.g., IL-10, compared to a reference cell, e.g., an unmodified cell otherwise similar to the source cell, or a Jurkat cell;

e. expression of soluble immunosuppressive proteins, e.g., PD-L1, e.g., detectable expression by a method described herein, e.g., more than 1.5-fold, 2-fold, 3-fold, 4-fold, 5-fold, 10-fold, or more expression of soluble immunosuppressive proteins, e.g., PD-L1, compared to a reference cell e.g., an unmodified cell otherwise similar to the source cell, or a Jurkat cell;

f. less than 50%, 40%, 30%, 20%, 15%, 10%, or 5% or lesser expression of soluble immune stimulating cytokines, e.g., IFN-gamma or TNF-a, compared to a reference cell, e.g., an unmodified cell otherwise similar to the source cell, or a U-266 cell;

g. less than 50%, 40%, 30%, 20%, 15%, 10%, or 5% or lesser expression of endogenous immune-stimulatory antigen, e.g., Zg16 or Hormad1, compared to a reference cell, e.g., an unmodified cell otherwise similar to the source cell, or an A549 cell or a SK-BR-3 cell;

h. expression of, e.g., detectable expression by a method described herein, HLA-E or HLA-G, compared to a reference cell, e.g., an unmodified cell otherwise similar to the source cell, or a Jurkat cell;

i. surface glycosylation profile, e.g., containing sialic acid, which acts to, e.g., suppress NK cell activation;

j. less than 50%, 40%, 30%, 20%, 15%, 10%, or 5% or lesser expression of TCRα/β, compared to a reference cell, e.g., an unmodified cell otherwise similar to the source cell, or a Jurkat cell;

k. less than 50%, 40%, 30%, 20%, 15%, 10%, or 5% or lesser expression of ABO blood groups, compared to a reference cell, e.g., an unmodified cell otherwise similar to the source cell, or a HeLa cell;

l. less than 50%, 40%, 30%, 20%, 15%, 10%, or 5% or lesser expression of Minor Histocompatibility Antigen (MHA), compared to a reference cell, e.g., an unmodified cell otherwise similar to the source cell, or a Jurkat cell; or

m. has less than 10%, 9%, 8%, 7%, 6%, 5%, 4%, 3%, 2%, 1%, or less, of mitochondrial MHAs, compared to a reference cell e.g., an unmodified cell otherwise similar to the source cell, or a Jurkat cell, or has no detectable mitochondrial MHAs.

In embodiments, the co-stimulatory protein is 4-1BB, B7, SLAM, LAG3, HVEM, or LIGHT, and the ref cell is HDLM-2. In some embodiments, the co-stimulatory protein is BY-H3 and the reference cell is HeLa. In some embodiments, the co-stimulatory protein is ICOSL or B7-H4, and the reference cell is SK-BR-3. In some embodiments, the co-stimulatory protein is ICOS or OX40, and the reference cell is MOLT-4. In some embodiments, the co-stimulatory protein is CD28, and the reference cell is U-266. In some embodiments, the co-stimulatory protein is CD30L or CD27, and the reference cell is Daudi. In some embodiments, the cytobiologic composition does not substantially elicit an immunogenic response by the immune system, e.g., innate immune system. In embodiments, an immunogenic response can be quantified, e.g., as described herein. In some embodiments, the an immunogenic response by the innate immune system comprises a response by innate immune cells including, but not limited to NK cells, macrophages, neutrophils, basophils, eosinophils, dendritic cells, mast cells, or gamma/delta T cells. In some embodiments, an immunogenic response by the innate immune system comprises a response by the complement system which includes soluble blood components and membrane bound components.

In some embodiments, the cytobiologic composition does not substantially elicit an immunogenic response by the immune system, e.g., adaptive immune system. In embodiments, an immunogenic response can be quantified, e.g., as described herein. In some embodiments, an immunogenic response by the adaptive immune system comprises an immunogenic response by an adaptive immune cell including, but not limited to a change, e.g., increase, in number or activity of T lymphocytes (e.g., CD4 T cells, CD8 T cells, and or gamma-delta T cells), or B lymphocytes. In some embodiments, an immunogenic response by the adaptive immune system includes increased levels of soluble blood components including, but not limited to a change, e.g., increase, in number or activity of cytokines or antibodies (e.g., IgG, IgM, IgE, IgA, or IgD).

In some embodiments, the cytobiologic composition is modified to have reduced immunogenicity. Immunogenicity can be quantified, e.g., as described herein. In some embodiments, the cytobiologic composition has an immunogenicity less than 5%, 10%, 20%, 30%, 40%, or 50% lesser than the immunogenicity of a reference cell, e.g., an unmodified cell otherwise similar to the source cell, or a Jurkat cell.

In some embodiments of any of the aspects described herein, the cytobiologic composition is derived from a source cell, e.g., a mammalian cell, having a modified genome, e.g., modified using a method described herein, to reduce, e.g., lessen, immunogenicity. Immunogenicity can be quantified, e.g., as described herein.

In some embodiments, the cytobiologic composition is derived from a mammalian cell depleted of, e.g., with a knock out of, one, two, three, four, five, six, seven or more of the following:
a. MHC class I, MHC class II or MHA;
b. one or more co-stimulatory proteins including but not limited to: LAG3, ICOS-L, ICOS, Ox40L, OX40, CD28, B7, CD30, CD30L 4-1BB, 4-1BBL, SLAM, CD27, CD70, HVEM, LIGHT, B7-H3, or B7-H4;
c. soluble immune-stimulating cytokines e.g., IFN-gamma or TNF-α;
d. endogenous immune-stimulatory antigen, e.g., Zg16 or Hormad1;
e. T-cell receptors (TCR);
f. The genes encoding ABO blood groups, e.g., ABO gene;
g. transcription factors which drive immune activation, e.g., NFkB;
h. transcription factors that control MHC expression e.g., class II trans-activator (CIITA), regulatory factor of the Xbox 5 (RFX5), RFX-associated protein (RFXAP), or RFX ankyrin repeats (RFXANK; also known as RFXB); or
i. TAP proteins, e.g., TAP2, TAP1, or TAPBP, which reduce MHC class I expression.

In some embodiments, the cytobiologic is derived from a source cell with a genetic modification which results in increased expression of an immunosuppressive agent, e.g., one, two, three or more of the following (e.g., wherein before the genetic modification the cell did not express the factor):

a. surface proteins which suppress macrophage engulfment, e.g., CD47; e.g., increased expression of CD47 compared to a reference cell, e.g., an unmodified cell otherwise similar to the source cell, or a Jurkat cell;

b. soluble immunosuppressive cytokines, e.g., IL-10, e.g., increased expression of IL-10 compared to a reference cell, e.g., an unmodified cell otherwise similar to the source cell, or a Jurkat cell;

c. soluble immunosuppressive proteins, e.g., PD-1, PD-L1, CTLA4, or BTLA; e.g., increased expression of immunosuppressive proteins compared to a reference cell, e.g., an unmodified cell otherwise similar to the cell source, or a Jurkat cell;

d. a tolerogenic protein, e.g., an ILT-2 or ILT-4 agonist, e.g., HLA-E or HLA-G or any other endogenous ILT-2 or ILT-4 agonist, e.g., increased expression of HLA-E, HLA-G, ILT-2 or ILT-4 compared to a reference cell, e.g., an unmodified cell otherwise similar to the source cell, or a Jurkat cell, or

e. surface proteins which suppress complement activity, e.g., complement regulatory proteins, e.g. proteins that bind decay-accelerating factor (DAF, CD55), e.g. factor H (FH)-like protein-1 (FHL-1), e.g. C4b-binding protein (C4BP), e.g. complement receptor 1 (CD35), e.g. Membrane cofactor protein (MCP, CD46), eg. Profectin (CD59), e.g. proteins that inhibit the classical and alternative compelement pathway CD/C5 convertase enzymes, e.g. proteins that regulate MAC assembly; e.g. increased expression of a complement regulatory protein compared to a reference cell, e.g. an umodified cell otherwise similar to the the source cell, or a Jurkat cell.

In some embodiments, the increased expression level is at least 5%, 10%, 20%, 30%, 40%, 50%, 60%, 70%, 80%, 90%, 2-fold, 3-fold, 5-fold, 10-fold, 20-fold, 50-fold, or 100-fold higher as compared to a reference cell.

In some embodiments, the cytobiologic is derived from a source cell modified to have decreased expression of an immune activating agent, e.g., one, two, three, four, five, six, seven, eight or more of the following:

a. less than 50%, 40%, 30%, 20%, 15%, 10%, or 5% or lesser expression of MHC class I or MHC class II, compared to a reference cell, e.g., an unmodified cell otherwise similar to the source cell, or a HeLa cell;

b. less than 50%, 40%, 30%, 20%, 15%, 10%, or 5% or lesser expression of one or more co-stimulatory proteins including but not limited to: LAG3, ICOS-L, ICOS, Ox40L, OX40, CD28, B7, CD30, CD30L 4-1BB, 4-1BBL, SLAM, CD27, CD70, HVEM, LIGHT, B7-H3, or B7-H4, compared to a reference cell, e.g., an unmodified cell otherwise similar to the source cell, or a reference cell described herein;

c. less than 50%, 40%, 30%, 20%, 15%, 10%, or 5% or lesser expression of soluble immune stimulating cytokines, e.g., IFN-gamma or TNF-α, compared to a reference cell, e.g., an unmodified cell otherwise similar to the source cell, or a U-266 cell;

d. less than 50%, 40%, 30%, 20%, 15%, 10%, or 5% or lesser expression of endogenous immune-stimulatory antigen, e.g., Zg16 or Hormad1, compared to a reference cell, e.g., an unmodified cell otherwise similar to the source cell, or an A549 cell or a SK-BR-3 cell;

e. less than 50%, 40%, 30%, 20%, 15%, 10%, or 5% or lesser expression of T-cell receptors (TCR) compared to a reference cell, e.g., an unmodified cell otherwise similar to the source cell, or a Jurkat cell;

f. less than 50%, 40%, 30%, 20%, 15%, 10%, or 5% or lesser expression of ABO blood groups, compared to a reference cell, e.g., an unmodified cell otherwise similar to the source cell, or a HeLa cell;

g. less than 50%, 40%, 30%, 20%, 15%, 10%, or 5% or lesser expression of transcription factors which drive immune activation, e.g., NFkB; compared to a reference cell, e.g., an unmodified cell otherwise similar to the source cell, or a Jurkat cell

h. less than 50%, 40%, 30%, 20%, 15%, 10%, or 5% or lesser expression of transcription factors that control MHC expression, e.g., class II trans-activator (CIITA), regulatory factor of the Xbox 5 (RFX5), RFX-associated protein (RFXAP), or RFX ankyrin repeats (RFXANK; also known as RFXB) compared to a reference cell, e.g., an unmodified cell otherwise similar to the source cell, or a Jurkat cell; or

i. less than 50%, 40%, 30%, 20%, 15%, 10%, or 5% or lesser expression of TAP proteins, e.g., TAP2, TAP1, or TAPBP, which reduce MHC class I expression compared to a reference cell, e.g., an unmodified cell otherwise similar to the source cell, or a HeLa cell.

In some embodiments, a cytobiologic composition derived from a mammalian cell, e.g., a mesenchymal stem cell, modified using shRNA expressing lentivirus to decrease MHC Class I expression, has lesser expression of MHC Class I compared to an unmodified cell, e.g., a mesenchymal stem cell that has not been modified. In some embodiments, a cytobiologic composition derived from a mammalian cell, e.g., a mesenchymal stem cell, modified using lentivirus expressing HLA-G to increase expression of HLA-G, has increased expression of HLA-G compared to an unmodified cell, e.g., a mesenchymal stem cell that has not been modified.

In some embodiments, the cytobiologic composition is derived from a source cell, e.g., a mammalian cell, which is not substantially immunogenic, wherein the source cells stimulate, e.g., induce, T-cell IFN-gamma secretion, at a level of 0 pg/mL to >0 pg/mL, e.g., as assayed in vitro, by IFN-gamma ELISPOT assay.

In some embodiments, the cytobiologic composition is derived from a source cell, e.g., a mammalian cell, wherein the mammalian cell is from a cell culture treated with an immunosuppressive agent, e.g., a glucocorticoid (e.g., dexamethasone), cytostatic (e.g., methotrexate), antibody (e.g., Muromonab-CD3), or immunophilin modulator (e.g., Ciclosporin or rapamycin).

In some embodiments, the cytobiologic composition is derived from a source cell, e.g., a mammalian cell, wherein the mammalian cell comprises an exogenous agent, e.g., a therapeutic agent.

In some embodiments, the cytobiologic composition is derived from a source cell, e.g., a mammalian cell, wherein the mammalian cell is a recombinant cell.

In some embodiments, the cytobiologic is derived from a mammalian cell genetically modified to express viral immunoevasins, e.g., hCMV US2, or US11.

In some embodiments, the surface of the cytobiologic, or the surface of the mammalian cell the cytobiologic is derived from, is covalently or non-covalently modified with a polymer, e.g., a biocompatible polymer that reduces immunogenicity and immune-mediated clearance, e.g., PEG.

In some embodiments, the surface of the cytobiologic, or the surface of the mammalian cell the cytobiologic is derived from is covalently or non-covalently modified with a sialic acid, e.g., a sialic acid comprising glycopolymers, which contain NK-suppressive glycan epitopes.

In some embodiments, the surface of the cytobiologic, or the surface of the mammalian cell the cytobiologic is derived from is enzymatically treated, e.g., with glycosidase enzymes, e.g., α-N-acetylgalactosaminidases, to remove ABO blood groups

In some embodiments, the surface of the cytobiologic, or the surface of the mammalian cell the cytobiologic is derived from is enzymatically treated, to give rise to, e.g., induce expression of, ABO blood groups which match the recipient's blood type.

### Parameters for Assessing Immunogenicity

In some embodiments, the cytobiologic composition is derived from a source cell, e.g., a mammalian cell which is not substantially immunogenic, or modified, e.g., modified using a method described herein, to have a reduction in immunogenicity. Immunogenicity of the source cell and the cytobiologic composition can be determined by any of the assays described herein.

In some embodiments, the cytobiologic composition has an increase, e.g., an increase of 1%, 5%, 10%, 20%, 30%, 40%, 50%, 60%, 70%, 80%, 90%, or more, in in vivo graft survival compared to a reference cell, e.g., an unmodified cell otherwise similar to the source cell. In some embodiments, graft survival is determined by an assay measuring in vivo graft survival as described herein, in an appropriate animal model, e.g., an animal model described herein.

In some embodiments, the cytobiologic composition has an increase, e.g., an increase of 1%, 5%, 10%, 20%, 30%, 40%, 50%, 60%, 70%, 80%, 90%, or more in teratoma formation compared to a reference cell, e.g., an unmodified cell otherwise similar to the source cell. In some embodiments, teratroma formation is determined by an assay measuring teratoma formation as described herein, in an appropriate animal model, e.g., in an animal model described herein.

In some embodiments, the cytobiologic composition has an increase, e.g., an increase of 1%, 5%, 10%, 20%, 30%, 40%, 50%, 60%, 70%, 80%, 90%, or more in teratoma survival compared to a reference cell, e.g., an unmodified cell otherwise similar to the source cell. In some embodiments, the cytobiologic composition survives for one or more days in an assay of teratoma survival. In some embodiments, teratroma survival is determined by an assay measuring teratoma survival as described herein, in an appropriate animal model, e.g., in an animal model described herein. In an embodiment, teratoma formation is measured by imaging analysis, e.g., IHC staining, fluorescent staining or H&E, of fixed tissue, e.g., frozen or formalin fixed, as described in the Examples. In some embodiments, fixed tissue can be stained with any one or all of the following antibodies: anti-human CD3, anti-human CD4, or anti-human CD8.

In some embodiments, the cytobiologic composition has a reduction, e.g., a reduction of 1%, 5%, 10%, 20%, 30%, 40%, 50%, 60%, 70%, 80%, 90%, or more in CD8+ T cell infiltration into a graft or teratoma compared to a reference cell, e.g., an unmodified cell otherwise similar to the source cell. In an embodiment, CD8 T cell infiltration is determined by an assay measuring CD8+ T cell infiltration as described herein, e.g., histological analysis, in an appropriate animal model, e.g., an animal model described herein. In some embodiments, teratomas derived from the cytobiologic composition have CD8+ T cell infiltration in 0%, 0.1%, 1% 5%, 10%, 20%, 30%, 40% 50%, 60%, 70%, 80%, 90%, or 100% of 50× image fields of a histology tissue section.

In some embodiments, the cytobiologic composition has a reduction, e.g., a reduction of 1%, 5%, 10%, 20%, 30%, 40%, 50%, 60%, 70%, 80%, 90%, or more in CD4+ T cell infiltration into a graft or teratoma compared to a reference cell, e.g., an unmodified cell otherwise similar to the source cell. In some embodiments, CD4 T cell infiltration is determined by an assay measuring CD4+ T cell infiltration as described herein, e.g., histological analysis, in an appropriate animal model, e.g., an animal model described herein. In some embodiments, teratomas derived from the cytobiologic composition have CD4+ T cell infiltration in 0%, 0.1%, 1% 5%, 10%, 20%, 30%, 40% 50%, 60%, 70%, 80%, 90%, or 100% of 50× image fields of a histology tissue section.

In some embodiments, the cytobiologic composition has a reduction, e.g., a reduction of 1%, 5%, 10%, 20%, 30%, 40%, 50%, 60%, 70%, 80%, 90%, or more in CD3+ NK cell infiltration into a graft or teratoma compared to a reference cell, e.g., an unmodified cell otherwise similar to the source cell. In an embodiment, CD3+ NK cell infiltration is determined by an assay measuring CD3+ NK cell infiltration as described herein, e.g., histological analysis, in an appropriate animal model, e.g., an animal model described herein. In some embodiments, teratomas derived from the cytobiologic composition have CD3+ NK T cell infiltration in 0%, 0.1%, 1% 5%, 10%, 20%, 30%, 40% 50%, 60%, 70%, 80%, 90%, or 100% of 50× image fields of a histology tissue section.

In some embodiments, the cytobiologic composition has a reduction in immunogenicity as measured by a reduction in humoral response following one or more implantation of the cytobiologic derived into an appropriate animal model, e.g., an animal model described herein, compared to a humoral response following one or more implantation of a reference cell, e.g., an unmodified cell otherwise similar to the source cell, into an appropriate animal model, e.g., an animal model described herein. In some embodiments, the reduction in humoral response is measured in a serum sample by an anti-cell antibody titre, e.g., anti-cytobiologic antibody titre, e.g., by ELISA. In some embodiments, the serum sample from animals administered the cytobiologic composition has a reduction of 1%, 5%, 10%, 20%, 30%, 40%, 50%, 60%, 70%, 80%, 90%, or more of an anti-cell antibody titer compared to the serum sample from animals administered an unmodified cell. In some embodiments, the serum sample from animals administered the cytobiologic composition has an increased anti-cell antibody titre, e.g., increased by 1%, 2%, 5%, 10%, 20%, 30%, or 40% from baseline, e.g., wherein baseline refers to serum sample from the same animals before administration of the cytobiologic composition.

In some embodiments, the cytobiologic composition has a reduction in macrophage phagocytosis, e.g., a reduction of 1%, 5%, 10%, 20%, 30%, 40%, 50%, 60%, 70%, 80%, 90%, or more in macrophage phagocytosis compared to a reference cell, e.g., an unmodified cell otherwise similar to the source cell, wherein the reduction in macrophage phagocytosis is determined by assaying the phagocytosis index in vitro, e.g., as described in Example 66. In some embodiments, the cytobiologic composition has a phagocytosis index of 0, 1, 10, 100, or more, e.g., as measured by an assay of Example 66, when incubated with macrophages in an in vitro assay of macrophage phagocytosis.

In some embodiments, the source cell has a reduction in cytotoxicity mediated cell lysis by PBMCs, e.g., a reduction of 1%, 5%, 10%, 20%, 30%, 40%, 50%, 60%, 70%, 80%, 90%, or more in cell lysis compared to a reference cell, e.g., an unmodified cell otherwise similar to the source cell or a mesenchymal stem cells, e.g., using an assay of Example 67. In embodiments, the source cell expresses exogenous HLA-G.

In some embodiments, the cytobiologic composition has a reduction in NK-mediated cell lysis, e.g., a reduction of 1%, 5%, 10%, 20%, 30%, 40%, 50%, 60%, 70%, 80%, 90%, or more in NK-mediated cell lysis compared to a reference cell, e.g., an unmodified cell otherwise similar to the source cell, wherein NK-mediated cell lysis is assayed in vitro, by a chromium release assay or europium release assay.

In some embodiments, the cytobiologic composition has a reduction in CD8+ T-cell mediated cell lysis, e.g., a reduction of 1%, 5%, 10%, 20%, 30%, 40%, 50%, 60%, 70%, 80%, 90%, or more in CD8 T cell mediated cell lysis compared to a reference cell, e.g., an unmodified cell otherwise similar to the source cell, wherein CD8 T cell mediated cell lysis is assayed in vitro, by a chromium release assay or europium release assay. In embodiments, activation and/or proliferation is measured as described in Example 69.

In some embodiments, the cytobiologic composition has a reduction in CD4+ T-cell proliferation and/or activation, e.g., a reduction of 1%, 5%, 10%, 20%, 30%, 40%, 50%, 60%, 70%, 80%, 90%, or more compared to a reference cell, e.g., an unmodified cell otherwise similar to the source cell, wherein CD4 T cell proliferation is assayed in vitro (e.g. co-culture assay of modified or unmodified mammalian source cell, and CD4+ T-cells with CD3/CD28 Dynabeads), e.g., as described in Example 70.

In some embodiments, the cytobiologic composition has a reduction in T-cell IFN-gamma secretion, e.g., a reduction of 1%, 5%, 10%, 20%, 30%, 40%, 50%, 60%, 70%, 80%, 90%, or more in T-cell IFN-gamma secretion compared to a reference cell, e.g., an unmodified cell otherwise similar to the source cell, wherein T-cell IFN-gamma secretion is assayed in vitro, e.g., by IFN-gamma ELISPOT.

In some embodiments, the cytobiologic composition has a reduction in secretion of immunogenic cytokines, e.g., a reduction of 1%, 5%, 10%, 20%, 30%, 40%, 50%, 60%, 70%, 80%, 90%, or more in secretion of immunogenic cytokines compared to a reference cell, e.g., an unmodified cell otherwise similar to the source cell, wherein secretion of immunogenic cytokines is assayed in vitro using ELISA or ELISPOT.

In some embodiments, the cytobiologic composition results in increased secretion of an immunosuppressive cytokine, e.g., an increase of 1%, 5%, 10%, 20%, 30%, 40%, 50%, 60%, 70%, 80%, 90%, or more in secretion of an immunosuppressive cytokine compared to a reference cell, e.g., an unmodified cell otherwise similar to the source cell, wherein secretion of the immunosuppressive cytokine is assayed in vitro using ELISA or ELISPOT.

In some embodiments, the cytobiologic composition has an increase in expression of HLA-G or HLA-E, e.g., an increase in expression of 1%, 5%, 10%, 20%, 30%, 40%, 50%, 60%, 70%, 80%, 90%, or more of HLA-G or HLA-E, compared to a reference cell, e.g., an unmodified cell otherwise similar to the source cell, wherein expression of HLA-G or HLA-E is assayed in vitro using flow cytometry, e.g., FACS. In some embodiments, the cytobiologic composition is derived from a source cell which is modified to have an increased expression of HLA-G or HLA-E, e.g., compared to an unmodified cell, e.g., an increased expression of 1%, 5%, 10%, 20%, 30%, 40%, 50%, 60%, 70%, 80%, 90%, or more of HLA-G or HLA-E, wherein expression of HLA-G or HLA-E is assayed in vitro using flow cytometry, e.g., FACS. In some embodiments, the cytobiologic composition derived from a modified cell with increased HLA-G expression demonstrates reduced immunogenicity, e.g., as measured by reduced immune cell infiltration, in a teratoma formation assay, e.g., a teratoma formation assay as described herein.

In some embodiments, the cytobiologic composition has an increase in expression of T cell inhibitor ligands (e.g. CTLA4, PD1, PD-L1), e.g., an increase in expression of 1%, 5%, 10%, 20%, 30%, 40%, 50%, 60%, 70%, 80%, 90%, or more of T cell inhibitor ligands as compared to a reference cell, e.g., an unmodified cell otherwise similar to the source cell, wherein expression of T cell inhibitor ligands is assayed in vitro using flow cytometry, e.g., FACS.

In some embodiments, the cytobiologic composition has a decrease in expression of co-stimulatory ligands, e.g., a decrease of 1%, 5%, 10%, 20%, 30%, 40%, 50%, 60%, 70%, 80%, 90%, or more in expression of co-stimulatory ligands compared to a reference cell, e.g., an unmodified cell otherwise similar to the source cell, wherein expression of co-stimulatory ligands is assayed in vitro using flow cytometry, e.g., FACS.

In some embodiments, the cytobiologic composition has a decrease in expression of MHC class I or MHC class II, e.g., a decrease in expression of 1%, 5%, 10%, 20%, 30%, 40%, 50%, 60%, 70%, 80%, 90%, or more of MHC Class I or MHC Class II compared to a reference cell, e.g., an unmodified cell otherwise similar to the source cell or a HeLa cell, wherein expression of MHC Class I or II is assayed in vitro using flow cytometry, e.g., FACS.

In some embodiments, the cytobiologic composition is derived from a cell source, e.g., a mammalian cell source, which is substantially non-immunogenic. In some embodiments, immunogenicity can be quantified, e.g., as described herein. In some embodiments, the mammalian cell source comprises any one, all or a combination of the following features:
a. wherein the source cell is obtained from an autologous cell source; e.g., a cell obtained from a recipient who will be receiving, e.g., administered, the cytobiologic composition;
b. wherein the source cell is obtained from an allogeneic cell source which is of matched, e.g., similar, gender to a recipient, e.g., a recipient described herein who will be receiving, e.g., administered; the cytobiologic composition;
c. wherein the source cell is obtained is from an allogeneic cell source is which is HLA matched with a recipient's HLA, e.g., at one or more alleles;
d. wherein the source cell is obtained is from an allogeneic cell source which is an HLA homozygote;
e. wherein the source cell is obtained is from an allogeneic cell source which lacks (or has reduced levels compared to a reference cell) MHC class I and II; or
f. wherein the source cell is obtained is from a cell source which is known to be substantially non-immunogenic including but not limited to a stem cell, a mesenchymal stem cell, an induced pluripotent stem cell, an embryonic stem cell, a sertoli cell, or a retinal pigment epithelial cell.

In some embodiments, the subject to be administered the cytobiologic composition has, or is known to have, or is tested for, a pre-existing antibody (e.g., IgG or IgM) reactive with a cytobiologic. In some embodiments, the subject to be administered the cytobiologic composition does not have detectable levels of a pre-existing antibody reactive with the cytobiologic. Tests for the antibody are described, e.g., in Example 62.

In some embodiments, a subject that has received the cytobiologic composition has, or is known to have, or is tested for, an antibody (e.g., IgG or IgM) reactive with a cytobiologic. In some embodiments, the subject that received the cytobiologic composition (e.g., at least once, twice, three times, four times, five times, or more) does not have detectable levels of antibody reactive with the cytobiologic. In embodiments, levels of antibody do not rise more than 1%, 2%, 5%, 10%, 20%, or 50% between two timepoints, the first timepoint being before the first administration of the cytobiologic, and the second timepoint being after one or more administrations of the cytobiologic. Tests for the antibody are described, e.g., in Example 63.

### Additional Therapeutic Agents

In some embodiments, the cytobiologic composition is co-administered with an additional agent, e.g., a therapeutic agent, to a subject, e.g., a recipient, e.g., a recipient described herein. In some embodiments, the co-administered therapeutic agent is an immunosuppressive agent, e.g., a glucocorticoid (e.g., dexamethasone), cytostatic (e.g., methotrexate), antibody (e.g., Muromonab-CD3), or immunophilin modulator (e.g., Ciclosporin or rapamycin). In embodiments, the immunosuppressive agent decreases immune mediated clearance of cytobiologics. In some embodiments the cytobiologic composition is co-administered with an immunostimulatory agent, e.g., an adjuvant, an interleukin, a cytokine, or a chemokine.

In some embodiments, the cytobiologic composition and the immunosuppressive agent are administered at the same time, e.g., contemporaneously administered. In some embodiments, the cytobiologic composition is administered before administration of the immunosuppressive agent. In some embodiments, the cytobiologic composition is administered after administration of the immunosuppressive agent.

In some embodiments, the immunosuppressive agent is a small molecule such as ibuprofen, acetaminophen, cyclosporine, tacrolimus, rapamycin, mycophenolate, cyclophosphamide, glucocorticoids, sirolimus, azathriopine, or methotrexate.

In some embodiments, the immunosuppressive agent is an antibody molecule, including but not limited to: muronomab (anti-CD3), Daclizumab (anti-IL12), Basiliximab, Infliximab (Anti-TNFa), or rituximab (Anti-CD20).

In some embodiments, co-administration of the cytobiologic composition with the immunosuppressive agent results in enhanced persistence of the cytobiologic composition in the subject compared to administration of the cytobiologic composition alone. In some embodiments, the enhanced persistence of the cytobiologic composition in the co-administration is at least 10%, 20%, 30%, 40%, 50%, 60%, 70%, 80%, 90% or longer, compared to persistence of the cytobiologic composition when administered alone. In some embodiments, the enhanced persistence of the cytobiologic composition in the co-administration is at least 1, 2, 3, 4, 5, 6, 7, 10, 15, 20, 25, or 30 days or longer, compared to survival of the cytobiologic composition when administered alone.

### Delivery

Compositions comprising the cytobiologics described herein may be administered or targeted to the circulatory system, hepatic system, renal system, cardio-pulmonary system, central nervous system, peripheral nervous system, musculoskeletal system, lymphatic system, immune system, sensory nervous systems (sight, hearing, smell, touch, taste), digestive system, endocrine systems (including adipose tissue metabolic regulation), and reproductive system.

In embodiments, a cytobiologic composition described herein is delivered ex-vivo to a cell or tissue, e.g., a human cell or tissue. In some embodiments, the composition is delivered to an ex vivo tissue that is in an injured state (e.g., from trauma, disease, hypoxia, ischemia or other damage).

In some embodiments, the cytobiologic composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). The composition improves viability, respiration, or other function of the transplant. The composition can be delivered to the tissue or organ before, during and/or after transplantation.

In some embodiments, a cytobiologic composition described herein is delivered ex-vivo to a cell or tissue derived from a subject. In some embodiments the cell or tissue is readministered to the subject (i.e., the cell or tissue is autologous).

The cytobiologics may act on a cell from any mammalian (e.g., human) tissue, e.g., from epithelial, connective, muscular, or nervous tissue or cells, and combinations thereof. The cytobiologics can be delivered to any eukaryotic (e.g., mammalian) organ system, for example, from the cardiovascular system (heart, vasculature); digestive system (esophagus, stomach, liver, gallbladder, pancreas, intestines, colon, rectum and anus); endocrine system (hypothalamus, pituitary gland, pineal body or pineal gland, thyroid, parathyroids, adrenal glands); excretory system (kidneys, ureters, bladder); lymphatic system (lymph, lymph nodes, lymph vessels, tonsils, adenoids, thymus, spleen); integumentary system (skin, hair, nails); muscular system (e.g., skeletal muscle); nervous system (brain, spinal cord, nerves); reproductive system (ovaries, uterus, mammary glands, testes, vas deferens, seminal vesicles, prostate); respiratory system (pharynx, larynx, trachea, bronchi, lungs, diaphragm); skeletal system (bone, cartilage), and combinations thereof.

In embodiments, the cytobiologic targets a tissue, e.g., liver, lungs, heart, spleen, pancreas, gastrointestinal tract, kidney, testes, ovaries, brain, reproductive organs, central nervous system, peripheral nervous system, skeletal muscle, endothelium, inner ear, adipose tissue (e.g., brown adipose tissue or white adipose tissue) or eye, when administered to a subject, e.g., wherein at least 0.1%, 0.5%, 1%, 1.5%, 2%, 2.5%, 3%, 4%, 5%, 10%, 20%, 30%, 40%, 50%, 60%, 70%, 80%, or 90% of the cytobiologics in a population of administered cytobiologics are present in the target tissue after 24, 48, or 72 hours, e.g., by an assay of Example 71.

In embodiments, the cytobiologics may act on a cell from a source of stem cells or progenitor cells, e.g., bone marrow stromal cells, marrow-derived adult progenitor cells (MAPCs), endothelial progenitor cells (EPC), blast cells, intermediate progenitor cells formed in the subventricular zone, neural stem cells, muscle stem cells, satellite cells, liver stem cells, hematopoietic stem cells, bone marrow stromal cells, epidermal stem cells, embryonic stem cells, mesenchymal stem cells, umbilical cord stem cells, precursor cells, muscle precursor cells, myoblast, cardiomyoblast, neural precursor cells, glial precursor cells, neuronal precursor cells, hepatoblasts.

### Methods of Use

The administration of a pharmaceutical composition described herein may be by way of oral, inhaled, transdermal or parenteral (including intravenous, intratumoral, intraperitoneal, intramuscular, intracavity, and subcutaneous) administration. The cytobiologics may be administered alone or formulated as a pharmaceutical composition.

The cytobiologics may be administered in the form of a unit-dose composition, such as a unit dose oral, parenteral, transdermal or inhaled composition. Such compositions are prepared by admixture and are suitably adapted for oral, inhaled, transdermal or parenteral administration, and as such may be in the form of tablets, capsules, oral liquid preparations, powders, granules, lozenges, reconstitutable powders, injectable and infusable solutions or suspensions or suppositories or aerosols.

In some embodiments, delivery of a cytobiologic composition described herein may induce or block cellular differentiation, de-differentiation, or trans-differentiation. The target mammalian cell may be a precursor cell. Alternatively, the target mammalian cell may be a differentiated cell, and the cell fate alteration includes driving de-differentiation into a pluripotent precursor cell, or blocking such de-differentiation. In situations where a change in cell fate is desired, effective amounts of a cytobiologic described herein encoding a cell fate inductive molecule or signal is introduced into a target cell under conditions such that an alteration in cell fate is induced. In some embodiments, a cytobiologic described herein is useful to reprogram a subpopulation of cells from a first phenotype to a second phenotype. Such a reprogramming may be temporary or permanent. Optionally, the reprogramming induces a target cell to adopt an intermediate phenotype.

Also provided are methods of reducing cellular differentiation in a target cell population. For example, a target cell population containing one or more precursor cell types is contacted with a cytobiologic composition described herein, under conditions such that the composition reduces the differentiation of the precursor cell. In certain embodiments, the target cell population contains injured tissue in a mammalian subject or tissue affected by a surgical procedure. The precursor cell is, e.g., a stromal precursor cell, a neural precursor cell, or a mesenchymal precursor cell.

A cytobiologic composition described herein, comprising a cargo, may be used to deliver such cargo to a cell tissue or subject. Delivery of a cargo by administration of a cytobiologic composition described herein may modify cellular protein expression levels. In certain embodiments, the administered composition directs upregulation of (via expression in the cell, delivery in the cell, or induction within the cell) of one or more cargo (e.g., a polypeptide or mRNA) that provide a functional activity which is substantially absent or reduced in the cell in which the polypeptide is delivered. For example, the missing functional activity may be enzymatic, structural, or regulatory in nature. In related embodiments, the administered composition directs up-regulation of one or more polypeptides that increases (e.g., synergistically) a functional activity which is present but substantially deficient in the cell in which the polypeptide is upregulated. In certain embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. For example, the upregulated functional activity may be enzymatic, structural, or regulatory in nature. In related embodiments, the administered composition directs down-regulation of one or more polypeptides that decreases (e.g., synergistically) a functional activity which is present or upregulated in the cell in which the polypeptide is downregulated. In certain embodiments, the administered composition directs upregulation of certain functional activities and downregulation of other functional activities.

In embodiments, the cytobiologic composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the cytobiologic composition comprises an exogenous protein), the effect lasts for less than 1, 2, 3, 4, 5, 6, or 7 days, 2, 3, or 4 weeks, or 1, 2, 3, 6, or 12 months.

### Ex-Vivo Applications

In embodiments, the cytobiologic composition described herein is delivered ex-vivo to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered to an ex vivo tissue that is in an injured state (e.g., from trauma, disease, hypoxia, ischemia or other damage).

In some embodiments, the composition is delivered to an ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). The composition can be delivered to the tissue or organ before, during and/or after transplantation.

In some embodiments, the composition is delivered, administered or contacted with a cell, e.g., a cell preparation. The cell preparation may be a cell therapy preparation (a cell preparation intended for administration to a human subject). In embodiments, the cell preparation comprises cells expressing a chimeric antigen receptor (CAR), e.g., expressing a recombinant CAR. The cells expressing the CAR may be, e.g., T cells, Natural Killer (NK) cells, cytotoxic T lymphocytes (CTL), regulatory T cells. In embodiments, the cell preparation is a neural stem cell preparation. In embodiments, the cell preparation is a mesenchymal stem cell (MSC) preparation. In embodiments, the cell preparation is a hematopoietic stem cell (HSC) preparation. In embodiments, the cell preparation is an islet cell preparation.

### In Vivo Uses

The cytobiologic compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein).

In some embodiments, the source of cytobiologics are from the same subject that is administered a cytobiologic composition. In other embodiments, they are different. For example, the source of cytobiologics and recipient tissue may be autologous (from the same subject) or heterologous (from different subjects). In either case, the donor tissue for cytobiologic compositions described herein may be a different tissue type than the recipient tissue. For example, the donor tissue may be muscular tissue and the recipient tissue may be connective tissue (e.g., adipose tissue). In other embodiments, the donor tissue and recipient tissue may be of the same or different type, but from different organ systems.

A cytobiologic composition described herein may be administered to a subject having a cancer, an autoimmune disease, an infectious disease, a metabolic disease, a neurodegenerative disease, or a genetic disease (e.g., enzyme deficiency). In some embodiments, the subject is in need of regeneration.

In some embodiments, the cytobiologic is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3:1462 DOI: 10.1038/srep01462). Thus, in some embodiments, the cytobiologic is co-administered with an inhibitor of sypressyn, e.g., a siRNA or inhibitory antibody.

### Non-Human Applications

Compositions described herein may also be used to similarly modulate the cell or tissue function or physiology of a variety of other organisms including but not limited to: farm or working animals (horses, cows, pigs, chickens etc.), pet or zoo animals (cats, dogs, lizards, birds, lions, tigers and bears etc.), aquaculture animals (fish, crabs, shrimp, oysters etc.), plants species (trees, crops, ornamentals flowers etc), fermentation species (Saccharomyces etc.). Cytobiologic compositions described herein can be made from such non-human sources and administered to a non-human target cell or tissue or subject.

Cytobiologic compositions can be autologous, allogeneic or xenogeneic to the target.

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used.

### EXAMPLES

### Example 1: Generating and Isolating Cytobiologics Through Vesicle Formation and Centrifugation

This example describes cytobiologic generation and isolation via vesiculation and centrifugation. This is one of the methods by which cytobiologics may be isolated.

Cytobiologics are prepared as follows. Approximately 4×106 HEK-293T cells are seeded in a 10 cm dish in complete media (DMEM+10% FBS+Pen/Strep). One day after seeding, 15 μg of transgene expressing plasmid or virus is delivered to cells. After a sufficient period of time for transgene expression, medium is carefully replaced by fresh medium supplemented with 100 μM ATP. Supernatants are harvested 48-72 hours after transgene expression, clarified by filtration through a 0.45 μm filter, and ultracentrifuged at 150,000×g for 1 h. Pelleted material is resuspended overnight in ice cold PBS. Cytobiologics are resuspended in desired buffer for experimentation.

See for example, Mangeot et al., Molecular Therapy, vol. 19 no. 9, 1656-1666, September 2011

### Example 2: Generating and Isolating Giant Plasma Membrane Cytobiologics

This example describes cytobiologic generation and isolation via vesiculation and centrifugation. This is one of the methods by which cytobiologics may be isolated. Cytobiologics are prepared as follows.

Briefly, HeLa cells that optionally express a transgene are washed twice in buffer (10 mM HEPES, 150 mM NaCl, 2 mM CaCl₂, pH 7.4), resuspended in a solution (1 mM DTT, 12.5 mM Paraformaldehyde, and 1 mM N-ethylmaleimide in GPMV buffer), and incubated at 37° C. for 1 h. Cytobiologics are clarified from cells by first removing cells by centrifugation at 100×g for 10 minutes, and then harvesting ytobiologics at 20,000×g for 1 h at 4° C. The cytobiologics are resuspended in desired buffer for experimentation.

See for example, Sezgin E et al. Elucidating membrane structure and protein behavior using giant membrane plasma vesicles. Nat. Protocols. 7(6):1042-51 2012.

### Example 3: Generating and Isolating Cytobiologic Ghosts

This example describes cytobiologic generation and isolation via hypotonic treatment and centrifugation. This is one of the methods by which cytobiologics may be produced.

First, cytobiologics are isolated from mesenchymal stem cells (10⁹ cells) primarily by using hypotonic treatment such that the cell ruptures and cytobiologics are formed. According to a specific embodiment, cells are resuspended in hypotonic solution, Tris-magnesium buffer (TM, e.g., pH 7.4 or pH 8.6 at 4° C., pH adjustment made with HCl). Cell swelling is monitored by phase-contrast microscopy. Once the cells swell and cytobiologics are formed, the suspension is placed in a homogenizer. Typically, about 95% cell rupture is sufficient as measured through cell counting and standard AOPI staining. The membranes/cytobiologics are then placed in sucrose (0.25 M or higher) for preservation. Alternatively, cytobiologics can be formed by other approaches known in the art to lyse cells, such as mild sonication (Arkhiv anatomii, gistologii i embriologii; 1979, August, 77(8) 5-13; PMID: 496657), freeze-thaw (Nature. 1999, Dec. 2; 402(6761):551-5; PMID: 10591218), French-press (Methods in Enzymology, Volume 541, 2014, Pages 169-176; PMID: 24423265), needle-passaging or solublization in detergent-containing solutions.

To avoid adherence, the cytobiologics are placed in plastic tubes and centrifuged. A laminated pellet is produced in which the topmost lighter gray lamina includes mostly cytobiologics. However, the entire pellet is processed, to increase yields. Centrifugation (e.g., 3,000 rpm for 15 min at 4° C.) and washing (e.g., 20 volumes of Tris magnesium/TM-sucrose pH 7.4) may be repeated.

In the next step, the cytobiologic fraction is separated by floatation in a discontinuous sucrose density gradient. A small excess of supernatant is left remaining with the washed pellet, which now includes cytobiologics, nuclei, and incompletely ruptured whole cells. An additional 60% w/w sucrose in TM, pH 8.6, is added to the suspension to give a reading of 45% sucrose on a refractometer. After this step, all solutions are TM pH 8.6. 15 ml of suspension are placed in SW-25.2 cellulose nitrate tubes and a discontinuous gradient is formed over the suspension by adding 15 ml layers, respectively, of 40% and 35% w/w sucrose, and then adding 5 ml of TM-sucrose (0.25 M). The samples are then centrifuged at 20,000 rpm for 10 min, 4° C. The nuclei sediment form a pellet, the incompletely ruptured whole cells are collected at the 40%-45% interface, and the cytobiologics are collected at the 35%-40% interface. The cytobiologics from multiple tubes are collected and pooled.

See for example, International patent publication, WO2011024172A2.

### Example 4: Generating Cytobiologics Through Extrusion

This example describes cytobiologic manufacturing by extrusion through a membrane.

Briefly, hematopoietic stem cells are in a 37° C. suspension at a density of 1×10⁶ cells/mL in serum-free media containing protease inhibitor cocktail (Set V, Calbiochem 539137-1ML). The cells are aspirated with a luer lock syringe and passed once through a disposable 5 mm syringe filter into a clean tube. If the membrane fouls and becomes clogged, it is set aside and a new filter is attached. After the entire cell suspension has passed through the filter, 5 mL of serum-free media is passed through all filters used in the process to wash any remaining material through the filter(s). The solution is then combined with the extruded cytobiologics in the filtrate.

Cytobiologics may be further reduced in size by continued extrusion following the same method with increasingly smaller filter pore sizes, ranging from 5 mm to 0.2 mm. When the final extrusion is complete, suspensions are pelleted by centrifugation (time and speed required vary by size) and resuspended in media.

Additionally, this process can be supplemented with the use of an actin cytoskeleton inhibitor in order to decrease the influence of the existing cytoskeletal structure on extrusion. Briefly, a 1×10⁶ cell/mL suspension is incubated in serum-free media with 500 nM Latrunculin B (ab144291, Abcam, Cambridge, Mass.) and incubated for 30 minutes at 37° C. in the presence of 5% CO₂. After incubation, protease inhibitor cocktail is added and cells are aspirated into a luer lock syringe, with the extrusion carried out as previously described.

Cytobiologics are pelleted and washed once in PBS to remove the cytoskeleton inhibitor before being resuspended in media.

### Example 5: Isolating Cytobiologic Microvesicles Freely Released from Cells

This example describes isolation of cytobiologics via centrifugation. This is one of the methods by which cytobiologics may be isolated.

Cytobiologics are isolated from cells by differential centrifugation. Culture media (DMEM+10% fetal bovine serum) is first clarified of small particles by ultracentrifugation at >100,000×g for 1 h. Clarified culture media is then used to grow Mouse Embryonic Fibroblasts. The cells are separated from culture media by centrifugation at 200×g for 10 minutes. Supernatants are collected and centrifuged sequentially twice at 500×g for 10 minutes, once at 2,000×g for 15 minutes, once at 10,000×g for 30 min, and once at 70,000×g for 60 minutes. Freely released cytobiologics are pelleted during the final centrifugation step, resuspended in PBS and repelleted at 70,000×g. The final pellet is resuspended in PBS.

See also, Wubbolts R et al. Proteomic and Biochemical Analyses of Human B Cell-derived Exosomes: Potential Implications for their Function and Multivesicular Body Formation. J. Biol. Chem. 278:10963-10972 2003.

### Example 6: Physical Enucleation of Source Cells to Produce Cytobiologics

This example describes enucleation to produce cytobiologics via cytoskeletal inactivation and centrifugation. This is one of the methods by which cytobiologics may be modified.

Cytobiologics are isolated from mammalian primary or immortalized cell lines. The cells are enucleated by treatment with an actin skeleton inhibitor and ultracentrifugation. Briefly, C2C12 cells are collected, pelleted, and resuspended in DMEM containing 12.5% Ficoll 400 (F2637, Sigma, St. Louis Mo.) and 500 nM Latrunculin B (ab144291, Abcam, Cambridge, Mass.) and incubated for 30 minutes at 37° C.+5% CO₂. Suspensions are carefully layered into ultracentrifuge tubes containing increasing concentrations of Ficoll 400 dissolved in DMEM (15%, 16%, 17%, 18%, 19%, 20%, 3 mL per layer) that have been equilibrated overnight at 37° C. in the presence of 5% CO₂. Ficoll gradients are spun in a Ti-70 rotor (Beckman-Coulter, Brea, Calif.) at 32,300 RPM for 60 minutes at 37 C. After ultracentrifugation, cytobiologics found between 16-18% Ficoll are removed, washed with DMEM, and resuspended in DMEM.

Staining for nuclear content with Hoechst 33342 as described in Example 23 followed by the use of flow cytometry and/or imaging will be performed to confirm the ejection of the nucleus.

### Example 7: Modifying Cytobiologics Via Irradiation

The following example describes modifying cytobiologics with gamma irradiation. Without being bound by theory, gamma irradiation may cause double stranded breaks in the DNA and drive cells to undergo apoptosis.

First, source cells are cultured in a monolayer on tissue culture flasks or plates below a confluent density (e.g. by culturing or plating cells). Then the medium is removed from confluent flasks, cells are rinsed with Ca2+ and Mg2+ free HBSS, and trypsinized to remove the cells from the culture matrix. The cell pellet is then resuspended in 10 ml of tissue-culture medium without penicillin/streptomycin and transferred to a 100-mm Petri dish. The number of cells in the pellet should be equivalent to what would be obtained from 10-15 confluent MEF cultures on 150 cm² flasks. The cells are then exposed to 4000 rads from a γ-radiation source to generate cytobiologics. The cytobiologics are then washed and resuspended in the final buffer or media to be used.

### Example 8: Modifying Cytobiologics Via Chemical Treatment

The following example describes modifying cytobiologics with mitomycin C treatment. Without being bound by any particular theory, mitomycin C treatment modifies cytobiologics by inactivating the cell cycle.

First, cells are cultured from a monolayer in tissue culture flasks or plates at a confluent density (e.g. by culturing or plating cells). One mg/ml mitomycin C stock solution is added to the medium to a final concentration of 10 μg/ml. The plates are then returned to the incubator for 2 to 3 hours. Then the medium is removed from confluent flasks, cells are rinsed with Ca2+ and Mg2+ free HBSS, and trypsinized to remove the cells from the culture matrix. The cells are then washed and resuspended in the final buffer or media to be used.

See for example, Mouse Embryo Fibroblast (MEF) Feeder Cell Preparation, Current Protocols in Molecular Biology. David A. Conner 2001.

### Example 9. Lack of Transcriptional Activity in Cytobiologics

This Example quantifies transcriptional activity in cytobiologics compared to parent cells, e.g., source cells, used for cytobiologics generation. In an embodiment, transcriptional activity will be low or absent in cytobiologics compared to the parent cells, e.g., source cells.

Cytobiologics are a chassis for the delivery of therapeutic agent. Therapeutic agents, such as miRNA, mRNAs, proteins and/or organelles that can be delivered to cells or local tissue environments with high efficiency could be used to modulate pathways that are not normally active or active at pathologically low or high levels in recipient tissue. In an embodiment, the observation that cytobiologics are not capable of transcription, or that cytobiologics have transcriptional activity of less than their parent cell, will demonstrate that removal of nuclear material has sufficiently occurred.

Cytobiologics are prepared by any one of the methods described in previous Examples. A sufficient number of cytobiologics and parent cells used to generate the cytobiologics are then plated into a 6 well low-attachment multiwell plate in DMEM containing 20% Fetal Bovine Serum, 1× Penicillin/Streptomycin and the fluorescent-taggable alkyne-nucleoside EU for 1 hr at 37° C. and 5% CO2. For negative controls, a sufficient number of cytobiologics and parent cells are also plated in multiwell plate in DMEM containing 20% Fetal Bovine Serum, 1× Penicillin/Streptomycin but with no alkyne-nucleoside EU.

After the 1 hour incubation the samples are processed following the manufacturer's instructions for an imaging kit (ThermoFisher Scientific). The cell and cytobiologics samples including the negative controls are washed thrice with 1×PBS buffer and resuspended in 1×PBS buffer and analyzed by flow cytometry (Becton Dickinson, San Jose, Calif., USA) using a 488 nm argon laser for excitation, and the 530+/−30 nm emission. BD FACSDiva software is used for acquisition and analysis. The light scatter channels are set on linear gains, and the fluorescence channels on a logarithmic scale, with a minimum of 10,000 cells analyzed in each condition.

In an embodiment, transcriptional activity as measured by 530+/−30 nm emission in the negative controls will be null due to the omission of the alkyne-nucleoside EU. In some embodiments, the cytobiologics will have less than about 70%, 60%, 50%, 40%, 30%, 20%, 10%, 5%, 4%, 3%, 2%, 1% or less transcriptional activity than the parental cells.

See also, Proc Natl Acad Sci USA, 2008, Oct. 14; 105(41):15779-84. doi: 10.1073/pnas.0808480105. Epub 2008 Oct. 7.

### Example 10: Lack of DNA Replication or Replication Activity in Cytobiologics

This Example quantifies DNA replication in cytobiologics. In an embodiment, cytobiologics will replicate DNA at a low rate compared to cells.

Cytobiologics are prepared by any one of the methods described in previous Examples. Cytobiologics and parental cell DNA replication activity is assessed by incorporation of a fluorescent-taggable nucleotide (ThermoFisher Scientific #C10632). Cytobiologics and an equivalent number of cells are incubated with EdU at a final concentration of 10 μM for 2 hr, after preparation of an EdU stock solution with in dimethylsulfoxide. The samples are then fixed for 15 min using 3.7% PFA, washed with 1×PBS buffer, pH 7.4 and permeabilized for 15 min in 0.5% detergent solution in 1×PBS buffer, pH 7.4.

After permeabilization, cytobiologics and cells in suspension in PBS buffer containing 0.5% detergent are washed with 1×PBS buffer, pH 7.4 and incubated for 30 min at 21° C. in reaction cocktail, 1×PBS buffer, CuSO4 (Component F), azide-fluor 488, 1× reaction buffer additive.

A negative control for cytobiologics and cell DNA replication activity is made with samples treated the same as above but with no azide-fluor 488 in the 1× reaction cocktail.

The cell and cytobiologic samples are then washed and resuspended in 1×PBS buffer and analyzed by flow cytometry. Flow cytometry is done with a FACS cytometer (Becton Dickinson, San Jose, Calif., USA) with 488 nm argon laser excitation, and a 530+/−30 nm emission spectrum is collected. FACS analysis software is used for acquisition and analysis. The light scatter channels are set on linear gains, and the fluorescence channels on a logarithmic scale, with a minimum of 10,000 cells analyzed in each condition. The relative DNA replication activity is calculated based on the median intensity of azide-fluor 488 in each sample. All events are captured in the forward and side scatter channels (alternatively, a gate can be applied to select only the cytobiologic population). The normalized fluorescence intensity value for the cytobiologics is determined by subtracting from the median fluorescence intensity value of the cytobiologic the median fluorescence intensity value of the respective negative control sample. Then the normalized relative DNA replication activity for the cytobiologic samples is normalized to the respective nucleated cell samples in order to generate quantitative measurements for DNA replication activity.

In an embodiment, cytobiologics have less DNA replication activity than parental cells. See, also, Salic, 2415-2420, doi: 10.1073/pnas.0712168105.

### Example 11: Electroporation to Modify Cytobiologics with Nucleic Acid Cargo

This example describes electroporation of cytobiologics with nucleic acid cargo.

Cytobiologics are prepared by any one of the methods described in a previous Example. Approximately 10⁹ cytobiologics and 1 μg of nucleic acids, e.g., RNA, are mixed in electroporation buffer (1.15 mM potassium phosphate pH 7.2, 25 mM potassium chloride, 60% iodixanol w/v in water). The cytobiologics are electroporated using a single 4 mm cuvette using an electroporation system (BioRad, 165-2081). The cytobiologics and nucleic acids are electroporated at 400 V, 125 μF and ∞ ohms, and the cuvette is immediately transferred to ice. After electroporation, cytobiologics are washed with PBS, resuspended in PBS, and kept on ice.

See, for example, Kamerkar et al., Exosomes facilitate therapeutic targeting of oncogenic KRAS in pancreatic cancer, Nature, 2017

### Example 12: Electroporation to Modify Cytobiologics with Protein Cargo

This example describes electroporation of cytobiologics with protein cargo.

Cytobiologics are prepared by any one of the methods described in a previous Example. Approximately 5×10⁶ cytobiologics are used for electroporation using an electroporation transfection system (Thermo Fisher Scientific). To set up a master mix, 24 μg of purified protein cargo is added to resuspension buffer (provided in the kit). The mixture is incubated at room temperature for 10 min. Meanwhile, cytobiologics are transferred to a sterile test tube and centrifuged at 500×g for 5 min. The supernatant is aspirated and the pellet is resuspended in 1 ml of PBS without Ca′ and Mg′. The buffer with the protein cargo is then used to resuspend the pellet of cytobiologics. A cytobiologic suspension is then used for optimization conditions, which vary in pulse voltage, pulse width and the number of pulses. After electroporation, cytobiologics are washed with PBS, resuspended in PBS, and kept on ice.

See, for example, Liang et al., Rapid and highly efficiency mammalian cell engineering via Cas9 protein transfection, Journal of Biotechnology 208: 44-53, 2015.

### Example 13: Chemical Treatment of Cytobiologics to Modify with Nucleic Acid Cargo

This example describes loading of nucleic acid cargo into a cytobiologic via chemical treatments.

Cytobiologics are prepared by any one of the methods described in previous Examples. Approximately 10⁶ cytobiologics are pelleted by centrifugation at 10,000 g for 5 min at 4 C. The pelleted cytobiologics are then resuspended in TE buffer (10 mM Tris-HCl (pH 8.0), 0.1 mM EDTA) with 20 μg DNA. The cytobiologic:DNA solution is treated with a mild detergent to increase DNA permeability across the cytobiologics membrane (Reagent B, Cosmo Bio Co., LTD, Cat #ISK-GN-001-EX). The solution is centrifuged again and the pellet is resuspended in buffer with a positively-charged peptide, such as protamine sulfate, to increase affinity between the DNA loaded cytobiologics and the target recipient cells (Reagent C, Cosmo Bio Co., LTD, Cat #ISK-GN-001-EX). After DNA loading, the loaded cytobiologics are kept on ice before use.

See, also, Kaneda, Y., et al., New vector innovation for drug delivery: development of fusigenic non-viral particles. Curr. Drug Targets, 2003

### Example 14: Chemical Treatment of Cytobiologics to Modify with Protein Cargo

This example describes loading of protein cargo into a cytobiologic via chemical treatments.

Cytobiologics are prepared by any one of the methods described in previous Examples. Approximately 10⁶ cytobiologics are pelleted by centrifugation at 10,000 g for 5 min at 4 C. The pelleted cytobiologics are then resuspended in buffer with positively-charged peptides, such as protamine sulfate, to increase the affinity between the cytobiologics and the cargo proteins (Reagent A, Cosmo Bio Co., LTD, Cat #ISK-GN-001-EX). Next 10 μg of cargo protein is added to the cytobiologic solution followed by addition of a mild detergent to increase protein permeability across the cytobiologic membrane (Reagent B, Cosmo Bio Co., LTD, Cat #ISK-GN-001-EX). The solution is centrifuged again and the pellet is resuspended in buffer with the positively-charged peptide, such as protamine sulfate, to increase affinity between the protein loaded cytobiologics and the target recipient cells (Reagent C, Cosmo Bio Co., LTD, Cat #ISK-GN-001-EX). After protein loading, the loaded cytobiologics are kept on ice before use.

See, also, Yasouka, E., et al., Needleless intranasal administration of HVJ-E containing allergen attenuates experimental allergic rhinitis. J. Mol. Med., 2007

### Example 15: Transfection of Cytobiologics to Modify with Nucleic Acid Cargo

This example describes transfection of nucleic acid cargo into a cytobiologic. Cytobiologics are prepared by any one of the methods described in previous Examples.

5×10⁶ cytobiologics are maintained in Opti-Mem. 0.5 μg of nucleic acid is mixed with 25 μl of Opti-MEM medium, followed by the addition of 25 μl of Opti-MEM containing 2 μl of lipid transfection reagent 2000. The mixture of nucleic acids, Opti-MEM, and lipid transfection reagent is maintained at room temperature for 15 minutes, then is added to the cytobiologics. The entire solution is mixed by gently swirling the plate and incubating at 37 C for 6 hours. Cytobiologics are then washed with PBS, resuspended in PBS, and kept on ice.

See, also, Liang et al., Rapid and highly efficiency mammalian cell engineering via Cas9 protein transfection, Journal of Biotechnology 208: 44-53, 2015.

### Example 16: Transfection of Cytobiologics to Modify with Protein Cargo

This example describes transfection of protein cargo into a cytobiologic.

Cytobiologics are prepared by any one of the methods described in previous Examples. 5×10⁶ cytobiologics are maintained in Opti-Mem. 0.5 μg of purified protein is mixed with 25 μl of Opti-MEM medium, followed by the addition of 25 μl of Opti-MEM containing 2 μl of lipid transfection reagent 3000. The mixture of protein, Opti-MEM, and lipid transfection reagent is maintained at room temperature for 15 minutes, then is added to the cytobiologics. The entire solution is mixed by gently swirling the plate and incubating at 37 C for 6 hours. Cytobiologics are then washed with PBS, resuspended in PBS, and kept on ice.

See, also, Liang et al., Rapid and highly efficiency mammalian cell engineering via Cas9 protein transfection, Journal of Biotechnology 208: 44-53, 2015.

### Example 17: Cytobiologics with Lipid Bilayer Structure

This Example describes the composition of cytobiologics. In an embodiment, a cytobiologic composition will comprise a lipid bilayer structure, with a lumen in the center.

Without wishing to be bound by theory, the lipid bilayer structure of a cytobiologic promotes fusion with a target cell, and allows cytobiologic to load different therapeutics.

Cytobiologics are freshly prepared using the methods described in the previous Examples. The positive control is the native cell line (HEK293), and the negative control is cold DPBS and membrane-disrupted HEK293 cell prep, which has been passed through 36 gauge needles for 50 times.

Samples are spin down in Eppendorf tube, and the supernatant is carefully removed. Then a pre-warmed fixative solution (2.5% glutaraldehyde in 0.05 M cacodylate buffer with 0.1M NaCl, pH 7.5; keep at 37° C. for 30 min before use) is added to the sample pellet and kept at room temperature for 20 minutes. The samples are washed twice with PBS after fixation. Osmium tetroxide solution is added to the sample pellet and incubated 30 minutes. After rinsing once with PBS, 30%, 50%, 70% and 90% hexylene glycol is added and washed with swirling, 15 minutes each. Then 100% hexylene glycol is added with swirling, 3 times, 10 minutes each.

Resin is combined with hexylene glycol at 1:2 ratio, and then added to the samples and incubated at room temperature for 2 hours. After incubation, the solution is replaced with 100% resin and incubated for 4-6 hours. This step is repeated one more time with fresh 100% resin. Then it is replaced with 100% fresh resin, the level is adjusted to ˜1-2 mm in depth, and baked for 8-12 hours. The Eppendorf tube is cut and pieces of epoxy cast with the sample is baked for an additional 16-24 hours. The epoxy cast is then cut into small pieces making note of the side with the cells. Pieces are glued to blocks for sectioning, using commercial 5-minute epoxy glue. A transmission electron microscope (JOEL, USA) is used to image the samples at a voltage of 80 kV.

In an embodiment, the cytobiologics will show a lipid bilayer structure similar to the positive control (HEK293 cells), and no obvious structure is observed in the DPBS control. In an embodiment no lumenal structures will be observed in the disrupted cell preparation.

### Example 18: Measuring the Average Size of Cytobiologics

This Example describes measurement of the average size of cytobiologics.

Cytobiologics are prepared by any one of the methods described in previous Examples. The cytobiologics are measured to determine the average size using commercially available systems (iZON Science). The system is used with software according to manufacturer's instructions and a nanopore designed to analyze particles within the 40 nm to 10 μm size range. Cytobiologics and parental cells are resuspended in phosphate-buffered saline (PBS) to a final concentration range of 0.01-0.1 μg protein/mL. Other instrument settings are adjusted as indicated in the following table:

TABLE 6
Cytobiologic measurement parameters and settings

| Measurement Parameter | Setting |
|---|---|
| Pressure | 6 |
| Nanopore type | NP300 |
| Calibration sample | CPC400_6P |
| Gold standard analysis | no |
| Capture assistant | none |

All cytobiologics are analyzed within 2 hours of isolation. In an embodiment, the cytobiologics will have a size within about 1%, 2%, 3%, 4%, 5%, 10%, 20%, 30%, 40%, 50%, 60%, 70%, 80%, 90%, or greater than the parental cells.

### Example 19: Measuring the Average Size Distribution of Cytobiologics

This Example describes measurement of the size distribution of cytobiologics.

Cytobiologics are generated by any one of the methods described in previous Examples, and are tested to determine the average size of particles using a commercially available system, such as described in a previous Example. In an embodiment, size thresholds for 10%, 50%, and 90% of the cytobiologics centered around the median are compared to parental cells to assess cytobiologic size distribution.

In an embodiment, the cytobiologics will have less than about 90%, 80%, 70%, 60%, 50%, 40%, 30%, 20%, 10%, 5%, or less of the parental cell's variability in size distribution within 10%, 50%, or 90% of the sample.

### Example 20: Average Volume of Cytobiologics

This Example describes measurement of the average volume of cytobiologics. Without wishing to be bound by theory, varying the size (e.g., volume) of cytobiologics can make them versatile for distinct cargo loading, therapeutic design or application.

Cytobiologics are prepared as described in previous Examples. The positive control is HEK293 cells or polystyrene beads with a known size. The negative control is HEK293 cells that are passed through a 36 gauge needle approximately 50 times.

Analysis with a transmission electron microscope, as described in a previous Example, is used to determine the size of the cytobiologics. The diameter of the cytobiologic is measured and volume is then calculated.

In an embodiment, cytobiologics will have an average size of approximately 50 nm or greater in diameter.

### Example 21: Average Density of Cytobiologics

Cytobiologic density is measured via a continuous sucrose gradient centrifugation assay as described in Théry et al., Curr Protoc Cell Biol. 2006 April; Chapter 3:Unit 3.22. Cytobiologics are obtained as described in previous Examples.

First, a sucrose gradient is prepared. A 2 M and a 0.25 sucrose solution are generated by mixing 4 ml HEPES/sucrose stock solution and 1 ml HEPES stock solution or 0.5 ml HEPES/sucrose stock solution and 4.5 ml HEPES stock solution, respectively. These two fractions are loaded into the gradient maker with all shutters closed, the 2 M sucrose solution in the proximal compartment with a magnetic stir bar, and the 0.25 M sucrose solution in the distal compartment. The gradient maker is placed on a magnetic stir plate, the shutter between proximal and distal compartments is opened and the magnetic stir plate is turned on. HEPES stock solution is made as follows: 2.4 g N-2-hydroxyethylpiperazine-N′-2-ethanesulfonic acid (HEPES; 20 mM final), 300 H2O, adjust pH to 7.4 with 10 N NaOH and finally adjust volume to 500 ml with H2O. HEPES/sucrose stock solution is made as follows: 2.4 g hydroxyethylpiperazine-N′-2-ethanesulfonic acid (HEPES; 20 mM final), 428 g protease-free sucrose (ICN; 2.5 M final), 150 ml H2O, adjust pH to 7.4 with 10 N NaOH and finally adjust volume to 500 ml with H2O.

The cytobiologics are resuspended in 2 nil of HEPES/sucrose stock solution and are poured on the bottom of an SW 41 centrifuge tube. The outer tubing is placed in the SW 41 tube, just above the 2 nil of cytobiologics. The outer shutter is opened, and a continuous 2 M (bottom) to 0.25 M (top) sucrose gradient is slowly poured on top of the cytobiologics. The SW 41 tube is lowered as the gradient is poured, so that the tubing is always slightly above the top of the liquid.

All tubes with gradients are balanced with each other, or with other tubes having the same weight of sucrose solutions. The gradients are centrifuged overnight (>14 hr) at 210,000×g, 4° C., in the SW 41 swinging-bucket rotor with the brake set on low.

With a micropipettor, eleven 1-ml fractions, from top to bottom, are collected and placed in a 3-ml tube for the TLA-100.3 rotor. The samples are set aside and, in separate wells of a 96-well plate, 50 μl of each fraction is used to measure the refractive index. The plate is covered with adhesive foil to prevent evaporation and stored for no more than 1 hour at room temperature. A refractometer is used to measure the refractive index (hence the sucrose concentration, and the density) of 10 to 20 μl of each fraction from the material saved in the 96-well plate.

A table for converting the refractive index into g/ml is available in the ultracentrifugation catalog downloadable from the Beckman website.

Each fraction is then prepared for protein content analysis. Two milliliters of 20 mM HEPES, pH 7.4, is added to each 1-ml gradient fraction, and mixed by pipetting up and down two to three times. One side of each tube is marked with a permanent marker, and the tubes are placed marked side up in a TLA-100.3 rotor.

The 3 nil-tubes with diluted fractions are centrifuged for 1 hr at 110,000×g, 4° C. The TLA-100.3 rotor holds six tubes, so two centrifugations for each gradient is performed with the other tubes kept at 4° C. until they can be centrifuged.

The supernatant is aspirated from each of the 3-nil tubes, leaving a drop on top of the pellet. The pellet most probably is not visible, but its location can be inferred from the mark on the tube. The invisible pellet is resuspended and transferred to microcentrifuge tubes. Half of each resuspended fraction is used for protein contentment analysis by bicinchoninic acid assay, described in another Example. This provides a distribution across the various gradient fractions of the cytobiologic preparation. This distribution is used to determine the average density of the cytobiologics. The second half volume fraction is stored at −80° C. and used for other purposes (e.g. functional analysis, or further purification by immunoisolation) once protein analysis has revealed the cytobiologic distribution across fractions.

In an embodiment, using this assay, the average density of the cytobiologics will be 1.25 g/ml+/−0.05 standard deviation. In an embodiment, the average density of the cytobiologics will be in the range of 1-1.1, 1.05-1.15, 1.1-1.2, 1.15-1.25, 1.2-1.3, or 1.25-1.35. In an embodiment, the average density of the cytobiologics will be less than 1 or more than 1.35.

### Example 22: Measuring Organelle Content in Cytobiologics

This Example describes detection of organelles in cytobiologics.

Cytobiologics were prepared as described herein. For detection of endoplasmic reticulum (ER) and mitochondria, cytobiologics or C2C12 cells were stained with 1 μM ER stain (E34251, Thermo Fisher, Waltham, Mass.) and 1 μM mitochondria stain (M22426, Thermo Fisher Waltham, Mass.). For detection of lysosomes, cytobiologics or cells were stained with 50 nM lysosome stain (L7526, Thermo Fisher, Waltham, Mass.).

Stained cytobiologics were run on a flow cytometer (Thermo Fisher, Waltham, Mass.) and fluorescence intensity was measured for each dye according to the table below. Validation for the presence of organelles was made by comparing fluorescence intensity of stained cytobiologics to unstained cytobiologics (negative control) and stained cells (positive control).

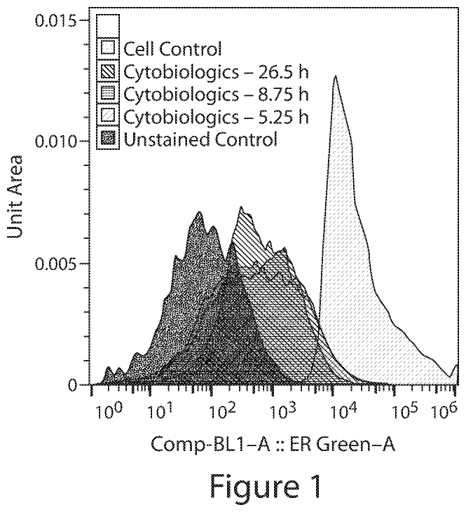

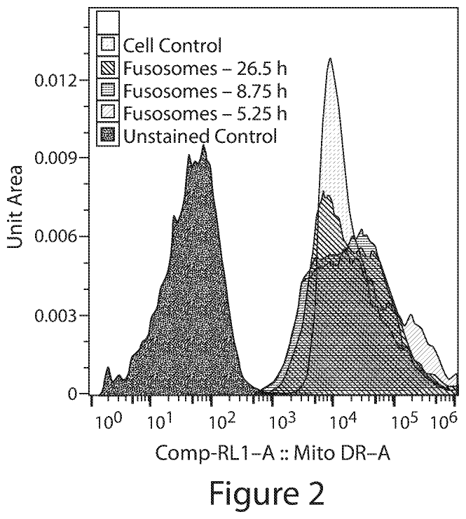

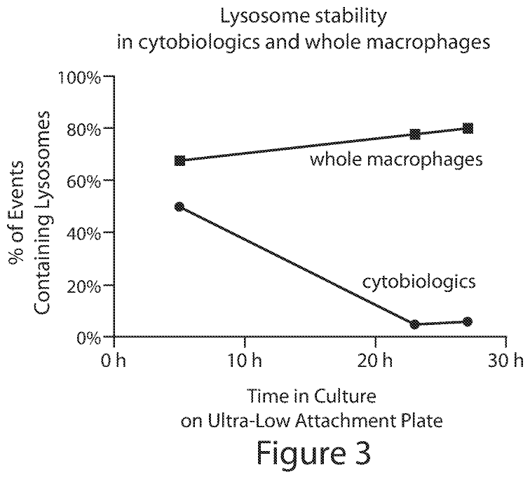

Cytobiologics stained positive for endoplasmic reticulum (FIG. 1), mitochondria (FIG. 2), and lysosomes (FIG. 3) 5 hours post-enucleation.

TABLE 7
Cytobiologic stains

| | Attune | Laser | Emission Filter |
|---|---|---|---|
| Stain | Laser/Filter | Wavelength | (nm) |
| Hoechst 33342 | VL1 | 405 | 450/40 |
| ER-Tracker Green | BL1 | 488 | 530/30 |
| MitoTracker Deep Red | RL1 | 638 | 670/14 |
| FM | | | |
| LysoTracker Green | BL1 | 488 | 530/30 |

### Example 23: Measuring Nuclear Content in Cytobiologics

This Example describes one embodiment of measuring nuclear content in a cytobiologics. To validate that cytobiologics do not contain nuclei, cytobiologics are stained with 1 μg·mL⁻¹ Hoechst 33342 and 1 μM CalceinAM (C3100MP, Thermo Fisher, Waltham, Mass.) and the stained cytobiologics are run on an Attune NXT Flow Cytometer (Thermo Fisher, Waltham, Mass.) to determine the fluorescence intensity of each dye according to the table below. In an embodiment, validation for the presence of cytosol (CalceinAM) and the absence of a nucleus (Hoechst 33342) will be made by comparing the mean fluorescence intensity of stained cytobiologics to unstained cytobiologics and stained cells.

TABLE 8
Flow cytometer settings

| | Attune | Laser | Emission Filter |
|---|---|---|---|
| Stain | Laser/Filter | Wavelength | (nm) |
| Hoechst 33342 | VL1 | 405 | 450/40 |
| Calcein AM | BL1 | 488 | 530/30 |

### Example 24: Measuring Nuclear Envelope Content in Cytobiologics

This Example describes a measurement of the nuclear envelope content in enucleated cytobiologics. The nuclear envelope isolates DNA from the cytoplasm of the cell.

In an embodiment, a purified cytobiologic composition comprises a mammalian cell, such as HEK-293 Ts (293 [HEK-293] (ATCC® CRL-1573™), that has been enucleated as described herein. This Example describes the quantification of different nuclear membrane proteins as a proxy to measure the amount of intact nuclear membrane that remains after cytobiologic generation.

In this Example, 10×10⁶HEK-293 Ts and the equivalent amount of cytobiologics prepared from 10×10⁶HEK-293 Ts are fixed for 15 min using 3.7% PFA, washed with 1×PBS buffer, pH 7.4 and permeabilized simultaneously, and then blocked for 15 min using 1×PBS buffer containing 1% Bovine Serum Albumin and 0.5% Triton® X-100, pH 7.4. After permeabilization, cytobiologics and cells are incubated for 12 hours at 4° C. with different primary antibodies, e.g. (anti-RanGAP1 antibody [EPR3295] (Abcam—ab92360), anti-NUP98 antibody [EPR6678]-nuclear pore marker (Abcam—ab124980), anti-nuclear pore complex proteins antibody [Mab414]-(Abcam—ab24609), anti-importin 7 antibody (Abcam—ab213670), at manufacturer suggested concentrations diluted in 1×PBS buffer containing 1% bovine serum albumin and 0.5% Triton® X-100, pH 7.4. Cytobiologics and cells are then washed with 1×PBS buffer, pH 7.4, and incubated for 2 hr at 21° C. with an appropriate fluorescent secondary antibody that detects the previous specified primary antibody at manufacturer suggested concentrations diluted in 1×PBS buffer containing 1% bovine serum albumin and 0.5% detergent, pH 7.4. Cytobiologics and cells are then washed with 1×PBS buffer, re-suspended in 300 μL of 1×PBS buffer, pH 7.4 containing 1 μg/ml Hoechst 33342, filtered through a 20 μm FACS tube and analyzed by flow cytometry.

Negative controls are generated using the same staining procedure but with no primary antibody added. Flow cytometry is performed on a FACS cytometer (Becton Dickinson, San Jose, Calif., USA) with 488 nm argon laser excitation, and a 530+/−30 nm emission spectrum is collected. FACS acquisition software is used for acquisition and analysis. The light scatter channels are set on linear gains, and the fluorescence channels on a logarithmic scale, with a minimum of 10,000 cells analyzed in each condition. The relative intact nuclear membrane content is calculated based on the median intensity of fluorescence in each sample. All events are captured in the forward and side scatter channels.

The normalized fluorescence intensity value for the cytobiologics is determined by subtracting from the median fluorescence intensity value of the cytobiologic the median fluorescence intensity value of the respective negative control sample. Then the normalized fluorescence for the cytobiologics samples is normalized to the respective nucleated cell samples in order to generate quantitative measurements of intact nuclear membrane content.

In an embodiment, enucleated cytobiologics will comprise less than 1%, 2%, 3%, 4%, 5%, 10%, 20%, 30%, 40%, 50%, 60%, 70%, 80%, or 90% fluorescence intensity or nuclear envelope content compared to the nucleated parental cells.

### Example 25: Measuring Chromatin Levels in Cytobiologics

This Example describes measurement of chromatin in enucleated cytobiologics.

DNA can be condensed into chromatin to allow it to fit inside the nucleus. In an embodiment, a purified cytobiologic composition as produced by any one of the methods described herein will comprise low levels of chromatin.

Enucleated cytobiologics prepared by any of the methods previously described and positive control cells (e.g., parental cells) are assayed for chromatin content using an ELISA with antibodies that are specific to histone protein H3 or histone protein H4. Histones are the chief protein component of chromatin, with H3 and H4 the predominant histone proteins.

Histones are extracted from the cytobiologic preparation and cell preparation using a commercial kit (e.g. Abcam Histone Extraction Kit (ab113476)) or other methods known in the art. These aliquots are stored at −80 C until use. A serial dilution of standard is prepared by diluting purified histone protein (either H3 or H4) from 1 to 50 ng/μl in a solution of the assay buffer. The assay buffer may be derived from a kit supplied by a manufacturer (e.g. Abcam Histone H4 Total Quantification Kit (ab156909) or Abcam Histone H3 total Quantification Kit (ab115091)). The assay buffer is added to each well of a 48- or 96-well plate, which is coated with an anti-histone H3 or anti-H4 antibody and sample or standard control is added to the well to bring the total volume of each well to 50 μl. The plate is then covered and incubated at 37 degrees for 90 to 120 minutes.

After incubation, any histone bound to the anti-histone antibody attached to the plate is prepared for detection. The supernatant is aspirated and the plate is washed with 150 μl of wash buffer. The capture buffer, which includes an anti-histone H3 or anti-H4 capture antibody, is then added to the plate in a volume of 50 μl and at a concentration of 1 μg/mL. The plate is then incubated at room temperature on an orbital shaker for 60 minutes.

Next, the plate is aspirated and washed 6 times using wash buffer. Signal reporter molecule activatable by the capture antibody is then added to each well. The plate is covered and incubated at room temperature for 30 minutes. The plate is then aspirated and washed 4 times using wash buffer. The reaction is stopped by adding stop solution. The absorbance of each well in the plate is read at 450 nm, and the concentration of histones in each sample is calculated according to the standard curve of absorbance at 450 nm vs. concentration of histone in standard samples.

In an embodiment, cytobiologic samples will comprise less than 1%, 2%, 3%, 4%, 5%, 10%, 20%, 30%, 40%, 50%, 60%, 70%, 80%, or 90% the histone concentration of the nucleated parental cells.

### Example 26: Measuring DNA Content in Cytobiologics

This Example describes quantification of the amount of DNA in a cytobiologic relative to nucleated counterparts. In an embodiment, cytobiologics will have less DNA than nucleated counterparts. Nucleic acid levels are determined by measuring total DNA or the level of a specific house-keeping gene. In an embodiment, cytobiologics having reduced DNA content or substantially lacking DNA will be unable to replicate, differentiate, or transcribe genes, ensuring that their dose and function is not altered when administered to a subject.

Cytobiologics are prepared by any one of the methods described in previous Examples. Preparations of the same mass, as measured by protein, of cytobiologic and source cells, are used to isolate total DNA (e.g. using a kit such as Qiagen DNeasy catalog #69504), followed by determination of DNA concentration using standard spectroscopic methods to assess light absorbance by DNA (e.g. with Thermo Scientific NanoDrop).

In an embodiment, the concentration of DNA in enucleated cytobiologics will be less than about 50%, 40%, 30%, 20%, 10%, 5%, 4%, 3%, 2%, 1% or less than in parental cells.

Alternatively, the concentration of a specific house-keeping gene, such as GAPDH, can be compared between nucleated cells and cytobiologics with semi-quantitative real-time PCR (RT-PCR). Total DNA is isolated from parental cells and cytobiologics and DNA concentration is measured as described herein. RT-PCR is carried out with a PCR kit (Applied Biosystems, catalog #4309155) using the following reaction template:

### SYBR Green Master Mix: 10 μL

### 0.45 μM Forward Primer: 1 μL

### 0.45 μM Reverse Primer: 1 μL

### DNA Template: 10 ng

### PCR-Grade Water: Variable

Forward and reverse primers are acquired from Integrated DNA Technologies. The table below details the primer pairs and their associated sequences:

TABLE 9
Primer sequences

| | Forward Primer | Reverse Primer |
|---|---|---|
| Target | Sequence (5′→3′) | Sequence (5′→3′) |
| Human nDNA | GGAGTCCACTG | GAGGCATTGCTG |
| (GAPDH) | GCGTCTTCAC | ATGATCTTGAGG |

A real-time PCR system (Applied Biosystems) is used to perform the amplification and detection with the following protocol:

### Denaturation, 94° C. 2 min

40 Cycles of the following sequence:

### Denaturation, 94° C. 15 sec

### Annealing, Extension, 60° C. 1 min

A standard curve of the Cₜ vs. DNA concentration is prepared with serial dilutions of GAPDH DNA and used to normalize the Ct nuclear value from cytobiologic PCR results to a specific amount (ng) of DNA.

In an embodiment, the concentration of GAPDH DNA in enucleated cytobiologics will be less than about 50%, 40%, 30%, 20%, 10%, 5%, 4%, 3%, 2%, 1% or less than in parental cells.

### Example 27: Measuring miRNA Content in Cytobiologics

This Example describes quantification of microRNAs (miRNAs) in cytobiologics. In an embodiment, a cytobiologic comprises miRNAs.

MiRNAs are regulatory elements that, among other activities, control the rate by which messenger RNAs (mRNAs) are translated into proteins. In an embodiment, cytobiologics carrying miRNA may be used to deliver the miRNA to target sites.

Cytobiologics are prepared by any one of the methods described in previous Examples. RNA from cytobiologics or parental cells is prepared as described previously. At least one miRNA gene is selected from the Sanger Center miRNA Registry at www.sanger.ac.uk/Software/Rfam/mirna/index.shtml. miRNA is prepared as described in Chen et al, Nucleic Acids Research, 33(20), 2005. All TaqMan miRNA assays are available through Thermo Fisher (A25576, Waltham, Mass.).

qPCR is carried out according to manufacturer's specifications on miRNA cDNA, and C_{T} values are generated and analyzed using a real-time PCR system as described herein.

In an embodiment, the miRNA content of cytobiologics will be at least 1%, 2%, 3%, 4%, 5%, 10%, 20%, 30%, 40%, 50%, 60%, 70%, 80%, 90%, or greater than that of their parental cells.

### Example 28: Quantifying Expression of an Endogenous RNA or Synthetic RNA in Cytobiologics

This Example describes quantification of levels of endogenous RNA with altered expression, or a synthetic RNA that is expressed in a cytobiologic.

The cytobiologic or parental cell is engineered to alter the expression of an endogenous or synthetic RNA that mediates a cellular function to the cytobiologics.

Transposase vectors (System Biosciences, Inc.) includes the open reading frame of the Puromycin resistance gene together with an open reading frame of a cloned fragment of a protein agent. The vectors are electroporated into 293 Ts using an electroporator (Amaxa) and a 293T cell line specific nuclear transfection kit (Lonza).

Following selection with puromycin for 3-5 days in DMEM containing 20% Fetal Bovine Serum and 1× Penicillin/Streptomycin, cytobiologics are prepared from the stably expressing cell line by any one of the methods described in previous Examples.

Individual cytobiologics are isolated and protein agent or RNA per cytobiologic is quantified as described in a previous Example.

In an embodiment, the cytobiologics will have at least 1, 2, 3, 4, 5, 10, 20, 50, 100, 500, 10³, 5.0×10³, 10⁴, 5.0×10⁴, 10⁵, 5.0×10⁵, 10⁶, 5.0×10⁶, or more of the RNA per cytobiologic.

### Example 29: Measuring Lipid Composition in Cytobiologics

This Example describes quantification of the lipid composition of cytobiologics. In an embodiment, the lipid composition of cytobiologics is similar to the cells that they are derived from. Lipid composition affects important biophysical parameters of cytobiologics and cells, such as size, electrostatic interactions, and colloidal behavior.

The lipid measurements are based on mass spectrometry. Cytobiologics are prepared by any one of the methods described in previous Examples.

Mass spectrometry-based lipid analysis is performed at a lipid analysis service (Dresden, Germany) as described (Sampaio, et al., Proc Natl Acad Sci, 2011, Feb. 1; 108(5):1903-7). Lipids are extracted using a two-step chloroform/methanol procedure (Ejsing, et al., Proc Natl Acad Sci, 2009, Mar. 17; 106(7):2136-41). Samples are spiked with an internal lipid standard mixture of: cardiolipin 16:1/15:0/15:0/15:0 (CL), ceramide 18:1; 2/17:0 (Cer), diacylglycerol 17:0/17:0 (DAG), hexosylceramide 18:1; 2/12:0 (HexCer), lysophosphatidate 17:0 (LPA), lyso-phosphatidylcholine 12:0 (LPC), lyso-phosphatidylethanolamine 17:1 (LPE), lyso-phosphatidylglycerol 17:1 (LPG), lyso-phosphatidylinositol 17:1 (LPI), lyso-phosphatidylserine 17:1 (LPS), phosphatidate 17:0/17:0 (PA), phosphatidylcholine 17:0/17:0 (PC), phosphatidylethanolamine 17:0/17:0 (PE), phosphatidylglycerol 17:0/17:0 (PG), phosphatidylinositol 16:0/16:0 (PI), phosphatidylserine 17:0/17:0 (PS), cholesterol ester 20:0 (CE), sphingomyelin 18:1; 2/12:0; 0 (SM) and triacylglycerol 17:0/17:0/17:0 (TAG).

After extraction, the organic phase is transferred to an infusion plate and dried in a speed vacuum concentrator. The first step dry extract is resuspended in 7.5 mM ammonium acetate in chloroform/methanol/propanol (1:2:4, V:V:V) and the second step dry extract is resuspended in 33% ethanol solution of methylamine in chloroform/methanol (0.003:5:1; V:V:V). All liquid handling steps are performed using a robotic platform for organic solvent with an anti-droplet control feature (Hamilton Robotics) for pipetting.

Samples are analyzed by direct infusion on a mass spectrometer (Thermo Scientific) equipped with an ion source (Advion Biosciences). Samples are analyzed in both positive and negative ion modes with a resolution of Rm/z=200=280000 for MS and Rm/z=200=17500 for tandem MS/MS experiments, in a single acquisition. MS/MS is triggered by an inclusion list encompassing corresponding MS mass ranges scanned in 1 Da increments (Surma, et al., Eur J lipid Sci Technol, 2015, October; 117(10):1540-9). Both MS and MS/MS data are combined to monitor CE, DAG and TAG ions as ammonium adducts; PC, PC O−, as acetate adducts; and CL, PA, PE, PE O−, PG, PI and PS as deprotonated anions. MS only is used to monitor LPA, LPE, LPE O−, LPI and LPS as deprotonated anions; Cer, HexCer, SM, LPC and LPC O− as acetate.

Data are analyzed with in-house developed lipid identification software as described in the following references (Herzog, et al., Genome Biol, 2011, Jan. 19; 12(1):R8; Herzog, et al., PLoS One, 2012, January; 7(1):e29851). Only lipid identifications with a signal-to-noise ratio >5, and a signal intensity 5-fold higher than in corresponding blank samples are considered for further data analysis.

Cytobiologic lipid composition is compared to parental cells' lipid composition. In an embodiment, cytobiologics and parental cells will have a similar lipid composition if >50% of the identified lipids in the parental cells are present in the cytobiologics, and of those identified lipids, the level in the cytobiologic will be >25% of the corresponding lipid level in the parental cell.

### Example 30: Measuring Proteomic Composition in Cytobiologics

This Example describes quantification of the protein composition of cytobiologics. In an embodiment, the protein composition of cytobiologics will be similar to the cells that they are derived from.

Cytobiologics are prepared by any one of the methods described in previous Examples. Cytobiologics are resuspended in lysis buffer (7M Urea, 2M Thiourea, 4% (w/v) Chaps in 50 mM Tris pH 8.0) and incubated for 15 minutes at room temperature with occasional vortexing. Mixtures are then lysed by sonication for 5 minutes in an ice bath and spun down for 5 minutes at 13,000 RPM. Protein content is determined by a colorimetric assay (Pierce) and protein of each sample is transferred to a new tube and the volume is equalized with 50 mM Tris pH 8.

Proteins are reduced for 15 minutes at 65 Celsius with 10 mM DTT and alkylated with 15 mM iodoacetamide for 30 minutes at room temperature in the dark. Proteins are precipitated with gradual addition of 6 volumes of cold (−20 Celsius) acetone and incubated overnight at −80 Celsius. Protein pellets are washed 3 times with cold (−20 Celsius) methanol. Proteins are resuspended in 50 mM Tris pH 8.3.

Next, trypsin/lysC is added to the proteins for the first 4 h of digestion at 37 Celsius with agitation. Samples are diluted with 50 mM Tris pH 8 and 0.1% sodium deoxycholate is added with more trypsin/lysC for digestion overnight at 37 Celsius with agitation. Digestion is stopped and sodium deoxycholate is removed by the addition of 2% v/v formic acid. Samples are vortexed and cleared by centrifugation for 1 minute at 13,000 RPM. Peptides are purified by reversed phase solid phase extraction (SPE) and dried down. Samples are reconstituted in 20 μl of 3% DMSO, 0.2% formic acid in water and analyzed by LC-MS.

To have quantitative measurements, a protein standard is also run on the instrument. Standard peptides (Pierce, equimolar, LC-MS grade, #88342) are diluted to 4, 8, 20, 40 and 100 fmol/ul and are analyzed by LC-MS/MS. The average AUC (area under the curve) of the 5 best peptides per protein (3 MS/MS transition/peptide) is calculated for each concentration to generate a standard curve.

Acquisition is performed with a high resolution mass spectrometer (ABSciex, Foster City, Calif., USA) equipped with an electrospray interface with a 25 μm iD capillary and coupled with micro-ultrahigh performance liquid chromatography (μUHPLC) (Eksigent, Redwood City, Calif., USA). Analysis software is used to control the instrument and for data processing and acquisition. The source voltage is set to 5.2 kV and maintained at 225° C., curtain gas is set at 27 psi, gas one at 12 psi and gas two at 10 psi. Acquisition is performed in Information Dependent Acquisition (IDA) mode for the protein database and in SWATH acquisition mode for the samples. Separation is performed on a reversed phase column 0.3 i.d., 2.7 μm particles, 150 mm long (Advance Materials Technology, Wilmington, Del.) which is maintained at 60° C. Samples are injected by loop overfilling into a 5 μL loop. For the 120 minute (samples) LC gradient, the mobile phase includes the following: solvent A (0.2% v/v formic acid and 3% DMSO v/v in water) and solvent B (0.2% v/v formic acid and 3% DMSO in EtOH) at a flow rate of 3 μL/min.

For the absolute quantification of the proteins, a standard curve (5 points, R2>0.99) is generated using the sum of the AUC of the 5 best peptides (3 MS/MS ion per peptide) per protein. To generate a database for the analysis of the samples, the DIAUmpire algorithm is run on each of the 12 samples and combined with the output MGF files into one database. This database is used with software (ABSciex) to quantify the proteins in each of the samples, using 5 transition/peptide and 5 peptide/protein maximum. A peptide is considered as adequately measured if the score computed is superior to 1.5 or had a FDR<1%. The sum of the AUC of each of the adequately measured peptides is mapped on the standard curve, and is reported as fmol.

The resulting protein quantification data is then analyzed to determine protein levels and proportions of known classes of proteins as follows: enzymes are identified as proteins that are annotated with an Enzyme Commission (EC) number; ER associated proteins are identified as proteins that had a Gene Ontology (GO; http://www.geneontology.org) cellular compartment classification of ER and not mitochondria; exosome associated proteins are identified as proteins that have a Gene Ontology cellular compartment classification of exosomes and not mitochondria; and mitochondrial proteins are identified as proteins that are identified as mitochondrial in the MitoCarta database (Calvo et al., NAR 20151 doi:10.1093/nar/gkv1003). The molar ratios of each of these categories are determined as the sum of the molar quantities of all the proteins in each class divided by the sum of the molar quantities of all identified proteins in each sample.

Cytobiologic proteomic composition is compared to parental cell proteomic composition. In an embodiment, a similar proteomic compositions between cytobiologics and parental cells will be observed when >50% of the identified proteins are present in the cytobiologic, and of those identified proteins the level is >25% of the corresponding protein level in the parental cell.

### Example 31: Quantifying an Endogenous or Synthetic Protein Level Per Cytobiologic

This Example describes quantification of an endogenous or synthetic protein cargo in cytobiologics. In an embodiment, cytobiologics comprise an endogenous or synthetic protein cargo.

The cytobiologic or parental cell is engineered to alter the expression of an endogenous protein or express a synthetic cargo that mediates a therapeutic or novel cellular function.

Transposase vectors (System Biosciences, Inc.) that include the open reading frame of the puromycin resistance gene together with an open reading frame of a cloned fragment of a protein agent, optionally translationally fused to the open reading frame of a green fluorescent protein (GFP). The vectors are electroporated into 293 Ts using an electroporator (Amaxa) and a 293T cell line specific nuclear transfection kit (Lonza).

Following selection with puromycin for 3-5 days in DMEM containing 20% fetal bovine serum and 1× penicillin/streptomycin, cytobiologics are prepared from the stably expressing cell line by any one of the methods described in previous Examples.

Altered expression levels of an endogenous protein or expression levels of a synthetic protein that are not fused to GFP are quantified by mass spectrometry as described above. In an embodiment, the cytobiologics will have at least 1, 2, 3, 4, 5, 10, 20, 50, 100, 500, 10³, 5.0×10³, 10⁴, 5.0×10⁴, 10⁵, 5.0×10⁵, 10⁶, 5.0×10⁶, or more protein agent molecules per cytobiologic.

Alternatively, purified GFP is serially diluted in DMEM containing 20% fetal bovine serum and 1× Penicillin/Streptomycin to generate a standard curve of protein concentration. GFP fluorescence of the standard curve and a sample of cytobiologics is measured in a fluorimeter (BioTek) using a GFP light cube (469/35 excitation filter and a 525/39 emission filter) to calculate the average molar concentration of GFP molecules in the cytobiologics. The molar concentration is then converted to number of GFP molecules and divided by the number of cytobiologics per sample to achieve an average number of protein agent molecules per cytobiologic.

In an embodiment, the cytobiologics will have at least 1, 2, 3, 4, 5, 10, 20, 50, 100, 500, 10³, 5.0×10³, 10⁴, 5.0×10⁴, 10⁵, 5.0×10⁵, 10⁶, 5.0×10⁶, or more protein agent molecules per cytobiologic.

### Example 32: Measuring Markers of Exosomal Proteins in Cytobiologics

This assay describes quantification of the proteomics makeup of the sample preparation, and quantifies the proportion of proteins that are known to be specific markers of exosomes.

Cytobiologics are pelleted and shipped frozen to the proteomics analysis center per standard biological sample handling procedures.

The cytobiologics are thawed for protein extraction and analysis. First, they are resuspended in lysis buffer (7M urea, 2M thiourea, 4% (w/v) chaps in 50 mM Tris pH 8.0) and incubated for 15 minutes at room temperature with occasional vortexing. The mixtures are then lysed by sonication for 5 minutes in an ice bath and spun down for 5 minutes at 13,000 RPM. Total protein content is determined by a colorimetric assay (Pierce) and 100 μg of protein from each sample is transferred to a new tube and the volume is adjusted with 50 mM Tris pH 8.

The proteins are reduced for 15 minutes at 65° Celsius with 10 mM DTT and alkylated with 15 mM iodoacetamide for 30 minutes at room temperature in the dark. The proteins are then precipitated with gradual addition of 6 volumes of cold (−20° Celsius) acetone and incubated over night at −80° Celsius.

The proteins are pelleted, washed 3 times with cold (−20° Celsius) methanol, and resuspended in 50 mM Tris pH 8. 3.33 μg of trypsin/lysC is added to the proteins for a first 4 h of digestion at 37° Celsius with agitation. The samples are diluted with 50 mM Tris pH 8 and 0.1% sodium deoxycholate is added with another 3.3 μg of trypsin/lysC for digestion overnight at 37° Celsius with agitation. Digestion is stopped and sodium deoxycholate is removed by the addition of 2% v/v formic acid. Samples are vortexed and cleared by centrifugation for 1 minute at 13,000 RPM.

The proteins are purified by reversed phase solid phase extraction (SPE) and dried down. The samples are reconstituted in 3% DMSO, 0.2% formic acid in water and analyzed by LC-MS as described previously.

The resulting protein quantification data is analyzed to determine protein levels and proportions of know exosomal marker proteins. Specifically: tetraspanin family proteins (CD63, CD9, or CD81), ESCRT-related proteins (TSG101, CHMP4A-B, or VPS4B), Alix, TSG101, MHCI, MHCII, GP96, actinin-4, mitofilin, syntenin-1, TSG101, ADAM10, EHD4, syntenin-1, TSG101, EHD1, flotillin-1, heat-shock 70-kDa proteins (HSC70/HSP73, HSP70/HSP72). The molar ratio these exosomal marker proteins relative to all proteins measured is determined as the molar quantity of each specific exosome marker protein listed above divided by the sum of the molar quantities of all identified proteins in each sample and expressed as a percent.

Similarly, the molar ratio for all exosomal marker proteins relative to all proteins measured is determined as the sum of the molar quantity of all specific exosome marker protein listed above divided by the sum of the molar quantities of all identified proteins in each sample and expressed as a percent of the total.

In an embodiment, using this approach, a sample will comprise less than 5% of any individual exosomal marker protein and less than 15% of total exosomal marker proteins.

In an embodiment, any individual exosomal marker protein will be present at less than 0.05%, 0.1%, 0.5%, 1%, 2%, 3%, 4%, 5%, or 10%.

In an embodiment, the sum of all exosomal marker proteins will be less than 0.05%, 0.1%, 0.5%, 1%, 2%, 3%, 4%, 5%, 10%, 15%, 20%, or 25%.

### Example 33: Measuring GAPDH in Cytobiologics

This assay describes quantification of the level of glyceraldehyde 3-phosphate dehydrogenase (GAPDH) in the cytobiologics, and the relative level of GAPDH in the cytobiologics compared to the parental cells.

GAPDH is measured in the parental cells and the cytobiologics using a standard commercially available ELISA for GAPDH (ab176642, Abcam) per the manufacturer's directions.

Total protein levels are similarly measured via bicinchoninic acid assay as previously described in the same volume of sample used to measure GAPDH. In embodiments, using this assay, the level of GAPDH per total protein in the cytobiologics will be <100 ng GAPDH/μg total protein. Similarly, the decrease in GAPDH levels relative to total protein from the parental cells to the cytobiologics will be greater than a 10% decrease.

In an embodiment, GAPDH content in the preparation in ng GAPDH/μg total protein will be less than 500, less than 250, less than 100, less than 50, less than 20, less than 10, less than 5, or less than 1.

In an embodiment, the decrease in GAPDH per total protein in ng/μg from the parent cell to the preparation will be more than 1%, more than 2.5%, more than 5%, more than 10%, more than 15%, more than 20%, more than 30%, more than 40%, more than 50%, more than 60%, more than 70%, more than 80%, or more than 90%.

### Example 34: Measuring Calnexin in Cytobiologics

This assay describes quantification of the level of calnexin (CNX) in the cytobiologics, and the relative level of CNX in the cytobiologics compared to the parental cells.

Calnexin is measured in the starting cells and the preparation using a standard commercially available ELISA for calnexin (MBS721668, MyBioSource) per the manufacturer's directions.

Total protein levels are similarly measured via bicinchoninic acid assay as previously described in the same volume of sample used to measure calnexin. In embodiments, using this assay, the level of calnexin per total protein in the cytobiologics will be <100 ng calnexin/μg total protein. Similarly, in embodiments, the increase in calnexin levels relative to total protein from the parental cell to the cytobiologics will be greater than a 10% increase.

In an embodiment, calnexin content in the preparation in ng calnexin/μg total protein will be less than 500, 250, 100, 50, 20, 10, 5, or 1.

In an embodiment, the decrease in calnexin per total protein in ng/μg from the parent cell to the preparation will be more than 1%, 2.5%, 5%, 10%, 15%, 20%, 30%, 40%, 50%, 60%, 70%, 80%, or 90%.

### Example 35: Comparison of Soluble to Insoluble Protein Mass in Cytobiologics

This Example describes quantification of the soluble:insoluble ratio of protein mass in cytobiologics. In an embodiment, the soluble:insoluble ratio of protein mass in cytobiologics will be similar to nucleated cells.

Cytobiologics are prepared by any one of the methods described in previous Examples. The cytobiologic preparation is tested to determine the soluble:insoluble protein ratio using a standard bicinchoninic acid assay (BCA) (e.g. using the commercially available Pierce™ BCA Protein Assay Kit, Thermo Fischer product #23225). Soluble protein samples are prepared by suspending the prepared cytobiologics or parental cells at a concentration of 1×10\hat{ }7 cells or cytobiologics/mL in PBS and centrifuging at 1600 g to pellet the cytobiologics or cells. The supernatant is collected as the soluble protein fraction.

The cytobiologics or cells in the pellet are lysed by vigorous pipetting and vortexing in PBS with 2% Triton-X-100. The lysed fraction represents the insoluble protein fraction.

A standard curve is generated using the supplied BSA, from 0 to 20 μg of BSA per well (in triplicate). The cytobiologic or cell preparation is diluted such that the quantity measured is within the range of the standards. The cytobiologic preparation is analyzed in triplicate and the mean value is used. The soluble protein concentration is divided by the insoluble protein concentration to yield the soluble:insoluble protein ratio.

In an embodiment, the cytobiologic soluble:insoluble protein ratio will be within 1%, 2%, 3%, 4%, 5%, 10%, 20%, 30%, 40%, 50%, 60%, 70%, 80%, 90%, or greater compared to the parental cells.

### Example 36: Measuring LPS in Cytobiologics

This Example describes quantification of levels of lipopolysaccharides (LPS) in cytobiologics as compared to parental cells. In an embodiment, cytobiologics will have lower levels of LPS compared to parental cells.

LPS are a component of bacterial membranes and potent inducer of innate immune responses.

The LPS measurements are based on mass spectrometry as described in the previous Examples.

In an embodiment, less than 5%, 1%, 0.5%, 0.01%, 0.005%, 0.0001%, 0.00001% or less of the lipid content of cytobiologics will be LPS.

### Example 37: Ratio of Lipids to Proteins in Cytobiologics

This Example describes quantification of the ratio of lipid mass to protein mass in cytobiologics. In an embodiment, cytobiologics will have a ratio of lipid mass to protein mass that is similar to nucleated cells.

Total lipid content is calculated as the sum of the molar content of all lipids identified in the lipidomics data set outlined in a previous Example. Total protein content of the cytobiologics is measured via bicinchoninic acid assay as described herein.

Alternatively, the ratio of lipids to proteins can be described as a ratio of a particular lipid species to a specific protein. The particular lipid species is selected from the lipidomics data produced in a previous Example. The specific protein is selected from the proteomics data produced in a previous Example. Different combinations of selected lipid species and proteins are used to define specific lipid:protein ratios.

### Example 38: Ratio of Proteins to DNA in Cytobiologics

This Example describes quantification of the ratio of protein mass to DNA mass in cytobiologics. In an embodiment, cytobiologics will have a ratio of protein mass to DNA mass that is much greater than cells.

Total protein content of the cytobiologics and cells is measured as described in in a previous Example. The DNA mass of cytobiologics and cells is measured as described in a previous Example. The ratio of proteins to total nucleic acids is then determined by dividing the total protein content by the total DNA content to yield a ratio within a given range for a typical cytobiologic preparation.

Alternatively, the ratio of proteins to nucleic acids is determined by defining nucleic acid levels as the level of a specific house-keeping gene, such as GAPDH, using semi-quantitative real-time PCR (RT-PCR).

The ratio of proteins to GAPDH nucleic acids is then determined by dividing the total protein content by the total GAPDH DNA content to define a specific range of protein:nucleic acid ratio for a typical cytobiologic preparation.

### Example 39: Ratio of Lipids to DNA in Cytobiologics

This Example describes quantification of the ratio of lipids to DNA in cytobiologics compared to parental cells. In an embodiment, cytobiologics will have a greater ratio of lipids to DNA compared to parental cells.

This ratio is defined as total lipid content (outlined in an Example above) or a particular lipid species. In the case of a particular lipid species, the range depends upon the particular lipid species selected. The particular lipid species is selected from the lipidomics data produced in the previously described Example. Nucleic acid content is determined as described in the previously described Example.

Different combinations of selected lipid species normalized to nucleic acid content are used to define specific lipid:nucleic acid ratios that are characteristic of a particular cytobiologic preparation.

### Example 40: Analyzing Surface Markers on Cytobiologics

This assay describes identification of surface markers on the cytobiologics.

Cytobiologics are pelleted and shipped frozen to the proteomics analysis center per standard biological sample handling procedures.

To identify surface marker presence or absence on the cytobiologics, they are stained with markers against phosphatidyl serine and CD40 ligand and analyzed by flow cytometry using a FACS system (Becton Dickinson). For detection of surface phosphatidylserine, the product is analyzed with an annexin V assay (556547, BD Biosciences) as described by the manufacturer.

Briefly, the cytobiologics are washed twice with cold PBS and then resuspended in 1× binding buffer at a concentration of 1×106\hat{ }6 cytobiologics/ml. 10% of the resuspension is transferred to a 5 ml culture tube and 5 μl of FITC annexin V is added. The cells are gently vortexed and incubated for 15 min at room temperature (25° C.) in the dark.

In parallel, a separate 10% of the resuspension is transferred to a different tube to act as an unstained control. 1× binding buffer is added to each tube. The samples are analyzed by flow cytometry within 1 hr.

In some embodiments, using this assay, the mean of the population of the stained cytobiologics will be determined to be above the mean of the unstained cells indicating that the cytobiologics comprise phosphatidyl serine.

Similarly, for the CD40 ligand, the following monoclonal antibody is added to another 10% of the washed cytobiologics: PE-CF594 mouse anti-human CD154 clone TRAP1 (563589, BD Pharmigen) as per the manufacturer's directions. Briefly, saturating amounts of the antibody are used. In parallel, a separate 10% of the cytobiologics are transferred to a different tube to act as an unstained control. The tubes are centrifuged for 5 min at 400×g, at room temperature. The supernatant is decanted and the pellet is washed twice with flow cytometry wash solution. 0.5 ml of 1% paraformaldehyde fixative is added to each tube. Each is briefly vortexed and stored at 4° C. until analysis on the flow cytometer.

In an embodiment, using this assay, the mean of the population of the stained cytobiologics will be above the mean of the unstained cells indicating that the cytobiologics comprise CD40 ligand.

### Example 41: Analysis of Viral Capsid Proteins in Cytobiologics

This assay describes analysis of the makeup of the sample preparation and assesses the proportion of proteins that are derived from viral capsid sources.

Cytobiologics are pelleted and shipped frozen to a proteomics analysis center per standard biological sample handling procedures.

The cytobiologics are thawed for protein extraction and analysis. First, they are resuspended in lysis buffer (7M urea, 2M thiourea, 4% (w/v) chaps in 50 mM Tris pH 8.0) and incubated for 15 minutes at room temperature with occasional vortexing. The mixtures are then lysed by sonication for 5 minutes in an ice bath and spun down for 5 minutes at 13,000 RPM. Total protein content is determined by a colorimetric assay (Pierce) and 100 μg of protein from each sample is transferred to a new tube and the volume is adjusted with 50 mM Tris pH 8.

The proteins are reduced for 15 minutes at 65° Celsius with 10 mM DTT and alkylated with 15 mM iodoacetamide for 30 minutes at room temperature in the dark. The proteins are then precipitated with gradual addition of 6 volumes of cold (−20° Celsius) acetone and incubated over night at −80° Celsius.

The proteins are pelleted, washed 3 times with cold (−20° Celsius) methanol, and resuspended in 50 mM Tris pH 8. 3.33 μg of trypsin/lysC is added to the proteins for a first 4 h of digestion at 37° Celsius with agitation. The samples are diluted with 50 mM Tris pH 8 and 0.1% sodium deoxycholate is added with another 3.3 μg of trypsin/lysC for digestion overnight at 37° Celsius with agitation. Digestion is stopped and sodium deoxycholate is removed by the addition of 2% v/v formic acid. Samples are vortexed and cleared by centrifugation for 1 minute at 13,000 RPM.

The proteins are purified by reversed phase solid phase extraction (SPE) and dried down. The samples are reconstituted in 3% DMSO, 0.2% formic acid in water and analyzed by LC-MS as described previously.

The molar ratio of the viral capsid proteins relative to all proteins measured is determined as the molar quantity of all viral capsid proteins divided by the sum of the molar quantities of all identified proteins in each sample and expressed as a percent.

In an embodiment, using this approach, the sample will comprise less than 10% viral capsid protein. In an embodiment, the sample will comprise less than 0.5%, 1%, 5%, 10%, 15%, 20%, 25%, 30%, 35%, 40%, 45%, 50%, 60%, 70%, 80%, or 90% viral capsid protein.

### Example 42: Measuring Extravasation of Cytobiologics from Blood Vessels

This Example describes quantification of cytobiologic extravasation across an endothelial monolayer as tested with an in vitro microfluidic system (J. S Joen et al. 2013, journals.plos.org/plosone/article?id=10.1371/journal.pone.0056910).

Cells extravasate from the vasculature into surrounding tissue. Without wishing to be bound by theory, extravasation is one way for cytobiologics to reach extravascular tissues.

The system includes three independently addressable media channels, separated by chambers into which an ECM-mimicking gel can be injected. In brief, the microfluidics system has molded PDMS (poly-dimethyl siloxane; Silgard 184; Dow Chemical, MI) through which access ports are bored and bonded to a cover glass to form microfluidic channels. Channel cross-sectional dimensions are 1 mm (width) by 120 μm (height). To enhance matrix adhesion, the PDMS channels are coated with a PDL (poly-D-lysine hydrobromide; 1 mg/ml; Sigma-Aldrich, St. Louis, Mo.) solution.

Next, collagen type I (BD Biosciences, San Jose, Calif., USA) solution (2.0 mg/ml) with phosphate-buffered saline (PBS; Gibco) and NaOH is injected into the gel regions of the device via four separate filling ports and incubated for 30 min to form a hydrogel. When the gel is polymerized, endothelial cell medium (acquired from suppliers such as Lonza or Sigma) is immediately pipetted into the channels to prevent dehydration of the gel. Upon aspirating the medium, diluted hydrogel (BD science) solution (3.0 mg/ml) is introduced into the cell channel and the excess hydrogel solution is washed away using cold medium.

Endothelial cells are introduced into the middle channel and allowed to settle to form an endothelium. Two days after endothelial cell seeding, cytobiologics or macrophage cells (positive control) are introduced into the same channel where endothelial cells had formed a complete monolayer. The cytobiologics are introduced so they adhere to and transmigrate across the monolayer into the gel region. Cultures are kept in a humidified incubator at 37° C. and 5% CO₂. A GFP-expressing version of the cytobiologic is used to enable live-cell imaging via fluorescent microscopy. On the following day, cells are fixed and stained for nuclei using DAPI staining in the chamber, and multiple regions of interest are imaged using confocal microscope to determine how many cytobiologics passed through the endothelial monolayer.

In an embodiment, DAPI staining will indicate that cytobiologics and positive control cells are able to pass through the endothelial barrier after seeding.

### Example 43: Measuring Chemotactic Cell Mobility of Cytobiologics

This Example describes quantification of cytobiologic chemotaxis. Cells can move towards or away from a chemical gradient via chemotaxis. In an embodiment, chemotaxis will allow cytobiologics to home to a site of injury, or track a pathogen. A purified cytobiologic composition as produced by any one of the methods described in previous Examples is assayed for its chemotactic abilities as follows.

A sufficient number of cytobiologics or macrophage cells (positive control) are loaded in a micro-slide well according to the manufacturer's provided protocol in DMEM media (ibidi.com/img/cms/products/labware/channel_slides/S_8032 X_Chemotaxis/IN_8032 X_Chemotaxis.pdf). Cytobiologics are left at 37° C. and 5% CO2 for 1 h to attach. Following cell attachment, DMEM (negative control) or DMEM containing MCP1 chemoattractant is loaded into adjacent reservoirs of the central channel and the cytobiologics are imaged continuously for 2 hours using a Zeiss inverted widefield microscope. Images are analyzed using ImageJ software (Rasband, W. S., Image), U. S. National Institutes of Health, Bethesda, Md., USA, http://rsb.info.nih.gov/ij/, 1997-2007). Migration co-ordination data for each observed cytobiologic or cell is acquired with the manual tracking plugin (Fabrice Cordelières, Institut Curie, Orsay, France). Chemotaxis plots and migration velocities is determined with the Chemotaxis and Migration Tool (ibidi).

In an embodiment, the average accumulated distance and migration velocity of cytobiologics will be within 1%, 2%, 3%, 4%, 5%, 10%, 20%, 30%, 40%, 50%, 60%, 70%, 80%, 90%, 100% or greater than the response of the positive control cells to chemokine. The response of cells to a chemokine is described, e.g., in Howard E. Gendelman et al., Journal of Neuroimmune Pharmacology, 4(1): 47-59, 2009.

### Example 44: Measuring Homing Potential of Cytobiologics

This Example describes homing of cytobiologics to a site of injury. Cells can migrate from a distal site and/or accumulate at a specific site, e.g., home to a site. Typically, the site is a site of injury. In an embodiment, cytobiologics will home to, e.g., migrate to or accumulate at, a site of injury.

Eight week old C57BL/6J mice (Jackson Laboratories) are dosed with notexin (NTX) (Accurate Chemical & Scientific Corp), a myotoxin, in sterile saline by intramuscular (IM) injection using a 30G needle into the right tibialis anterior (TA) muscle at a concentration of 2 μg/mL. The skin over the tibialis anterior (TA) muscle is prepared by depilating the area using a chemical hair remover for 45 seconds, followed by 3 rinses with water. This concentration is chosen to ensure maximum degeneration of the myofibers, as well as minimal damage to their satellite cells, the motor axons and the blood vessels.

On day 1 after NTX injection, mice receive an IV injection of cytobiologics or cells that express firefly luciferase. Cytobiologics are produced from cells that stably express firefly luciferase by any one of the methods described in previous Examples. A bioluminescent imaging system (Perkin Elmer) is used to obtain whole animal images of bioluminescence at 0, 1, 3, 7, 21, and 28 post injection.

Five minutes before imaging, mice receive an intraperitoneal injection of bioluminescent substrate (Perkin Elmer) at a dose of 150 mg/kg in order to visualize luciferase. The imaging system is calibrated to compensate for all device settings. The bioluminescent signal is measured using Radiance Photons, with Total Flux used as a measured value. The region of interest (ROI) is generated by surrounding the signal of the ROI in order to give a value in photons/second. An ROI is assessed on both the TA muscle treated with NTX and on the contralateral TA muscle, and the ratio of photons/second between NTX-treated and NTX-untreated TA muscles is calculated as a measure of homing to the NTX-treated muscle.

In an embodiment, the ratio of photons/second between NTX-treated and NTX-untreated TA muscles in cytobiologics and cells will be greater than 1 indicating site specific accumulation of luciferase-expressing cytobiologics at the injury.

See, for example, Plant et al., Muscle Nerve 34(5)L 577-85, 2006.

### Example 45: Measuring Phagocytic Activity of Cytobiologics

This Example demonstrates phagocytic activity of cytobiologics. In an embodiment, cytobiologics have phagocytic activity, e.g., are capable of phagocytosis. Cells engage in phagocytosis, engulfing particles, enabling the sequestration and destruction of foreign invaders, like bacteria or dead cells.

A purified cytobiologic composition as produced by any one of the methods described in previous Examples comprising a cytobiologic from a mammalian macrophage having partial or complete nuclear inactivation was capable of phagocytosis assayed via pathogen bioparticles. This estimation was made by using a fluorescent phagocytosis assay according to the following protocol.

Macrophages (positive control) and cytobiologics were plated immediately after harvest in separate confocal glass bottom dishes. The macrophages and cytobiologics were incubated in DMEM+10% FBS+1% P/S for 1 h to attach. Fluorescein-labeled E. coli K12 and non-fluorescein-labeled Escherichia coli K-12 (negative control) were added to the macrophages/cytobiologics as indicated in the manufacturer's protocol, and were incubated for 2 h. After 2 h, free fluorescent particles were quenched by adding Trypan blue. Intracellular fluorescence emitted by engulfed particles was imaged by confocal microscopy at 488 excitation. The number of phagocytotic positive cytobiologic were quantified using image J software.

The average number of phagocytotic cytobiologics was at least 30% 2 h after bioparticle introduction, and was greater than 30% in the positive control macrophages.

### Example 46: Measuring Ability of Cytobiologics to Cross a Cell Membrane or the Blood Brain Barrier

This Example describes quantification of cytobiologics crossing the blood brain barrier. In an embodiment, cytobiologics will cross, e.g., enter and exit, the blood brain barrier, e.g., for delivery to the central nervous system.

Eight week old C57BL/6J mice (Jackson Laboratories) are intravenously injected with cytobiologics or leukocytes (positive control) that express firefly luciferase. Cytobiologics are produced from cells that stably express firefly luciferase or cells that do not express luciferase (negative control) by any one of the methods described in previous Examples. A bioluminescent imaging system (Perkin Elmer) is used to obtain whole-animal images of bioluminescence at one, two, three, four, five, six, eight, twelve, and twenty-four hours after cytobiologic or cell injection.

Five minutes before imaging, mice receive an intraperitoneal injection of bioluminescent substrate (Perkin Elmer) at a dose of 150 mg/kg in order to visualize luciferase. The imaging system is calibrated to compensate for all device settings. The bioluminescent signal is measured using Radiance Photons, with total flux used as a measured value. The region of interest (ROI) is generated by surrounding the signal of the ROI in order to give a value in photons/second. The ROI selected is the head of the mouse around the area that includes the brain.

In an embodiment, the photons/second in the ROI will be greater in the animals injected with cells or cytobiologics that express luciferase than the negative control cytobiologics that do not express luciferase indicating accumulation of luciferase-expressing cytobiologics in or around the brain.

### Example 47: Measuring Potential for Protein Secretion of Cytobiologics

This Example describes quantification of secretion by cytobiologics. In an embodiment, cytobiologics will be capable of secretion, e.g., protein secretion. Cells can dispose or discharge of material via secretion. In an embodiment, cytobiologics will chemically interact and communicate in their environment via secretion.

The capacity of cytobiologics to secrete a protein at a given rate is determined using the Gaussia luciferase flash assay from ThermoFisher Scientific (catalog #16158). Mouse embryonic fibroblast cells (positive control) or cytobiologics as produced by any one of the methods described in previous Examples are incubated in growth media and samples of the media are collected every 15 minutes by first pelleting the cytobiologics at 1600 g for 5 min and then collecting the supernatant. The collected samples are pipetted into a clear-bottom 96-well plate. A working solution of assay buffer is then prepared according to the manufacturer's instructions.

Briefly, colenterazine, a luciferin or light-emitting molecule, is mixed with flash assay buffer and the mixture is pipetted into each well of the 96 well plate containing samples. Negative control wells that lack cells or cytobiologics include growth media or assay buffer to determine background Gaussia luciferase signal. In addition, a standard curve of purified Gaussia luciferase (Athena Enzyme Systems, catalog #0308) is prepared in order to convert the luminescence signal to molecules of Gaussia luciferase secretion per hour.

The plate is assayed for luminescence, using 500 msec integration. Background Gaussia luciferase signal is subtracted from all samples and then a linear best-fit curve is calculated for the Gaussia luciferase standard curve. If sample readings do not fit within the standard curve, they are diluted appropriately and re-assayed. Using this assay, the capacity for cytobiologics to secrete Gaussia luciferase at a rate (molecules/hour) within a given range is determined.

In an embodiment, cytobiologics will be capable of secreting proteins at a rate that is 1%, 2%, 3%, 4%, 5%, 10%, 20%, 30%, 40%, 50%, 60%, 70%, 80%, 90%, 100% or greater than the positive control cells.

### Example 48: Measuring Signal Transduction Potential of Cytobiologics

This Example describes quantification of signal transduction in cytobiologics. In an embodiment, cytobiologics are capable of signal transduction. Cells can send and receive molecular signals from the extracellular environment through signaling cascades, such as phosphorylation, in a process known as signal transduction. A purified cytobiologic composition as produced by any one of the methods described in previous Examples comprising a cytobiologic from a mammalian cell having partial or complete nuclear inactivation is capable of signal transduction induced by insulin. Signal transduction induced by insulin is assessed by measuring AKT phosphorylation levels, a key pathway in the insulin receptor signaling cascade, and glucose uptake in response to insulin.

To measure AKT phosphorylation, cells, e.g., Mouse Embryonic Fibroblasts (MEFs) (positive control), and cytobiologics are plated in 48-well plates and left for 2 hours in a humidified incubator at 37° C. and 5% CO₂. Following cell adherence, insulin (e.g. at 10 nM), or a negative control solution without insulin, is add to the well containing cells or cytobiologics for 30 min. After 30 minutes, protein lysate is made from the cytobiologics or cells, and phospho-AKT levels are measured by western blotting in insulin stimulated and control unstimulated samples.

Glucose uptake in response to insulin or negative control solution is measured as it is explained in the glucose uptake section by using labeled glucose (2-NBDG). (S. Galic et al., Molecular Cell Biology 25(2): 819-829, 2005).

In an embodiment, cytobiologics will enhance AKT phosphorylation and glucose uptake in response to insulin over the negative controls by at least 1%, 2%, 3%, 4%, 5%, 10%, 20%, 30%, 40%, 50%, 60%, 70%, 80%, 90%, 100% or greater.

### Example 49: Measuring the Ability of Cytobiologics to Transport Glucose Across Cell Membrane

This Example describes quantification of the levels of a 2-NBDG (2-(N-(7-Nitrobenz-2-oxa-1,3-diazol-4-yl)Amino)-2-Deoxyglucose) a fluorescent glucose analog that can be used to monitor glucose uptake in live cells, and thus measure active transport across the lipid bilayer. In an embodiment, this assay can be used to measure the level of glucose uptake and active transport across the lipid bilayer of the cytobiologic.

A cytobiologic composition is produced by any one of the methods described in previous Examples. A sufficient number of cytobiologics are then incubated in DMEM with no glucose, 20% Fetal Bovine Serum and 1× Penicillin/Streptomycin for 2 hr at 37° C. and 5% CO₂. After a 2 hr glucose starvation period, the medium is changed such that it includes DMEM with no glucose, 20% Fetal Bovine Serum, 1× Penicillin/Streptomycin and 20 uM 2-NBDG (ThermoFisher) and incubated for an additional 2 hr at 37° C. and 5% CO₂.

Negative control cytobiologics are treated the same, except an equal amount of DMSO is added in place of 2-NBDG.

The cytobiologics are then washed thrice with 1×PBS and re-suspended in an appropriate buffer, and transferred to a 96 well imaging plate. 2-NBDG fluorescence is then measured in a fluorimeter using a GFP light cube (469/35 excitation filter and a 525/39 emission filter) to quantify the amount of 2-NBDG that has been transported across the cytobiologic membrane and accumulated in the cytobiologic in the 1 hr loading period.

In an embodiment, 2-NBDG fluorescence will be higher in the cytobiologic with 2-NBDG treatment as compared to the negative (DMSO) control. Fluorescence measure with a 525/39 emission filter will correlate with to the number of 2-NBDG molecules present.

### Example 50: Lumen of Cytobiologics are Miscible with Aqueous Solutions

This Example assesses the miscibility of a cytobiologic lumen with aqueous solutions, such as water.

The cytobiologics are prepared as described in previous Examples. The controls are dialysis membranes with either hypotonic solution, hyperosmotic solution or normal osmotic solutions.

Cytobiologics, positive control (normal osmotic solution) and negative control (hypotonic solution) are incubated with hypotonic solution (150 mOsmol). The cell size is measured under a microscope after exposing each sample to the aqueous solution. In an embodiment, the cytobiologic and positive control sizes in the hypotonic solution increase in comparison to the negative control.

Cytobiologics, positive control (normal osmotic solution) and negative control (hyperosmotic solution) are incubated with a hyperosmotic solution (400 mOsmol). The cell size is measured under a microscope after exposing each sample to the aqueous solution. In an embodiment, the cytobiologic and positive control sizes in the hyperosmotic solution will decrease in comparison to the negative control.

Cytobiologics, positive control (hypotonic or hyperosmotic solution) and negative control (normal osmotic) are incubated with a normal osmotic solution (290 mOsmol). The cell size is measured under a microscope after exposing each sample to the aqueous solution. In an embodiment, the cytobiologic and positive control sizes in the normal osmotic solution will remain substantially the same in comparison to the negative control.

### Example 51: Measuring Esterase Activity in the Cytosol of Cytobiologics

This Example describes quantification of esterase activity, as a surrogate for metabolic activity, in cytobiologics. The cytosolic esterase activity in cytobiologics is determined by quantitative assessment of calcein-AM staining (Bratosin et al., Cytometry 66(1): 78-84, 2005).

The membrane-permeable dye, calcein-AM (Molecular Probes, Eugene Oreg. USA), is prepared as a stock solution of 10 mM in dimethylsulfoxide and as a working solution of 100 mM in PBS buffer, pH 7.4. Cytobiologics as produced by any one of the methods described in previous Examples or positive control parental Mouse Embryonic Fibroblast cells are suspended in PBS buffer and incubated for 30 minutes with calcein-AM working solution (final concentration in calcein-AM: 5 mM) at 37° C. in the dark and then diluted in PBS buffer for immediate flow cytometric analysis of calcein fluorescence retention.

Cytobiologics and control parental Mouse Embryonic Fibroblast cells are experimental permeabilized as a negative control for zero esterase activity with saponin as described in (Jacob et al., Cytometry 12(6): 550-558, 1991). Cytobiologics and cells are incubated for 15 min in 1% saponin solution in PBS buffer, pH 7.4, containing 0.05% sodium azide. Due to the reversible nature of plasma membrane permeabilization, saponin is included in all buffers used for further staining and washing steps. After saponin permeabilization, cytobiologics and cells are suspended in PBS buffer containing 0.1% saponin and 0.05% sodium azide and incubated (37 C in the dark for 45 min) with calcein-AM to a final concentration of 5 mM, washed three times with the same PBS buffer containing 0.1% saponin and 0.05% sodium azide, and analyzed by flow cytometry. Flow cytometric analyses are performed on a FACS cytometer (Becton Dickinson, San Jose, Calif., USA) with 488 nm argon laser excitation and emission is collected at 530+/−30 nm. FACS software is used for acquisition and analysis. The light scatter channels are set on linear gains, and the fluorescence channels are set on a logarithmic scale, with a minimum of 10,000 cells analyzed in each condition. Relative esterase activities are calculated based on the intensity of calcein-AM in each sample. All events are captured in the forward and side scatter channels (alternatively, a gate can be applied to select only the cytobiologic population). The fluorescence intensity (FI) value for the cytobiologics is determined by subtracting the FI value of the respective negative control saponin-treated sample. The normalized esterase activity for the cytobiologics samples are normalized to the respective positive control cell samples in order to generate quantitative measurements for cytosolic esterase activities.

In an embodiment, a cytobiologic preparation will have within 1%, 2%, 3%, 4%, 5%, 10%, 20%, 30%, 40%, 50%, 60%, 70%, 80%, 90%, 100% or greater esterase activity compared to the positive control cell.

See also, Bratosin D, Mitrofan L, Palii C, Estaquier J, Montreuil J. Novel fluorescence assay using calcein-AM for the determination of human erythrocyte viability and aging. Cytometry A. 2005 July; 66(1):78-84; and Jacob B C, Favre M, Bensa J C. Membrane cell permeabilisation with saponin and multiparametric analysis by flow cytometry. Cytometry 1991; 12:550-558.

### Example 52: Measuring Acetylcholinesterase Activity in Cytobiologics

Acetylcholinesterase activity is measured using a kit (MAK119, SIGMA) that follows a procedure described previously (Ellman, et al., Biochem. Pharmacol. 7, 88, 1961) and following the manufacturer's recommendations.

Briefly, cytobiologics are suspended in 1.25 mM acetylthiocholine in PBS, pH 8, mixed with 0.1 mM 5,5-dithio-bis(2-nitrobenzoic acid) in PBS, pH 7. The incubation is performed at room temperature but the cytobiologics and the substrate solution are pre-warmed at 37° C. for 10 min before starting the optical density readings.

Changes in absorption are monitored at 450 nm for 10 min with a plate reader spectrophotometer (ELX808, BIO-TEK instruments, Winooski, Vt., USA). Separately, a sample is used for determining the protein content of the cytobiologics via bicinchoninic acid assay for normalization. Using this assay, the cytobiologics are determined to have <100 AChE activity units/μg of protein.

In an embodiment, AChE activity units/μg of protein values will be less than 0.001, 0.01, 0.1, 1, 10, 100, or 1000.

### Example 53: Measuring Metabolic Activity Level in Cytobiologics

This Example describes quantification of the measurement of citrate synthase activity in cytobiologics.

Citrate synthase is an enzyme within the tricarboxylic acid (TCA) cycle that catalyzes the reaction between oxaloacetate (OAA) and acetyl-CoA to generate citrate. Upon hydrolysis of acetyl-CoA, there is a release of CoA with a thiol group (CoA-SH). The thiol group reacts with a chemical reagent, 5,5-Dithiobis-(2-nitrobenzoic acid) (DTNB), to form 5-thio-2-nitrobenzoic acid (TNB), which is a yellow product that can be measured spectrophotometrically at 412 nm (Green 2008). Commercially-available kits, such as the Abcam Human Citrate Synthase Activity Assay Kit (Product #ab119692) provide all the necessary reagents to perform this measurement.

The assay is performed as per the manufacturer's recommendations. Cytobiologic sample lysates are prepared by collecting the cytobiologics as produced by any one of the methods described in previous Examples and solubilizing them in Extraction Buffer (Abcam) for 20 minutes on ice. Supernatants are collected after centrifugation and protein content is assessed by bicinchoninic acid assay (BCA, ThermoFisher Scientific) and the preparation remains on ice until the following quantification protocol is initiated.

Briefly, cytobiologic lysate samples are diluted in 1× Incubation buffer (Abcam) in the provided microplate wells, with one set of wells receiving only 1× Incubation buffer. The plate is sealed and incubated for 4 hours at room temperature with shaking at 300 rpm. The buffer is then aspirated from the wells and 1× Wash buffer is added. This washing step is repeated once more. Then, 1× Activity solution is added to each well, and the plate is analyzed on a microplate reader by measuring absorbance at 412 nm every 20 seconds for 30 minutes, with shaking between readings.

Background values (wells with only 1× Incubation buffer) are subtracted from all wells, and the citrate synthase activity is expressed as the change in absorbance per minute per μg of cytobiologic lysate sample loaded (ΔmOD@412 nm/min/ug protein). Only the linear portion from 100-400 seconds of the kinetic measurement is used to calculate the activity.

In an embodiment, a cytobiologic preparation will have within 1%, 2%, 3%, 4%, 5%, 10%, 20%, 30%, 40%, 50%, 60%, 70%, 80%, 90%, 100% or greater synthase activity compared to the control cell.

See, for example, Green H J et al. Metabolic, enzymatic, and transporter response in human muscle during three consecutive days of exercise and recovery. Am J Physiol Regul Integr Comp Physiol 295: R1238-R1250, 2008.

### Example 54: Measuring Respiration Levels in Cytobiologics

This Example describes quantification of the measurement of respiration level in cytobiologics. Respiration level in cells can be a measure of oxygen consumption, which powers metabolism. Cytobiologic respiration is measured for oxygen consumption rates by a Seahorse extracellular flux analyzer (Agilent) (Zhang 2012).

Cytobiologics as produced by any one of the methods described in previous Examples or cells are seeded in a 96-well Seahorse microplate (Agilent). The microplate is centrifuged briefly to pellet the cytobiologics and cells at the bottom of the wells. Oxygen consumption assays are initiated by removing growth medium, replacing with a low-buffered DMEM minimal medium containing 25 mM glucose and 2 mM glutamine (Agilent) and incubating the microplate at 37° C. for 60 minutes to allow for temperature and pH equilibrium.

The microplate is then assayed in an extracellular flux analyzer (Agilent) that measures changes in extracellular oxygen and pH in the media immediately surrounding adherent cytobiologics and cells. After obtaining steady state oxygen consumption (basal respiration rate) and extracellular acidification rates, oligomycin (5 μM), which inhibits ATP synthase, and proton ionophore FCCP (carbonyl cyanide 4-(trifluoromethoxy) phenylhydrazone; 2 μM), which uncouples mitochondria, are added to each well in the microplate to obtain values for maximal oxygen consumption rates.

Finally, 5 μM antimycin A (inhibitor of mitochondria complex III) is added to confirm that respiration changes are due mainly to mitochondrial respiration. The minimum rate of oxygen consumption after antimycin A addition is subtracted from all oxygen consumption measurements to remove the non-mitochondrial respiration component. Cell samples that do not appropriately respond to oligomycin (at least a 25% decrease in oxygen consumption rate from basal) or FCCP (at least a 50% increase in oxygen consumption rate after oligomycin) are excluded from the analysis. Cytobiologics respiration level is then measured as pmol 02/min/1e4 cytobiologics.

This respiration level is then normalized to the respective cell respiration level. In an embodiment, cytobiologics will have at least 1%, 2%, 3%, 4%, 5%, 10%, 20%, 30%, 40%, 50%, 60%, 70%, 80%, 90%, 100% or greater respiration level compared to the respective cell samples.

See, for example, Zhang J, Nuebel E, Wisidagama D R R, et al. Measuring energy metabolism in cultured cells, including human pluripotent stem cells and differentiated cells. Nature protocols. 2012; 7(6):10.1038/nprot.2012.048. doi:10.1038/nprot.2012.048.

### Example 55: Measuring Phosphatidylserine Levels of Cytobiologics

This Example describes quantification of the level of annexin-V binding to the surface of cytobiologics.

Dying cells can display phosphatidylserine on the cell surface which is a marker of apoptosis in the programmed cell death pathway. Annexin-V binds to phosphatidylserine, and thus, annexin-V binding is a proxy for viability in cells.

Cytobiologics were produced as described herein. For detection of apoptosis signals, cytobiologics or positive control cells were stained with 5% annexin V fluor 594 (A13203, Thermo Fisher, Waltham, Mass.). Each group (detailed in the table below) included an experimental arm that was treated with an apoptosis-inducer, menadione. Menadione was added at 100 μM menadione for 4 h. All samples were run on a flow cytometer (Thermo Fisher, Waltham, Mass.) and fluorescence intensity was measured with the YL1 laser at a wavelength of 561 nm and an emission filter of 585/16 nm. The presence of extracellular phophatidyl serine was quantified by comparing fluorescence intensity of annexin V in all groups.

The negative control unstained cytobiologics were not positive for annexin V staining.

In an embodiment, cytobiologics were capable of upregulating phosphatidylserine display on the cell surface in response to menadione, indicating that non-menadione stimulated cytobiologics are not undergoing apoptosis. In an embodiment, positive control cells that were stimulated with menadione demonstrated higher-levels of annexin V staining than cytobiologics not stimulated with menadione.

TABLE 10
Annexin V staining parameter

| | Mean Fluorescence Intensity |
|---|---|
| | of Annexin V Signal |
| Experimental Arm | (and standard deviation) |
| Unstained Cytobiologics (negative control) | 941 (937) |
| Stained Cytobiologics | 11257 (15826) |
| Stained Cytobiologics + Menadione | 18733 (17146) |
| Stained Macrophages + Menadione | 14301 (18142) |
| (positive control) | |

### Example 56: Measuring Juxtacrine-Signaling Levels in Cytobiologics

This Example describes quantification of juxtacrine-signaling in cytobiologics.

Cells can form cell-contact dependent signaling via juxtacrine signaling. In an embodiment, presence of juxtacrine signaling in cytobiologics will demonstrate that cytobiologics can stimulate, repress, and generally communicate with cells in their immediate vicinity.

Cytobiologics produced by any one of the methods described in previous Examples from mammalian bone marrow stromal cells (BMSCs) having partial or complete nuclear inactivation trigger IL-6 secretion via juxtacrine signaling in macrophages. Primary macrophages and BMSCs are co-cultured. Bone marrow-derived macrophages are seeded first into 6-well plates, and incubated for 24 h, then primary mouse BMSC-derived cytobiologics or BMSC cells (positive control parental cells) are placed on the macrophages in a DMEM medium with 10% FBS. The supernatant is collected at different time points (2, 4, 6, 24 hours) and analyzed for IL-6 secretion by ELISA assay. (Chang J. et al., 2015).

In an embodiment, the level of juxtacrine signaling induced by BMSC cytobiologics is measured by an increase in macrophage-secreted IL-6 levels in the media. In an embodiment, the level of juxtacrine signaling will be at least 1%, 2%, 3%, 4%, 5%, 10%, 20%, 30%, 40%, 50%, 60%, 70%, 80%, 90%, 100% or greater than the levels induced by the positive control bone marrow stromal cells (BMSCs).

### Example 57: Measuring Paracrine-Signaling Levels in Cytobiologics

This Example describes quantification of paracrine signaling in cytobiologics.

Cells can communicate with other cells in the local microenvironment via paracrine signaling. In an embodiment, cytobiologics will be capable of paracrine signaling, e.g., to communicate with cells in their local environment. In an embodiment, the ability of cytobiologics to trigger Ca²⁺ signaling in endothelial cells via paracrine-derived secretion with the following protocol will measure Ca²⁺ signaling via the calcium indicator, fluo-4 AM.

To prepare the experimental plate, murine pulmonary microvascular endothelial cells (MPMVECs) are plated on a 0.2% gelatin coated 25 mm glass bottom confocal dish (80% confluence). MPMVECs are incubated at room temperature for 30 min in ECM containing 2% BSA and 0.003% pluronic acid with 5 μM fluo-4 AM (Invitrogen) final concentration to allow loading of fluo-4 AM. After loading, MPMVECs are washed with experimental imaging solution (ECM containing 0.25% BSA) containing sulfinpyrazone to minimize dye loss. After loading fluo-4, 500 μl of pre-warmed experimental imaging solution is added to the plate, and the plate is imaged by a Zeiss confocal imaging system.

In a separate tube, freshly isolated murine macrophages are either treated with 1 μg/ml LPS in culture media (DMEM+10% FBS) or not treated with LPS (negative control). After stimulation, cytobiologics are generated from macrophages by any one of the methods described in previous Examples.

Cytobiologics or parental macrophages (positive control) are then labeled with cell tracker red, CMTPX (Invitrogen), in ECM containing 2% BSA and 0.003% pluronic acid. Cytobiologics and macrophages are then washed and resuspended in experimental imaging solution. Labeled cytobiologics and macrophages are added onto the fluo-4 AM loaded MPMVECs in the confocal plate.

Green and red fluorescence signal is recorded every 3 s for 10-20 min using Zeiss confocal imaging system with argon ion laser source with excitation at 488 and 561 nm for fluo-4 AM and cell tracker red fluorescence respectively. Fluo-4 fluorescence intensity changes are analyzed using imaging software (Mallilankaraman, K. et al., J Vis Exp. (58): 3511, 2011). The level of Fluo-4 intensity measured in negative control cytobiologic and cell groups is subtracted from LPS-stimulated cytobiologic and cell groups.

In an embodiment, cytobiologics, e.g., activated cytobiologics, will induce an increase in Fluo-4 fluorescence intensity that is at least 1%, 2%, 3%, 4%, 5%, 10%, 20%, 30%, 40%, 50%, 60%, 70%, 80%, 90%, 100% or greater than the positive control cell groups.

### Example 58: Measuring Ability to Polymerize Actin for Mobility of Cytobiologics

This Example describes quantification of cytoskeletal components, such as actin, in cytobiologics. In an embodiment, cytobiologics comprise cytoskeletal components such as actin, and are capable of actin polymerization.

Cells use actin, which is a cytoskeletal component, for motility and other cytoplasmic processes. The cytoskeleton is essential to creating motility driven forces and coordinating the process of movement

C2C12 cells were enucleated as described herein. Cytobiologics obtained from the 12.5% and 15% Ficoll layers were pooled and labeled ‘Light’, while cytobiologics from the 16-17% layers were pooled and labeled ‘Medium’. Cytobiologics or cells (parental C2C12 cells, positive control) were resuspended in DMEM+Glutamax+10% Fetal Bovine Serum (FBS), plated in 24-well ultra-low attachment plates (#3473, Corning Inc, Corning, N.Y.) and incubated at 37° C.+5% CO₂. Samples were taken periodically (5.25 hr, 8.75 hr, 26.5 hr) and stained with 165 μM rhodamine phalloidin (negative control was not stained) and measured on a flow cytometer (#A24858, Thermo Fisher, Waltham, Mass.) with a FC laser YL1 (561 nm with 585/16 filter) to measure F-actin cytoskeleton content. The fluorescence intensity of rhodamine phalloidin in cytobiologics was measured along with unstained cytobiologics and stained parental C2C12 cells.

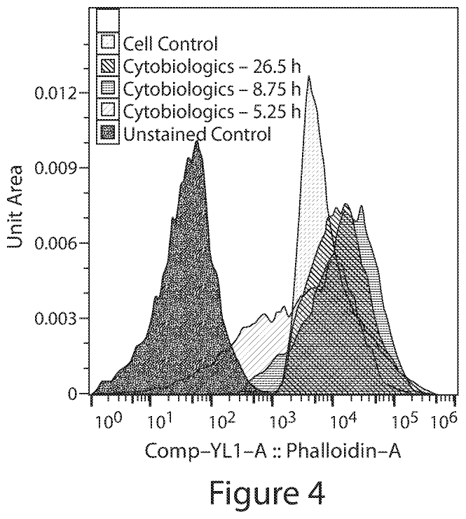

Cytobiologic fluorescence intensity was greater (FIG. 4) than the negative control at all timepoints, and cytobiologics were capable of polymerizing actin at a similar rate to the parental C2C12 cells.

Additional cytoskeletal components, such as those listed in the table below, are measured via a commercially available ELISA systems (Cell Signaling Technology and MyBioSource), according to manufacturer's instructions.

TABLE 11
Cytoskeletal components

| Cytoskeletal protein | | |
|---|---|---|
| measured | Commercial Kit Type | Kit ID |
| Actin | Path Scan Total B- | Cell Signaling, |
| | Actin Sandwich | 7880 |
| | ELISA Kit | |
| Arp2/3 | Human Actin Related | MyBioSource, |
| | protein 2/3 complex | MBS7224740 |
| | subunit(APRC2) | |
| | ELISA KIT | |
| Formin | Formin Binding | MyBioSource, |
| | Protein 1 (FNBP1), | MBS9308864 |
| | ELISA Kit | |
| Coronin | Human Coronin 1A | MyBioSource, |
| | ELISA Kit | MBS073640 |
| Dystrophin | Human dystrophin | MyBioSource |
| | ELISA Kit | MBS722223 |
| Keratin | Human Keratin 5 | MyBioSource, |
| | ELISA Kit | MBS081200 |
| Myosin | Human Myosin IG | MyBioSource, |
| | (MYO1G) ELISA Kit | MBS9312965 |
| Tubulin | Human Tubulin Beta 3 | MyBioSource, |
| | ELISA Kit | MBS097321 |

Then 100 uL of appropriately-diluted lysate is added to the appropriate well from the microwell strips. The microwells are sealed with tape and incubated for 2 hrs at 37 C. After incubation, the sealing tape is removed and the contents are discarded. Each microwell is washed four times with 200 uL of 1× Wash Buffer. After each individual wash, plates are struck onto an absorbent cloth so that the residual wash solution is removed from each well. However, wells are not completely dry at any time during the experiment.

Next, 100 ul of the reconstituted Detection Antibody (green) is added each individual well, except for negative control wells. Then wells are sealed and incubated for 1 hour at 37° C. The washing procedure is repeated after incubation is complete. 100 uL of reconstituted HRP-Linked secondary antibody (red) is added to each of the wells. The wells are sealed with tape and incubated for 30 minutes at 37° C. The sealing tape is then removed and the washing procedure is repeated. 100 uL of TMB Substrate is then added to each well. The wells are sealed with tape, then incubated for 10 minutes at 37° C. Once this final incubation is complete, 100 uL of STOP solution is added to each of the wells and the plate is shaken gently for several seconds.

Spectrophotometric analysis of the assay is conducted within 30 minutes of adding the STOP solution. The underside of the wells is wiped with lint-free tissue and then absorbance is read at 450 nm. In an embodiment, cytobiologic samples that have been stained with the detection antibody will absorb more light at 450 nm that negative control cytobiologic samples, and absorb less light than cell samples that have been stained with the detection antibody.

### Example 59: Measuring Average Membrane Potential of Cytobiologics

This Example describes quantification of the mitochondrial membrane potential of cytobiologics. In an embodiment, cytobiologics comprising a mitochondrial membrane will maintain mitochondrial membrane potential.

Mitochondrial metabolic activity can be measured by mitochondrial membrane potential. The membrane potential of the cytobiologic preparation is quantified using a commercially available dye, TMRE, for assessing mitochondrial membrane potential (TMRE: tetramethyl rhodamine, ethyl ester, perchlorate, Abcam, Cat #T669).

Cytobiologics are generated by any one of the methods described in previous Examples. Cytobiologics or parental cells are diluted in growth medium (phenol-red free DMEM with 10% fetal bovine serum) in 6 aliquots (untreated and FCCP-treated triplicates). One aliquot of the samples is incubated with FCCP, an uncoupler that eliminates mitochondrial membrane potential and prevents TMRE staining. For FCCP-treated samples, 2 μM FCCP is added to the samples and incubated for 5 minutes prior to analysis. Cytobiologics and parental cells are then stained with 30 nM TMRE. For each sample, an unstained (no TMRE) sample is also prepared in parallel. Samples are incubated at 37° C. for 30 minutes. The samples are then analyzed on a flow cytometer with 488 nm argon laser, and excitation and emission is collected at 530+/−30 nm.

Membrane potential values (in millivolts, mV) are calculated based on the intensity of TMRE. All events are captured in the forward and side scatter channels (alternatively, a gate can be applied to exclude small debris). The fluorescence intensity (FI) value for both the untreated and FCCP-treated samples are normalized by subtracting the geometric mean of the fluorescence intensity of the unstained sample from the geometric mean of the untreated and FCCP-treated sample. The membrane potential state for each preparation is calculated using the normalized fluorescent intensity values with a modified Nernst equation (see below) that can be used to determine mitochondrial membrane potential of the cytobiologics or cells based on TMRE fluorescence (as TMRE accumulates in mitochondria in a Nernstian fashion).

Cytobiologic or cell membrane potential is calculated with the following formula: (mV)=−61.5*log(Fluntreated-normalized/FIFCCP-treated-normalized). In an embodiment, using this assay on cytobiologic preparations from C2C12 mouse myoblast cells, the membrane potential state of the cytobiologic preparation will be within about 1%, 2%, 3%, 4%, 5%, 10%, 20%, 30%, 40%, 50%, 60%, 70%, 80%, 90%, 100% or greater than the parental cells. In an embodiment, the range of membrane potential is about −20 to −150 mV.

### Example 60: Measuring Persistence Half-Life in a Subject of Cytobiologics

This Example describes the measurement of cytobiologic half-life.

Cytobiologics are derived from cells that express gaussia-luciferase produced by any one of the methods described in previous Examples, and pure, 1:2, 1:5, and 1:10 dilutions in buffered solution are made. A buffered solution lacking cytobiologics is used as a negative control.

Each dose is administered to three eight week old male C57BL/6J mice (Jackson Laboratories) intravenously. Blood is collected from the retro-orbital vein at 1, 2, 3, 4, 5, 6, 12, 24, 48, and 72 hours after intravenous administration of the cytobiologics. The animals are sacrificed at the end of the experiment by CO₂ inhalation.

Blood is centrifuged for 20 min at room temperature. The serum samples are immediately frozen at −80° C. until bioanalysis. Then, each blood sample is used to carry out a Gaussia-luciferase activity assay after mixing the samples with Gaussia-luciferase substrate (Nanolight, Pinetop, Ariz.). Briefly, colenterazine, a luciferin or light-emitting molecule, is mixed with flash assay buffer and the mixture is pipetted into wells containing blood samples in a 96 well plate. Negative control wells that lack blood contain assay buffer to determine background Gaussia luciferase signal.

In addition, a standard curve of positive-control purified Gaussia luciferase (Athena Enzyme Systems, catalog #0308) is prepared in order to convert the luminescence signal to molecules of Gaussia luciferase secretion per hour. The plate is assayed for luminescence, using 500 msec integration. Background Gaussia luciferase signal is subtracted from all samples and then a linear best-fit curve is calculated for the Gaussia luciferase standard curve. If sample readings do not fit within the standard curve, they are diluted appropriately and re-assayed. The luciferase signal from samples taken at 1, 2, 3, 4, 5, 6, 12, 24, 48, and 72 hours is interpolated to the standard curve. The elimination rate constant kₑ (h⁻¹) is calculated using the following equation of a one-compartment model: C(t)=C₀×e^{-kext}, in which C(t) (ng/mL) is the concentration of cytobiologics at time t (h) and C₀ the concentration of cytobiologics at time=0 (ng/mL). The elimination half-life t_{1/2,e} (h) is calculated as ln(2)/kₑ.

In an embodiment, cytobiologics will have a half-life of at least 1%, 2%, 3%, 4%, 5%, 10%, 20%, 30%, 40%, 50%, 60%, 70%, 80%, 90%, 100% or greater than the negative control cells.

### Example 61: Cytobiologic Longevity with Immunosuppression

This Example describes quantification of the immunogenicity of a cytobiologic composition when it is co-administered with an immunosuppressive drug.

Therapies that stimulate an immune response can sometimes reduce the therapeutic efficacy or cause toxicity to the recipient. In an embodiment, the cytobiologics will be substantially non-immunogenic.

A purified composition of cytobiologics as produced by any one of the methods described in previous Examples is co-administered with an immunosuppressive drug, and immunogenic properties are assayed by the longevity of the cytobiologic in vivo. A sufficient number of cytobiologics, labeled with luciferase, are injected locally into the gastrocnemius muscle of a normal mouse with tacrolimus (TAC, 4 mg/kg/day; Sigma Aldrich), or vehicle (negative control), or without any additional agent (positive control). The mice are then subjected to in vivo imaging at 1, 2, 3, 4, 5, 6, 12, 24, 48, and 72 hours post injection.

Briefly, mice are anesthetized with isoflurane and D-luciferin is administered intraperitoneally at a dose of 375 mg per kilogram of body weight. At the time of imaging, animals are placed in a light-tight chamber, and photons emitted from luciferase expressing cytobiologics transplanted into the animals are collected with integration times of 5 sec to 5 min, depending on the intensity of the bioluminescence emission. The same mouse is scanned repetitively at the various timepoints set forth above. BLI signal is quantified in units of photons per second (total flux) and presented as log [photons per second]. The data is analyzed by comparing the intensity and cytobiologic injection with and without TAC.

In embodiments, the assay will show an increase in cytobiologic longevity in the TAC co-administered group relative to the cytobiologic alone and vehicle groups at the final timepoint. In addition to the increase in cytobiologic longevity, in some embodiments, an increase in BLI signal from the cytobiologic plus TAC arm versus the cytobiologic plus vehicle or cytobiologics alone at each of the time points will be observed.

### Example 62: Measuring Pre-Existing IgG and IgM Antibodies Reactive Against Cytobiologics

This Example describes quantification of pre-existing anti-cytobiologic antibody titers measured using flow cytometry.

A measure of immunogenicity for cytobiologics is antibody responses. Antibodies that recognize cytobiologics can bind in manner that can limit cytobiologic activity or longevity. In an embodiment, some recipients of a cytobiologic described herein will have pre-existing antibodies which bind to and recognize cytobiologics.

In this Example, anti-cytobiologic antibody titers are tested using cytobiologics produced using a xenogeneic source cell by any one of the methods described in a previous Example. In this Example, a cytobiologic naïve mouse is assessed for the presence of anti-cytobiologic antibodies. Notably, the methods described herein may be equally applicable to humans, rats, monkeys with optimization to the protocol.

The negative control is mouse serum which has been depleted of IgM and IgG, and the positive control is serum derived from a mouse that has received multiple injections of cytobiologics generated from a xenogeneic source cell.

To assess the presence of pre-existing antibodies which bind to cytobiologics, sera from cytobiologic-naïve mice is first decomplemented by heating to 56° C. for 30 min and subsequently diluted by 33% in PBS containing 3% FCS and 0.1% NaN3. Equal amounts of sera and cytobiologics (1×10²-1×10⁸ cytobiologics per mL) suspensions are incubated for 30 min at 4° C. and washed with PBS through a calf-serum cushion.

IgM xenoreactive antibodies are stained by incubation of the cells with PE-conjugated goat antibodies specific for the Fc portion of mouse IgM (BD Bioscience) at 4° C. for 45 min. Notably, anti-mouse IgG1 or IgG2 secondary antibodies may also be used. Cells from all groups are washed twice with PBS containing 2% FCS and then analyzed on a FACS system (BD Biosciences). Fluorescence data are collected by use of logarithmic amplification and expressed as mean fluorescent intensity.

In an embodiment, the negative control serum will show negligible fluorescence comparable to the no serum or secondary alone controls. In an embodiment, the positive control will show more fluorescence than the negative control, and more than the no serum or secondary alone controls. In an embodiment, in cases where immunogenicity occurs, serum from cytobiologic-naïve mice will show more fluorescence than the negative control. In an embodiment, in cases where immunogenicity does not occur, serum from cytobiologic-naïve mice will show similar fluorescence compared to the negative control.

### Example 63: Measuring IgG and IgM Antibody Responses after Multiple Administrations of Cytobiologics

This Example describes quantification of the humoral response of a modified cytobiologic following multiple administrations of the modified cytobiologic. In an embodiment, a modified cytobiologic, e.g., modified by a method described herein, will have a reduced (e.g., reduced compared to administration of an unmodified cytobiologic) humoral response following multiple (e.g., more than one, e.g., 2 or more), administrations of the modified cytobiologic.

A measure of immunogenicity for cytobiologics is the antibody responses. In an embodiment, repeated injections of a cytobiologic can lead to the development of anti-cytobiologic antibodies, e.g., antibodies that recognize cytobiologics. In an embodiment, antibodies that recognize cytobiologics can bind in a manner that can limit cytobiologic activity or longevity.

In this Example, anti-cytobiologic antibody titers are examined after one or more administrations of cytobiologics. Cytobiologics are produced by any one of the previous Examples. Cytobiologics are generated from: unmodified mesenchymal stem cells (hereafter MSCs), mesenchymal stem cells modified with a lentiviral-mediated expression of HLA-G (hereafter MSC-HLA-G), and mesenchymal stem cells modified with a lentiviral-mediated expression of an empty vector (hereafter MSC-empty vector). Serum is drawn from the different cohorts: mice injected systemically and/or locally with 1, 2, 3, 5, 10 injections of vehicle (Cytobiologic naïve group), MSC cytobiologics, MSC-HLA-G cytobiologics, or MSC-empty vectors cytobiologics.

To assess the presence and abundance of anti-cytobiologics antibodies, sera from the mice is first decomplemented by heating to 56° C. for 30 min and subsequently diluted by 33% in PBS with 3% FCS and 0.1% NaN3. Equal amounts of sera and cytobiologics (1×10²-1×10⁸ cytobiologics per mL) are incubated for 30 min at 4° C. and washed with PBS through a calf-serum cushion.

Cytobiologic reactive IgM antibodies are stained by incubation of the cells with PE-conjugated goat antibodies specific for the Fc portion of mouse IgM (BD Bioscience) at 4° C. for 45 min. Notably, anti-mouse IgG1 or IgG2 secondary antibodies may also be used. Cells from all groups are washed twice with PBS containing 2% FCS and then analyzed on a FACS system (BD Biosciences). Fluorescence data are collected by use of logarithmic amplification and expressed as mean fluorescent intensity.

In an embodiment, MSC-HLA-G cytobiologics will have decreased anti-cytobiologic IgM (or IgG1/2) antibody titers (as measured by fluorescence intensity on FACS) after injections, as compared to MSC cytobiologics or MSC-empty vector cytobiologics.

### Example 64: Modification of Cytobiologic Source Cells to Express Tolerogenic Protein to Reduce Immunogenicity

This Example describes quantification of immunogenicity in cytobiologics derived from a modified cell source. In an embodiment, cytobiologics derived from a modified cell source have reduced immunogenicity in comparison to the cytobiologics derived from an unmodified cell source.

Therapies that stimulate an immune response can sometimes reduce the therapeutic efficacy or cause toxicity to the recipient. In an embodiment, substantially non-immunogenic cytobiologics are administered to a subject. In an embodiment, immunogenicity of the cell source can be assayed as a proxy for cytobiologic immunogenicity.

iPS cells modified using lentiviral mediated expression of HLA-G or expressing an empty vector (Negative control) are assayed for immunogenic properties as follows. A sufficient number of iPS cells, as a potential cytobiologic cell source, are injected into C57/B6 mice, subcutaneously in the hind flank and are given an appropriate amount of time to allow for teratomas to form.

Once teratomas are formed, tissues are harvested. Tissues prepared for fluorescent staining are frozen in OCT, and those prepared for immunohistochemistry and H&E staining are fixed in 10% buffered formalin and embedded in paraffin. The tissue sections are stained with antibodies, polyclonal rabbit anti-human CD3 anti-body (DAKO), mouse anti-human CD4 mAb (RPA-T4, BD PharMingen), mouse anti-human CD8 mAb (RPA-T8, BD PharMingen), in accordance with general immunohistochemistry protocols. These are detected by using an appropriate detection reagent, namely an anti-mouse secondary HRP (Thermofisher), or anti-rabbit secondary HRP (Thermofisher).

Detection is achieved using peroxidase-based visualization systems (Agilent). The data is analyzed by taking the average number of infiltrating CD4+ T-cells, CD8+ T-cells, CD3+ NK-cells present in 25, 50 or 100 tissue sections examined in a 20× field using a light microscope. In an embodiment, iPSCs which are not modified or iPSCs expressing an empty vector will have a higher number of infiltrating CD4+ T-cells, CD8+ T-cells, CD3+ NK-cells present in the fields examined as compared to iPSCs that express HLA-G.

In an embodiment, a cytobiologic's immunogenic properties will be substantially equivalent to that of the source cell. In an embodiment, cytobiologics derived from iPS cells modified with HLA-G will have reduced immune cell infiltration versus their unmodified counterparts.

### Example 65: Modification of Cytobiologic Source Cells to Knockdown Immunogenic Protein to Reduce Immunogenicity

This Example describes quantification of the generation of a cytobiologic composition derived from a cell source, which has been modified to reduce expression of a molecule which is immunogenic. In an embodiment, a cytobiologic can be derived from a cell source, which has been modified to reduce expression of a molecule which is immunogenic.

Therapies that stimulate an immune response can reduce the therapeutic efficacy or cause toxicity to the recipient. Thus, immunogenicity is an important property for a safe and effective therapeutic cytobiologics. Expression of certain immune activating agents can create an immune response. MHC class I represents one example of an immune activating agent.

In this Example, cytobiologics are generated by any one of the methods described in previous Examples. Cytobiologics are generated from: unmodified mesenchymal stem cells (hereafter MSCs, positive control), mesenchymal stem cells modified with a lentiviral-mediated expression of an shRNA targeting MHC class I (hereafter MSC-shMHC class I), and mesenchymal stem cells modified with a lentiviral-mediated expression of a non-targeted scrambled shRNA (hereafter MSC-scrambled, negative control).

Cytobiologics are assayed for expression of MHC class I using flow cytometry. An appropriate number of cytobiologics are washed and resuspended in PBS, held on ice for 30 minutes with 1:10-1:4000 dilution of fluorescently conjugated monoclonal antibodies against MHC class I (Harlan Sera-Lab, Belton, UK). Cytobiologics are washed three times in PBS and resuspended in PBS. Nonspecific fluorescence is determined, using equal aliquots of cytobiologics preparation incubated with and appropriate fluorescently conjugated isotype control antibody at equivalent dilutions. Cytobiologics are assayed in a flow cytometer (FACSort, Becton-Dickinson) and the data is analyzed with flow analysis software (Becton-Dickinson).

The mean fluorescence data of the cytobiologics derived from MSCs, MSCs-shMHC class I, MSC-scrambled, is compared. In an embodiment, cytobiologics derived from MSCs-shMHC class I will have lower expression of MHC class I compared to MSCs and MSC-scrambled.

### Example 66: Modification of Cytobiologic Source Cells to Evade Macrophage Phagocytosis

This Example describes quantification of the evasion of phagocytosis by modified cytobiologics. In an embodiment, modified cytobiologics will evade phagocytosis by macrophages.

Cells engage in phagocytosis, engulfing particles, enabling the sequestration and destruction of foreign invaders, like bacteria or dead cells. In some embodiments, phagocytosis of cytobiologics by macrophages would reduce their activity.

Cytobiologics are generated by any one of the methods described in previous Examples. Cytobiologics are created from: CSFE-labelled mammalian cells which lack CD47 (hereafter NMC, positive control), CSFE-labelled cells that are engineered to express CD47 using lentiviral mediated expression of a CD47 cDNA (hereafter NMC-CD47), and CSFE-labelled cells engineered using lentiviral mediated expression of an empty vector control (hereafter NMC-empty vector, negative control).

Reduction of macrophage mediate immune clearance is determined with a phagocytosis assay according to the following protocol. Macrophages are plated immediately after harvest in confocal glass bottom dishes. Macrophages are incubated in DMEM+10% FBS+1% P/S for 1 h to attach. An appropriate number of cytobiologics derived from NMC, NMC-CD47, NMC-empty vector are added to the macrophages as indicated in the protocol, and are incubated for 2 h.

After 2 h, the dish is gently washed and intracellular fluorescence is examined Intracellular fluorescence emitted by engulfed particles is imaged by confocal microscopy at 488 excitation. The number of phagocytotic positive macrophage is quantified using imaging software. The data is expressed as the phagocytic index=(total number of engulfed cells/total number of counted macrophages)×(number of macrophages containing engulfed cells/total number of counted macrophages)×100.

In an embodiment, the phagocytic index will be reduced when macrophages are incubated with cytobiologics derived from NMC-CD47, versus those derived from NMC, or NMC-empty vector.

### Example 67: Modification of Cytobiologic Source Cells for Decreased Cytotoxicity Mediated by PBMC Cell Lysis

This Example described the generation of cytobiologics derived from cells modified to have decreased cytotoxicity due to cell lysis by PBMCs.

In an embodiment, cytotoxicity mediated cell lysis of source cells or cytobiologics by PBMCs is a measure of immunogenicity for cytobiologics, as lysis will reduce, e.g., inhibit or stop, the activity of a cytobiologic.

In this Example, cytobiologics are generated by any one of the methods described in a previous Example. Cytobiologics are created from: unmodified mesenchymal stem cells (hereafter MSCs, positive control), mesenchymal stem cells modified with a lentiviral-mediated expression of HLA-G (hereafter MSC-HLA-G), and mesenchymal stem cells modified with a lentiviral-mediated expression of an empty vector (hereafter MSC-empty vector, negative control).

PMBC mediated lysis of a cytobiologic is determined by europium release assays as described in Bouma, et al. Hum. Immunol. 35(2):85-92; 1992 & van Besouw et al. Transplantation 70(1):136-143; 2000. PBMCs (hereafter effector cells) are isolated from an appropriate donor, and stimulated with allogeneic gamma irradiated PMBCs and 2001 U/mL IL-2 (proleukin, Chiron BV Amsterdam, The Netherlands) in a round bottom 96 well plate for 7 days at 37 C. The cytobiologics are labeled with europium-diethylenetriaminepentaacetate (DTPA) (sigma, St. Louis, Mo., USA).

At day 7 cytotoxicity-mediated lysis assays is performed by incubating ⁶³Eu-labelled cytobiologics with effector cells in a 96-well plate for 1, 2, 3, 4, 5, 6, 8, 10, 15, 20, 24, 48 hours after plating at effector/target ratios ranging from 1000:1-1:1 and 1:1.25-1:1000. After incubation, the plates are centrifuged and a sample of the supernatant is transferred to 96-well plates with low background fluorescence (fluoroimmunoplates, Nunc, Roskilde, Denmark).

Subsequently, enhancement solution (PerkinElmer, Groningen, The Netherlands) is added to each well. The released europium is measured in a time-resolved fluorometer (Victor 1420 multilabel counter, LKB-Wallac, Finland). Fluorescence is expressed in counts per second (CPS). Maximum percent release of europium by a target cytobiologic is determined by incubating an appropriate number (1×10²-1×10⁸) of cytobiologics with 1% triton (sigma-aldrich) for an appropriate amount of time. Spontaneous release of europium by target cytobiologics is measured by incubation of labeled target cytobiologics without effector cells. Percentage leakage is then calculated as: (spontaneous release/maximum release)×100%. Finally, the percentage of cytotoxicity mediated lysis is calculated as % lysis=[(measured lysis−spontaneous lysis−spontaneous release)/(maximum release-spontaneous release)]×100%. The data is analyzed by looking at the percentage of lysis as a function of different effector target ratios.

In an embodiment, cytobiologics generated from MSC-HLA-G cells will have a decreased percentage of lysis by target cells, at specific timepoints as compared to MSCs or MSC-scrambled generated cytobiologics.

### Example 68: Modification of Cytobiologic Source Cells for Decreased NK Lysis Activity

This Example describes the generation of a cytobiologic composition derived from a cell source, which has been modified to decrease cytotoxicity mediated cell lysis by NK cells. In an embodiment cytotoxicity mediated cell lysis of source cells or cytobiologics by NK cells is a measure of immunogenicity for cytobiologics.

In this Example, cytobiologics are generated by any one of the methods described in a previous Example. Cytobiologics are created from: unmodified mesenchymal stem cells (hereafter MSCs, positive control), mesenchymal stem cells modified with a lentiviral-mediated expression of HLA-G (hereafter MSC-HLA-G), and mesenchymal stem cells modified with a lentiviral-mediated expression of an empty vector (hereafter MSC-empty vector, negative control).

NK cell mediated lysis of a cytobiologic is determined by europium release assays as described in Bouma, et al. Hum. Immunol. 35(2):85-92; 1992 & van Besouw et al. Transplantation 70(1):136-143; 2000. NK cells (hereafter effector cells) are isolated from an appropriate donor according to the methods in Crop et al. Cell transplantation (20):1547-1559; 2011, and stimulated with allogeneic gamma irradiated PMBCs and 2001 U/mL IL-2 (proleukin, Chiron BV Amsterdam, The Netherlands) in a round bottom 96 well plate for 7 days at 37 C. The cytobiologics are labeled with europium-diethylenetriaminepentaacetate (DTPA) (sigma, St. Louis, Mo., USA).

At day 7 cytotoxicity-mediated lysis assays is performed by incubating ⁶³Eu-labelled cytobiologics with effector cells in a 96-well plate for 1, 2, 3, 4, 5, 6, 8, 10, 15, 20, 24, 48 hours after plating at effector/target ratios ranging from 1000:1-1:1 and 1:1.25-1:1000. After incubation, the plates are centrifuged and a sample of the supernatant is transferred to 96-well plates with low background fluorescence (fluoroimmunoplates, Nunc, Roskilde, Denmark).

Subsequently, enhancement solution (PerkinElmer, Groningen, The Netherlands) is added to each well. The released europium is measured in a time-resolved fluorometer (Victor 1420 multilabel counter, LKB-Wallac, Finland). Fluorescence is expressed in counts per second (CPS). Maximum percent release of europium by a target cytobiologic is determined by incubating an appropriate number (1×10²-1×10⁸) of cytobiologics with 1% triton (Sigma-Aldrich) for an appropriate amount of time. Spontaneous release of europium by target cytobiologics is measured by incubation of labeled target cytobiologics without effector cells. Percentage leakage is then calculated as: (spontaneous release/maximum release)×100%. Finally, the percentage of cytotoxicity mediated lysis is calculated as % lysis=[(measured lysis−spontaneous lysis−spontaneous release)/(maximum release-spontaneous release)]×100%. The data is analyzed by looking at the percentage of lysis as a function of different effector target ratios.

In an embodiment, cytobiologics generated from MSC-HLA-G cells will have a decreased percentage of lysis at appropriate timepoints as compared to MSCs or MSC-scrambled generated cytobiologics.

### Example 69: Modification of Cytobiologic Source Cells for Decreased CD8 Killer T Cell Lysis

This Example describes the generation of a cytobiologic composition derived from a cell source, which has been modified to decrease cytotoxicity mediated cell lysis by CD8+ T-cells. In an embodiment, cytotoxicity mediated cell lysis of source cells or cytobiologics by CD8+ T-cells is a measure of immunogenicity for cytobiologics.

In this Example, cytobiologics are generated by any one of the methods described in a previous Example. Cytobiologics are created from: unmodified mesenchymal stem cells (hereafter MSCs, positive control), mesenchymal stem cells modified with a lentiviral-mediated expression of HLA-G (hereafter MSC-HLA-G), and mesenchymal stem cells modified with a lentiviral-mediated expression of an empty vector (hereafter MSC-empty vector, negative control).

CD8+ T cell mediated lysis of a cytobiologic is determined by europium release assays as described in Bouma, et al. Hum. Immunol. 35(2):85-92; 1992 & van Besouw et al. Transplantation 70(1):136-143; 2000. CD8+ T-cells (hereafter effector cells) are isolated from an appropriate donor according to the methods in Crop et al. Cell transplantation (20):1547-1559; 2011, and stimulated with allogeneic gamma irradiated PMBCs and 2001 U/mL IL-2 (proleukin, Chiron BV Amsterdam, The Netherlands.) in a round bottom 96 well plate for 7 days at 37 C. The cytobiologics are labeled with europium-diethylenetriaminepentaacetate (DTPA) (sigma, St. Louis, Mo., USA).

At day 7 cytotoxicity-mediated lysis assays is performed by incubating ⁶³Eu-labelled cytobiologics with effector cells in a 96-well plate for 1, 2, 3, 4, 5, 6, 8, 10, 15, 20, 24, 48 hours after plating at effector/target ratios ranging from 1000:1-1:1 and 1:1.25-1:1000. After incubation, the plates are centrifuged and 20 ul of the supernatant is transferred to 96-well plates with low background fluorescence (fluoroimmunoplates, Nunc, Roskilde, Denmark).

Subsequently, enhancement solution (PerkinElmer, Groningen, The Netherlands) is added to each well. The released europium is measured in a time-resolved fluorometer (Victor 1420 multilabel counter, LKB-Wallac, Finland). Fluorescence is expressed in counts per second (CPS). Maximum percent release of europium by a target cytobiologic is determined by incubating an appropriate number (1×10²-1×10⁸) of cytobiologics with 1% triton (sigma-aldrich) for an appropriate amount of time. Spontaneous release of europium by target cytobiologics is measured by incubation of labeled target cytobiologics without effector cells. Percentage leakage is then calculated as: (spontaneous release/maximum release)×100%. Finally, the percentage of cytotoxicity mediated lysis is calculated as % lysis=[(measured lysis−spontaneous lysis−spontaneous release)/(maximum release-spontaneous release)]×100%. The data is analyzed by looking at the percentage of lysis as a function of different effector target ratios.

In an embodiment, cytobiologics generated from MSC-HLA-G cells will have a decreased percentage of lysis at appropriate timepoints as compared to MSCs or MSC-scrambled generated cytobiologics.

### Example 70: Modification of Cytobiologic Source Cells for Decreased T-Cell Activation

This Example describes the generation of modified cytobiologics that will have reduced T cell activation and proliferation as assessed by a mixed lymphocyte reaction (MLR).

T-cell proliferation and activation are measures of immunogenicity for cytobiologics. Stimulation of T cell proliferation in an MLR reaction by a cytobiologic composition, could suggest a stimulation of T cell proliferation in vivo.

In an embodiment, cytobiologics generated from modified source cells have reduced T cell activation and proliferation as assessed by a mixed lymphocyte reaction (MLR). In an embodiment, cytobiologics generated from modified source cells do not generate an immune response in vivo, thus maintaining the efficacy of the cytobiologic composition.

In this Example, cytobiologics are generated by any one of the methods described in a previous Example. Cytobiologics are generated from: unmodified mesenchymal stem cells (hereafter MSCs, positive control), mesenchymal stem cells modified with a lentiviral-mediated expression of IL-10 (hereafter MSC-IL-10), and mesenchymal stem cells modified with a lentiviral-mediated expression of an empty vector (hereafter MSC-empty vector, negative control).

BALB/c and C57BL/6 splenocytes are used as either stimulator or responder cells. Notably, the source of these cells can be exchanged with commonly used human-derived stimulator/responder cells. Additionally, any mammalian purified allogeneic CD4+ T cell population, CD8+ T-cell population, or CD4−/CD8− may be used as responder population.

Mouse Splenocytes are isolated by mechanical dissociation using fully frosted slides followed by red blood cell lysis with lysing buffer (Sigma-Aldrich, St-Louis, Mo.). Prior to the experiment, stimulator cells are irradiated with 20 Gy of gray to prevent them from reacting against responder cells. A co-culture is then made by adding equal numbers of stimulator and responder cells (or alternative concentrations while maintaining a 1:1 ratio) to a round bottom 96-well plate in complete DMEM-10 media. An appropriate number of cytobiologics (at several concentrations from a range of 1×10¹-1×10⁸) are added to the co-culture at different time intervals, t=0, 6, 12, 24, 36, 48 h.

Proliferation is assessed by adding 1 μCi of [³H]-thymidine (Amersham, Buckinghamshire, UK) to allow for incorporation. [³H]-thymidine is added to the MLR at t=2, 6, 12, 24, 36, 48, 72 h, and the cells are harvested onto glass fiber filters using a 96 well cell harvester (Inoteck, Bertold, Japan) after 2, 6, 12, 18, 24, 36 and 48 h of extended culture. All of the T-cell proliferation experiments are done in triplicate. [³H]-thymidine incorporation is measured using a microbeta lLuminescence counter (Perkin Elmer, Wellesley, Mass.). The results can be represented as counts per minute (cpm).

In an embodiment, MSC-IL10 cytobiologics will show a decrease in T-cell proliferation as compared to the MSC-Empty vector or the MSC unmodified cytobiologic controls.

### Example 71: Measuring Targeting Potential of Cytobiologics in a Subject

This Example assesses the ability of a cytobiologic to target a specific body site. In an embodiment, a cytobiologic can target a specific body site. Targeting is a way to restrict activity of a therapeutic to one or more relevant therapeutic sites.

Eight week old C57BL/6J mice (Jackson Laboratories) are intravenously injected with cytobiologics or cells that express firefly luciferase. Cytobiologics are produced from cells that stably express firefly luciferase or cells that do not express luciferase (negative control) by any one of the methods described in previous Examples. Groups of mice are euthanized at one, two, three, four, five, six, eight, twelve, and twenty-four hours after cytobiologic or cell injection.

Five minutes before euthanization, mice receive an IP injection of bioluminescent substrate (Perkin Elmer) at a dose of 150 mg/kg in order to visualize luciferase. The bioluminescent imaging system is calibrated to compensate for all device settings. Mice are then euthanized and liver, lungs, heart, spleen, pancreas, GI, and kidney are collected. The imaging system (Perkin Elmer) is used to obtain images of bioluminescence of these ex vivo organs. The bioluminescent signal is measured using Radiance Photons, with Total Flux used as a measured value. The region of interest (ROI) is generated by surrounding the ex vivo organ in order to give a value in photons/second. The ratio of photons/second between target organs (e.g. liver) and non-target organs (e.g. the sum of photons/second from lungs, heart, spleen, pancreas, GI, and kidney) is calculated as a measure of targeting to the liver.

In an embodiment, in both cytobiologics and cells, the ratio of photons/second between liver and the other organs will be greater than 1, which would indicate that cytobiologics target the liver. In an embodiment, negative control animals will display much lower photons/second in all organs.

### Example 72: Measuring Delivery of an Exogenous Agent by Cytobiologics in a Subject

This Example describes quantification of delivery of cytobiologics comprising an exogenous agent in a subject. Cytobiologics are prepared from cells expressing Gaussia-luciferase or from cells not expressing luciferase (negative control) by any one of the methods described in previous Examples.

Positive control cells or cytobiologics are intravenously injected into mice. Cytobiologics or cells are delivered within 5-8 seconds using a 26-gauge insulin syringe-needle. In vivo bioluminescent imaging is performed on mice 1, 2, or 3 days after injection using an in vivo imaging system (Xenogen Corporation, Alameda, Calif.).

Immediately before use, coelenterazine, a luciferin or light-emitting molecule, (5 mg/ml) is prepared in acidified methanol and injected immediately into the tail vein of the mice. Mice are under continuous anesthesia on a heated stage using the XGI-8 Gas Anesthesia System.

Bioluminescence imaging is obtained by acquiring photon counts over 5 min immediately after intravenous tail-vein injection of coelenterazine (4 μg/g body weight). Acquired data are analyzed using software (Xenogen) with the overlay on light-view image. Regions of interest (ROI) are created using an automatic signal intensity contour tool and normalized with background subtraction of the same animal A sequential data acquisition using three filters at the wavelengths of 580, 600 and 620 nm with exposure time 3-10 min is conducted to localize bioluminescent light sources inside a mouse.

Furthermore, at each timepoint, urine samples are collected by abdominal palpation.

Blood samples (50 μl) are obtained from the tail vein of each mouse into heparinized or EDTA tubes. For plasma isolation, blood samples are centrifuged for 25 min at 1.3×g at 4° C.

Then, 5 μl of blood, plasma or urine sample are used to carry out a Gaussia-luciferase activity assay after mixing the samples with 50 μM Gaussia-luciferase substrate (Nanolight, Pinetop, Ariz.).

In an embodiment, the negative control samples will be negative for luciferase, and positive control samples will be from animals administered cells. In an embodiment, the samples from animals administered cytobiologics expressing Gaussia-luciferase will be positive for luciferase in each sample.

See, for example, El-Amouri S S et al., Molecular biotechnology 53(1): 63-73, 2013.

### Example 73: Active Transport Across a Lipid Bilayer of a Cytobiologic

This Example describes quantification of the level of 2-NBDG (2-(N-(7-Nitrobenz-2-oxa-1,3-diazol-4-yl)Amino)-2-Deoxyglucose), a fluorescent glucose analog that can be used to monitor glucose uptake in live cells and thus active transport across the lipid bilayer. In an embodiment, this assay can be used to measure the level of glucose uptake and active transport across the lipid bilayer of the cytobiologic.

A cytobiologic composition as produced by any one of the methods described in previous Examples. A sufficient number of cytobiologics are then incubated in DMEM containing no glucose, 20% Fetal Bovine Serum and 1× Penicillin/Streptomycin for 2 hr at 37° C. and 5% CO₂. After the 2 hr glucose starvation period, the medium is changed such that it includes DMEM with no glucose, 20% Fetal Bovine Serum, 1× Penicillin/Streptomycin, and 20 uM 2-NBDG (ThermoFisher) and incubated for 2 hr at 37° C. and 5% CO₂. Negative control cytobiologics are treated the same, except an equal amount of DMSO, the vehicle for 2-NBDG is added in place of 2-NBDG.

The cytobiologics are then washed thrice with 1×PBS and re-suspended in an appropriate buffer, and transferred to a 96 well imaging plate. 2-NBDG fluorescence is then measured in a fluorimeter using a GFP light cube (469/35 excitation filter and a 525/39 emission filter) to quantify the amount of 2-NBDG that has transported across the cytobiologic membrane and accumulated in the cytobiologic in the 1 hr loading period.

In an embodiment, 2-NBDG fluorescence will be higher in the cytobiologics with 2-NBDG treatment as compared to the negative (DMSO) control. Fluorescence measure with a 525/39 emission filter will be relatively to the number of 2-NBDG molecules present.

### Example 74: Assessment of Teratoma Formation after Administration of Cytobiologics

This Example describes the absence of teratoma formation with a cytobiologic. In an embodiment, a cytobiologic will not result in teratoma formation when administered to a subject.

The cytobiologics are produced by any one of the methods described in a previous Example. Cytobiologics, tumor cells (positive control) or vehicle (negative control) are subcutaneously injected in PBS into the left flank of mice (12-20 weeks old). Teratoma, e.g., tumor, growth is analyzed 2-3 times a week by determination of tumor volume by caliper measurements for eight weeks after cytobiologic, tumor cell, or vehicle injection.

In an embodiment, mice administered cytobiologics or vehicle will not have a measurable tumor formation, e.g., teratoma, via caliper measurements. In an embodiment, positive control animals treated with tumor cells will demonstrate an appreciable tumor, e.g., teratoma, size as measured by calipers over the eight weeks of observation.

### Example 75: Cytobiologics Deliver Active Protein to Recipient Cells of a Subject In Vivo

This Example demonstrates that cytobiologics can deliver a protein to a subject in vivo. This is exemplified by delivery of the nuclear editing protein Cre. Once inside of a cell, Cre translocates to the nucleus, where it recombines and excises DNA between two LoxP sites. Cre-mediated recombination can be measured microscopically when the DNA between the two LoxP sites is a stop codon and is upstream of a distal fluorescent protein, such as the red fluorescent protein tdTomato.

Cytobiologics that contain CRE and the fusogen VSV-G, purchased from Takara (Cre Recombinase Gesicles, Takara product 631449), were injected into B6.Cg-Gt(ROSA)26Sor^{tm9(CAG-tdTomato)Hze}/J mice (Jackson Laboratories strain 007909). Animals were injected at the anatomical sites, injection volumes, and injection sites as described in Table 14. Mice that do not have tdTomato (FVB.129S6(B6)-GT(ROSA)26Sor^{tm1(Luc)Kael}/J Jackson Laboratories strain 005125) and were injected with cytobiologics and B6.Cg-Gt(ROSA)26Sor^{tm14(CAG-tdTomato)Hze}/J mice that were not injected with cytobiologics were used as negative controls.

TABLE 14
Injection parameters

| | | | |
|---|---|---|---|
| Brain | 10 | ul | anterior posterior axis: −2 |
| | | | Lateral/medial axis: 1.8 |
| | | | ventral: 1.5 side: right |
| Eye | 1 | ul | intravitreal |
| Liver | 25 | ul | center of frontal lobe |
| Spleen | 10 | ul | approximately in the center, |
| | | | both lengthwise and widthwise |
| Kidney | 20 | ul | center of left kidney |
| Small intestine | 10 | ul | loop of small intestine nearest |
| lining | | | the peritoneal wall was isolated |
| | | | outside peritoneum, and |
| | | | injected into lining. |
| Heart | 5 | ul | near apex |
| White Adipose | 25 | ul | left, top and central |
| (Epididymal fat | | | |
| pad) | | | |
| Brown adipose | 25 | ul | left lobe, as central as possible |
| (intrascapular) | | | |
| Lung | 10 | ul | inferior lobe right lung |
| Testis | 10 | ul | left testis, as central as possible |
| Ovary | 1 | ul | left ovary, as central as possible |

Two days after injections, the animals were sacrificed and samples were collected. The samples were fixed for 8 hours in 2% PFA, fixed overnight in 30% sucrose, and shipped for immediate embedding in OCT and sectioning to slides. Slides were stained for nuclei with DAPI. DAPI and tdTomato fluorescence was imaged microscopically.

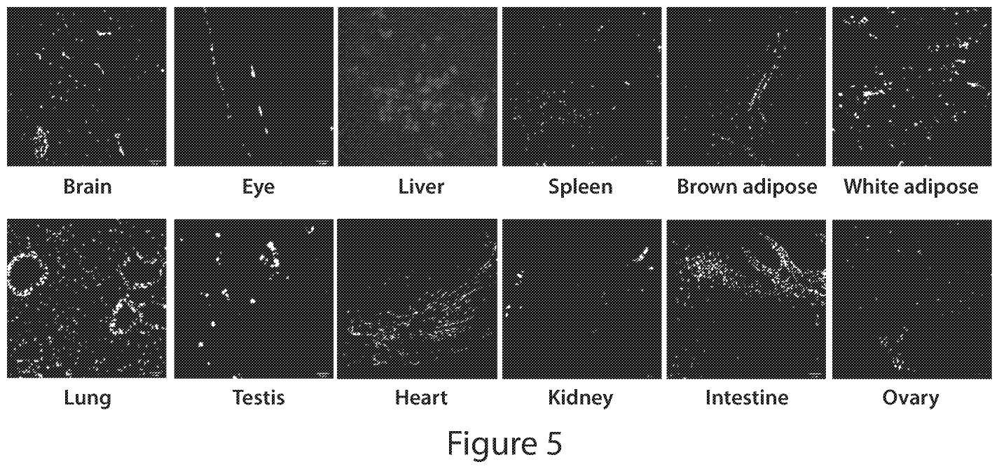

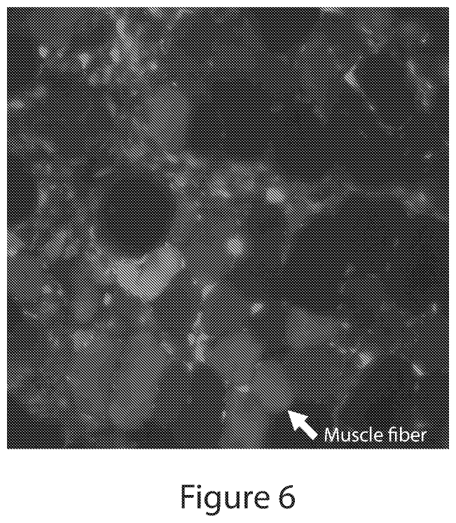

All anatomical sites listed in Table 14 demonstrated tdTomato fluorescence (FIG. 5). In addition, delivery to muscle tissue was confirmed using fluorescence microscopy for tdTomato (FIG. 6). Negative control mice did not have any tissues with tdTomato fluorescence. This result demonstrates that cytobiologics are capable of turning on tdTomato fluorescence in the cells of a mouse at various anatomical sites, and that this does not occur if the mice are not treated with cytobiologics or if the mice do not have tdTomato in their genome. Hence, fusogenic cytobiologics deliver active Cre recombinase to the nucleus of mouse cells in vivo.

It was also shown that different routes of administration can deliver deliver cytobiologics to tissue in vivo. Cytobiologics that contain CRE and the fusogen VSV-G, purchased from Takara (Cre Recombinase Gesicles, Takara product 631449), were injected into FVB.129S6(B6)-GT(ROSA)26Sor^{tm1(Luc)Kael}/J (Jackson Laboratories strain 005125) intramuscularly (in 50 ul to the right tibialis anterior muscle), intraperitoneally (in 50 ul to the peritoneal cavity), and subcutaneously (in 50 ul under the dorsal skin).

The legs, ventral side, and dorsal skin was prepared for intramuscular, intraperitoneal, and subcutaneous injection, respectively, by depilating the area using a chemical hair remover for 45 seconds, followed by 3 rinses with water.

On day 3 after injection, an in vivo imaging system (Perkin Elmer) was used to obtain whole animal images of bioluminescence. Five minutes before imaging, mice received an intraperitoneal injection of bioluminescent substrate (Perkin Elmer) at a dose of 150 mg/kg in order to visualize luciferase. The imaging system was calibrated to compensate for all device settings.

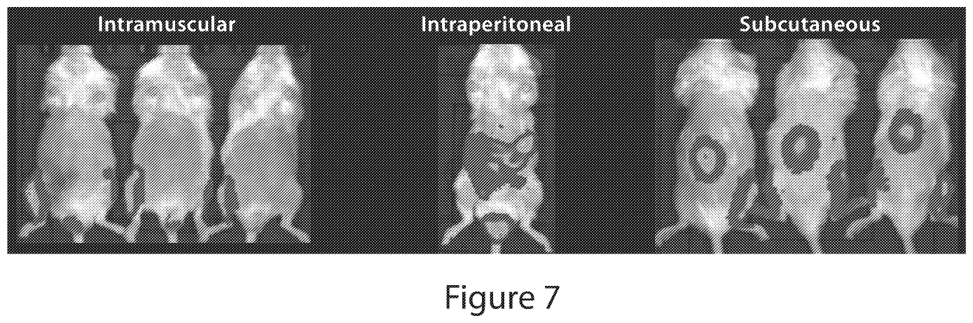

Administration by all three routes resulted in luminescence (FIG. 7) indicating successful delivery of active Cre recombinase to mouse cells in vivo.

In conclusion, cytobiologics are capable of delivering active protein to cells of a subject in vivo.

### Example 76: Sonication-Mediated Loading of Nucleic Acid in Cytobiologics

This Example describes loading of nucleic acid payloads into a cytobiologic via sonication. Sonication methods are disclosed e.g., in Lamichhane, T N, et al., Oncogene Knockdown via Active Loading of Small RNAs into Extracellular Vesicles by Sonication. Cell Mol Bioeng, (2016), the entire contents of which are hereby incorporated by reference.

Cytobiologics are prepared by any one of the methods described in a previous Example. Approximately 10⁶ cytobiologics are mixed with 5-20 μg nucleic acid and incubated at room temperature for 30 minutes. The cytobiologic/nucleic acid mixture is then sonicated for 30 seconds at room temperature using a water bath sonicator (Brason model #1510R-DTH) operated at 40 kHz. The mixture is then placed on ice for one minute followed by a second round of sonication at 40 kHz for 30 seconds. The mixture is then centrifuged at 16,000 g for 5 minutes at 4° C. to pellet the cytobiologics containing nucleic acid. The supernatant containing unincorporated nucleic acid is removed and the pellet is resuspended in phosphate-buffered saline. After DNA loading, the cytobiologics are kept on ice before use.

### Example 77: Sonication-Mediated Loading of Protein in Cytobiologics

This Example describes loading of protein payloads into a cytobiologic via sonication. Sonication methods are disclosed e.g., in Lamichhane, T N, et al., Oncogene Knockdown via Active Loading of Small RNAs into Extracellular Vesicles by Sonication. Cell Mol Bioeng, (2016), the entire contents of which are hereby incorporated by reference.

Cytobiologics are prepared by any one of the methods described in a previous Example. Approximately 10⁶ cytobiologics are mixed with 5-20 μg protein and incubated at room temperature for 30 minutes. The cytobiologic/protein mixture is then sonicated for 30 seconds at room temperature using a water bath sonicator (Brason model #1510R-DTH) operated at 40 kHz. The mixture is then placed on ice for one minute followed by a second round of sonication at 40 kHz for 30 seconds. The mixture is then centrifuged at 16,000 g for 5 minutes at 4 C to pellet the cytobiologics containing protein. The supernatant containing unincorporated protein is removed and the pellet is resuspended in phosphate-buffered saline. After protein loading, the cytobiologics are kept on ice before use.

### Example 78: Hydrophobic-Carrier Mediated Loading of Nucleic Acid in Cytobiologics

This Example describes loading of nucleic acid payloads into a cytobiologic via hydrophobic carriers. Exemplary methods of hydrophobic loading are disclosed, e.g., in Didiot et al., Exosome-mediated Delivery of Hydrophobically Modified siRNA for Huntingtin mRNA Silencing, Molecular Therapy 24(10): 1836-1847, (2016), the entire contents of which are hereby incorporated by reference.

Cytobiologics are prepared by any one of the methods described in an Example herein. The 3′ end of a RNA molecule is conjugated to a bioactive hydrophobic conjugate (triethylene glycol-Cholesterol). Approximately 10⁶ cytobiologics are mixed in 1 ml with 10 μmol/l of siRNA conjugate in PBS by incubation at 37° C. for 90 minutes with shaking at 500 rpm. The hydrophobic carrier mediates association of the RNA with the membrane of the cytobiologic. In some embodiments, some RNA molecules are incorporated into the lumen of the cytobiologic, and some are present on the surface of the cytobiologic. Unloaded cytobiologics are separated from RNA-loaded cytobiologics by ultracentrifugation for 1 hour at 100,000 g, 4° C. in a tabletop ultracentrifuge using a TLA-110 rotor. Unloaded cytobiologics remain in the supernatant and RNA-loaded cytobiologics form a pellet. The RNA-loaded cytobiologics are resuspended in 1 ml PBS and kept on ice before use.

### Example 79: Processing Cytobiologics

This Example described the processing of cytobiologics. Cytobiologics produced via any of the described methods in the previous Examples may be further processed.

In some embodiments, cytobiologics are first homogenized, e.g., by sonication. For example, the sonication protocol includes a 5 second sonication using an MSE sonicator with microprobe at an amplitude setting of 8 (Instrumentation Associates, N.Y.). In some embodiments, this short period of sonication is enough to cause the plasma membrane of the cytobiologics to break up into homogenously sized cytobiologics. Under these conditions, organelle membranes are not disrupted and these are removed by centrifugation (3,000 rpm, 15 min 4° C.). Cytobiologics are then purified by differential centrifugation as described in Example 6.

Extrusion of cytobiologics through a commercially available polycarbonate membrane (e.g., from Sterlitech, Washington) or an asymmetric ceramic membrane (e.g., Membralox), commercially available from Pall Execia, France, is an effective method for reducing cytobiologic sizes to a relatively well defined size distribution. Typically, the suspension is cycled through the membrane one or more times until the desired cytobiologic size distribution is achieved. The cytobiologics may be extruded through successively smaller pore membranes (e.g., 400 nm, 100 nm and/or 50 nm pore size) to achieve a gradual reduction in size and uniform distribution.

In some embodiments, at any step of cytobiologic production, though typically prior to the homogenization, sonication and/or extrusion steps, a pharmaceutical agent (such as a therapeutic), may be added to the reaction mixture such that the resultant cytobiologic encapsulates the pharmaceutical agent.

### Example 80: Measuring Total RNA in a Cytobiologic and Source Cell

This Example describes a method to quantify the amount of RNA in a cytobiologic relative to a nucleated counterpart (e.g., a source cell). In an embodiment, a cytobiologic will have similar RNA levels to nucleated counterparts. In this assay, RNA levels are determined by measuring total RNA.

Cytobiologics are prepared by any one of the methods described in previous Examples. Preparations of the same mass as measured by protein of cytobiologic and source cells are used to isolate total RNA (e.g., using a kit such as Qiagen RNeasy catalog #74104), followed by determination of RNA concentration using standard spectroscopic methods to assess light absorbance by RNA (e.g. with Thermo Scientific NanoDrop).

In an embodiment, the concentration of RNA in cytobiologics will be 5%, 10%, 20%, 30%, 40%, 50%, 60%, 70%, 80%, 90%, 95% of that of source cells per mass of protein.

What is claimed is:

1. A purified cytobiologic composition comprising a cytobiologic from a source cell, e.g., a mammalian source cell, e.g., a human source cell, wherein the cytobiologic has partial or complete nuclear inactivation (e.g., nuclear removal), wherein the cytobiologic is not from an erythroid cell or a platelet, and
wherein one or more of:
i) the cytobiologic comprises an exogenous agent or a therapeutic agent (e.g., an exogenous therapeutic agent), e.g., at a copy number of at least 1,000 copies, e.g., as measured by an assay of Example 31;
ii) the cytobiologic comprises a lipid wherein one or more of CL, Cer, DAG, HexCer, LPA, LPC, LPE, LPG, LPI, LPS, PA, PC, PE, PG, PI, PS, CE, SM and TAG is within 75% of the corresponding lipid level in the source cell;
iii) the cytobiologic comprises a proteomic composition similar to that of the source cell, e.g., using an assay of Example 30;
iv) the cytobiologic is capable of signal transduction, e.g., transmitting an extracellular signal, e.g., AKT phosphorylation in response to insulin, or glucose (e.g., labeled glucose, e.g., 2-NBDG) uptake in response to insulin, e.g., by at least 10% more than a negative control, e.g., an otherwise similar cytobiologic in the absence of insulin, e.g., using an assay of Example 48;
v) the cytobiologic targets a tissue, e.g., liver, lungs, heart, spleen, pancreas, gastrointestinal tract, kidney, testes, ovaries, brain, reproductive organs, central nervous system, peripheral nervous system, skeletal muscle, endothelium, inner ear, or eye, when administered to a subject, e.g., a mouse, e.g., wherein at least 0.1%, or 10%, of the cytobiologics in a population of administered cytobiologics are present in the target tissue after 24 hours, e.g., by an assay of Example 71; or
vi) the source cell is selected from a neutrophil, a granulocyte, a mesenchymal stem cell, a bone marrow stem cell, an induced pluripotent stem cell, an embryonic stem cell, a myeloblast, a myoblast, a hepatocyte, or a neuron e.g., retinal neuronal cell.

2. A purified cytobiologic composition comprising a cytobiologic and an exogenous agent, e.g., a therapeutic agent, wherein:
i) the cytobiologic is from a source cell, e.g., a mammalian source cell,
ii) the cytobiologic is an enucleated cell or a cell having partial or complete nuclear inactivation (e.g., nuclear removal), and
iii) the cytobiologic is not from an erythroid cell or a platelet.

3. A purified cytobiologic composition, e.g., a frozen cytobiologic composition, comprising a cytobiologic, wherein:
i) the cytobiologic is from a source cell, e.g., a mammalian source cell,
ii) the cytobiologic is an enucleated cell or a cell having partial or complete nuclear inactivation (e.g., nuclear removal), and
iii) the cytobiologic is not from an erythroid cell or a platelet.
which is at a temperature of less than 4, 0, −4, −10, −12, −16, −20, −80, or −160 C.

4. The cytobiologic composition of any of the preceding claims, wherein one or more of:
i) the cytobiologic comprises a ratio of lipids to proteins that is within 10%, 20%, 30%, 40%, or 50% of the corresponding ratio in the source cell, e.g., as measured using an assay of Example 37;
ii) the cytobiologic comprises a ratio of proteins to nucleic acids (e.g., DNA) that is within 10%, 20%, 30%, 40%, or 50% of the corresponding ratio in the source cell, e.g., as measured using an assay of Example 38;
iii) the cytobiologic comprises a ratio of lipids to nucleic acids (e.g., DNA) that is within 10%, 20%, 30%, 40%, or 50% of the corresponding ratio in the source cell, e.g., as measured using an assay of Example 39;
iv) the cytobiologic has a half-life in a subject, e.g., in a mouse, that is within 90% of the half life of a reference cell, e.g., the source cell, e.g., by an assay of Example 60;
v) the cytobiologic transports glucose (e.g., labeled glucose, e.g., 2-NBDG) across a membrane, e.g., by at least 10% more than a negative control, e.g., an otherwise similar cytobiologic in the absence of glucose, e.g., as measured using an assay of Example 49;
vi) the cytobiologic comprises esterase activity in the lumen that is within 90% of that of the esterase activity in a reference cell, e.g., the source cell or a mouse embryonic fibroblast, e.g., using an assay of Example 51;
vii) the cytobiologic comprises a metabolic activity level that is within 90% of the metabolic activity (e.g., citrate synthase activity) in a reference cell, e.g., the source cell, e.g., as described in Example 53;
viii) the cytobiologic comprises a respiration level (e.g., oxygen consumption rate) that is within 90% of the respiration level in a reference cell, e.g., the source cell, e.g., as described in Example 54;
ix) the cytobiologic comprises an Annexin-V staining level of at most 18,000, 17,000, 16,000, 15,000, 14,000, 13,000, 12,000, 11,000, or 10,000 MFI, e.g., using an assay of Example 55, or wherein the cytobiologic comprises an Annexin-V staining level at least 5%, 10%, 20%, 30%, 40%, or 50% lower than the Annexin-V staining level of an otherwise similar cytobiologic treated with menadione in the assay of Example 55, or wherein the cytobiologic comprises an Annexin-V staining level at least 5%, 10%, 20%, 30%, 40%, or 50% lower than the Annexin-V staining level of a macrophage treated with menadione in the assay of Example 55;
x) the cytobiologic has a miRNA content level of at least 1% than that of the source cell, e.g., by an assay of Example 27;
xi) the cytobiologic has a soluble:non-soluble protein ratio is within 90% of that of the source cell, e.g., by an assay of Example 35;
xii) the cytobiologic has an LPS level less than 5% of the lipid content of the cytobiologic, e.g., as measured by an assay of Example 36;
xiii) the cytobiologic has juxtacrine-signaling level of at least 5% greater than the level of juxtacrine signaling induced by a reference cell, e.g., the source cell or a bone marrow stromal cell (BMSC), e.g., by an assay of Example 56;
xiv) the cytobiologic has paracrine-signaling level of at least 5% greater than the level of paracrine signaling induced by a reference cell, e.g., the source cell or a macrophage, e.g., by an assay of Example 57;
xv) the cytobiologic polymerizes actin at a level within 5% compared to the level of polymerized actin in a reference cell, e.g., the source cell or a C2C12 cell, e.g., by the assay of Example 58;
xvi) the cytobiologic has a membrane potential within about 5% of the membrane potential of a reference cell, e.g., the source cell or a C2C12 cell, e.g., by an assay of Example 59, or wherein the cytobiologic has a membrane potential of about −20 to −150 mV, −20 to −50 mV, −50 to −100 mV, or −100 to −150 mV;
xvii) the cytobiologic is capable of extravasation from blood vessels, e.g., at a rate at least 1%, 2%, 5%, 10%, 20%, 30%, 40%, 50%, 60%, 70%, 80%, or 90% the rate of extravasation of a cell of the same type as the source cell, e.g., using an assay of Example 42, e.g., wherein the source cell is a neutrophil, lymphocyte, B cell, macrophage, or NK cell;
xviii) the cytobiologic is capable of crossing a cell membrane, e.g., an endothelial cell membrane or the blood brain barrier, e.g., at a rate at least 1%, 2%, 5%, 10%, 20%, 30%, 40%, 50%, 60%, 70%, 80%, or 90% that of a cell of the same type as the source cell;
xix) the cytobiologic is capable of secreting a protein, e.g., at a rate at least 5% greater than a reference cell, e.g., a mouse embryonic fibroblast, e.g., using an assay of Example 47;
xx) the cytobiologic meets a pharmaceutical or good manufacturing practices (GMP) standard;
xxi) the cytobiologic was made according to good manufacturing practices (GMP);
xxii) the cytobiologic has a pathogen level below a predetermined reference value, e.g., is substantially free of pathogens;
xxiii) the cytobiologic has a contaminant level below a predetermined reference value, e.g., is substantially free of contaminants;
xxiv) the cytobiologic has low immunogenicity, e.g., as described herein;
xxv) the source cell is other than a 293 cell, HEK cell, human endothelial cell, or a human epithelial cell, monocyte, macrophage, dendritic cell, or stem cell;
xxvi) the cytobiologic, composition, or preparation has a density of other than between 1.08 g/ml and 1.12 g/ml;
xxvii) the cytobiologic, composition, or preparation has a density of 1.25 g/ml+/−0.05, e.g., as measured by an assay of Example 21;
xxviii) the cytobiologic is not captured by the scavenger system in circulation or by Kupffer cells in the sinus of the liver; or
xxix) the cytobiologic has a diameter of greater than 5 um, 6 um, 7 um, 8 um, 10 um, 20 um, 50 um, 100 um, 150 um, or 200 um.

5. The cytobiologic composition of any of the preceding claims, which comprises a cargo, e.g., a therapeutic agent, e.g., an endogenous therapeutic agent or an exogenous therapeutic agent.

6. The cytobiologic composition of claim 5, wherein the therapeutic agent is chosen from one or more of a protein, e.g., an enzyme, a transmembrane protein, a receptor, an antibody; a nucleic acid, e.g., DNA, a chromosome (e.g. a human artificial chromosome), RNA, mRNA, siRNA, miRNA, or a small molecule.

7. The cytobiologic composition of claim 5, wherein the therapeutic agent is an organelle e.g., an organelle selected from: a mitochondrion, a Golgi apparatus, lysosome, endoplasmic reticulum, vacuole, endosome, acrosome, autophagosome, centriole, glycosome, glyoxysome, hydrogenosome, melanosome, mitosome, cnidocyst, peroxisome, proteasome, vesicle, and stress granule.

8. The cytobiologic composition of any of the preceding claims, which has a density of <1, 1-1.1, 1.05-1.15, 1.1-1.2, 1.15-1.25, 1.2-1.3, 1.25-1.35, or >1.35 g/ml, e.g., by an assay of Example 21.

9. The cytobiologic composition of any of the preceding claims, which is an enucleated cell.

10. The cytobiologic composition of any of the preceding claims, which comprises an exogenous therapeutic agent chosen from one or more of a protein, e.g., an enzyme, a transmembrane protein, a receptor, an antibody; a nucleic acid, e.g., DNA, a chromosome (e.g. a human artificial chromosome), RNA, mRNA, siRNA, miRNA, or a small molecule.

11. The cytobiologic composition of any of the preceding claims, which comprises less than 0.01%, 0.05%, 0.1%, 0.5%, 1%, 1.5%, 2%, 2.5%, 3%, 4%, 5%, or 10% source cells by protein mass or less than 0.01%, 0.05%, 0.1%, 0.5%, 1%, 1.5%, 2%, 2.5%, 3%, 4%, 5%, or 10% of cells have a functional nucleus.

12. The cytobiologic composition of claim 3, which has been maintained at said temperature for at least 1, 2, 3, 6, or 12 hours; 1, 2, 3, 4, 5, or 6 days; 1, 2, 3, or 4 weeks; 1, 2, 3, or 6 months; or 1, 2, 3, 4, or 5 years.

13. The cytobiologic composition of claim 3, which has an activity of at least 50%, 60%, 70%, 80%, 90%, 95%, or 99% of the activity of the population before maintenance at said temperature, e.g., using an assay described herein.

14. The cytobiologic composition of claim 3, which is stable at a temperature of less than 4 C for at least 1, 2, 3, 6, or 12 hours; 1, 2, 3, 4, 5, or 6 days; 1, 2, 3, or 4 weeks; 1, 2, 3, or 6 months; or 1, 2, 3, 4, or 5 years.

15. The cytobiologic composition of claim 3, which is stable at a temperature of less than −20 C for at least 1, 2, 3, 6, or 12 hours; 1, 2, 3, 4, 5, or 6 days; 1, 2, 3, or 4 weeks; 1, 2, 3, or 6 months; or 1, 2, 3, 4, or 5 years.

16. The cytobiologic composition of claim 3, which is stable at a temperature of less than −80 C for at least 1, 2, 3, 6, or 12 hours; 1, 2, 3, 4, 5, or 6 days; 1, 2, 3, or 4 weeks; 1, 2, 3, or 6 months; or 1, 2, 3, 4, or 5 years.

17. The cytobiologic composition of any of the preceding claims, wherein one or more of:
i) the source cell is other than a 293 cell;
ii) the source cell is not transformed or immortalized;
iii) the source cell is transformed or immortalized using a method other than adenovirus-mediated immortalization, e.g., immortalized by spontaneous mutation or telomerase expression;
iv) the therapeutic agent is other than Cre or EGFP;
v) the therapeutic agent is a nucleic acid (e.g., RNA, e.g., mRNA, miRNA, or siRNA) or an exogenous protein (e.g., an antibody, e.g., an antibody), e.g., in the lumen; or
vi) the cytobiologic does not comprise mitochondria.

18. The cytobiologic composition of any of the preceding claims, wherein one or more of:
i) the source cell is other than a 293 or HEK cell;
ii) the source cell is not transformed or immortalized;
iii) the source cell is transformed or immortalized using a method other than adenovirus-mediated immortalization, e.g., immortalized by spontaneous mutation or telomerase expression; or
iv) the cytobiologic has a size of other than between 40 and 150 nm, e.g., greater than 150 nm, 200 nm, 300 n, 400 nm, or 500 nm.

19. The cytobiologic composition of any of the preceding claims, wherein one or more of:
i) the therapeutic agent is a soluble protein expressed by the source cell;
ii) the cytobiologic comprises in its lumen a polypeptide selected from an enzyme, antibody, or anti-viral polypeptide;
iii) the cytobiologic does not comprise an exogenous therapeutic transmembrane protein; or
iv) the cytobiologic does not comprise CD63 or GLUT4.

20. The cytobiologic composition of any of the preceding claims, wherein the cytobiologic:
i) does not comprise a virus, is not infectious, or does not propagate in a host cell;
ii) is not a VLP (virus like particle);
iii) does not comprise a viral structural protein, e.g., a viral capsid protein, e.g., a viral nucleocapsid protein, or wherein the amount of viral capsid protein is less than 10%, 5%, 4%, 3%, 2%, 1%, 0.5%, 0.2%, or 0.1% of total protein, e.g., by an assay of Example 41;
iv) does not comprise a viral matrix protein;
v) does not comprise a viral non-structural protein;
vi) comprises less than 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, 1,000,000,000 copies of a viral structural protein; or
vii) is not a virosome.

21. The cytobiologic composition of any of the preceding claims, wherein one or both of:
i) the ratio of the copy number of the exogenous agent to the copy number of viral structural protein on the cytobiologic is at least 1000000:1, 100000:1, 10000:1, 1000:1, 100:1 and 50:1, 50:1 and 20:1, 20:1 and 10:1, 10:1 and 5:1, or 1:1;
ii) the ratio of the copy number of the exogenous agent to the copy number of viral matrix protein on the cytobiologic is at least 1000000:1, 100000:1, 10000:1, 1000:1, 100:1 and 50:1, 50:1 and 20:1, 20:1 and 10:1, 10:1 and 5:1, or 1:1.

22. The cytobiologic composition of any of the preceding claims, which is unilamellar or multilamellar.

23. The cytobiologic composition of any of the preceding claims, wherein:
i) the cytobiologic does not comprise a water-immiscible droplet;
ii) the cytobiologic comprises an aqueous lumen and a hydrophilic exterior; or
iii) the organelle is selected from a mitochondrion, Golgi apparatus, lysosome, endoplasmic reticulum, vacuole, endosome, acrosome, autophagosome, centriole, glycosome, glyoxysome, hydrogenosome, melanosome, mitosome, cnidocyst, peroxisome, proteasome, vesicle, and stress granule.

24. The cytobiologic composition of any of the preceding claims, wherein:
i) the cytobiologic was not made by loading the cytobiologic with a therapeutic or diagnostic substance;
ii) the source cell was not loaded with a therapeutic or diagnostic substance;
iii) the cytobiologic does not comprise doxorubicin, dexamethasone, cyclodextrin;
polyethylene glycol, a micro RNA e.g., miR125, VEGF receptor, ICAM-1, E-selectin, iron oxide, a fluorescent protein e.g., GFP or RFP, a nanoparticle, or an RNase, or does not comprise an exogenous form of any of the foregoing; or
iv) the cytobiologic further comprises an exogenous therapeutic agent having one or more post-translational modifications, e.g., glycosylation.

25. The cytobiologic composition of any of the preceding claims, wherein the cytobiologic has a size that is about 0.01%-0.05%, 0.05%-0.1%, 0.1%-0.5%, 0.5%-1%, 1%-2%, 2%-3%, 3%-4%, 4%-5%, 5%-10%, 10%-20%, 20%-30%, 30%-40%, 40%-50%, 50%-60%, 60%-70%, 70%-80%, or 80%-90% the size of the source cell, e.g., as measured by an assay of Example 18.

26. The cytobiologic composition of any of the preceding claims, wherein the cytobiologic has a diameter of at least about 10 nm, 20 nm, 30 nm, 40 nm, 50 nm, 60 nm, 70 nm, 80 nm, 90 nm, 100 nm, 150 nm, or 200 nm, e.g., as measured by an assay of Example 20.

27. The cytobiologic composition of any of the preceding claims, wherein:
i) the cytobiologic is not an exosome;
ii) the cytobiologic is a microvesicle;
iii) the cytobiologic has a size of at least 80 nm, 100 nm, 200 nm, 500 nm, 1000 nm, 1200 nm, 1400 nm, or 1500 nm, or a population of cytobiologics has an average size of at least 80 nm, 100 nm, 200 nm, 500 nm, 1000 nm, 1200 nm, 1400 nm, or 1500 nm;
iv) the cytobiologic comprises one or more organelles, e.g., a mitochondrion, Golgi apparatus, lysosome, endoplasmic reticulum, vacuole, endosome, acrosome, autophagosome, centriole, glycosome, glyoxysome, hydrogenosome, melanosome, mitosome, cnidocyst, peroxisome, proteasome, vesicle, and stress granule;
v) the cytobiologic comprises a cytoskeleton or a component thereof, e.g., actin, Arp2/3, formin, coronin, dystrophin, keratin, myosin, or tubulin;
vi) the cytobiologic, composition, or preparation does not have a flotation density of 1.08-1.22 g/ml, or has a density of at least 1.18-1.25 g/ml, or 1.05-1.12 g/ml, e.g., in a sucrose gradient centrifugation assay, e.g., as described in Théry et al., “Isolation and characterization of exosomes from cell culture supernatants and biological fluids.” Curr Protoc Cell Biol. 2006 April; Chapter 3:Unit 3.22;
vii) the cytobiologic comprises a lipid bilayer that is enriched for ceramides or sphingomyelins or a combination thereof compared to the source cell, or the lipid bilayer is not enriched (e.g., is depleted) for glycolipids, free fatty acids, or phosphatidylserine, or a combination thereof, compared to the source cell;
viii) the cytobiologic comprises Phosphatidyl serine (PS) or CD40 ligand or both of PS and CD40 ligand, e.g., when measured in an assay of Example 40;
ix) the cytobiologic is enriched for PS compared to the source cell, e.g., in a population of cytobiologics at least 5%, 10% 20%, 30%, 40%, 50%, 60%, 70%, 80%, or 90% are positive for PS;
x) the cytobiologic is substantially free of acetylcholinesterase (AChE), or contains less than 0.001, 0.002, 0.005, 0.01, 0.02, 0.05, 0.1, 0.2, 0.5, 1, 2, 5, 10, 20, 50, 100, 200, 500, or 1000 AChE activity units/ug of protein, e.g., by an assay of Example 52;
xi) the cytobiologic is substantially free of a Tetraspanin family protein (e.g., CD63, CD9, or CD81), an ESCRT-related protein (e.g., TSG101, CHMP4A-B, or VPS4B), Alix, TSG101, MHCI, MHCII, GP96, actinin-4, mitofilin, syntenin-1, TSG101, ADAM10, EHD4, syntenin-1, TSG101, EHD1, flotillin-1, heat-shock 70-kDa proteins (HSC70/HSP73, HSP70/HSP72), or any combination thereof, or contains less than 0.05%, 0.1%, 0.5%, 1%, 2%, 3%, 4%, 5%, 5%, or 10% of any individual exosomal marker protein and/or less than 0.05%, 0.1%, 0.5%, 1%, 2%, 3%, 4%, 5%, 10%, 15%, 20%, or 25% of total exosomal marker proteins of any of said proteins, or is de-enriched for any one or more of these proteins compared to the source cell, or is not enriched for any one or more of these proteins, e.g., by an assay of Example 32;
xii) the cytobiologic comprises a level of Glyceraldehyde 3-phosphate dehydrogenase (GAPDH) that is below 500, 250, 100, 50, 20, 10, 5, or 1 ng GAPDH/ug total protein or below the level of GAPDH in the source cell, e.g., less than 1%, 2.5%, 5%, 10%, 15%, 20%, 30%, 40%, 50%, 60%, 70%, 80%, or 90%, less than the level of GAPDH per total protein in ng/ug in the source cell, e.g., using an assay of Example 33;
xiii) the cytobiologic is enriched for one or more endoplasmic reticulum proteins (e.g., calnexin), one or more proteasome proteins, or one or more mitochondrial proteins, or any combination thereof, e.g., wherein the amount of calnexin is greater than 500, 250, 100, 50, 20, 10, 5, or 1 ng Calnexin/ug total protein, or wherein the cytobiologic comprises more Calnexin per total protein in ng/ug compared to the source cell by 1%, 2.5%, 5%, 10%, 15%, 20%, 30%, 40%, 50%, 60%, 70%, 80%, or 90%, e.g., using an assay of Example 34;
xiv) the cytobiologic comprises an exogenous agent (e.g., an exogenous protein, mRNA, or siRNA) e.g., as measured using an assay of Example 27 or 28; or
xv) the cytobiologic can be immobilized on a mica surface by atomic force microscopy for at least 30 min.

28. The cytobiologic composition of any of the preceding claims, wherein:
i) the cytobiologic is an exosome;
ii) the cytobiologic is not a microvesicle;
iii) the cytobiologic has a size of less than 80 nm, 100 nm, 200 nm, 500 nm, 1000 nm, 1200 nm, 1400 nm, or 1500 nm, or a population of cytobiologics has an average size of at least 80 nm, 100 nm, 200 nm, 500 nm, 1000 nm, 1200 nm, 1400 nm, or 1500 nm;
iv) the cytobiologic does not comprise an organelle;
v) the cytobiologic does not comprise a cytoskeleton or a component thereof, e.g., actin, Arp2/3, formin, coronin, dystrophin, keratin, myosin, or tubulin;
vi) the cytobiologic, composition, or preparation has a flotation density of 1.08-1.22 g/ml, e.g., in a sucrose gradient centrifugation assay;
vii) the cytobiologic comprises lipid bilayer that is not enriched (e.g., is depleted) for ceramides or sphingomyelins or a combination thereof compared to the source cell, or the lipid bilayer is enriched for glycolipids, free fatty acids, or phosphatidylserine, or a combination thereof, compared to the source cell;
viii) the cytobiologic does not comprise, or is depleted for relative to the source cell, Phosphatidyl serine (PS) or CD40 ligand or both of PS and CD40 ligand, e.g., when measured in an assay of Example 40;
ix) the cytobiologic is not enriched (e.g., is depleted) for PS compared to the source cell, e.g., in a population of cytobiologics less than 20%, 30%, 40%, 50%, 60%, 70%, 80%, or 90% are positive for PS;
x) the cytobiologic comprises acetylcholinesterase (AChE), e.g. at least 0.001, 0.002, 0.005, 0.01, 0.02, 0.05, 0.1, 0.2, 0.5, 1, 2, 5, 10, 20, 50, 100, 200, 500, or 1000 AChE activity units/ug of protein, e.g., by an assay of Example 52;
xi) the cytobiologic comprises a Tetraspanin family protein (e.g., CD63, CD9, or CD81), an ESCRT-related protein (e.g., TSG101, CHMP4A-B, or VPS4B), Alix, TSG101, MHCI, MHCII, GP96, actinin-4, mitofilin, syntenin-1, TSG101, ADAM10, EHD4, syntenin-1, TSG101, EHD1, flotillin-1, heat-shock 70-kDa proteins (HSC70/HSP73, HSP70/HSP72), or any combination thereof, e.g., contains more than 0.05%, 0.1%, 0.5%, 1%, 2%, 3%, 4%, 5%, 5%, or 10% of any individual exosomal marker protein and/or less than 0.05%, 0.1%, 0.5%, 1%, 2%, 3%, 4%, 5%, 10%, 15%, 20%, or 25% of total exosomal marker proteins of any of said proteins, or is enriched for any one or more of these proteins compared to the source cell, e.g., by an assay of Example 32;
xii) the cytobiologic comprises a level of Glyceraldehyde 3-phosphate dehydrogenase (GAPDH) that is above 500, 250, 100, 50, 20, 10, 5, or 1 ng GAPDH/ug total protein or below the level of GAPDH in the source cell, e.g., at least 1%, 2.5%, 5%, 10%, 15%, 20%, 30%, 40%, 50%, 60%, 70%, 80%, or 90%, greater than the level of GAPDH per total protein in ng/ug in the source cell, e.g., using an assay of Example 33;
xiii) the cytobiologic is not enriched for (e.g., is depleted for) one or more endoplasmic reticulum proteins (e.g., calnexin), one or more proteasome proteins, or one or more mitochondrial proteins, or any combination thereof, e.g., wherein the amount of calnexin is less than 500, 250, 100, 50, 20, 10, 5, or 1 ng Calnexin/ug total protein, or wherein the cytobiologic comprises less Calnexin per total protein in ng/ug compared to the source cell by 1%, 2.5%, 5%, 10%, 15%, 20%, 30%, 40%, 50%, 60%, 70%, 80%, or 90%, e.g., using an assay of Example 34; or
xiv) the cytobiologic can not be immobilized on a mica surface by atomic force microscopy for at least 30 min.

29. The cytobiologic composition of any of the preceding claims, wherein:
i) the cytobiologic does not comprise a VLP;
ii) the cytobiologic does not comprise a virus;
iii) the cytobiologic does not comprise a replication-competent virus;
iv) the cytobiologic does not comprise a viral protein, e.g., a viral structural protein, e.g., a capsid protein or a viral matrix protein;
v) the cytobiologic does not comprise a capsid protein from an enveloped virus;
vi) the cytobiologic does not comprise a nucleocapsid protein; or
vii) the cytobiologic does not comprise a viral fusogen.

30. The cytobiologic of any of the preceding claims, wherein the cytobiologic comprises cytosol.

31. The cytobiologic of any of the preceding claims, wherein:
i) the cytobiologic does not form a teratoma when implanted into subject, e.g., by an assay of Example 74;
ii) the cytobiologic is capable of chemotaxis, e.g., at a speed at least 1%, 2%, 3%, 4%, 5%, 10%, 20%, 30%, 40%, 50%, 60%, 70%, 80%, 90%, 100% compared to a reference cell, e.g., a macrophage, e.g., using an assay of Example 43;
iii) the cytobiologic is capable of homing, e.g., at the site of an injury, wherein the cytobiologic is from a human cell, e.g., using an assay of Example 44, e.g., wherein the source cell is a neutrophil; or
iv) the cytobiologic is capable of phagocytosis, e.g., wherein phagocytosis by the cytobiologic is detectable within 0.5, 1, 2, 3, 4, 5, or 6 hours in using an assay of Example 45, e.g., wherein the source cell is a macrophage.

32. The cytobiologic composition of any of the preceding claims, which retains one, two, three, four, five six or more of any of the characteristics for 5 days or less, e.g., 4 days or less, 3 days or less, 2 days or less, 1 day or less, e.g., about 12-72 hours, after administration into a subject, e.g., a human subject.

33. The cytobiologic composition of any of the preceding claims, wherein the cytobiologic has one or more of the following characteristics:
a) comprises one or more endogenous proteins from a source cell, e.g., membrane proteins or cytosolic proteins;
b) comprises at least 10, 20, 50, 100, 200, 500, 1000, 2000, or 5000 different proteins;
c) comprises at least 1, 2, 5, 10, 20, 50, or 100 different glycoproteins;
d) at least 10%, 20%, 30%, 40%, 50%, 60%, 70%, 80%, or 90% by mass of the proteins in the cytobiologic are naturally-occurring proteins;
e) comprises at least 10, 20, 50, 100, 200, 500, 1000, 2000, or 5000 different RNAs; or
f) comprises at least 2, 3, 4, 5, 10, or 20 different lipids, e.g., selected from CL, Cer, DAG, HexCer, LPA, LPC, LPE, LPG, LPI, LPS, PA, PC, PE, PG, PI, PS, CE, SM and TAG.

34. The cytobiologic composition of any of the preceding claims, wherein the cytobiologic has been manipulated to have, or wherein the cytobiologic is not a naturally occurring cell and has, or wherein the nucleus is not naturally one, two, three, four, five or more of the following properties:
a) the partial nuclear inactivation results in a reduction of at least 50%, 60%, 70%, 80%, 90% or more in nuclear function, e.g., a reduction in transcription or DNA replication, or both, e.g., wherein transcription is measured by an assay of Example 9 and DNA replication is measured by an assay of Example 10;
b) the cytobiologic is not capable of transcription or has transcriptional activity of less than 1%, 2.5% 5%, 10%, 20%, 30%, 40%, 50%, 60%, 70%, 80%, or 90% of that of the transcriptional activity of a reference cell, e.g., the source cell, e.g., using an assay of Example 9;
c) the cytobiologic is not capable of nuclear DNA replication or has nuclear DNA replication of less than 1%, 2.5% 5%, 10%, 20%, 30%, 40%, 50%, 60%, 70%, 80%, or 90% of the nuclear DNA replication of a reference cell, e.g., the source cell, e.g., using an assay of Example 10;
d) the cytobiologic lacks chromatin or has a chromatin content of less than 1%, 2.5% 5%, 10%, 20%, 30%, 40%, 50%, 60%, 70%, 80%, or 90% of the of the chromatin content of a reference cell, e.g., the source cell, e.g., using an assay of Example 25;
e) the cytobiologic lacks a nuclear membrane or has less than 50%, 40%, 30%, 20%, 10%, 5%, 4%, 3%, 2%, or 1% the amount of nuclear membrane of a reference cell, e.g., the source cell or a Jurkat cell, e.g., by an assay of Example 24;
f) the cytobiologic lacks functional nuclear pore complexes or has reduced nuclear import or export activity, e.g., by at least 50%, 40%, 30%, 20%, 10%, 5%, 4%, 3%, 2%, or 1% by an assay of Example 24, or the cytobiologic lacks on or more of a nuclear pore protein, e.g., NUP98 or Importin 7;
g) the cytobiologic does not comprise histones or has histone levels less than 1%, of the histone level of the source cell (e.g., of H1, H2a, H2b, H3, or H4), e.g., by an assay of Example 25;
h) the cytobiologic comprises less than 20, 10, 5, 4, 3, 2, or 1 chromosome;
i) nuclear function is eliminated;
j) the cytobiologic is an enucleated mammalian cell;
k) the nucleus is removed or inactivated, e.g., extruded by mechanical force, by radiation or by chemical ablation; or
l) the cytobiologic is from a mammalian cell having DNA that is completely or partially removed, e.g., during interphase or mitosis.

35. The cytobiologic composition of any of the preceding claims, wherein the cytobiologic comprises mtDNA or vector DNA.

36. The cytobiologic composition of any of the preceding claims, which does not comprise DNA.

37. The cytobiologic composition of any of the preceding claims, wherein the source cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), or an immortalized cell (e.g., HeLa, HEK293, HFF-1, MRC-5, WI-38, IMR 90, IMR 91, PER.C6, HT-1080, or BJ cell).

38. The cytobiologic composition of any of the preceding claims, wherein the source cell is a primary cell, immortalized cell, or a cell line (e.g., myelobast cell line, e.g., C2C12).

39. The cytobiologic composition of any of the preceding claims, wherein the cytobiologic is from a source cell having a modified genome, e.g., having reduced immunogenicity (e.g., by genome editing to remove MHC complexes).

40. The cytobiologic composition of any of the preceding claims, wherein the source cell is from a cell culture treated with an anti-inflammatory signal.

41. The cytobiologic composition of any of the preceding claims, wherein the source cell is substantially non-immunogenic, e.g., using an assay described herein.

42. The cytobiologic composition of any of the preceding claims, wherein the source cell comprises an exogenous agent, e.g., a therapeutic agent.

43. The cytobiologic composition of any of the preceding claims, wherein the source cell is a recombinant cell.

44. The cytobiologic composition of any of the preceding claims, wherein the cytobiologic further comprises an exogenous agent, e.g., a therapeutic agent, e.g., a protein or a nucleic acid (e.g., an RNA, e.g., an mRNA or miRNA).

45. The cytobiologic composition of any of the preceding claims, wherein the exogenous agent is present at at least, or no more than, 10, 20, 50, 100, 200, 500, 1,000, 2,000, 5,000, 10,000, 20,000, 50,000, 100,000, 200,000, 500,000 or 1,000,000 copies comprised by the cytobiologic, or is present at an average level of at least, or no more than, 10, 20, 50, 100, 200, 500, 1,000, 2,000, 5,000, 10,000, 20,000, 50,000, 100,000, 200,000, 500,000 or 1,000,000 copies per cytobiologic.

46. The cytobiologic composition of any of the preceding claims, wherein the cytobiologic has an altered, e.g., increased or decreased level of one or more endogenous molecule, e.g., protein or nucleic acid, e.g., due to treatment of the source cell, e.g., mammalian source cell, with a siRNA or gene editing enzyme.

47. The cytobiologic composition of any of the preceding claims, wherein the endogenous molecule is present at at least, or no more than, 10, 20, 50, 100, 200, 500, 1,000, 2,000, 5,000, 10,000, 20,000, 50,000, 100,000, 200,000, 500,000 or 1,000,000 copies comprised by the cytobiologic, or is present at an average level of at least, or no more than, 10, 20, 50, 100, 200, 500, 1,000, 2,000, 5,000, 10,000, 20,000, 50,000, 100,000, 200,000, 500,000 or 1,000,000 copies per cytobiologic.

48. The cytobiologic composition of any of the preceding claims, wherein the endogenous molecule (e.g., an RNA or protein) is present at a concentration of at least 1, 2, 3, 4, 5, 10, 20, 50, 100, 500, 10³, 5.0×10³, 10⁴, 5.0×10⁴, 10⁵, 5.0×10⁵, 10⁶, 5.0×10⁶, 1.0×10⁷, 5.0×10⁷, or 1.0×10⁸ greater than its concentration in the source cell.

49. The cytobiologic composition of any of the preceding claims, wherein the agent, e.g., therapeutic agent, is selected from a protein, protein complex (e.g., comprising at least 2, 3, 4, 5, 10, 20, or 50 proteins, e.g., at least at least 2, 3, 4, 5, 10, 20, or 50 different proteins) polypeptide, nucleic acid (e.g., DNA, chromosome, or RNA, e.g., mRNA, siRNA, or miRNA) or small molecule.

50. The cytobiologic composition of any of the preceding claims, wherein the exogenous agent comprises a site-specific nuclease, e.g., Cas9 molecule, TALEN, or ZFN.

51. The cytobiologic composition of any of the preceding claims, wherein the cytobiologic does not comprise a fusogen.

52. The cytobiologic composition of any of claims 1-50, which comprises a fusogen.

53. The cytobiologic composition of claim 52, wherein the fusogen is disposed in the membrane of the cytobiologic.

54. The cytobiologic composition of claim 52, wherein the fusogen is a protein fusogen, a lipid fusogen, a chemical fusogen, or a small molecule fusogen.

55. The cytobiologic composition of any of the preceding claims, wherein the cytobiologic binds to or acts on a target cell.

56. The cytobiologic composition of claim 55, wherein the target cell is other than a HeLa cell, or the target cell is not transformed or immortalized.

57. A cytobiologic composition, comprising a plurality of cytobiologics according to any of the preceding claims.

58. The cytobiologic composition of claim 57, wherein the plurality of cytobiologics are the same.

59. The cytobiologic composition of claim 57, wherein the plurality of cytobiologics are different.

60. The cytobiologic composition of any of claims 57-59, wherein the plurality of cytobiologics are from one or more source cells.

61. The cytobiologic composition of any of claims 57-60, wherein at least 50% of cytobiologics in the plurality have a diameter within 10%, 20%, 30%, 40%, or 50% of the mean diameter of the cytobiologics in the cytobiologic composition.

62. The cytobiologic composition of any of claims 57-61, wherein at least 50% of cytobiologic in the plurality have a volume within 10%, 20%, 30%, 40%, or 50% of the mean volume of the cytobiologics in the cytobiologic composition.

63. The cytobiologic composition of any of claims 57-62, wherein at least 50% of cytobiologics in the plurality have a copy number of the therapeutic agent within 10%, 20%, 30%, 40%, or 50% of the mean therapeutic agent copy number in the cytobiologics in the cytobiologic composition.

64. The cytobiologic composition of any of claims 57-63 which comprises at least 10⁵, 10⁶, 10⁷, 10⁸, 10⁹, or 10¹⁰ cytobiologics.

65. The cytobiologic composition of any of claims 57-64, which is in a volume of at least 1 ul, 2 ul. 5 ul, 10 ul, 20 ul, 50 ul, 100 ul, 200 ul, 500 ul, 1 ml, 2 ml, 5 ml, or 10 ml.

66. A pharmaceutical composition comprising the cytobiologic composition of any of the preceding claims and pharmaceutically acceptable carrier.

67. A pharmaceutical composition suitable for administration to a human subject, comprising a cytobiologic and a pharmaceutically acceptable carrier, wherein:
i) the cytobiologic is from a source cell, e.g., a mammalian source cell,
ii) the cytobiologic is an enucleated cell or a cell having partial or complete nuclear inactivation (e.g., nuclear removal), and
iii) the cytobiologic is not from an erythroid cell or a platelet.

68. The pharmaceutical composition of claim 66 or 67, having one or more of the following characteristics:
a) the pharmaceutical composition meets a pharmaceutical or good manufacturing practices (GMP) standard;
b) the pharmaceutical composition was made according to good manufacturing practices (GMP);
c) the pharmaceutical composition has a pathogen level below a predetermined reference value, e.g., is substantially free of pathogens;
d) the pharmaceutical composition has a contaminant level below a predetermined reference value, e.g., is substantially free of contaminants; or
e) the pharmaceutical composition has low immunogenicity, e.g., as described herein.

69. A method of administering a cytobiologic composition to a subject, e.g., a human subject, comprising administering to the subject a cytobiologic composition of any of claims 1-65 or a pharmaceutical composition of any of claims 66-68, thereby administering the cytobiologic composition to the subject.

70. A method of administering a cytobiologic composition to a subject, e.g., a human subject, comprising administering to the subject a cytobiologic composition wherein:
i) the cytobiologic is from a source cell, e.g., a mammalian source cell,
ii) the cytobiologic is an enucleated cell or a cell having partial or complete nuclear inactivation (e.g., nuclear removal), and
iii) the cytobiologic is not from an erythroid cell or a platelet,
thereby administering the cytobiologic composition to the subject.

71. A method of delivering a therapeutic agent to a subject, comprising administering to the subject a cytobiologic composition comprising a cytobiologic composition of any of claims 1-65 or a pharmaceutical composition of any of claims 66-68, wherein the cytobiologic composition is administered in an amount and/or time such that the therapeutic agent is delivered.

72. A method of delivering a therapeutic agent to a subject, comprising administering to the subject a cytobiologic composition wherein:
i) the cytobiologic is from a source cell, e.g., a mammalian source cell,
ii) the cytobiologic is an enucleated cell or a cell having partial or complete nuclear inactivation (e.g., nuclear removal),
iii) the cytobiologic is not from an erythroid cell or a platelet, and
iv) the cytobiologic comprises the therapeutic agent,
thereby delivering the therapeutic agent to the subject

73. A method of modulating, e.g., enhancing, a biological function in a subject, comprising administering to the subject a cytobiologic composition of any of claims 1-65 or a pharmaceutical composition of any of claims 66-68, thereby modulating the biological function in the subject.

74. A method of modulating, e.g., enhancing, a biological function in a subject, comprising administering to the subject a cytobiologic composition wherein:
i) the cytobiologic is from a source cell, e.g., a mammalian source cell,
ii) the cytobiologic is an enucleated cell or a cell having partial or complete nuclear inactivation (e.g., nuclear removal), and
iii) the cytobiologic is not from an erythroid cell or a platelet,
thereby modulating the biological function in the subject

75. The method of claim 73 or 74, wherein the biological function is selected from:
a) modulating, e.g., inhibiting or stimulating, an enzyme;
b) modulating, e.g., increasing or decreasing levels of, a molecule (e.g., a protein, nucleic acid, or metabolite, drug, or toxin) in the subject, e.g., by inhibiting or stimulating synthesis or by inhibiting or stimulating degradation of the factor;
c) modulating, e.g., increasing or decreasing, viability of a target cell or tissue; or
d) modulating a protein state, e.g., increasing or decreasing phosphorylation of the protein, or modulating the protein conformation;
e) promoting healing of an injury;
f) modulating, e.g., increasing or decreasing, an interaction between two cells;
g) modulating, e.g., promoting or inhibiting, cell differentiation;
h) altering distribution of a factor (e.g., a protein, nucleic acid, metabolite, drug, or toxin) in the subject;
i) modulating, e.g., increasing or decreasing, an immune response; or
j) modulating, e.g. increasing or decreasing, recruitment of cells to a target tissue.

76. A method of delivering a function to a subject, comprising administering to the subject a cytobiologic composition comprising a cytobiologic composition of any of claims 1-65 or a pharmaceutical composition of any of claims 66-68, wherein the cytobiologic composition is administered in an amount and/or time such that the function in the subject is delivered.

77. A method of delivering a function to a subject, comprising administering to the subject a cytobiologic composition wherein:
i) the cytobiologic is from a source cell, e.g., a mammalian source cell,
ii) the cytobiologic is an enucleated cell or a cell having partial or complete nuclear inactivation (e.g., nuclear removal), and
iii) the cytobiologic is not from an erythroid cell or a platelet,
thereby delivering the function to the subject.

78. A method of targeting a function to a subject, comprising administering to the subject a cytobiologic composition comprising a a cytobiologic composition of any of claims 1-65 or a pharmaceutical composition of any of claims 66-68, wherein the cytobiologic composition is administered in an amount and/or time such that the function in the subject is targeted.

79. A method of targeting a function to a subject, comprising administering to the subject a cytobiologic composition wherein:
i) the cytobiologic is from a source cell, e.g., a mammalian source cell,
ii) the cytobiologic is an enucleated cell or a cell having partial or complete nuclear inactivation (e.g., nuclear removal), and
iii) the cytobiologic is not from an erythroid cell or a platelet,
thereby targeting the function to the subject.

80. The method of any of claims 69-79, wherein the subject is a human subject.

81. The method of any of claims 69-80, wherein the plurality of cytobiologics has a local effect.

82. The method of any of claims 69-80, wherein the plurality of cytobiologics has a distal effect.

83. The method of any of claims 69-82, wherein the subject has a cancer, an inflammatory disorder, autoimmune disease, a chronic disease, inflammation, damaged organ function, an infectious disease, a degenerative disorder, a genetic disease (e.g., a recessive genetic disorder or a dominant genetic disorder), or an injury.

84. The method of claim 83, wherein the subject has a cancer and the cytobiologic comprises a neoantigen.

85. The method of claim 83, wherein the subject has an infectious disease and the cytobiologic comprises an antigen for the infectious disease.

86. The method of claim 83, wherein the subject has a genetic deficiency and the cytobiologic comprises a protein for which the subject is deficient, or a nucleic acid (e.g., mRNA) encoding the protein.

87. The method of claim 83, wherein the subject has a dominant genetic disorder, and the cytobiologic comprises a nucleic acid inhibitor (e.g., siRNA or miRNA) of the dominant mutant allele.

88. The method of any of claims 69-87, wherein the subject is in need of vaccination.

89. The method of any of claims 69-88, wherein the subject is in need of regeneration, e.g., of an injured site.

90. The method of any of claims 69-89, wherein the cytobiologic composition is administered to the subject at least 1, 2, 3, 4, or 5 times.

91. The method of any of claims 69-90, wherein the cytobiologic composition is administered to the subject systemically (e.g., orally, parenterally, subcutaneously, intravenously, intramuscularly, intraperitoneally) or locally.

92. The method of any of claims 69-91, wherein the cytobiologic composition is administered to the subject such that the cytobiologic composition reaches a target tissue selected from liver, lungs, heart, spleen, pancreas, gastrointestinal tract, kidney, testes, ovaries, brain, reproductive organs, central nervous system, peripheral nervous system, skeletal muscle, endothelium, inner ear, or eye.

93. The method of any of claims 69-92, wherein the cytobiologic composition is co-administered with an immunosuppressive agent, e.g., a glucocorticoid, cytostatic, antibody, or immunophilin modulator.

94. The method of any of claims 69-92, wherein the cytobiologic composition is co-administered with an immunostimulatory agent, e.g., an adjuvant, interleukin, cytokine, or chemokine.

95. The method of any of claims 69-94, wherein administration of the cytobiologic composition results in upregulation or downregulation of a gene in a target cell in the subject, e.g., wherein the cytobiologic comprises a transcriptional activator or repressor, a translational activator or repressor, or an epigenetic activator or repressor of transcription.

96. A method of manufacturing a pharmaceutical cytobiologic composition, comprising:
a) providing a source cell, e.g., mammalian source cell;
b) producing a cytobiologic from the source cell; and
c) formulating the cytobiologic, e.g., as a pharmaceutical composition suitable for administration to a subject.

97. The method of claim 96, which comprises inactivating the nucleus of the source cell.

98. A method of manufacturing a cytobiologic composition, comprising:
a) providing a plurality of source cells, e.g., mammalian source cells;
b) producing at least 10⁵, 10⁶, 10⁷, 10⁸, 10⁹, 10¹⁰, 10¹¹, 10¹², 10¹³, 10¹⁴, or 10¹⁵ cytobiologics from the plurality of source cells, e.g., by enucleation.

99. A method of manufacturing a pharmaceutical cytobiologic composition, comprising:
a) providing a cytobiologic composition according to any of claims 1-65 or a pharmaceutical composition of any of claims 66-68; and
b) formulating the cytobiologic composition, e.g., as a pharmaceutical composition suitable for administration to a subject.

100. The method of claim 99, wherein the cytobiologic composition comprises at least 10⁵, 10⁶, 10⁷, 10⁸, 10⁹, 10¹⁰, 10¹¹, 10¹², 10¹³, 10¹⁴, or 10¹⁵ cytobiologics.

101. The method of claim 99 or 100, wherein the cytobiologic composition comprises at least 10 ml, 20 ml, 50 ml, 100 ml, 200 ml, 500 ml, 1 L, 2 L, 5 L, 10 L, 20 L, or 50 L.

102. The method of any of claims 99-101, which comprises enucleating the mammalian source cell, e.g., by chemical enucleation, use of mechanical force e.g. use of a filter or centrifuge, least partial disruption of the cytoskeleton.

103. The method of any of claims 96-102, which comprises one or more of: vesiculation, hypotonic treatment, extrusion, or centrifugation.

104. The method of any of claims 96-103, which comprises genetically expressing an exogenous agent in the cell or loading the exogenous agent into the source cell or cytobiologic.

105. The method of any of claims 96-104, which comprises contacting the source cell with DNA encoding a polypeptide agent, e.g., before inactivating the nucleus, e.g., enucleating the source cell.

106. The method of any of claims 96-105, which comprises contacting the source cell with RNA encoding a polypeptide agent, e.g., before or after inactivating the nucleus, e.g., enucleating the source cell.

107. The method of any of claims 96-106, which comprises introducing a therapeutic agent (e.g., a nucleic acid or protein) into a cytobiologic, e.g., by electroporation.

108. The method of any of claims 96-107, wherein the source cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), or an immortalized cell (e.g., HeLa, HEK293, HFF-1, MRC-5, WI-38, IMR 90, IMR 91, PER.C6, HT-1080, or BJ cell).

109. The method of any of claims 96-108, wherein the cytobiologic is from a mammalian cell having a modified genome, e.g., having reduced immunogenicity (e.g., by genome editing to remove MHC complexes).

110. The method of any of claims 96-109, wherein the source cell is from a cell culture treated with an anti-inflammatory signal.

111. The method of any of claims 96-110, further comprising contacting the source cell of step a) with an anti-inflammatory signal, e.g., before or after inactivating the nucleus, e.g., enucleating the cell.

112. A method of manufacturing a cytobiologic composition, comprising:
a) providing, e.g., producing, a cytobiologic composition according to any of claims 1-65; and
b) assaying one or more cytobiologics from the cytobiologic composition to determine whether one or more (e.g., 2, 3, or all) of the following standards are met:
i) the cytobiologic comprises a therapeutic agent at a copy number of at least 1,000 copies, e.g., as measured by an assay of Example 31;
ii) the cytobiologic comprises a lipid composition wherein one or more of CL, Cer, DAG, HexCer, LPA, LPC, LPE, LPG, LPI, LPS, PA, PC, PE, PG, PI, PS, CE, SM and TAG is within 75% of the corresponding lipid level in the source cell;
iii) the cytobiologic comprises a proteomic composition similar to that of the source cell, e.g., using an assay of Example 30;
iv) the cytobiologic comprises a ratio of lipids to proteins that is within 10%, 20%, 30%, 40%, or 50% of the corresponding ratio in the source cell, e.g., as measured using an assay of Example 37;
v) the cytobiologic comprises a ratio of proteins to nucleic acids (e.g., DNA) that is within 10%, 20%, 30%, 40%, or 50% of the corresponding ratio in the source cell, e.g., as measured using an assay of Example 38;
vi) the cytobiologic comprises a ratio of lipids to nucleic acids (e.g., DNA) that is within 10%, 20%, 30%, 40%, or 50% of the corresponding ratio in the source cell, e.g., as measured using an assay of Example 39;
vii) the cytobiologic has a half-life in a subject, e.g., in a mouse, that is within 90% of the half life of a reference cell, e.g., the source cell, e.g., by an assay of Example 60;
viii) the cytobiologic transports glucose (e.g., labeled glucose, e.g., 2-NBDG) across a membrane, e.g., by at least 10% more than a negative control, e.g., an otherwise similar cytobiologic in the absence of glucose, e.g., as measured using an assay of Example 49;
ix) the cytobiologic comprises esterase activity in the lumen that is within 90% of that of the esterase activity in a reference cell, e.g., the source cell or a mouse embryonic fibroblast, e.g., using an assay of Example 51;
x) the cytobiologic comprises a metabolic activity level that is within 90% of the metabolic activity (e.g., citrate synthase activity) in a reference cell, e.g., the source cell, e.g., as described in Example 53;
xi) the cytobiologic comprises a respiration level (e.g., oxygen consumption rate) that is within 90% of the respiration level in a reference cell, e.g., the source cell, e.g., as described in Example 54;
xii) the cytobiologic comprises an Annexin-V staining level of at most 18,000, 17,000, 16,000, 15,000, 14,000, 13,000, 12,000, 11,000, or 10,000 MFI, e.g., using an assay of Example 55, or wherein the cytobiologic comprises an Annexin-V staining level at least 5%, 10%, 20%, 30%, 40%, or 50% lower than the Annexin-V staining level of an otherwise similar cytobiologic treated with menadione in the assay of Example 55, or wherein the cytobiologic comprises an Annexin-V staining level at least 5%, 10%, 20%, 30%, 40%, or 50% lower than the Annexin-V staining level of a macrophage treated with menadione in the assay of Example 55;
xiii) the cytobiologic has a miRNA content level of at least 1% than that of the source cell, e.g., by an assay of Example 27;
xiv) the cytobiologic has a soluble:non-soluble protein ratio is within 90% of that of the source cell, e.g., by an assay of Example 35;
xv) the cytobiologic has an LPS level less than 5% of the lipid content of cytobiologics, e.g., as measured by an assay of Example 36;
xvi) the cytobiologic is capable of signal transduction, e.g., transmitting an extracellular signal, e.g., AKT phosphorylation in response to insulin, or glucose (e.g., labeled glucose, e.g., 2-NBDG) uptake in response to insulin, e.g., by at least 10% more than a negative control, e.g., an otherwise similar cytobiologic in the absence of insulin, e.g., using an assay of Example 48;
xvii) the cytobiologic has juxtacrine-signaling level of at least 5% greater than the level of juxtacrine signaling induced by a reference cell, e.g., the source cell or a bone marrow stromal cell (BMSC), e.g., by an assay of Example 56;
xviii) the cytobiologic has paracrine-signaling level of at least 5% greater than the level of paracrine signaling induced by a reference cell, e.g., the source cell or a macrophage, e.g., by an assay of Example 57;
xix) the cytobiologic polymerizes actin at a level within 5% compared to the level of polymerized actin in a reference cell, e.g., the source cell or a C2C12 cell, e.g., by the assay of Example 58;
xx) the cytobiologic has a membrane potential within about 5% of the membrane potential of a reference cell, e.g., the source cell or a C2C12 cell, e.g., by an assay of Example 59, or wherein the cytobiologic has a membrane potential of about −20 to −150 mV, −20 to −50 mV, −50 to −100 mV, or −100 to −150 mV;
xxi) the cytobiologic is capable of secreting a protein, e.g., at a rate at least 5% greater than a reference cell, e.g., a mouse embryonic fibroblast, e.g., using an assay of Example 47; or
xxii) the cytobiologic has low immunogenicity, e.g., as described herein; and
c) (optionally) approving the cytobiologic composition for release if one or more of the standards is met.

113. A method of manufacturing a cytobiologic composition, comprising:
a) providing, e.g., producing, a cytobiologic composition according to any of claims 1-65; and
b) assaying one or more cytobiologics from the cytobiologic composition to determine the presence or level of one or more of the following factors:
i) an immunogenic molecule, e.g., an immunogenic protein, e.g., as described herein;
ii) a pathogen, e.g., a bacterium or virus; or
iii) a contaminant; and
c) (optionally) approving the cytobiologic composition for release if one or more of the factors is below a reference value.

114. The method of claim 113, wherein if a detectable level, e.g., a value above a reference value, is determined, a sample containing the cytobiologic composition is discarded.
